(19) United States
(12) Patent Application Publication
(10) Pub. No.: US 2004/0097728 A1
(43) Pub. Date: May 20, 2004

(54) Method for producing anellated tetrahydro-{1h}-triazoles

(76) Inventors: Gerhard Hamprecht, Weinheim (DE); Olaf Menke, Alteiningen (DE); Robert Reinhard, Ludwigshafen (DE); Michael Puhl, Lampertheim (DE); Ingo Sagasser, Dannstadt-Schauernheim (DE); Cyrill Zagar, Mannheim (DE); Matthias Witschel, Bad Durkheim (DE); Helmut Walter, Obrigheim (DE)

Correspondence Address: KEIL & WEINKAUF, 1350 CONNECTICUT AVENUE, N.W., WASHINGTON, DC, 20036, US

(21) Appl. No.: 10/363,661

(22) PCT Filed: Sep. 7, 2001
(86) PCT No.: PCT/EP01/10352

(30) Foreign Application Priority Data
Sep. 8, 2000 (DE) 100444457.1

(51) Int. Cl. C07D 498/02; C07D 491/02

(52) U.S. Cl. 544/10; 544/66

(57) ABSTRACT

The present invention relates to a process for preparing fused tetrahydro-[1H]-triazoles of the formula I

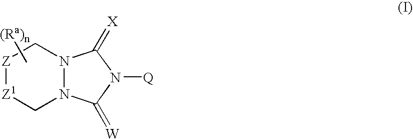

where the variables R^{a}, Z, Z¹, X, W, n and Q are as defined in claim 1, by cyclization of compounds of the formula II

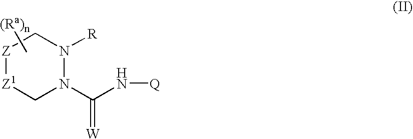

where R is C(X)OR² or C(X)SR², where X is oxygen or sulfur, and R² is as defined in claim 1, in the presence of a base.

The invention also relates to compounds of the formula I where W is sulfur if Z is a methylene group optionally substituted by R^{a}, and furthermore to compounds of the formula I where Q is a benzoxazole or benzothiazole radical, and to the use of these compounds as herbicides.

The present invention relates to a process for preparing fused tetrahydro-[1H]-triazoles of the formula I

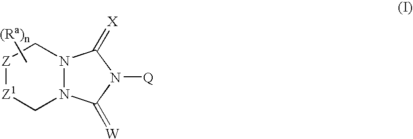

where the variables R^{a}, W, X, n and Q are as defined below:

R^{a} is hydroxyl, CO₂R¹, halogen, cyano, C(O)N(R¹)₂, where the radicals R¹ may be different fom one another, OR^{1a}, C₁-C₆-alkyl, C₁-C₆-haloalkyl, C₃-C₆-alkenyl, C₃-C₆-alkynyl, COR¹, S(O)ₙR¹ where n=0, 1 or 2 or C(O)SR¹; where

R¹ is hydrogen, C₁-C₆-alkyl, C₁-C₆-haloalkyl, C₁-C₃-alkoxy-C₁-C₃-alkyl, C₃-C₆-alkenyl or C₃-C₆-alkynyl; and

R^{1a} is C₁-C₆-alkyl, C₃-C₆-alkenyl, C₃-C₆-alkynyl which may be partially or fully halogenated or substituted, C₃-C₆-cycloalkyl, benzyl or phenethyl which may be substituted on the phenyl ring, and also optionally substituted phenyl or optionally substituted pyridyl;

n has the value 0, 1, 2 or 3;

X,W independently of one another are S or O;

Q is phenyl which has 1, 2, 3 or 4 substituents, where substituents attached to two adjacent carbon atoms may, together with these atoms, form a 5- or 6-membered saturated or unsaturated carbocycle or a 5- or 6-membered saturated or unsaturated heterocycle which has 1, 2 or 3 heteromatoms selected from the group consisting of O, N and S and which for its part may be substituted or unsubstituted;

where one of the groups Z or Z¹ is a methylene group which is optionally substituted by R^{a} and the other group Z or Z¹ is O, S, S═O or SO₂.

WO 94/10173 and WO 00/01700 describe a process for preparing fused tetrahydro-[1H]-triazoles of the formula b (hereinbelow also referred to as triazolinediones) where, according to Scheme 1, a substituted urea of the formula a is cyclized with phosgene or a phosgene substitute such as diphosgene. In Scheme 1, Ph is a substituted phenyl ring. X is oxygen or sulfur. However, owing to its high toxicity, the use of phosgene is problematic.

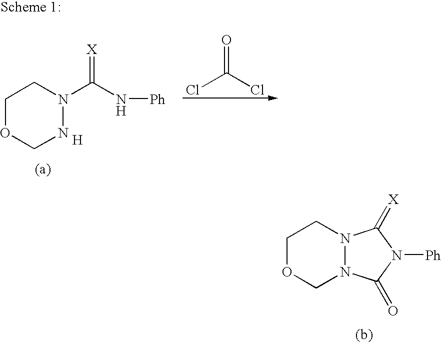

A further disadvantage is the fact that, by this route, it is not possible to prepare derivatives b′ of the triazolinedione b in which the carbonyl group in the triazole ring is replaced by a thiocarbonyl group. For example, it was not possible to cyclize the compound a from Scheme 1 to the compound b′ shown in Scheme 2 analogously to the process described in WO 94/10173 and WO 00/01700 using thiophosgene or a thiophosgene equivalent. Additional experiments of the applicant have shown that, even with particularly effective sulfurizing agents such as phosphorus pentasulfide/sodium carbonate (see Denis Brillon, Synth. Commun. 20, (1990) p. 3085), it is not possible to convert triazolinediones b according to Scheme 2 into the corresponding thiocarbonyl compounds of the formula b′.

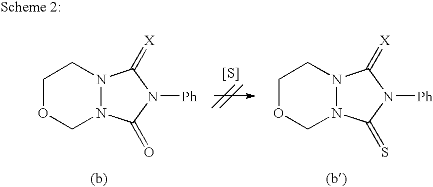

It is an object of the present invention to provide a process for preparing the compounds of the formula I defined at the outset, which process does not require phosgene or a phosgene substitute.

We have found that this object is achieved, surprisingly, by reacting substituted urea derivatives of the formula II

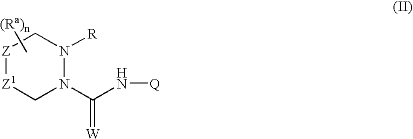

in which the variables R^{a}, Z, Z¹, W, X, n and Q are as defined above and

R is C(X)OR² or C(X)SR², where

X is oxygen or sulfur and

R² is C₁-C₆-alkyl, C₃-C₈-cycloalkyl, C₂-C₆-alkenyl, C₃-C₆-alkynyl which may be partially or fully halogenated or substituted, P(O)(OR¹)₂, aryl or heteroaryl which may optionally be substituted, where R¹ is as defined above;

with a base.

Accordingly, the present invention relates to a process for preparing compounds of the formula I defined above, which process comprises reacting a compound II with a base.

The substituted ureas of the formula II used as starting materials form part of the subject matter of the earlier international application PCT/EP 00/05794, which is incorporated herein by way of reference.

The organic moieties mentioned in the definitions of R^{a}, R¹ and R²⁸ and as radicals on phenyl, cycloalkyl and heterocyclyl rings are collective terms for individual enumerations of the individual group members. All carbon chains, i.e. all (optionally substituted) alkyl, alkenyl or alkynyl moieties can be straight-chain or branched. Halogenated substituents preferably carry one to five identical or different halogen atoms.

The term “halogen” denotes in each case fluorine, bromine, chlorine or iodine, in particular fluorine or chlorine.

Examples of other meanings are:

C₁-C₄-alkyl: CH₃, C₂H₅, n-propyl, CH(CH₃)₂, n-butyl, CH(CH₃)-C₂H₅, 2-methylpropyl or C(CH₃)₃, in particular CH₃, C₂H₅ or CH(CH₃)₂;

C₁-C₄-haloalkyl: a C₁-C₄-alkyl radical as mentioned above which is partially or fully substituted by fluorine, chlorine, bromine and/or iodine, i.e., for example, CH₂F, CHF₂, CF₃, CH₂Cl, dichloromethyl, trichloromethyl, chlorofluoromethyl, dichlorofluoromethyl, chlorodifluoromethyl, 2-fluoroethyl, 2-chloroethyl, 2-bromoethyl, 2-iodoethyl, 2,2-difluoroethyl, 2,2,2-trifluoroethyl, 2-chloro-2-fluoroethyl, 2-chloro-2,2-difluoroethyl, 2,2-dichloro-2-fluoroethyl, 2,2,2-trichloroethyl, C₂F₅, 2-fluoropropyl, 3-fluoropropyl, 2,2-difluoropropyl, 2,3-difluoropropyl, 2-chloropropyl, 3-chloropropyl, 2,3-dichloropropyl, 2-bromopropyl, 3-bromopropyl, 3,3,3-trifluoropropyl, 3,3,3-trichloropropyl, 2,2,3,3,3-pentafluoropropyl, heptafluoropropyl, 1-(fluoromethyl)-2-fluoroethyl, 1-(chloromethyl)-2-chloroethyl, 1-(bromomethyl)-2-bromoethyl, 4-fluorobutyl, 4-chlorobutyl, 4-bromobutyl or nonafluorobutyl, in particular CH₂F, CHF₂, CF₃, CH₂Cl, 2-fluoroethyl, 2-chloroethyl or 2,2,2-trifluoroethyl;

C₁-C₆-alkyl: C₁-C₄-alkyl as mentioned above, and also, for example, n-pentyl, 1-methylbutyl, 2-methylbutyl, 3-methylbutyl, 2,2-dimethylpropyl, 1-ethylpropyl, n-hexyl, 1,1-dimethylpropyl, 1,2-dimethylpropyl, 1-methylpentyl, 2-methylpentyl, 3-methylpentyl, 4-methylpentyl, 1,1-dimethylbutyl, 1,2-dimethylbutyl, 1,3-dimethylbutyl, 1,2,2-dimethylbutyl, 2,3-dimethylbutyl, 3,3-dimethylbutyl, 1-ethylbutyl, 2-ethylbutyl, 1,1,2-trimethylpropyl, 1,2,2-trimethylpropyl, 1-ethyl-1-methylpropyl or 1-ethyl-2-methylpropyl, in particular CH₃, C₂H₅, n-propyl, CH(CH₃)₂, n-butyl, C(CH₃)₃, n-pentyl or n-hexyl;

C₁-C₆-haloalkyl: C₁-C₆-alkyl as mentioned above which is partially or fully substituted by fluorine, chlorine, bromine and/or iodine, i.e., for example, one of the radicals mentioned under C₁-C₄-haloalkyl or 5-fluoro-1-pentyl, 5-chloro-1-pentyl, 5-bromo-1-pentyl, 5-iodo-1-pentyl, 5,5,5-trichloro-1-pentyl, undecafluoropentyl, 6-fluoro-1-hexyl, 6-chloro-1-hexyl, 6-bromo-1-hexyl, 6-iodo-1-hexyl, 6,6,6-trichloro-1-hexyl or dodecafluorohexyl, in particular chloromethyl, fluoromethyl, difluoromethyl, trifluoromethyl, 2-fluoroethyl, 2-chloroethyl or 2,2,2-trifluoroethyl;

hydroxy-C₁-C₆-alkyl: for example hydroxymethyl, 2-hydroxyeth-1-yl, 2-hydroxyprop-1-yl, 3-hydroxyprop-1-yl, 1-hydroxyprop-2-yl, 2-hydroxybut-1-yl, 3-hydroxybut-1-yl, 4-hydroxybut-1-yl, 1-hydroxybut-2-yl, 1-hydroxybut-3-yl, 2-hydroxybut-3-yl, 1-hydroxy-2-methylprop-3-yl, 2-hydroxy-2-methylprop-3-yl or 2-hydroxymethylprop-2-yl, in particular 2-hydroxyethyl;

cyano-C₁-C₆-alkyl: for example cyanomethyl, 1-cyanoeth-1-yl, 2-cyanoeth-1-yl, 1-cyanoprop-1-yl, 2-cyanoprop-1-yl, 3-cyanoprop-1-yl, 1-cyanoprop-2-yl, 2-cyanoprop-2-yl, 1-cyanobut-1-yl, 2-cyanobut-1-yl, 3-cyanobut-1-yl, 4-cyanobut-1-yl, 1-cyanobut-2-yl, 2-cyanobut-2-yl, 1-cyanobut-3-yl, 2-cyanobut-3-yl, 1-cyano-2-methylprop-3-yl, 2-cyano-2-methylprop-3-yl, 3-cyano-2-methylprop-3-yl or 2-cyanomethylprop-2-yl, in particular cyanomethyl or 2-cyanoethyl;

phenyl-C₁-C₆-alkyl: for example benzyl, 1-phenylethyl, 2-phenylethyl, 1-phenylprop-1-yl, 2-phenylprop-1-yl, 3-phenylprop-1-yl, 1-phenylbut-1-yl, 2-phenylbut-1-yl, 3-phenylbut-1-yl, 4-phenylbut-1-yl, 1-phenylbut-2-yl, 2-phenylbut-2-yl, 3-phenylbut-2-yl, 4-phenylbut-2-yl, 1-(phenylmethyl)eth-1-yl, 1-(phenylmethyl)-1-(methyl)eth-1-yl or 1-(phenylmethyl)prop-1-yl, in particular benzyl or 2-phenylethyl;

phenyl-(C₁-C₆-alkyl)carbonyloxy: for example benzylcarbonyloxy, 1-phenylethylcarbonyloxy, 2-phenylethylcarbonyloxy, 1-phenylprop-1-ylcarbonyloxy, 2-phenylprop-1-ylcarbonyloxy, 3-phenylprop-1-ylcarbonyloxy, 1-phenylbut-1-ylcarbonyloxy, 2-phenylbut-1-ylcarbonyloxy, 3-phenylbut-1-ylcarbonyloxy, 4-phenylbut-1-ylcarbonyloxy, 1-phenylbut-2-ylcarbonyloxy, 2-phenylbut-2-ylcarbonyloxy, 3-phenylbut-2-ylcarbonyloxy, 4-phenylbut-2-ylcarbonyloxy, 1-(phenylmethyl)eth-1-ylcarbonyloxy, 1-(phenylmethyl)-1-(methyl)eth-1-ylcarbonyloxy or 1-(phenylmethyl)prop-1-ylcarbonyloxy, in particular benzylcarbonyloxy or 2-phenylethylcarbonyloxy;

phenyl-C₁-C₆-alkylsulfonyloxy: for example benzylsulfonyloxy, 1-phenylethylsulfonyloxy, 2-phenylethylsulfonyloxy, 1-phenylprop-1-ylsulfonyloxy, 2-phenylprop-1-ylsulfonyloxy, 3-phenylprop-1-ylsulfonyloxy, 1-phenylbut-1-ylsulfonyloxy, 2-phenylbut-1-ylsulfonyloxy, 3-phenylbut-1-ylsulfonyloxy, 4-phenylbut-1-ylsulfonyloxy, 1-phenylbut-2-ylsulfonyloxy, 2-phenylbut-2-ylsulfonyloxy, 3-phenylbut-2-ylsulfonyloxy, 4-phenylbut-2-ylsulfonyloxy, 1-(phenylmethyl)eth-1-ylsulfonyloxy, 1-(phenylmethyl)-1-(methyl)eth-1-ylsulfonyloxy or 1-(phenylmethyl)prop-1-ylsulfonyloxy, in particular benzylsulfonyloxy or 2-phenylethylsulfonyloxy;

(C₁-C₆-alkyl)carbonyl: CO—CH₃, CO—C₂H₅, n-propylcarbonyl, 1-methylethylcarbonyl, n-butylcarbonyl, 1-methylpropylcarbonyl, 2-methylpropylcarbonyl, 1,1-dimethylethylcarbonyl, n-pentylcarbonyl, 1-methylbutylcarbonyl, 2-methylbutylcarbonyl, 3-methylbutylcarbonyl, 1,1-dimethylpropylcarbonyl, 1,2-dimethylpropylcarbonyl, 2,2-dimethylpropylcarbonyl, 1-ethylpropylcarbonyl, n-hexylcarbonyl, 1-methylpentylcarbonyl, 2-methylpentylcarbonyl, 3-methylpentylcarbonyl, 4-methylpentylcarbonyl, 1,1-dimethylbutylcarbonyl, 1,2-dimethylbutylcarbonyl, 1,3-dimethylbutylcarbonyl, 2,2-dimethylbutylcarbonyl, 2,3-dimethylbutylcarbonyl, 3,3-dimethylbutylcarbonyl, 1-ethylbutylcarbonyl, 2-ethylbutylcarbonyl, 1,1,2-trimethylpropylcarbonyl, 1,2,2-trimethylpropylcarbonyl, 1-ethyl-1-methylpropylcarbonyl or 1-ethyl-2-methylpropylcarbonyl, in particular CO—CH₃, CO—C₂H₅ or CO—CH(CH₃)₂;

(C₁-C₆-alkyl)carbonyl-C₁-C₆-alkyl: C₁-C₆-alkyl which is substituted by (C₁-C₆-alkyl)carbonyl as mentioned above, i.e., for example methylcarbonylmethyl;

(C₁-C₆-haloalkyl)carbonyl: a (C₁-C₆-alkyl)carbonyl radical as mentioned above which is partially or fully substituted by fluorine, chlorine, bromine and/or iodine, i.e., for example, chloroacetyl, dichloroacetyl, trichloroacetyl, fluoroacetyl, difluoroacetyl, trifluoroacetyl, chlorofluoroacetyl, dichlorofluoroacetyl, chlorodifluoroacetyl, 2-fluoroethylcarbonyl, 2-chloroethylcarbonyl, 2-bromoethylcarbonyl, 2-iodoethylcarbonyl, 2,2-difluoroethylcarbonyl, 2,2,2-trifluoroethylcarbonyl, 2-chloro-2-fluoroethylcarbonyl, 2-chloro-2,2-difluoroethylcarbonyl, 2,2-dichloro-2-fluoroethylcarbonyl, 2,2,2-trichloroethylcarbonyl, pentafluoroethylcarbonyl, 2-fluoropropylcarbonyl, 3-fluoropropylcarbonyl, 2,2-difluoropropylcarbonyl, 2,3-difluoropropylcarbonyl, 2-chloropropylcarbonyl, 3-chloropropylcarbonyl, 2,3-dichloropropylcarbonyl, 2-bromopropylcarbonyl 3-bromopropylcarbonyl, 3,3,3-trifluoropropylcarbonyl, 3,3,3-trichloropropylcarbonyl, 2,2,3,3,3-pentafluoropropylcarbonyl, heptafluoropropylcarbonyl, 1-(fluoromethyl)-2-fluoroethylcarbonyl, 1-(chloromethyl)-2-chloroethylcarbonyl, 1-(bromomethyl)-2-bromoethylcarbonyl, 4-fluorobutylcarbonyl, 4-chlorobutylcarbonyl, 4-bromobutylcarbonyl, monafluorobutylcarbonyl, 4-(5-fluoro-1-pentyl)carbonyl, (5-chloro-1-pentyl)carbonyl, (5-bromo-1-pentyl)carbonyl, (5-iodo-1-pentyl)carbonyl, (5,5,5-trichloro-1-pentyl)carbonyl, undecafluoropentylcarbonyl, (6-fluoro-1-hexyl)carbonyl, (6-chloro-1-hexyl)carbonyl, (6-bromo-1-hexyl)carbonyl, (6-iodo-1-hexyl)carbonyl, (6,6,6-trichloro-1-hexyl)carbonyl or dodecafluorohexylcarbonyl, in particular trifluoroacetyl;

(C₁-C₆-alkyl)carbonyloxy; acetyloxy, ethylcarbonyloxy, n-propylcarbonyloxy, 1-methylethylcarbonyloxy, n-butylcarbonyloxy, 1-methylpropylcarbonyloxy, 2-methylpropylcarbonyloxy, 1,1-dimethylethylcarbonyloxy, n-pentylcarbonyloxy, 1-methylbutylcarbonyloxy, 2-methylbutylcarbonyloxy, 3-methylbutylcarbonyloxy, 1,1-dimethylpropylcarbonyloxy, 1,2-dimethylpropylcarbonyloxy, 2,2-dimethylpropylcarbonyloxy, 1-ethylpropycarbonyloxy, n-hexylcarbonyloxy, 1-methylpentylcarbonyloxy, 2-methylpentylcarbonyloxy, 3-methylpentylcarbonyloxy, 4-methylpentylcarbonyloxy, 1,1-dimethylbutylcarbonyloxy, 1,2-dimethylbutylcarbonyloxy, 1,3-dimethylbutylcarbonyloxy, 2,2-dimethylbutylcarbonyloxy, 2,3-dimethylbutylcarbonyloxy, 3,3-dimethylbutylcarbonyloxy, 1-ethylbutylcarbonyloxy, 2-ethylbutylcarbonyloxy, 1,1,2-trimethylpropylcarbonyloxy, 1,2,2-trimethylpropylcarbonyloxy, 1-ethyl-1methylpropylcarbonyloxy or 1-ethyl-2-methylpropylcarbonyloxy, in particular acetyloxy;

(C₁-C₆-haloalkyl)carbonyloxy; a (C₁-C₆-alkyl)carbonyloxy radical as mentioned above which is partially or fully substituted by fluorine, chlorine, boromine and/or iodine, i.e., for example, chloroacetyloxy, dichloroacetyloxy, trichloroacetyloxy, fluoroacetyloxy, difluoroacetyloxy, trifluoroacetyloxy, chlorofluoroacetyloxy, dichlorofluoroacetyloxy, chlorodifluoroacetyloxy, 2-fluoroethylcarbonyloxy, 2-chloroethylcarbonyloxy, 2-bromoethylcarbonyloxy, 2-iodoethylcarbonyloxy, 2,2-difluoroethylcarbonyloxy, 2,2,2-trifluoroethylcarbonyloxy, 2-chloro-2-fluoroethylcarbonyloxy, 2-chloro-2,2-difluoroethylcarbonyloxy, 2,2-dichloro-2-fluoroethylcarbonyloxy, 2,2,2-trichloroethylcarbonyloxy, pentafluoroethylcarbonyloxy, 2-fluoropropylcarbonyloxy, 3-fluoropropylcarbonyloxy, 2,2-difluoropropylcarbonyloxy, 2,3-difluoropropylcarbonyloxy, 2-chloropropylcarbonyloxy, 3-chloropropylcarbonyloxy, 2,3-dichloropropylcarbonyloxy, 2-bromopropylcarbonyloxy, 3-bromopropylcarbonyloxy, 3,3,3-trifluoropropylcarbonyloxy, 3,3,3-trichloropropylcarbonyloxy, 2,2,3,3,3-pentafluoropropylcarbonyloxy, heptafluoropropylcarbonyloxy, 1-(fluoromethyl)-2-fluoroethylcarbonyloxy, 1-(chloromethyl)-2-chloroethylcarbonyloxy, 1-(bromomethyl)-2-bromoethylcarbonyloxy, 4-fluorobutylcarbonyloxy, 4-chlorobutylcarbonyloxy, 4-bromobutyl or nonafluorobutyl, in particular trifluoroacetoxy;

(C₁-C₆-alkyl)carbonyloxy-C₁-C₆-alkyl: C₁-C₆-alkyl which is substituted by (C₁-C₆-alkyl)carbonyloxy as mentioned above, i.e., for example, methylcarbonyloxymethyl, ethylcarbonyloxymethyl, 1-(methylcarbonyloxy)ethyl, 2-(methylcarbonyloxy)ethyl, 2-(ethylcarbonyloxy)ethyl, 3-(methylcarbonyloxy)propyl, 4-(methoxycarbonyloxy)butyl, 5-(methoxycarbonyloxy)pentyl or 6-(methoxycarbonyloxy)hexyl;

(C₁-C₆-alkyl)carbonylthio: acetylthio, ethylcarbonylthio, n-propylcarbonylthio, 1-methylethylcarbonylthio, n-butylcarbonylthio, 1-methylpropylcarbonylthio, 2-methylpropylcarbonylthio, 1,1-dimethylethylcarbonylthio, n-pentylcarbonylthio, 1-methylbutylcarbonylthio, 2-methylbutylcarbonylthio, 3-methylbutylcarbonylthio, 1,1-dimethylpropylcarbonylthio, 1,2-dimethylpropylcarbonylthio, 2,2-dimethylpropylcarbonylthio, 1-ethylpropylcarbonylthio, n-hexylcarbonylthio, 1-methylpentylcarbonylthio, 2-methylpentylcarbonylthio, 3-methylpentylcarbonylthio, 4-methylpentylcarbonylthio, 1,1-dimethylbutylcarbonylthio, 1,2-dimethylbutylcarbonylthio, 1,3-dimethylbutylcarbonylthio, 2,2-dimethylbutylcarbonylthio, 2,3-dimethylbutylcarbonylthio, 3,3-dimethylbutylcarbonylthio, 1-ethylbutylcarbonylthio, 2-ethylbutylcarbonylthio, 1,1,2-trimethylpropylcarbonylthio, 1,2,2-trimethylpropylcarbonylthio, 1-ethyl-1-methylpropylcarbonylthio or 1-ethyl-2-methylpropylcarbonylthio, in particular acetylthio;

(C₁-C₆-haloalkyl)carbonylthio: a (C₁-C₆-alkyl)carbonylthio radical as mentioned above which is partially or fully substituted by fluorine, chlorine, bromine and/or iodine, i.e., for example, chloroacetylthio, dichloroacetylthio, trichloroacetylthio, fluoroacetylthio, difluoroacetylthio, trifluoroacetylthio, chlorofluoroacetylthio, dichlorofluoroacetylthio, chlorodifluoroacetylthio, 2-fluoroethylcarbonylthio, 2-chloroethylcarbonylthio, 2-bromoethylcarbonylthio, 2-iodoethylcarbonylthio, 2,2-difluoroethylcarbonylthio, 2,2,2-trifluoroethylcarbonylthio, 2-chloro-2-fluoroethylcarbonylthio, 2-chloro-2,2-difluoroethylcarbonylthio, 2,2-dichloro-2-fluoroethylcarbonylthio, 2,2,2-trichloroethylcarbonylthio, pentafluoroethylcarbonylthio, 2-fluoropropylcarbonylthio, 3-fluoropropylcarbonylthio, 2,2-difluoropropylcarbonylthio, 2,3-difluoropropylcarbonylthio, 2-chloropropylcarbonylthio, 3-chloropropylcarbonylthio, 2,3-dichloropropylcarbonylthio, 2-bromopropylcarbonylthio, 3-bromopropylcarbonylthio, 3,3,3-trifluoropropylcarbonylthio, 3,3,3-trichloropropylcarbonylthio, 2,2,3,3,3-pentafluoropropylcarbonylthio, heptafluoropropylcarbonylthio, 1-(fluoromethyl)-2-fluoroethylcarbonylthio, 1-(chloromethyl)-2-chloroethylcarbonylthio, 1-(bromomethyl)-2-bromoethylcarbonylthio, 4-fluorobutylcarbonylthio, 4-chlorobutylcarbonylthio, 4-bromobutylthio or nonafluorobutylthio, in particular trifluoroacetylthio;

(C₁-C₆-alkyl)carbamoyloxy: methylcarbamoyloxy, ethylcarbamoyloxy, n-propylcarbamoyloxy, 1-methylethylcarbamoyloxy, n-butylcarbamoyloxy, 1-methylpropylcarbamoyloxy, 2-methylpropylcarbamoyloxy, 1,1-dimethylethylcarbamoyloxy, n-pentylcarbamoyloxy, 1-methylbutylcarbamoyloxy, 2-methylbutylcarbamoyloxy, 3-methylbutylcarbamoyloxy, 1,1-dimethylpropylcarbamoyloxy, 1,2-dimethylpropylcarbamoyloxy, 2,2-dimethylpropylcarbamoyloxy, 1-ethylpropylcarbamoyloxy, n-hexylcarbamoyloxy, 1-methylpentylcarbamoyloxy, 2-methylpentylcarbamoyloxy, 3-methylpentylcarbamoyloxy, 4-methylpentylcarbamoyloxy, 1,1-dimethylbutylcarbamoyloxy, 1,2-dimethylbutylcarbamoyloxy, 1,3-dimethylbutylcarbamoyloxy, 2,2-dimethylbutylcarbamoyloxy, 2,3-dimethylbutylcarbamoyloxy, 3,3-dimethylbutylcarbamoyloxy, 1-ethylbutylcarbamoyloxy, 2-ethylbutylcarbamoyloxy, 1,1,2-trimethylpropylcarbamoyloxy, 1,2,2-trimethylpropylcarbamoyloxy, 1-ethyl-1-methylpropylcarbamoyloxy or 1-ethyl-2-methylpropylcarbamoyloxy, in particular methylcarbamoyloxy;

(C₁-C₆-haloalkyl)carbamoyloxy: a (C₁-C₆-alkyl)carbamoyloxy radical as mentioned above which is partially or fully substituted by fluorine, chlorine, bromine and/or iodine, i.e., for example, chloromethylcarbamoyloxy, dichloromethylcarbamoyloxy, trichloromethylcarbamoyloxy, fluoromethylcarbamoyloxy, difluoromethylcarbamoyloxy, trifluoromethylcarbamoyloxy, chlorofluoromethylcarbamoyloxy, dichlorofluoromethylcarbamoyloxy, chlorodifluoromethylcarbamoyloxy, 2-fluoroethylcarbamoyloxy, 2-chloroethylcarbamoyloxy, 2-bromoethylcarbamoyloxy, 2-iodoethylcarbamoyloxy, 2,2-difluoroethylcarbamoyloxy, 2,2,2-trifluoroethylcarbamoyloxy, 2-chloro-2-fluoroethylcarbamoyloxy, 2-chloro-2,2-difluoroethylcarbamoyloxy, 2,2-dichloro-2-fluoroethylcarbamoyloxy, 2,2,2-trichloroethylcarbamoyloxy, pentafluoroethylcarbamoyloxy, 2-fluoropropylcarbamoyloxy, 3-fluoropropylcarbamoyloxy, 2,2-difluoropropylcarbamoyloxy, 2,3-difluoropropylcarbamoyloxy, 2-chloropropylcarbamoyloxy, 3-chloropropylcarbamoyloxy, 2,3-dichloropropylcarbamoyloxy, 2-bromopropylcarbamoyloxy, 3-bromopropylcarbamoyloxy, 3,3,3-trifluoropropylcarbamoyloxy, 3,3,3-trichloropropylcarbamoyloxy, 2,2,3,3,3-pentafluoropropylcarbamoyloxy, heptafluoropropylcarbamoyloxy, 1-(fluoromethyl)-2-fluoroethylcarbamoyloxy, 1-(chloromethyl)-2-chloroethylcarbamoyloxy, 1-(bromomethyl)-2-bromoethylcarbamoyloxy, 4-fluorobutylcarbamoyloxy, 4-chlorobutylcarbamoyloxy, 4-bromobutylcarbamoyloxy or nonafluorobutylcarbamoyloxy, in particular trifluoromethylcarbamoyloxy;

C₁-C₆-alkoxy: for example OCH₃, OC₂H₅, OCH₂-C₂H₅, OCH(CH₃)₂, n-butoxy, OCH(CH₃)—C₂H₅, OCH₂—CH(CH₃)₂, OC(CH₃)₃, n-pentoxy, 1-methylbutoxy, 2-methylbutoxy, 3-methylbutoxy, 1,1-dimethylpropoxy, 1,2-dimethylpropoxy, 2,2-dimethylpropoxy, 1-ethylpropoxy, n-hexoxy, 1-methylpentoxy, 2-methylpentoxy, 3-methylpentoxy, 4-methylpentoxy, 1,1-dimethylbutoxy, 1,2-dimethylbutoxy, 1,3-dimethylbutoxy, 2,2-dimethylbutoxy, 2,3-dimethylbutoxy, 3,3-dimethylbutoxy, 1-ethylbutoxy, 2-ethylbutoxy, 1,1,2-trimethylpropoxy, 1,2,2-trimethylpropoxy, 1-ethyl-1-methylpropoxy or 1-ethyl-2-methylpropoxy, in particular OCH₃, OC₂H₅ or OCH(CH₃)₂;

C₁-C₄-haloalkoxy: a C₁-C₄-alkoxy radical as mentioned above which is partially or fully substituted by fluorine, chlorine, bromine and/or iodine, i.e., for example, chloromethoxy, dichloromethoxy, trichloromethoxy, fluoromethoxy, difluoromethoxy, trifluoromethoxy, chlorofluoromethoxy, dichlorofluoromethoxy, chlorodifluoromethoxy, 2-fluoroethoxy, 2-chloroethoxy, 2-bromoethoxy, 2-iodoethoxy, 2,2-difluoroethoxy, 2,2,2-trifluoroethoxy, 2-chloro-2-fluoroethoxy, 2-chloro-2,2-difluoroethoxy, 2,2-dichloro-2-fluoroethoxy, 2,2,2-trichloroethoxy, pentafluoroethoxy, 2-fluoropropoxy, 3-fluoropropoxy, 2,2-difluoropropoxy, 2,3-difluoropropoxy, 2-chloropropoxy, 3-chloropropoxy, 2,3-dichloropropoxy, 2-bromopropoxy, 3-bromopropoxy, 3,3,3-trifluoropropoxy, 3,3,3-trichloropropoxy, 2,2,3,3,3-pentafluoropropoxy, heptafluoropropoxy, 1-(fluoromethyl)-2-fluoroethoxy, 1-(chloromethyl)-2-chloroethoxy, 1-(bromomethyl)-2-bromoethoxy, 4-fluorobutoxy, 4-chlorobutoxy, 4-bromobutoxy or nonafluorobutoxy, in particular 2-chloroethoxy or 2,2,2-trifluoroethoxy;

C₁-C₆-haloalkoxy: a C₁-C₆-alkoxy radical as mentioned above which is partially or fully substituted by fluorine, chlorine, bromine and/or iodine, i.e., for example, one of the radicals mentioned under C₁-C₄-haloalkoxy or 5-fluoro-1-pentoxy, 5-chloro-1-pentoxy, 5-bromo-1-pentoxy, 5-iodo-1-pentoxy, 5,5,5-trichloro-1-pentoxy, undecafluoropentoxy, 6-fluoro-1-hexoxy, 6-chloro-1-hexoxy, 6-bromo-1-hexoxy, 6-iodo-1-hexoxy, 6,6,6-trichloro-1-hexoxy or dodecafluorohexoxy, in particular chloromethoxy, fluoromethoxy, difluoromethoxy, trifluoromethoxy, 2-fluoroethoxy, 2-chloroethoxy or 2,2,2-trifluoroethoxy;

hydroxy-C₁-C₆-alkoxy: for example OCH₂—OH, OCH(CH₃)—OH, OCH₂—CH₂—OH, OCH(C₂H₅)—OH, OCH₂—CH(CH₃)—OH, 3-hydroxyprop-1-yloxy, 1-hydroxybut-1-yloxy, 2-hydroxybut-1-yloxy, 3-hydroxybut-1-yloxy, 4-hydroxybut-1-yloxy, 1-hydroxybut-2-yloxy, 2-hydroxybut-2-yloxy, 3-hydroxybut-2-yloxy, 4-hydroxybut-2-yloxy, 1-(CH₂—OH)-eth-1-yloxy, 1-(CH₂—OH)-1-(CH₃)-eth-1-yloxy or 1-(CH₂—OH)-prop-1-yloxy, in particular OCH₂—OH or OCH₂—CH₂—OH;

cyano-C₁-C₆-alkoxy: for example OCH₂—CN, OCH(CH₃)—CN, OCH₂—CH₂—CN, OCH(C₂H₅)—OH, OCH₂—CH(CH₃)—CN, 3-cyanoprop-1-yloxy, 1-cyanobut-1-yloxy, 2-cyanobut-1-yloxy, 3-cyanobut-1-yloxy, 4-cyanobut-1-yloxy, 1-cyanobut-2-yloxy, 2-cyanobut-2-yloxy, 3-cyanobut-2-yloxy, 4-cyanobut-2-yloxy, 1-(CH₂—CN)-eth-1-yloxy, 1-(CH₂—CN)-1-(CH₃)-eth-1-yloxy or 1-(CH₂—CN)-prop-1-yloxy, in particular OCH₂—CN or OCH₂—CH₂—CN;

phenyl-C₁-C₆-alkoxy: for example benzyloxy, 1-phenylethoxy, 2-phenylethoxy, 1-phenylprop-1-yloxy, 2-phenylprop-1-yloxy, 3-phenylprop-1-yloxy, 1-phenylbut-1-yloxy, 2-phenylbut-1-yloxy, 3-phenylbut-1-yloxy, 4-phenylbut-1-yloxy, 1-phenylbut-2-yloxy, 2-phenylbut-2-yloxy, 3-phenylbut-2-yloxy, 4-phenylbut-2-yloxy, 1-(benzyl)eth-1-yloxy, 1-(benzyl)-1-(methyl)eth-1-yloxy or 1-(benzyl)prop-1-yloxy, in particular benzyloxy or 2-phenylethoxy;

heterocyclyl-C₁-C₆-alkoxy: for example heterocyclylmethoxy, 1-(heterocyclyl)ethoxy, 2-(heterocyclyl)ethoxy, 1-(heterocyclyl)prop-1-yloxy, 2-(heterocyclyl)prop-1-yloxy, 3-(heterocyclyl)prop-1-yloxy, 1-(heterocyclyl)but-1-yloxy, 2-(heterocyclyl)but-1-yloxy, 3-(heterocyclyl)but-1-yloxy, 4-(heterocyclyl)but-1-yloxy, 1-(heterocyclyl)but-2-yloxy, 2-(heterocyclyl)but-2-yloxy, 3-(heterocyclyl)but-2-yloxy, 4-(heterocyclyl)but-2-yloxy, 1-(heterocyclylmethyl)eth-1-yloxy, 1-(heterocyclylmethyl)-1-(methyl)eth-1-yloxy or 1-(heterocyclylmethyl)prop-1-yloxy, in particular heterocyclylmethoxy or 2-(heterocyclyl)ethoxy;

phenyl-C₁-C₆-alkylthio: for example benzylthio, 1-phenylethylthio, 2-phenylethylthio, 1-phenylprop-1-ylthio, 2-phenylprop-1-ylthio, 3-phenylprop-1-ylthio, 1-phenylbut-1-ylthio, 2-phenylbut-1-ylthio, 3-phenylbut-1-ylthio, 4-phenylbut-1-ylthio, 1-phenylbut-2-ylthio, 2-phenylbut-2-ylthio, 3-phenylbut-2-ylthio, 4-phenylbut-2-ylthio, 1-(phenylmethyl)eth-1-ylthio, 1-(phenylmethyl)-1-(methyl)eth-1-ylthio or 1-(phenylmethyl)prop-1-ylthio, in particular benzylthio or 2-phenylethylthio;

(C₁-C₆-alkoxy)carbonyl: for example CO—OCH₃, CO—OC₂H₅, COO—CH₂—C₂H₅, CO—OCH(CH₃)₂, n-butoxycarbonyl, CO—OCH(CH₃)—C₂H₅, CO—OCH₂—CH(CH₃)₂, CO—OC(CH₃)₃, n-pentoxycarbonyl, 1-methylbutoxycarbonyl, 2-methylbutoxycarbonyl, 3-methylbutoxycarbonyl, 2,2-dimethylpropoxycarbonyl, 1-ethylpropoxycarbonyl, n-hexoxycarbonyl, 1,1-dimethylpropoxycarbonyl, 1,2-dimethylpropoxycarbonyl, 1-methylpentoxycarbonyl, 2-methylpentoxycarbonyl, 3-methylpentoxycarbonyl, 4-methylpentoxycarbonyl, 1,1-dimethylbutoxycarbonyl, 1,2-dimethylbutoxycarbonyl, 1,3-dimethylbutoxycarbonyl, 2,2-dimethylbutoxycarbonyl, 2,3-dimethylbutoxycarbonyl, 3,3-dimethylbutoxycarbonyl, 1-ethylbutoxycarbonyl, 2-ethylbutoxycarbonyl, 1,1,2-trimethylpropoxycarbonyl, 1,2,2-trimethylpropoxycarbonyl, 1-ethyl-1-methylpropoxycarbonyl or 1-ethyl-2-methylpropoxycarbonyl, in particular CO—OCH₃, CO—OC₂H₅, CO—OCH(CH₃)₂ or CO—CH₂—CH(CH₃)₂;

(C₁-C₆-alkoxy)carbonyloxy: methoxycarbonyloxy, ethoxycarbonyloxy, n-propoxycarbonyloxy, 1-methylethoxycarbonyloxy, n-butoxycarbonyloxy, 1-methylpropoxycarbonyloxy, 2-methylpropoxycarbonyloxy, 1,1-dimethylethoxycarbonyloxy, n-pentoxycarbonyloxy, 1-methylbutoxycarbonyloxy, 2-methylbutoxycarbonyloxy, 3-methylbutoxycarbonyloxy, 2,2-dimethylpropoxycarbonyloxy, 1-ethylpropoxycarbonyloxy, n-hexoxycarbonyloxy, 1,1-dimethylpropoxycarbonyloxy, 1,2-dimethylpropoxycarbonyloxy, 1-methylpentoxycarbonyloxy, 2-methylpentoxycarbonyloxy, 3-methylpentoxycarbonyloxy, 4-methylpentoxycarbonyloxy, 1,1-dimethylbutoxycarbonyloxy, 1,2-dimethylbutoxycarbonyloxy, 1,3-dimethylbutoxycarbonyloxy, 2,2-dimethylbutoxycarbonyloxy, 2,3-dimethylbutoxycarbonyloxy, 3,3-dimethylbutoxycarbonyloxy, 1-ethylbutoxycarbonyloxy, 2-ethylbutoxycarbonyloxy, 1,1,2-trimethylpropoxycarbonyloxy, 1,2,2-trimethylpropoxycarbonyloxy, 1-ethyl-1-methylpropoxycarbonyloxy or 1-ethyl-2-methylpropoxycarbonyloxy, in particular methoxycarbonyloxy, ethoxycarbonyloxy or 1-methylethoxycarbonyloxy;

(C₁-C₆-alkoxy)carbonylthio: methoxycarbonylthio, ethoxycarbonylthio, n-propoxycarbonylthio, 1-methylethoxycarbonylthio, n-butoxycarbonylthio, 1-methylpropoxycarbonylthio, 2-methylpropoxycarbonylthio, 1,1-dimethylethoxycarbonylthio, n-pentoxycarbonylthio, 1-methylbutoxycarbonylthio, 2-methylbutoxycarbonylthio, 3-methylbutoxycarbonylthio, 2,2-dimethylpropoxycarbonylthio, 1-ethylpropoxycarbonylthio, n-hexoxycarbonylthio, 1,1-dimethylpropoxycarbonylthio, 1,2-dimethylpropoxycarbonylthio, 1-methylpentoxycarbonylthio, 2-methylpentoxycarbonylthio, 3-methylpentoxycarbonylthio, 4-methylpentoxycarbonylthio, 1,1-dimethylbutoxycarbonylthio, 1,2-dimethylbutoxycarbonylthio, 1,3-dimethylbutoxycarbonylthio, 2,2-dimethylbutoxycarbonylthio, 2,3-dimethylbutoxycarbonylthio, 3,3-dimethylbutoxycarbonylthio, 1-ethylbutoxycarbonylthio, 2-ethylbutoxycarbonylthio, 1,1,2-trimethylpropoxycarbonylthio, 1,2,2-trimethylpropoxycarbonylthio, 1-ethyl-1-methylpropoxycarbonylthio or 1-ethyl-2-methylpropoxycarbonylthio, in particular methoxycarbonylthio, ethoxycarbonylthio or 1-methylethoxycarbonylthio;

C₁-C₆-alkylthio: SCH₃, SC₂H₅, SCH₂—C₂H₅, SCH(CH₃)₂, n-butylthio, 1-methylpropylthio, 2-methylpropylthio, SC(CH₃)₃, n-pentylthio, 1-methylbutylthio, 2-methylbutylthio, 3-methylbutylthio, 2,2-dimethylpropylthio, 1-ethylpropylthio, n-hexylthio, 1,1-dimethylpropylthio, 1,2-dimethylpropylthio, 1-methylpentylthio, 2-methylpentylthio, 3-methylpentylthio, 4-methylpentylthio, 1,1-dimethylbutylthio, 1,2-dimethylbutylthio, 1,3-dimethylbutylthio, 2,2-dimethylbutylthio, 2,3-dimethylbutylthio, 3,3-dimethylbutylthio, 1-ethylbutylthio, 2-ethylbutylthio, 1,1,2-trimethylpropylthio, 1,2,2-trimethylpropylthio, 1-ethyl-1-methylpropylthio or 1-ethyl-2-methylpropylthio, in particular SCH₃ or SC₂H₅;

C₁-C₆-haloalkylthio: C₁-C₆-alkylthio as mentioned above which is partially or fully substituted by fluorine, chlorine, bromine and/or iodine, i.e., for example, SCHF₂, SCF₃, chlorodifluoromethylthio, bromodifluoromethylthio, 2-fluoroethylthio, 2-chloroethylthio, 2-bromoethylthio, 2-iodoethylthio, 2,2-difluoroethylthio, 2,2,2-trifluoroethylthio, 2,2,2-trichloroethylthio, 2-chloro-2-fluoroethylthio, 2-chloro-2,2-difluoroethylthio, 2,2-dichloro-2-fluoroethylthio, SC₂F₅, 2-fluoropropylthio, 3-fluoropropylthio, 2-chloropropylthio, 3-chloropropylthio, 2-bromopropylthio, 3-bromopropylthio, 2,2-difluoropropylthio, 2,3-difluoropropylthio, 2,3-dichloropropylthio, 3,3,3-trifluoropropylthio, 3,3,3-trichloropropylthio, 2,2,3,3,3-pentafluoropropylthio, heptafluoropropylthio, 1-(fluoromethyl)-2-fluoroethylthio, 1-(chloromethyl)-2-chloroethylthio, 1-(bromomethyl)-2-bromoethylthio, 4-fluorobutylthio, 4-chlorobutylthio, 4-bromobutylthio, nonafluorobutylthio, 5-fluoropentylthio, 5-chloropentylthio, 5-bromopentylthio, 5-iodopentylthio, undecafluoropentylthio, 6-fluorohexylthio or 6-chlorohexylthio, in particular SCH₂F, SCHF₂, SCF₃, SCH₂Cl, 2-fluoroethylthio, 2-chloroethylthio or 2,2,2-trifluoroethylthio;

C₁-C₆-alkylsulfinyl: SO—CH₃, SO—C₂H₅, n-propylsulfinyl, 1-methylethylsulfinyl, n-butylsulfinyl, 1-methylpropylsulfinyl, 2-methylpropylsulfinyl, 1,1-dimethylethylsulfinyl, n-pentylsulfinyl, 1-methylbutylsulfinyl, 2-methylbutylsulfinyl, 3-methylbutylsulfinyl, 1,1-dimethylpropylsulfinyl, 1,2-dimethylpropylsulfinyl, 2,2-dimethylpropylsulfinyl, 1-ethylpropylsulfinyl, n-hexylsulfinyl, 1-methylpentylsulfinyl, 2-methylpentylsulfinyl, 3-methylpentylsulfinyl, 4-methylpentylsulfinyl, 1,1-dimethylbutylsulfinyl, 1,2-dimethylbutylsulfinyl, 1,3-dimethylbutylsulfinyl, 2,2-dimethylbutylsulfinyl, 2,3-dimethylbutylsulfinyl, 3,3-dimethylbutylsulfinyl, 1-ethylbutylsulfinyl, 2-ethylbutylsulfinyl, 1,1,2-trimethylpropylsulfinyl, 1,2,2-trimethylpropylsulfinyl, 1-ethyl-1-methylpropylsulfinyl or 1-ethyl-2-methylpropylsulfinyl, in particular SO—CH₃;

C₁-C₆-alkylsulfonyl: SO₂—CH₃, SO₂—C₂H₅, n-propylsulfonyl, SO₂—CH(CH₃)₂, n-butylsulfonyl, 1-methylpropylsulfonyl, 2-methylpropylsulfonyl, SO₂—C(CH₃) 3, n-pentylsulfonyl, 1-methylbutylsulfonyl, 2-methylbutylsulfonyl, 3-methylbutylsulfonyl, 1,1-dimethylpropylsulfonyl, 1,2-dimethylpropylsulfonyl, 2,2-dimethylpropylsulfonyl, 1-ethylpropylsulfonyl, n-hexylsulfonyl, 1-methylpentylsulfonyl, 2-methylpentylsulfonyl, 3-methylpentylsulfonyl, 4-methylpentylsulfonyl, 1,1-dimethylbutylsulfonyl, 1,2-dimethylbutylsulfonyl, 1,3-dimethylbutylsulfonyl, 2,2-dimethylbutylsulfonyl, 2,3-dimethylbutylsulfonyl, 3,3-dimethylbutylsulfonyl, 1-ethylbutylsulfonyl, 2-ethylbutylsulfonyl, 1,1,2-trimethylpropylsulfonyl, 1,2,2-trimethylpropylsulfonyl, 1-ethyl-1-methylpropylsulfonyl or 1-ethyl-2-methylpropylsulfonyl, in particular SO₂—CH₃;

C₁-C₆-alkylsulfonyloxy: O—SO₂—CH₃, O—SO₂—C₂H₅, n-propylsulfonyloxy, O—SO₂—CH(CH₃)₂, n-butylsulfonyloxy, 1-methylpropylsulfonyloxy, 2-methylpropylsulfonyloxy, O—SO₂—C(CH₃)₃, n-pentylsulfonyloxy, 1-methylbutylsulfonyloxy, 2-methylbutylsulfonyloxy, 3-methylbutylsulfonyloxy, 1,1-dimethylpropylsulfonyloxy, 1,2-dimethylpropylsulfonyloxy, 2,2-dimethylpropylsulfonyloxy, 1-ethylpropylsulfonyloxy, n-hexylsulfonyloxy, 1-methylpentylsulfonyloxy, 2-methylpentylsulfonyloxy, 3-methylpentylsulfonyloxy, 4-methylpentylsulfonyloxy, 1,1-dimethylbutylsulfonyloxy, 1,2-dimethylbutylsulfonyloxy, 1,3-dimethylbutylsulfonyloxy, 2,2-dimethylbutylsulfonyloxy, 2,3-dimethylbutylsulfonyloxy, 3,3-dimethylbutylsulfonyloxy, 1-ethylbutylsulfonyloxy, 2-ethylbutylsulfonyloxy, 1,1,2-trimethylpropylsulfonyloxy, 1,2,2-trimethylpropylsulfonyloxy, 1-ethyl-1-methylpropylsulfonyloxy or 1-ethyl-2-methylpropylsulfonyloxy, in particular methylsulfonyloxy;

C₁-C₆-haloalkylsulfonyloxy: C₁-C₆-alkylsulfonyloxy as mentioned above which is partially or fully substituted by fluorine, chlorine, bromine and/or iodine, i.e, for example, ClCH₂—SO₂—O—, CH(Cl)₂—SO₂—O—, C(Cl)₃—SO₂—O—, FCH₂—SO₂—O—, CHF₂—SO₂—O—, CF₃—SO₂—O—, chlorofluoromethyl-SO₂—O—, dichlorofluoromethyl-SO₂—O—, chlorodifluoromethyl-SO₂—O—, 1-fluoroethyl-SO₂—O—, 2-fluoroethyl-SO₂—O—, 2-chloroethyl-SO₂—O—, 2-bromoethyl-SO₂—O—, 2-iodoethyl-SO₂—O—, 2,2-difluoroethyl-SO₂—O—, 2,2,2-trifluoroethyl-SO₂—O—, 2-chloro-2-fluoroethyl-SO₂—O—, 2-chloro-2,2-difluoroethyl-SO₂—O—, 2,2-dichloro-2-fluoroethyl-SO₂—O—, 2,2,2-trichloroethyl-SO₂—O—, C₂F₅—SO₂—O—, 2-fluoropropyl-SO₂—O—, 3-fluoropropyl-SO₂—O—, 2,2-difluoropropyl-SO₂—O—, 2,3-difluoropropyl-SO₂—O—, 2-chloropropyl-SO₂—O—, 3-chloropropyl-SO₂—O—, 2,3-dichloropropyl-SO₂—O—, 2-bromopropyl-SO₂—O—, 3-bromopropyl-SO₂—O—, 3,3,3-trifluoropropyl-SO₂—O—, 3,3,3-trichloropropyl-SO₂—O—, 2,2,3,3,3-pentafluoropropyl-SO₂—O—, C₂F₅—CF₂—SO₂—O—, 1-(fluoromethyl)-2-fluoroethyl-SO₂—O—, 1-(chloromethyl)-2-chloroethyl-SO₂—O—, 1-(bromomethyl)-2-bromoethyl-SO₂—O—, 4-fluorobutyl-SO₂—O—, 4-chlorobutyl-SO₂—O—, 4-bromobutyl-SO₂—O—, C₂F₅-CF₂—CF₂—SO₂—O—, 5-fluoropentyl-SO₂—O—, 5-chloropentyl-SO₂—O—, 5-bromopentyl-SO₂—O—, 5-iodopentyl-SO₂—O—, 5,5,5-trichloropentyl-SO₂—O—, C₂F₅—CF₂—CF₂—CF₂—SO₂—O—, 6-fluorohexyl-SO₂—O—, 6-chlorohexyl-SO₂—O—, 6-bromohexyl-SO₂—O—, 6-iodohexyl-SO₂—O—, 6,6,6-trichlorohexyl-SO₂—O— or dodecafluorohexyl-SO₂—O—, in particular CF₃—SO₂—O—;

(C₁-C₆-alkyl)aminocarbonyl: (C₁-C₄-alkyl)aminocarbonyl as mentioned above, and also, for example, n-pentylaminocarbonyl, 1-methylbutylaminocarbonyl, 2-methylbutylaminocarbonyl, 3-methylbutylaminocarbonyl, 2,2-dimethylpropylaminocarbonyl, 1-ethylpropylaminocarbonyl, n-hexylaminocarbonyl, 1,1-dimethylpropylaminocarbonyl, 1,2-dimethylpropylaminocarbonyl, 1-methylpentylaminocarbonyl, 2-methylpentylaminocarbonyl, 3-methylpentylaminocarbonyl, 4-methylpentylaminocarbonyl, 1,1-dimethylbutylaminocarbonyl, 1,2-dimethylbutylaminocarbonyl, 1,3-dimethylbutylaminocarbonyl, 2,2-dimethylbutylaminocarbonyl, 2,3-dimethylbutylaminocarbonyl, 3,3-dimethylbutylaminocarbonyl, 1-ethylbutylaminocarbonyl, 2-ethylbutylaminocarbonyl, 1,1,2-trimethylpropylaminocarbonyl, 1,2,2-trimethylpropylaminocarbonyl, 1-ethyl-1-methylpropylaminocarbonyl or 1-ethyl-2-methylpropylaminocarbonyl, in particular CO—NH—CH₃, CO—NH—C₂H₅ or CO—NH—CH(CH₃)₂;

di(C₁-C₆-alkyl)aminocarbonyl: for example N,N-dimethylaminocarbonyl, N,N-diethylaminocarbonyl, N,N-dipropylaminocarbonyl, N,N-di-(1-methylethyl)aminocarbonyl, N,N-dibutylaminocarbonyl, N,N-di-(1-methylpropyl)aminocarbonyl, N,N-di-(2-methylpropyl)aminocarbonyl, N,N-di-(1,1-dimethylethyl)aminocarbonyl, N-ethyl-N-methylaminocarbonyl, N-methyl-N-propylaminocarbonyl, N-methyl-N-(1-methylethyl)aminocarbonyl, N-butyl-N-methylaminocarbonyl, N-methyl-N-(1-methylpropyl)aminocarbonyl, N-methyl-N-(2-methylpropyl)aminocarbonyl, N-(1,1-dimethylethyl)-N-methylaminocarbonyl, N-ethyl-N-propylaminocarbonyl, N-ethyl-N-(1-methylethyl)aminocarbonyl, N-butyl-N-ethylaminocarbonyl, N-ethyl-N-(1-methylpropyl)aminocarbonyl, N-ethyl-N-(2-methylpropyl)aminocarbonyl, N-ethyl-N-(1,1-dimethylethyl)aminocarbonyl, N-(1-methylethyl)-N-propylaminocarbonyl, N-butyl-N-propylaminocarbonyl, N-(1-methylpropyl)-N-propylaminocarbonyl, N-(2-methylpropyl)-N-propylaminocarbonyl, N-(1,1-dimethylethyl)-N-propylaminocarbonyl, N-butyl-N-(1-methylethyl)aminocarbonyl, N-(1-methylethyl)-N-(1-methylpropyl)aminocarbonyl, N-(1-methylethyl)-N-(2-methylpropyl)aminocarbonyl, N-(1,1-dimethylethyl)-N-(1-methylethyl)aminocarbonyl, N-butyl-N-(1-methylpropyl)aminocarbonyl, N-butyl-N-(2-methylpropyl)aminocarbonyl, N-butyl-N-(1,1-dimethylethyl)aminocarbonyl, N-(1-methylpropyl)-N-(2-methylpropyl)aminocarbonyl, N-(1,1-dimethylethyl)-N-(1-methylpropyl)aminocarbonyl or N-(1,1-dimethylethyl)-N-(2-methylpropyl)aminocarbonyl, in particular N,N-dimethylaminocarbonyl or N,N-diethylaminocarbonyl;

(C₁-C₆-alkyl)iminooxycarbonyl: methyliminooxycarbonyl, ethyliminooxycarbonyl, n-propyliminooxycarbonyl, 1-methylethyliminooxycarbonyl, n-butyliminooxycarbonyl, 1-methylpropyliminooxycarbonyl, 2-methylpropyliminooxycarbonyl, 1,1-dimethylethyliminooxycarbonyl, n-pentyliminooxycarbonyl, 1-methylbutyliminooxycarbonyl, 2-methylbutyliminooxycarbonyl, 3-methylbutyliminooxycarbonyl, 1,1-dimethylpropyliminooxycarbonyl, 1,2-dimethylpropyliminooxycarbonyl, 2,2-dimethylpropyliminooxycarbonyl, 1-ethylpropyliminooxycarbonyl, n-hexyliminooxycarbonyl, 1-methylpentyliminooxycarbonyl, 2-methylpentyliminooxycarbonyl, 3-methylpentyliminooxycarbonyl, 4-methylpentyliminooxycarbonyl, 1,1-dimethylbutyliminooxycarbonyl, 1,2-dimethylbutyliminooxycarbonyl, 1,3-dimethylbutyliminooxycarbonyl, 2,2-dimethylbutyliminooxycarbonyl, 2,3-dimethylbutyliminooxycarbonyl, 3,3-dimethylbutyliminooxycarbonyl, 1-ethylbutyliminooxycarbonyl, 2-ethylbutyliminooxycarbonyl, 1,1,2-trimethylpropyliminooxycarbonyl, 1,2,2-trimethylpropyliminooxycarbonyl, 1-ethyl-1-methylpropyliminooxycarbonyl or 1-ethyl-2-methylpropyliminooxycarbonyl, in particular methyliminooxycarbonyl, ethyliminooxycarbonyl or 1-methylethyliminooxycarbonyl;

C₁-C₆-alkylideneaminooxy: 1-propylideneaminooxy, 2-propylideneaminooxy, 1-butylideneaminooxy, 2-butylideneaminooxy or 2-hexylideneaminooxy, in particular butylideneminooxy or 2-propylideneaminooxy;

C₁-C₆-alkyliminooxy: methyliminooxy, ethyliminooxy, n-propyliminooxy, 1-methylethyliminooxy, n-butyliminooxy, 1-methylpropyliminooxy, 2-methylpropyliminooxy, n-pentyliminooxy, n-hexyliminooxy, 1-methylpentyliminooxy, 2-methylpentyliminooxy, 3-methylpentyliminooxy or 4-methylpentyliminooxy, in particular methyliminooxy, ethyliminooxy or 1-methylethyliminooxy;

C₁-C₆-alkoxy-(C₁-C₆-alkyl)aminocarbonyl: (C₁-C₆-alkyl)aminocarbonyl such as CO—NH—CH₃, CO—NH—C₂H₅, CO—NH—CH₂—C₂H₅, CO—NH—CH(CH₃)₂, CO—NH—(CH₂)₃—CH₃, CO—NH—CH(CH₃)—C₂H₅, CO—NH—CH₂—CH(CH₃)₂, CO—NH—C(CH₃)₃, CO—NH—(CH₂)₄—CH₃, 1-methylbutylaminocarbonyl, 2-methylbutylaminocarbonyl, 3-methylbutylaminocarbonyl, 2,2-dimethylpropylaminocarbonyl, 1-ethylpropylaminocarbonyl, n-hexylaminocarbonyl, 1,1-dimethylpropylaminocarbonyl, 1,2-dimethylpropylaminocarbonyl, 1-methylpentylaminocarbonyl, 2-methylpentylaminocarbonyl, 3-methylpentylaminocarbonyl, 4-methylpentylaminocarbonyl, 1,1-dimethylbutylaminocarbonyl, 1,2-dimethylbutylaminocarbonyl, 1,3-dimethylbutylaminocarbonyl, 2,2-dimethylbutylaminocarbonyl, 2,3-dimethylbutylaminocarbonyl, 3,3-dimethylbutylaminocarbonyl, 1-ethylbutylaminocarbonyl, 2-ethylbutylaminocarbonyl, 1,1,2-trimethylpropylaminocarbonyl, 1,2,2-trimethylpropylaminocarbonyl, 1-ethyl-1-methylpropylaminocarbonyl and 1-ethyl-2-methylpropylaminocarbonyl, preferably (C₁-C₄-alkyl)aminocarbonyl, which is substituted by C₁-C₆-alkoxy as mentioned above, i.e., for example, CO—NH—CH₂—OCH₃ or CO—NH—CH₂—OC₂H₅;

C₁-C₆-alkoxyamino-C₁-C₆-alkyl: for example CH₂—NH—OCH₃, CH₂—NH—OC₂H₅, CH₂—NH—OCH₂—C₂H₅, CH₂—NH—OCH(CH₃)₂, CH₂—NH—OCH₂—CH₂—C₂H₅, CH₂—NH—OCH(CH₃)—C₂H₅, CH₂—NH—OCH₂—CH(CH₃)₂, CH₂—NH—OC(CH₃)₃, CH₂—NH—OCH₂—(CH₂)₃—CH₃, (1-methylbutoxyamino)methyl, (2-methylbutoxyamino)methyl, (3-methylbutoxyamino)methyl, (2,2-dimethylpropoxyamino)methyl, (1-ethylpropoxyamino)methyl, n-hexoxyaminomethyl, (1,1-dimethylpropoxyamino)methyl, (1,2-dimethylpropoxyamino)methyl, (1-methylpentoxyamino)methyl, (2-methylpentoxyamino)methyl, (3-methylpentoxyamino)methyl, (4-methylpentoxyamino)methyl, (1,1-dimethylbutoxyamino)methyl, (1,2-dimethylbutoxyamino)methyl, (1,3-dimethylbutoxyamino)methyl, (2,2-dimethylbutoxyamino)methyl, (2,3-dimethylbutoxyamino)methyl, (3,3-dimethylbutoxyamino)methyl, (1-ethylbutoxyamino)methyl, (2-ethylbutoxyamino)methyl, (1,1,2-trimethylpropoxyamino)methyl, (1,2,2-trimethylpropoxyamino)methyl, (1-ethyl-1-methylpropoxyamino)methyl, (1-ethyl-2-methylpropoxyamino)methyl, methoxyaminoethyl, ethoxyaminoethyl, n-propoxyaminoethyl, (1-methylethoxyamino)ethyl, n-butoxyaminoethyl, (1-methylpropoxyamino)ethyl, (2-methylpropoxyamino)ethyl, (1,1-dimethylethoxyamino)ethyl, n-pentoxyaminoethyl, (1-methylbutoxyamino)ethyl, (2-methylbutoxyamino)ethyl, (3-methylbutoxyamino)ethyl, (2,2-dimethylpropoxyamino)ethyl, (1-ethylpropoxyamino)ethyl, n-hexoxyaminoethyl, (1,1-dimethylpropoxyamino)ethyl, (1,2-dimethylpropoxyamino)ethyl, (1-methylpentoxyamino)ethyl, (2-methylpentoxyamino)ethyl, (3-methylpentoxyamino)ethyl, (4-methylpentoxyamino)ethyl, (1,1-dimethylbutoxyamino)ethyl, (1,2-dimethylbutoxyamino)ethyl, (1,3-dimethylbutoxyamino)ethyl, (2,2-dimethylbutoxyamino)ethyl, (2,3-dimethylbutoxyamino)ethyl, (3,3-dimethylbutoxyamino)ethyl, (1-ethylbutoxyamino)ethyl, (2-ethylbutoxyamino)ethyl, (1,1,2-trimethylpropoxyamino)ethyl, (1,2,2-trimethylpropoxyamino)ethyl, (1-ethyl-1-methylpropoxyamino)ethyl, (1-ethyl-2-methylpropoxyamino)ethyl, 2-(methoxyamino)propyl, 3-(methoxyamino)propyl or 2-(ethoxyamino)propyl, preferably C₁-C₆-alkoxyamino-C₁-C₂-alkyl;

C₁-C₆-alkoxy-C₁-C₆-alkylamino-C₁-C₆-alkyl: C₁-C₆-alkylamino-C₁-C₆-alkyl such as CH₂—NH—CH₃, CH₂—NH—C₂H₅, CH₂—NH—CH₂—C₂H₅, CH₂—NH—CH(CH₃) 2, CH₂—NH—(CH₂)₃—CH₃, CH₂—NH—CH(CH₃)—C₂H₅, CH₂—NH—CH₂—CH(CH₃)₂, CH₂—NH—C(CH₃)₃, CH₂—NH—(CH₂)₄—CH₃, (1-methylbutylamino)methyl, (2-methylbutylamino)methyl, (3-methylbutylamino)methyl, (2,2-dimethylpropylamino)methyl, (1-ethylpropylamino)methyl, n-hexylaminomethyl, (1,1-dimethylpropylamino)methyl, (1,2-dimethylpropylamino)methyl, (1-methylpentylamino)methyl, (2-methylpentylamino)methyl, (3-methylpentylamino)methyl, (4-methylpentylamino)methyl, (1,1-dimethylbutylamino)methyl, (1,2-dimethylbutylamino)methyl, (1,3-dimethylbutylamino)methyl, (2,2-dimethylbutylamino)methyl, (2,3-dimethylbutylamino)methyl, (3,3-dimethylbutylamino)methyl, (1-ethylbutylamino)methyl, (2-ethylbutylamino)methyl, (1,1,2-trimethylpropylamino)methyl, (1,2,2-trimethylpropylamino)methyl, (1-ethyl-1-methylpropylamino)methyl, (1-ethyl-2-methylpropylamino)methyl, methylaminoethyl, ethylaminoethyl, n-propylaminoethyl, (1-methylethylamino)ethyl, n-butylaminoethyl, (1-methylpropylamino)ethyl, (2-methylpropylamino)ethyl, (1,1-dimethylethylamino)ethyl, n-pentylaminoethyl, (1-methylbutylamino)ethyl, (2-methylbutylamino)ethyl, (3-methylbutylamino)ethyl, (2,2-dimethylpropylamino)ethyl, (1-ethylpropylamino)ethyl, n-hexylaminoethyl, (1,1-dimethylpropylamino)ethyl, (1,2-dimethylpropylamino)ethyl, (1-methylpentylamino)ethyl, (2-methylpentylamino)ethyl, (3-methylpentylamino)ethyl, (4-methylpentylamino)ethyl, (1,1-dimethylbutylamino)ethyl, (1,2-dimethylbutylamino)ethyl, (1,3-dimethylbutylamino)ethyl, (2,2-dimethylbutylamino)ethyl, (2,3-dimethylbutylamino)ethyl, (3,3-dimethylbutylamino)ethyl, (1-ethylbutylamino)ethyl, (2-ethylbutylamino)ethyl, (1,1,2-trimethylpropylamino)ethyl, (1,2,2-trimethylpropylamino)ethyl, (1-ethyl-1-methylpropylamino)ethyl, (1-ethyl-2-methylpropylamino)ethyl, 2-(methylamino)propyl, 3-(methylamino)propyl and 2-(ethylamino)propyl, preferably C₁-C₆-alkylamino-C₁-C₂-alkyl, which is substituted by C₁-C₆-alkoxy as mentioned above, i.e., for example, CH₂—NH—CH₂—OCH₃ or CH₂—NH—CH₂—OC₂H₅;

C₁-C₆-alkyloximino-C₁-C₆-alkyl: C₁-C₆-alkyl which is substituted by C₁-C₆-alkyloximino such as methoxyimino, ethoxyimino, 1-propoxyimino, 2-propoxyimino, 1-methylethoxyimino, n-butoxyimino, sec-butoxyimino, tert-butoxyimino, 1-methyl-1-propoxyimino, 2-methyl-1-propoxyimino, 1-methyl-2-propoxyimino, 2-methyl-2-propoxyimino, n-pentoxyimino, 2-pentoxyimino, 3-pentoxyimino, 4-pentoxyimino, 1-methyl-1-butoxyimino, 2-methyl-1-butoxyimino, 3-methyl-1-butoxyimino, 1-methyl-2-butoxyimino, 2-methyl-2-butoxyimino, 3-methyl-2-butoxyimino, 1-methyl-3-butoxyimino, 2-methyl-3-butoxyimino, 3-methyl-3-butoxyimino, 1,1-dimethyl-2-propoxyimino, 1,2-dimethyl-1-propoxyimino, 1,2-dimethyl-2-propoxyimino, 1-ethyl-1-propoxyimino, 1-ethyl-2-propoxyimino, n-hexoxyimino, 2-hexoxyimino, 3-hexoxyimino, 4-hexoxyimino, 5-hexoxyimino, 1-methyl-1-pentoxyimino, 2-methyl-1-pentoxyimino, 3-methyl-1-pentoxyimino, 4-methyl-1-pentoxyimino, 1-methyl-2-pentoxyimino, 2-methyl-2-pentoxyimino, 3-methyl-2-pentoxyimino, 4-methyl-2-pentoxyimino, 1-methyl-3-pentoxyimino, 2-methyl-3-pentoxyimino, 3-methyl-3-pentoxyimino, 4-methyl-3-pentoxyimino, 1-methyl-4-pentoxyimino, 2-methyl-4-pentoxyimino, 3-methyl-4-pentoxyimino, 4-methyl-4-pentoxyimino, 1,1-dimethyl-2-butoxyimino, 1,1-dimethyl-3-butoxyimino, 1,2-dimethyl-1-butoxyimino, 1,2-dimethyl-2-butoxyimino, 1,2-dimethyl-3-butoxyimino, 1,3-dimethyl-1-butoxyimino, 1,3-dimethyl-2-butoxyimino, 1,3-dimethyl-3-butoxyimino, 2,2-dimethyl-3-butoxyimino, 2,3-dimethyl-1-butoxyimino, 2,3-dimethyl-2-butoxyimino, 2,3-dimethyl-3-butoxyimino, 3,3-dimethyl-1-butoxyimino, 3,3-dimethyl-2-butoxyimino, 1-ethyl-1-butoxyimino, 1-ethyl-2-butoxyimino, 1-ethyl-3-butoxyimino, 2-ethyl-1-butoxyimino, 2-ethyl-2-butoxyimino, 2-ethyl-3-butoxyimino, 1,1,2-trimethyl-2-propoxyimino, 1-ethyl-1-methyl-2-propoxyimino, 1-ethyl-2-methyl-1-propoxyimino and 1-ethyl-2-methyl-2-propoxyimino, i.e., for example, methoxyiminomethyl;

C₁-C₆-alkoxy-C₁-C₆-alkyl: C₁-C₆-alkyl which is substituted by C₁-C₆-alkoxy as mentioned above, i.e., for example, CH₂—OCH₃, CH₂—OC₂H₅, n-propoxymethyl, CH₂—OCH(CH₃)₂, n-butoxymethyl, (1-methylpropoxy)methyl, (2-methylpropoxy)methyl, CH₂—OC(CH₃)₃, 2-(methoxy)ethyl, 2-(ethoxy)ethyl, 2-(n-propoxy)ethyl, 2-(1-methylethoxy)ethyl, 2-(n-butoxy)ethyl, 2-(1-methylpropoxy)ethyl, 2-(2-methylpropoxy)ethyl, 2-(1,1-dimethylethoxy)ethyl, 2-(methoxy)propyl, 2-(ethoxy)propyl, 2-(n-propoxy)propyl, 2-(1-methylethoxy)propyl, 2-(n-butoxy)propyl, 2-(1-methylpropoxy)propyl, 2-(2-methylpropoxy)propyl, 2-(1,1-dimethylethoxy)propyl, 3-(methoxy)propyl, 3-(ethoxy)propyl, 3-(n-propoxy)propyl, 3-(1-methylethoxy)propyl, 3-(n-butoxy)propyl, 3-(1-methylpropoxy)propyl, 3-(2-methylpropoxy)propyl, 3-(1,1-dimethylethoxy)propyl, 2-(methoxy)butyl, 2-(ethoxy)butyl, 2-(n-propoxy)butyl, 2-(1-methylethoxy)butyl, 2-(n-butoxy)butyl, 2-(1-methylpropoxy)butyl, 2-(2-methylpropoxy)butyl, 2-(1,1-dimethylethoxy)butyl, 3-(methoxy)butyl, 3-(ethoxy)butyl, 3-(n-propoxy)butyl, 3-(1-methylethoxy)butyl, 3-(n-butoxy)butyl, 3-(1-methylpropoxy)butyl, 3-(2-methylpropoxy)butyl, 3-(1,1-dimethylethoxy)butyl, 4-(methoxy)butyl, 4-(ethoxy)butyl, 4-(n-propoxy)butyl, 4-(1-methylethoxy)butyl, 4-(n-butoxy)butyl, 4-(1-methylpropoxy)butyl, 4-(2-methylpropoxy)butyl or 4-(1,1-dimethylethoxy)butyl, in particular CH₂—OCH₃ or 2-methoxyethyl;

di(C₁-C₆-alkoxy)—C₁-C₆-alkyl: for example 2,2-dimethoxyethyl or 2,2-diethoxyethyl;

C₁-C₆-alkoxy-C₁-C₆-alkoxy: C₁-C₆-alkoxy which is substituted by C₁-C₆-alkoxy as mentioned above, i.e., for example, OCH₂—OCH₃, OCH₂—OC₂H₅, n-propoxymethoxy, OCH₂—OCH(CH₃)₂, n-butoxymethoxy, (1-methylpropoxy)methoxy, (2-methylpropoxy)methoxy, OCH₂—OC(CH₃)₃, 2-(methoxy)ethoxy, 2-(ethoxy)ethoxy, 2-(n-propoxy)ethoxy, 2-(1-methylethoxy)ethoxy, 2-(n-butoxy)ethoxy, 2-(1-methylpropoxy)ethoxy, 2-(2-methylpropoxy)ethoxy, 2-(1,1-dimethylethoxy)ethoxy, 2-(methoxy)propoxy, 2-(ethoxy)propoxy, 2-(n-propoxy)propoxy, 2-(1-methylethoxy)propoxy, 2-(n-butoxy)propoxy, 2-(1-methylpropoxy)propoxy, 2-(2-methylpropoxy)propoxy, 2-(1,1-dimethylethoxy)propoxy, 3-(methoxy)propoxy, 3-(ethoxy)propoxy, 3-(n-propoxy)propoxy, 3-(1-methylethoxy)propoxy, 3-(n-butoxy)propoxy, 3-(1-methylpropoxy)propoxy, 3-(2-methylpropoxy)propoxy, 3-(1,1-dimethylethoxy)propoxy, 2-(methoxy)butoxy, 2-(ethoxy)butoxy, 2-(n-propoxy)butoxy, 2-(1-methylethoxy)butoxy, 2-(n-butoxy)butoxy, 2-(1-methylpropoxy)butoxy, 2-(2-methylpropoxy)butoxy, 2-(1,1-dimethylethoxy)butoxy, 3-(methoxy)butoxy, 3-(ethoxy)butoxy, 3-(n-propoxy)butoxy, 3-(1-methylethoxy)butoxy, 3-(n-butoxy)butoxy, 3-(1-methylpropoxy)butoxy, 3-(2-methylpropoxy)butoxy, 3-(1,1-dimethylethoxy)butoxy, 4-(methoxy)butoxy, 4-(ethoxy)butoxy, 4-(n-propoxy)butoxy, 4-(1-methylethoxy)butoxy, 4-(n-butoxy)butoxy, 4-(1-methylpropoxy)butoxy, 4-(2-methylpropoxy)butoxy, 4-(1,1-dimethylethoxy)butoxy, 5-(methoxy)pentoxy, 5-(ethoxy)pentoxy, 5-(n-propoxy)pentoxy, 5-(1-methylethoxy)pentoxy, 5-(n-butoxy)pentoxy, 5-(1-methylpropoxy)pentoxy, 5-(2-methylpropoxy)pentoxy, 5-(1,1-dimethylethoxy)pentoxy, 6-(methoxy)hexoxy, 6-(ethoxy)hexoxy, 6-(n-propoxy)hexoxy, 6-(1-methylethoxy)hexoxy, 6-(n-butoxy)hexoxy, 6-(1-methylpropoxy)hexoxy, 6-(2-methylpropoxy)hexoxy or 6-(1,1-dimethylethoxy)hexoxy, in particular OCH₂—OCH₃ or OCH₂—OC₂H₅;

(C₁-C₆-alkyl)carbonyl-C₁-C₆-alkoxy: C₁-C₆-alkoxy which is substituted by (C₁-C₆-alkyl)carbonyl as mentioned above, i.e., for example, OCH₂—CO—CH₃, OCH₂—CO—C₂H₅, OCH₂—CO—CH₂—C₂H₅, OCH₂—CO—CH(CH₃)₂, n-butylcarbonylmethoxy, 1-(CO—CH₃)ethoxy, 2-(CO—CH₃)ethoxy, 2-(CO—C₂H₅)ethoxy, 2-(CO—CH₂—C₂H₅)ethoxy, 2-(n-butylcarbonyl)ethoxy, 3-(CO—CH₃)propoxy, 3-(CO—C₂H₅)propoxy, 3-(CO—CH₂—C₂H₅)propoxy, 3-(n-butylcarbonyl)propoxy, 4-(CO—CH₃)butoxy, 4-(CO—C₂H₅)butoxy, 4-(CO—CH₂—C₂H₅)butoxy, 4-(n-butylcarbonyl)butoxy, 5—(CO—CH₃)pentoxy, 5—(CO—C₂H₅)pentoxy, 5—(CO—CH₂—C₂H₅)pentoxy, 5-(n-butylcarbonyl)butoxy, 6-(CO—CH₃)hexoxy, 6-(CO—C₂H₅)hexoxy, 6-(CO—CH₂—C₂H₅)hexoxy or 6-(n-butylcarbonyl)hexoxy, in particular OCH₂—OCH₃ or 1-(CO—CH₃)ethoxy;

(C₁-C₆-alkoxy)carbonyl-C₁-C₆-alkoxy: C₁-C₆-alkoxy which is substituted by (C₁-C₆-alkoxy)carbonyl as mentioned above, i.e., for example, OCH₂—CO—OCH₃, OCH₂—CO—OC₂H₅, OCH₂—CO—OCH₂—C₂H₅, OCH₂—CO—OCH(CH₃)₂, n-butoxycarbonylmethoxy, 1-(methoxycarbonyl)ethoxy, 2-(methoxycarbonyl)ethoxy, 2-(ethoxycarbonyl)ethoxy, 2-(n-propoxycarbonyl)ethoxy, 2-(n-butoxycarbonyl)ethoxy, 3-(methoxycarbonyl)propoxy, 3-(ethoxycarbonyl)propoxy, 3-(n-propoxycarbonyl)propoxy, 3-(n-butoxycarbonyl)propoxy, 4-(methoxycarbonyl)butoxy, 4-(ethoxycarbonyl)butoxy, 4-(n-propoxycarbonyl)butoxy, 4-(n-butoxycarbonyl)butoxy, 5-(methoxycarbonyl)pentoxy, 5-(ethoxycarbonyl)pentoxy, 5-(n-propoxycarbonyl)pentoxy, 5-(n-butoxycarbonyl)butoxy, 6-(methoxycarbonyl)hexoxy, 6-(ethoxycarbonyl)hexoxy, 6-(n-propoxycarbonyl)hexoxy or 6-(n-butoxycarbonyl)hexoxy, in particular OCH₂—CO—OCH₃ or 1-(methoxycarbonyl)ethoxy;

(C₁-C₆-alkoxy)carbonyl-C₁-C₆-alkyl: C₁-C₆-alkyl which is substituted by (C₁-C₆-alkoxy)carbonyl as mentioned above, i.e., for example, methoxycarbonylmethyl, ethoxycarbonylmethyl, 1-(methoxycarbonyl)ethyl, 2-(methoxycarbonyl)ethyl, 2-(ethoxycarbonyl)ethyl, 3-(methoxycarbonyl)propyl, 4-(methoxycarbonyl)butyl, 5-(methoxycarbonyl)pentyl or 6-(methoxycarbonyl)hexyl;

(C₁-C₆-alkoxy)carbonyl-C₁-C₆-alkylsulfonyl: C₁-C₆-alkylsulfonyl which is substituted by (C₁-C₆-alkoxy)carbonyl as mentioned above, i.e., for example, methoxycarbonylmethylsulfonyl, ethoxycarbonylmethylsulfonyl, 1-(methoxycarbonyl)ethylsulfonyl, 2-(methoxycarbonyl)ethylsulfonyl, 2-(ethoxycarbonyl)ethylsulfonyl, 3-(methoxycarbonyl)propylsulfonyl, 4-(methoxycarbonyl)butylsulfonyl, 5-(methoxycarbonyl)pentylsulfonyl or 6-(methoxycarbonyl)hexylsulfonyl;

C₁-C₆-alkylthio-C₁-C₆-alkyl: C₁-C₆-alkyl which is substituted by C₁-C₆-alkylthio as mentioned above, i.e., for example, CH₂—SCH₃, CH₂—SC₂H₅, CH₂—SCH₂—C₂H₅, CH₂—SCH(CH₃)₂, n-butylthiomethyl, CH₂—SCH(CH₃)—C₂H₅, CH₂—SCH₂—CH(CH₃)₂, CH₂—SC(CH₃)₃, 2-(SCH₃)ethyl, 2-(SC₂H₅)ethyl, 2-(SCH₂—C₂H₅)ethyl, 2-[SCH(CH₃)₂]ethyl, 2-(n-butylthio)ethyl, 2-[SCH(CH₃)—C₂H₅]ethyl, 2-(2-methylpropylthio)ethyl, 2-[SC(CH₃)₃]ethyl, 2-(SCH₃)propyl, 3-(SCH₃)propyl, 2-(SC₂H₅)propyl, 3-(SC₂H₅)propyl, 3-(SCH₂—C₂H₅)propyl, 3-(butylthio)propyl, 4-(SCH₃)butyl, 4-(SC₂H₅)butyl, 4-(SCH₂—C₂H₅)butyl or 4-(n-butylthio)butyl, in particular 2-(SCH₃)ethyl;

C₁-C₆-alkylthio-C₁-C₆-alkoxy: C₁-C₆-alkoxy which is substituted by C₁-C₆-alkylthio as mentioned above, i.e., for example, OCH₂—SCH₃, OCH₂—SC₂H₅, OCH₂—SCH₂—C₂H₅, OCH₂—SCH(CH₃)₂, n-butylthiomethoxy, OCH₂—SCH(CH₃)—C₂H₅, OCH₂—SCH₂—CH(CH₃)₂, OCH₂—SC(CH₃)₃, 2-(SCH₃)ethoxy, 2-(SC₂H₅)ethoxy, 2-(SCH₂—C₂H₅)ethoxy, 2-[SCH(CH₃)₂]ethoxy, 2-(n-butylthio)ethoxy, 2-[SCH(CH₃)—C₂H₅]ethoxy, 2-(2-methylpropylthio)ethoxy, 2-[SC(CH₃)₃]ethoxy, 2-(SCH₃)propoxy, 3-(SCH₃)propoxy, 2-(SC₂H₅)propoxy, 3-(SC₂H₅)propoxy, 3-(SCH₂—C₂H₅)propoxy, 3-(butylthio)propoxy, 4-(SCH₃)butoxy, 4-(SC₂H₅)butoxy, 4-(CH₂—C₂H₅)butoxy or 4-(n-butylthio)butoxy, in particular 2-(SCH₃)ethoxy;

C₁-C₆-alkylthio-(C₁-C₆-alkyl)carbonyl: (C₁-C₆-alkyl)carbonyl which is substituted by C₁-C₆-alkylthio as mentioned above, preferably by SCH₃ or SC₂H₅, i.e., for example, methylthiomethylcarbonyl, ethylthiomethylcarbonyl, 1-(methylthio)ethylcarbonyl, 2-(methylthio)ethylcarbonyl, 3-(methylthio)propylcarbonyl, 4-(methylthio)butylcarbonyl, 5-(methylthio)pentylcarbonyl or 6-(methylthio)hexylcarbonyl, in particular CO—CH₂—SCH₃ or CO—CH(CH₃)—SCH₃;

di(C₁-C₆-alkyl)amino-C₁-C₆-alkoxy: C₁-C₆-alkoxy which is substituted by di(C₁-C₆-alkyl)amino such as N(CH₃)₂, N(C₂H₅)₂, N,N-dipropylamino, N,N-di-(1-methylethyl)amino, N,N-dibutylamino, N,N-di-(1-methylpropyl)amino, N,N-di-(2-methylpropyl)amino, N[C(CH₃)₃]₂, N-ethyl-N-methylamino, N-methyl-N-propylamino, N-methyl-N-(1-methylethyl)amino, N-butyl-N-methylamino, N-methyl-N-(1-methylpropyl)amino, N-methyl-N-(2-methylpropyl)amino, N-(1,1-dimethylethyl)-N-methylamino, N-ethyl-N-propylamino, N-ethyl-N-(1-methylethyl)amino, N-butyl-N-ethylamino, N-ethyl-N-(1-methylpropyl)amino, N-ethyl-N-(2-methylpropyl)amino, N-ethyl-N-(1,1-dimethylethyl)amino, N-(1-methylethyl)-N-propylamino, N-butyl-N-propylamino, N-(1-methylpropyl)-N-propylamino, N-(2-methylpropyl)-N-propylamino, N-(1,1-dimethylethyl)-N-propylamino, N-butyl-N-(1-methylethyl)amino, N-(1-methylethyl)-N-(1-methylpropyl)amino, N-(1-methylethyl)-N-(2-methylpropyl)amino, N-(1,1-dimethylethyl)-N-(1-methylethyl)amino, N-butyl-N-(1-methylpropyl)amino, N-butyl-N-(2-methylpropyl)amino, N-butyl-N-(1,1-dimethylethyl)amino, N-(1-methylpropyl)-N-(2-methylpropyl)amino, N-(1,1-dimethylethyl)-N-(1-methylpropyl)amino or N-(1,1-dimethylethyl)-N-(2-methylpropyl)amino, preferably N,N-dimethylamino or N,N-diethylamino, i.e., for example, OCH₂—N(CH₃)₂, OCH₂—N(C₂H₅)₂, OCH(CH₃)—N(CH₃)₂, 2-(dimethylamino)ethoxy, OCH(CH₃)—N(C₂H₅)₂, 3-(dimethylamino)propoxy, 4-(dimethylamino)butoxy, 5-(dimethylamino)pentoxy or 6-(dimethylamino)hexoxy, in particular OCH₂—N(CH₃)₂ or OCH(CH₃)—N(CH₃)₂;

C₃-C₆-alkenyl: for example prop-2-en-1-yl, n-buten-4-yl, 1-methylprop-2-en-1-yl, 2-methylprop-2-en-1-yl, 2-buten-1-yl, n-penten-3-yl, n-penten-4-yl, 1-methylbut-2-en-1-yl, 2-methylbut-2-en-1-yl, 3-methylbut-2-en-1-yl, 1-methylbut-3-en-1-yl, 2-methylbut-3-en-1-yl, 3-methylbut-3-en-1-yl, 1,1-dimethylprop-2-en-1-yl, 1,2-dimethylprop-2-en-1-yl, 1-ethylprop-2-en-1-yl, n-hex-3-en-1-yl, n-hex-4-en-1-yl, n-hex-5-en-1-yl, 1-methylpent-3-en-1-yl, 2-methylpent-3-en-1-yl, 3-methylpent-3-en-1-yl, 4-methylpent-3-en-1-yl, 1-methylpent-4-en-1-yl, 2-methylpent-4-en-1-yl, 3-methylpent-4-en-1-yl, 4-methylpent-4-en-1-yl, 1,1-dimethylbut-2-en-1-yl, 1,1-dimethylbut-3-en-1-yl, 1,2-dimethylbut-2-en-1-yl, 1,2-dimethylbut-3-en-1-yl, 1,3-dimethylbut-2-en-1-yl, 1,3-dimethylbut-3-en-1-yl, 2,2-dimethylbut-3-en-1-yl, 2,3-dimethylbut-2-en-1-yl, 2,3-dimethylbut-3-en-1-yl, 3,3-dimethylbut-2-en-1-yl, 1-ethylbut-2-en-1-yl, 1-ethylbut-3-en-1-yl, 2-ethylbut-2-en-1-yl, 2-ethylbut-3-en-1-yl, 1,1,2-trimethylprop-2-en-1-yl, 1-ethyl-1-methylprop-2-en-1-yl or 1-ethyl-2-methylprop-2-en-1-yl, in particular prop-2-en-1-yl or n-buten-4-yl;

C₃-C₆-haloalkenyl: C₃-C₆-alkenyl as mentioned above which is partially or fully substituted by fluorine, chlorine and/or bromine, i.e., for example, 2-chloroallyl, 3-chloroallyl, 2,3-dichloroallyl, 3,3-dichloroallyl, 2,3,3-trichloroallyl, 2,3-dichlorobut-2-enyl, 2-bromoallyl, 3-bromoallyl, 2,3-dibromoallyl, 3,3-dibromoallyl, 2,3,3-tribromoallyl or 2,3-dibromobut-2-enyl, in particular 2-chloroallyl or 3,3-dichloroallyl; C₂-C₆-alkenyl: ethenyl or one of the radicals mentioned under C₃-C₆-alkenyl, in particular ethenyl or prop-2-en-1-yl;

C₃-C₆-alkenyloxy: prop-1-en-1-yloxy, prop-2-en-1-yloxy, 1-methylethenyloxy, n-buten-1-yloxy, n-buten-2-yloxy, n-buten-3-yloxy, 1-methylprop-1-en-1-yloxy, 2-methylprop-1-en-1-yloxy, 1-methylprop-2-en-1-yloxy, 2-methylprop-2-en-1-yloxy, n-penten-1-yloxy, n-penten-2-yloxy, n-penten-3-yloxy, n-penten-4-yloxy, 1-methylbut-1-en-1-yloxy, 2-methylbut-1-en-1-yloxy, 3-methylbut-1-en-1-yloxy, 1-methylbut-2-en-1-yloxy, 2-methylbut-2-en-1-yloxy, 3-methylbut-2-en-1-yloxy, 1-methylbut-3-en-1-yloxy, 2-methylbut-3-en-1-yloxy, 3-methylbut-3-en-1-yloxy, 1,1-dimethylprop-2-en-1-yloxy, 1,2-dimethylprop-1-en-1-yloxy, 1,2-dimethylprop-2-en-1-yloxy, 1-ethylprop-1-en-2-yloxy, 1-ethylprop-2-en-1-yloxy, n-hex-1-en-1-yloxy, n-hex-2-en-1-yloxy, n-hex-3-en-1-yloxy, n-hex-4-en-1-yloxy, n-hex-5-en-1-yloxy, 1-methylpent-1-en-1-yloxy, 2-methylpent-1-en-1-yloxy, 3-methylpent-1-en-1-yloxy, 4-methylpent-1-en-1-yloxy, 1-methylpent-2-en-1-yloxy, 2-methylpent-2-en-1-yloxy, 3-methylpent-2-en-1-yloxy, 4-methylpent-2-en-1-yloxy, 1-methylpent-3-en-1-yloxy, 2-methylpent-3-en-1-yloxy, 3-methylpent-3-en-1-yloxy, 4-methylpent-3-en-1-yloxy, 1-methylpent-4-en-1-yloxy, 2-methylpent-4-en-1-yloxy, 3-methylpent-4-en-1-yloxy, 4-methylpent-4-en-1-yloxy, 1,1-dimethylbut-2-en-1-yloxy, 1,1-dimethylbut-3-en-1-yloxy, 1,2-dimethylbut-1-en-1-yloxy, 1,2-dimethylbut-2-en-1-yloxy, 1,2-dimethylbut-3-en-1-yloxy, 1,3-dimethylbut-1-en-1-yloxy, 1,3-dimethylbut-2-en-1-yloxy, 1,3-dimethylbut-3-en-1-yloxy, 2,2-dimethylbut-3-en-1-yloxy, 2,3-dimethylbut-1-en-1-yloxy, 2,3-dimethylbut-2-en-1-yloxy, 2,3-dimethylbut-3-en-1-yloxy, 3,3-dimethylbut-1-en-1-yloxy, 3,3-dimethylbut-2-en-1-yloxy, 1-ethylbut-1-en-1-yloxy, 1-ethylbut-2-en-1-yloxy, 1-ethylbut-3-en-1-yloxy, 2-ethylbut-1-en-1-yloxy, 2-ethylbut-2-en-1-yloxy, 2-ethylbut-3-en-1-yloxy, 1,1,2-trimethylprop-2-en-1-yloxy, 1-ethyl-1-methylprop-2-en-1-yloxy, 1-ethyl-2-methylprop-1-en-1-yloxy or 1-ethyl-2-methylprop-2-en-1-yloxy, in particular prop-2-en-1-yloxy;

C₂-C₆-alkenyloxy: ethenyloxy or one of the radicals mentioned under C₃-C₆-alkenyloxy, in particular ethenyloxy or prop-2-en-1-yloxy;

C₃-C₆-haloalkenyloxy: C₃-C₆-alkenyloxy as mentioned above which is partially or fully substituted by fluorine, chlorine and/or bromine, i.e., for example, 2-chloroallyloxy, 3-chloroallyloxy, 2,3-dichloroallyloxy, 3,3-dichloroallyloxy, 2,3,3-trichloroallyloxy, 2,3-dichlorobut-2-enyloxy, 2-bromoallyloxy, 3-bromoallyloxy, 2,3-dibromoallyloxy, 3,3-dibromoallyloxy, 2,3,3-tribromoallyloxy or 2,3-dibromobut-2-enyloxy, in particular 2-chloroallyloxy or 3,3-dichloroallyloxy;

phenyl-C₃-C₆-alkenyloxy: for example 3-phenylallyloxy, 4-phenylbut-2-enyloxy, 4-phenylbut-3-enyloxy or 5-phenylpent-4-enyloxy, preferably 3-phenylallyloxy or 4-phenylbut-2-enyloxy, in particular 3-phenylallyloxy;

heterocyclyl-C₃-C₆-alkenyloxy: for example 3-heterocyclylallyloxy, 4-heterocyclylbut-2-enyloxy, 4-heterocyclylbut-3-enyloxy or 5-heterocyclylpent-4-enyloxy, preferably 3-heterocyclylallyloxy or 4-heterocyclylbut-2-enyloxy, in particular 3-heterocyclylallyloxy;

C₂-C₆-alkenylthio: ethenylthio, prop-1-en-1-ylthio, prop-2-en-1-ylthio, 1-methylethenylthio, n-buten-1-ylthio, n-buten-2-ylthio, n-buten-3-ylthio, 1-methyl-prop-1-en-1-ylthio, 2-methylprop-1-en-1-ylthio, 1-methylprop-2-en-1-ylthio, 2-methylprop-2-en-1-ylthio, n-penten-1-ylthio, n-penten-2-ylthio, n-penten-3-ylthio, 4-methylpent-1-yn-1-yl, 4-methylpent-2-yn-4-yl or 4-methylpent-2-yn-5-yl, in particular prop-2-yn-1-yl;

C₂-C₆-alkynyl: ethynyl or one of the radicals mentioned under C₃-C₆-alkynyl, in particular ethynyl or prop-2-yn-1-yl;

C₃-C₆-alkynyloxy: prop-1-yn-1-yloxy, prop-2-yn-1-yloxy, n-but-1-yn-1-yloxy, n-but-1-yn-3-yloxy, n-but-1-yn-4-yloxy, n-but-2-yn-1-yloxy, n-pent-1-yn-1-yloxy, n-pent-1-yn-3-yloxy, n-pent-1-yn-4-yloxy, n-pent-1-yn-5-yloxy, n-pent-2-yn-1-yloxy, n-pent-2-yn-4-yloxy, n-pent-2-yn-5-yloxy, 3-methylbut-1-yn-3-yloxy, 3-methylbut-1-yn-4-yloxy, n-hex-1-yn-1-yloxy, n-hex-1-yn-3-yloxy, n-hex-1-yn-4-yloxy, n-hex-1-yn-5-yloxy, n-hex-1-yn-6-yloxy, n-hex-2-yn-1-yloxy, n-hex-2-yn-4-yloxy, n-hex-2-yn-5-yloxy, n-hex-2-yn-6-yloxy, n-hex-3-yn-1-yloxy, n-hex-3-yn-2-yloxy, 3-methylpent-1-yn-1-yloxy, 3-methylpent-1-yn-3-yloxy, 3-methylpent-1-yn-4-yloxy, 3-methylpent-1-yn-5-yloxy, 4-methylpent-1-yn-1-yloxy, 4-methylpent-2-yn-4-yloxy or 4-methylpent-2-yn-5-yloxy, in particular prop-2-yn-1-yloxy;

C₂-C₆-alkynyloxy: ethynyloxy or one of the radicals mentioned under C₃-C₆-alkynyloxy, in particular ethynyloxy or prop-2-yn-1-yloxy;

phenyl-C₃-C₆-alkynyloxy: for example 3-phenylprop-2-yn-1-yloxy, 4-phenylbut-2-yn-1-yloxy, 3-phenylbut-3-yn-2-yloxy, 5-phenylpent-3-yn-1-yloxy or 6-phenylhex-4-yn-1-yloxy, in particular 3-phenylprop-2-yn-1-yloxy or 3-phenylbut-3-yn-2-yloxy;

heterocyclyl-C₃-C₆-alkynyloxy: for example 3-(heterocyclyl)prop-2-yn-1-yloxy, 4-(heterocyclyl)but-2-yn-1-yloxy, 3-(heterocyclyl)but-3-yn-2-yloxy, 5-(heterocyclyl)pent-3-yn-1-yloxy or 6-(heterocyclyl)hex-4-yn-1-yloxy, in particular 3-(heterocyclyl)prop-2-yn-1-yloxy or 3-(heterocyclyl)but-3-yn-2-yloxy;

C₃-C₆-alkynylthio: prop-1-yn-1-ylthio, prop-2-yn-1-ylthio, n-but-1-yn-1-ylthio, n-but-1-yn-3-ylthio, n-but-1-yn-4-ylthio, n-but-2-yn-1-ylthio, n-pent-1-yn-1-ylthio, n-pent-1-yn-3-ylthio, n-pent-1-yn-4-ylthio, n-pent-1-yn-5-ylthio, n-pent-2-yn-1-ylthio, n-pent-2-yn-4-ylthio, n-pent-2-yn-5-ylthio, 3-methylbut-1-yn-3-ylthio, 3-methylbut-1-yn-4-ylthio, n-hex-1-yn-1-ylthio, n-hex-1-yn-3-ylthio, n-hex-1-yn-4-ylthio, n-hex-1-yn-5-ylthio, n-hex-1-yn-6-ylthio, n-hex-2-yn-1-ylthio, n-hex-2-yn-4-ylthio, n-hex-2-yn-5-ylthio, n-hex-2-yn-6-ylthio, n-hex-3-yn-1-ylthio, n-hex-3-yn-2-ylthio, 3-methylpent-1-yn-1-ylthio, 3-methylpent-1-yn-3-ylthio, 3-methylpent-1-yn-4-ylthio, 3-methylpent-1-yn-5-ylthio, 4-methylpent-1-yn-1-ylthio, 4-methylpent-2-yn-4-ylthio or 4-methylpent-2-yn-5-ylthio, in particular prop-2-yn-1-ylthio;

C₂-C₆-alkynylthio: ethynylthio or one of the radicals mentioned under C₃-C₆-alkynylthio, in particular ethynylthio or prop-2-yn-1-ylthio;

(C₃-C₆-alkenyloxy)carbonyl: prop-1-en-1-yloxycarbonyl, prop-2-en-1-yloxycarbonyl, 1-methylethenyloxycarbonyl, n-buten-1-yloxycarbonyl, n-buten-2-yloxycarbonyl, n-buten-3-yloxycarbonyl, 1-methylprop-1-en-1-yloxycarbonyl, 2-methylprop-1-en-1-yloxycarbonyl, 1-methylprop-2-en-1-yloxycarbonyl, 2-methylprop-2-en-1-yloxycarbonyl, n-penten-1-yloxycarbonyl, n-penten-2-yloxycarbonyl, n-penten-3-yloxycarbonyl, n-penten-4-yloxycarbonyl, 1-methylbut-1-en-1-yloxycarbonyl, 2-methylbut-1-en-1-yloxycarbonyl, 3-methylbut-1-en-1-yloxycarbonyl, 1-methylbut-2-en-1-yloxycarbonyl, 2-methylbut-2-en-1-yloxycarbonyl, 3-methylbut-2-en-1-yloxycarbonyl, 1-methylbut-3-en-1-yloxycarbonyl, 2-methylbut-3-en-1-yloxycarbonyl, 3-methylbut-3-en-1-yloxycarbonyl, 1,1-dimethylprop-2-en-1-yloxycarbonyl, 1,2-dimethylprop-1-en-1-yloxycarbonyl, 1,2-dimethylprop-2-en-1-yloxycarbonyl, 1-ethylprop-1-en-2-yloxycarbonyl, 1-ethylprop-2-en-1-yloxycarbonyl, n-hex-1-en-1-yloxycarbonyl, n-hex-2-en-1-yloxycarbonyl, n-hex-3-en-1-yloxycarbonyl, n-hex-4-en-1-yloxycarbonyl, n-hex-5-en-1-yloxycarbonyl, 1-methylpent-1-en-1-yloxycarbonyl, 2-methylpent-1-en-1-yloxycarbonyl, 3-methylpent-1-en-1-yloxycarbonyl, 4-methylpent-1-en-1-yloxycarbonyl, 1-methylpent-2-en-1-yloxycarbonyl, 2-methylpent-2-en-1-yloxycarbonyl, 3-methylpent-2-en-1-yloxycarbonyl, 4-methylpent-2-en-1-yloxycarbonyl, 1-methylpent-3-en-1-yloxycarbonyl, 2-methylpent-3-en-1-yloxycarbonyl, 3-methylpent-3-en-1-yloxycarbonyl, 4-methylpent-3-en-1-yloxycarbonyl, 1-methylpent-4-en-1-yloxycarbonyl, 2-methylpent-4-en-1-yloxycarbonyl, 3-methylpent-4-en-1-yloxycarbonyl, 4-methylpent-4-en-1-yloxycarbonyl, 1,1-dimethylbut-2-en-1-yloxycarbonyl, 1,1-dimethylbut-3-en-1-yloxycarbonyl, 1,2-dimethylbut-1-en-1-yloxycarbonyl, 1,2-dimethylbut-2-en-1-yloxycarbonyl, 1,2-dimethylbut-3-en-1-yloxycarbonyl, 1,3-dimethylbut-1-en-1-yloxycarbonyl, 1,3-dimethylbut-2-en-1-yloxycarbonyl, 1,3-dimethylbut-3-en-1-yloxycarbonyl, 2,2-dimethylbut-3-en-1-yloxycarbonyl, 2,3-dimethylbut-1-en-1-yloxycarbonyl, 2,3-dimethylbut-2-en-1-yloxycarbonyl, 2,3-dimethylbut-3-en-1-yloxycarbonyl, 3,3-dimethylbut-1-en-1-yloxycarbonyl, 3,3-dimethylbut-2-en-1-yloxycarbonyl, 1-ethylbut-1-en-1-yloxycarbonyl, 1-ethylbut-2-en-1-yloxycarbonyl, 1-ethylbut-3-en-1-yloxycarbonyl, 2-ethylbut-1-en-1-yloxycarbonyl, 2-ethylbut-2-en-1-yloxycarbonyl, 2-ethylbut-3-en-1-yloxycarbonyl, 1,1,2-trimethylprop-2-en-1-yloxycarbonyl, 1-ethyl-1-methylprop-2-en-1-yloxycarbonyl, 1-ethyl-2-methylprop-1-en-1-yloxycarbonyl or 1-ethyl-2-methylprop-2-en-1-yloxycarbonyl, in particular prop-2-en-1-yloxycarbonyl;

(C₃-C₆-alkenyloxy)carbonyl-C₁-C₆-alkyl: C₁-C₆-alkyl which is substituted by (C₃-C₆-alkenyloxy)carbonyl as mentioned above, preferably by prop-2-en-1-yloxycarbonyl, i.e., for example, prop-2-en-1-yloxycarbonylmethyl;

(C₂-C₆-alkenyl)carbonyloxy: ethenylcarbonyloxy, prop-1-en-1-ylcarbonyloxy, prop-2-en-1-ylcarbonyloxy, 1-methylethenylcarbonyloxy, n-buten-1-ylcarbonyloxy, n-buten-2-ylcarbonyloxy, n-buten-3-ylcarbonyloxy, 1-methylprop-1-en-1-ylcarbonyloxy, 2-methylprop-1-en-1-ylcarbonyloxy, 1-methylprop-2-en-1-ylcarbonyloxy, 2-methylprop-2-en-1-ylcarbonyloxy, n-penten-1-ylcarbonyloxy, n-penten-2-ylcarbonyloxy, n-penten-3-ylcarbonyloxy, n-penten-4-ylcarbonyloxy, 1-methylbut-1-en-1-ylcarbonyloxy, 2-methylbut-1-en-1-ylcarbonyloxy, 3-methylbut1-en-1-ylcarbonyloxy, 1-methylbut-2-en-1-ylcarbonyloxy, 2-methylbut-2-en-1-ylcarbonyloxy, 3-methylbut-3-en-1-ylcarbonyloxy, 1-methylbut-3-en-1-ylcarbonyloxy, 2-methylbut-3-en-1-ylcarbonyloxy, 3-methylbut-3-en-1-ylcarbonyloxy, 1,1-dimethylprop-2-en-1-ylcarbonyloxy, 1,2-dimethylprop-1-en-1-ylcarbonyloxy, 1,2-dimethylprop-2-en-1-ylcarbonyloxy, 1-ethylprop-1-en-2-ylcarbonyloxy, 1-ethylprop-2-en-1-ylcarbonyloxy, n-hex-1-en-1-ylcarbonyloxy, n-hex-2-en-1-ylcarbonyloxy, n-hex-3-en-1-ylcarbonyloxy, n-hex-4-en-1-ylcarbonyloxy, n-hex-5-en-1-ylcarbonyloxy, 1-methylpent-1-en-1-ylcarbonyloxy, 2-methylpent-1-en-1-ylcarbonyloxy, 3-methylpent-1-en-1-ylcarbonyloxy, 4-methylpent-1-en-1-ylcarbonyloxy, 1-methylpent-2-en-1-ylcarbonyloxy, 2-methylpent-2-en-1-ylcarbonyloxy, 3-methylpent-2-en-1-ylcarbonyloxy, 4-methylpent-2-en-1-ylcarbonyloxy, 1-methylpent-3-en-1-ylcarbonyloxy, 2-methylpent-3-en-1-ylcarbonyloxy, 3-methylpent-3-en-1-ylcarbonyloxy, 4-methylpent-3-en-1-ylcarbonyloxy, 1-methylpent-4-en-1-ylcarbonyloxy, 2-methylpent-4-en-1-ylcarbonyloxy, 3-methylpent-4-en-1-ylcarbonyloxy, 4-methylpent-4-en-1-ylcarbonyloxy, 1,1-dimethylbut-2-en-1-ylcarbonyloxy, 1,1-dimethylbut-3-en-1-ylcarbonyloxy, 1,2-dimethylbut-1-en-1-ylcarbonyloxy, 1,2-dimethylbut-2-en-1-ylcarbonyloxy, 1,2-dimethylbut-3-en-1-ylcarbonyloxy, 1,3-dimethylbut-1-en-1-ylcarbonyloxy, 1,3-dimethylbut-2-en-1-ylcarbonyloxy, 1,3-dimethylbut-3-en-1-ylcarbonyloxy, 2,2-dimethylbut-3-en-1-ylcarbonyloxy, 2,3-dimethylbut-1-en-1-ylcarbonyloxy, 2,3-dimethylbut-2-en-1-ylcarbonyloxy, 2,3-dimethylbut-3-en-1-ylcarbonyloxy, 3,3-dimethylbut-1-en-1-ylcarbonyloxy, 3,3-dimethylbut-2-en-1-ylcarbonyloxy, 1-ethylbut-1-en-1-ylcarbonyloxy, 1-ethylbut-2-en-1-ylcarbonyloxy, 1-ethylbut-3-en-1-ylcarbonyloxy, 2-ethylbut-1-en-1-ylcarbonyloxy, 2-ethylbut-2-en-1-ylcarbonyloxy, 2-ethylbut-3-en-1-ylcarbonyloxy, 1,1,2-trimethylprop-2-en-1-ylcarbonyloxy, 1-ethyl-1-methylprop-2-en-1-ylcarbonyloxy, 1-ethyl-2-methylprop-1-en-1-ylcarbonyloxy or 1-ethyl-2-methylprop-2-en-1-ylcarbonyloxy, in particular ethenylcarbonyloxy or prop-2-en-1-ylcarbonyloxy;

(C₂-C₆-alkenyl)carbonylthio: ethenylcarbonylthio, prop-1-en-1-ylcarbonylthio, prop-2-en-1-ylcarbonylthio, 1-methylethenylcarbonylthio, n-buten-1-ylcarbonylthio, n-buten-2-ylcarbonylthio, n-buten-3-ylcarbonylthio, 1-methylprop-1-en-1-ylcarbonylthio, 2-methylprop-1-en-1-ylcarbonylthio, 1-methylprop-2-en-1-ylcarbonylthio, 2-methylprop-2-en-1-ylcarbonylthio, n-penten-1-ylcarbonylthio, n-penten-2-ylcarbonylthio, n-penten-3-ylcarbonylthio, n-penten-4-ylcarbonylthio, 1-methylbut-1-en-1-ylcarbonylthio, 2-methylbut-1-en-1-ylcarbonylthio, 3-methylbut-1-en-1-ylcarbonylthio, 1-methylbut-2-en-1-ylcarbonylthio, 2-methylbut-2-en-1-ylcarbonylthio, 3-methylbut-2-en-1-ylcarbonylthio, 1-methylbut-3-en-1-ylcarbonylthio, 2-methylbut-3-en-1-ylcarbonylthio, 3-methylbut-3-en-1-ylcarbonylthio, 1,1-dimethylprop-2-en-1-ylcarbonylthio, 1,2-dimethylprop-1-en-1-ylcarbonylthio, 1,2-dimethylprop-2-en-1-ylcarbonylthio, 1-ethylprop-1-en-2-ylcarbonylthio, 1-ethylprop-2-en-1-ylcarbonylthio, n-hex-1-en-1-ylcarbonylthio, n-hex-2-en-1-ylcarbonylthio, n-hex-3-en-1-ylcarbonylthio, n-hex-4-en-1-ylcarbonylthio, n-hex-5-en-1-ylcarbonylthio, 1-methylpent-1-en-1-ylcarbonylthio, 2-methylpent-1-en-1-ylcarbonylthio, 3-methylpent-1-en-1-ylcarbonylthio, 4-methylpent-1-en-1-ylcarbonylthio, 1-methylpent-2-en-1-ylcarbonylthio, 2-methylpent-2-en-1-ylcarbonylthio, 3-methylpent-2-en-1-ylcarbonylthio, 4-methylpent-2-en-1-ylcarbonylthio, 1-methylpent-3-en-1-ylcarbonylthio, 2-methylpent-3-en-1-ylcarbonylthio, 3-methylpent-3-en-1-ylcarbonylthio, 4-methylpent-3-en-1-ylcarbonylthio, 1-methylpent-4-en-1-ylcarbonylthio, 2-methylpent-4-en-1-ylcarbonylthio, 3-methylpent-4-en-1-ylcarbonylthio, 4-methylpent-4-en-1-ylcarbonylthio, 1,1-dimethylbut-2-en-1-ylcarbonylthio, 1,1-dimethylbut-3-en-1-ylcarbonylthio, 1,2-dimethylbut-1-en-1-ylcarbonylthio, 1,2-dimethylbut-2-en-1-ylcarbonylthio, 1,2-dimethylbut-3-en-1-ylcarbonylthio, 1,3-dimethylbut-1-en-1-ylcarbonylthio, 1,3-dimethylbut-2-en-1-ylcarbonylthio, 1,3-dimethylbut-3-en-1-ylcarbonylthio, 2,2-dimethylbut-3-en-1-ylcarbonylthio, 2,3-dimethylbut-1-en-1-ylcarbonylthio, 2,3-dimethylbut-2-en-1-ylcarbonylthio, 2,3-dimethylbut-3-en-1-ylcarbonylthio, 3,3-dimethylbut-1-en-1-ylcarbonylthio, 3,3-dimethylbut-2-en-1-ylcarbonylthio, 1-ethylbut-1-en-1-ylcarbonylthio, 1-ethylbut-2-en-1-ylcarbonylthio, 1-ethylbut-3-en-1-ylcarbonylthio, 2-ethylbut-1-en-1-ylcarbonylthio, 2-ethylbut-2-en-1-ylcarbonylthio, 2-ethylbut-3-en-1-ylcarbonylthio, 1,1,2-trimethylprop-2-en-1-ylcarbonylthio, 1-ethyl-1-methylprop-2-en-1-ylcarbonylthio, 1-ethyl-2-methylprop-1-en-1-ylcarbonylthio or 1-ethyl-2-methylprop-2-en-1-ylcarbonylthio, in particular ethenylcarbonylthio or prop-2-en-1-yl-carbonylthio;

(C₂-C₆-alkynyl)carbonyloxy: ethynylcarbonyloxy, prop-1-yn-1-ylcarbonyloxy, prop-2-yn-1-ylcarbonyloxy, n-but-1-yn-1-ylcarbonyloxy, n-but-1-yn-3-ylcarbonyloxy, n-but-1-yn-4-ylcarbonyloxy, n-but-2-yn-1-ylcarbonyloxy, n-pent-1-yn-1-ylcarbonyloxy, n-pent-1-yn-3-ylcarbonyloxy, n-pent-1-yn-4-ylcarbonyloxy, n-pent-1-yn-5-ylcarbonyloxy, n-pent-2-yn-1-ylcarbonyloxy, n-pent-2-yn-4-ylcarbonyloxy, n-pent-2-yn-5-ylcarbonyloxy, 3-methylbut-1-yn-3-ylcarbonyloxy, 3-methylbut-1-yn-4-ylcarbonyloxy, n-hex-1-yn-1-ylcarbonyloxy, n-hex-1-yn-3-ylcarbonyloxy, n-hex-1-yn-4-ylcarbonyloxy, n-hex-1-yn-5-ylcarbonyloxy, n-hex-1-yn-6-ylcarbonyloxy, n-hex-2-yn-1-ylcarbonyloxy, n-hex-2-yn-4-ylcarbonyloxy, n-hex-2-yn-5-ylcarbonyloxy, n-hex-2-yn-6-ylcarbonyloxy, n-hex-3-yn-1-ylcarbonyloxy, n-hex-3-yn-2-ylcarbonyloxy, 3-methylpent-1-yn-1-ylcarbonyloxy, 3-methylpent-1-yn-3-ylcarbonyloxy, 3-methylpent-1-yn-4-ylcarbonyloxy, 3-methylpent-1-yn-5-ylcarbonyloxy, 4-methylpent-1-yn-1-ylcarbonyloxy, 4-methylpent-2-yn-4-ylcarbonyloxy or 4-methylpent-2-yn-5-ylcarbonyloxy, in particular ethynylcarbonyloxy or prop-2-yn-1-ylcarbonyloxy;

C₃-₆-alkynylsulfonyloxy: prop-1-yn-1-ylsulfonyloxy, prop-2-yn-1-ylsulfonyloxy, n-but-1-yn-1-ylsulfonyloxy, n-but-1-yn-3-ylsulfonyloxy, n-but-1-yn-4-ylsulfonyloxy, n-but-2-yn-1-ylsulfonyloxy, n-pent-1-yn-1-ylsulfonyloxy, n-pent-1-yn-3-ylsulfonyloxy, n-pent-1-yn-4-ylsulfonyloxy, n-pent-1-yn-5-ylsulfonyloxy, n-pent-2-yn-1-ylsulfonyloxy, n-pent-2-yn-4-ylsulfonyloxy, n-pent-2-yn-5-ylsulfonyloxy, 3-methylbut-1-yn-3-ylsulfonyloxy, 3-methylbut-1-yn-4-ylsulfonyloxy, n-hex-1-yn-1-ylsulfonyloxy, n-hex-1-yn-3-ylsulfonyloxy, n-hex-1-yn-4-ylsulfonyloxy, n-hex-1-yn-5-ylsulfonyloxy, n-hex-1-yn-6-ylsulfonyloxy, n-hex-2-yn-1-ylsulfonyloxy, n-hex-1-2-yn-4-ylsulfonyloxy, n-hex-2-yn-5-ylsulfonyloxy, n-hex-2-yn-6-ylsulfonyloxy, n-hex-3-yn-1-ylsulfonyloxy, n-hex-3-yn-2-ylsulfonyloxy, 3-methylpent-1-yn-1-ylsulfonyloxy, 3-methylpent-1-yn-3-ylsulfonyloxy, 3-methylpent-1-yn-4-ylsulfonyloxy, 3-methylpent-1-yn-5-ylsulfonyloxy, 4-methylpent-1-yn-1-ylsulfonyloxy, 4-methylpent-2-yn-4-ylsulfonyloxy or 4-methylpent-2-yn-5-ylsulfonyloxy, in particular prop-2-yn-1-ylsulfonyloxy;

(C₂-C₆-alkynyl)carbonylthio: ethynylcarbonylthio, prop-1-yn-1-ylcarbonylthio, prop-2-yn-1-ylcarbonylthio, n-but-1-yn-1-ylcarbonylthio, n-but-1-yn-3-ylcarbonylthio, n-but-1-yn-4-ylcarbonylthio, n-but-2-yn-1-ylcarbonylthio, n-pent-1-yn-1-ylcarbonylthio, n-pent-1-yn-3-ylcarbonylthio, n-pent-1-yn-4-ylcarbonylthio, n-pent-1-yn-5-ylcarbonylthio, n-pent-2-yn-1-ylcarbonylthio, n-pent-2-yn-4-ylcarbonylthio, n-pent-2-yn-5-ylcarbonylthio, 3-methylbut-1-yn-3-ylcarbonylthio, 3-methylbut-1-yn-4-ylcarbonylthio, n-hex-1-yn-1-ylcarbonylthio, n-hex-1-yn-3-ylcarbonylthio, n-hex-1-yn-4-ylcarbonylthio, n-hex-1-yn-5-ylcarbonylthio, n-hex-1-yn-6-ylcarbonylthio, n-hex-2-yn-1-ylcarbonylthio, n-hex-2-yn-4-ylcarbonylthio, n-hex-2-yn-5-ylcarbonylthio, n-hex-2-yn-6-ylcarbonylthio, n-hex-3-yn-1-ylcarbonylthio, n-hex-3-yn-2-ylcarbonylthio, 3-methylpent-1-yn-1-ylcarbonylthio, 3-methylpent-1-yn-3-ylcarbonylthio, 3-methylpent-1-yn-4-ylcarbonylthio, 3-methylpent-1-yn-5-ylcarbonylthio, 4-methylpent-1-yn-1-ylcarbonylthio, 4-methylpent-2-yn-4-ylcarbonylthio or 4-methylpent-2-yn-5-ylcarbonylthio, in particular prop-2-yn-1-ylcarbonylthio;

(C₁-C₆-alkoxy)carbonyl-C₂-C₆-alkenyl: C₂-C₆-alkenyl which is substituted by (C₁-C₆-alkoxy)carbonyl as mentioned above, i.e., for example, methoxycarbonylprop-2-en-1-yl;

(C₁-C₆-alkoxy)carbonyl-C₂-C₆-alkenyloxy: C₂-C₆-alkenyloxy which is substituted by (C₁-C₆-alkoxy)carbonyl as mentioned above, i.e., for example, 1-methoxycarbonylethen-1-yloxy and methoxycarbonylprop-2-en-1-yloxy;

C₁-C₆-alkoxy-C₃-C₆-alkenyloxy: C₃-C₆-alkenyloxy which is substituted by C₁-C₆-alkoxy as mentioned above, i.e., for example, methylprop-2-en-1-yloxy;

C₃-C₆-alkenyloxy-C₁-C₆-alkyl: C₁-C₆-alkyl which is substituted by C₃-C₆-alkenyloxy as mentioned above, preferably by allyloxy, 2-methylprop-2-en-1-yloxy, but-1-en-3-yloxy, but-1-en-4-yloxy or but-2-en-1-yloxy, i.e., for example, allyloxymethyl, 2-allyloxyethyl or but-1-en-4-yloxymethyl;

C₃-C₆-alkynyloxy-C₁-C₆-alkyl: C₁-C₆-alkyl which is substituted by C₃-C₆-alkynyloxy as mentioned above, preferably by propargyloxy, but-1-yn-3-yloxy, but-1-yn-4-yloxy or but-2-yn-1-yloxy, i.e., for example, propargyloxymethyl or 2-propargyloxyethyl;

C₃-C₆-cycloalkyl: cyclopropyl, cyclobutyl, cyclopentyl or cyclohexyl;

C₃-C₆-cycloalkyl-C₁-C₆-alkoxy: for example cyclopropylmethoxy, cyclobutylmethoxy, cyclopentylmethoxy, cyclohexylmethoxy, 1-(cyclopropyl)ethoxy, 1-(cyclobutyl)ethoxy, 1-(cyclopentyl)ethoxy, 1-(cyclohexyl)ethoxy, 2-(cyclopropyl)ethoxy, 2-(cyclobutyl)ethoxy, 2-(cyclopentyl)ethoxy, 2-(cyclohexyl)ethoxy, 3-(cyclopropyl)propoxy, 3-(cyclobutyl)propoxy, 3-(cyclopentyl)propoxy, 3-(cyclohexyl)propoxy, 4-(cyclopropyl)butoxy, 4-(cyclobutyl)butoxy, 4-(cyclopentyl)butoxy, 4-(cyclohexyl)butoxy, 5-(cyclopropyl)pentoxy, 5-(cyclobutyl)pentoxy, 5-(cyclopentyl)pentoxy, 5-(cyclohexyl)pentoxy, 6-(cyclopropyl)hexoxy, 6-(cyclobutyl)hexoxy, 6-(cyclopentyl)hexoxy or 6-(cyclohexyl)hexoxy, in particular cyclopentylmethoxy or cyclohexylmethoxy;

C₃-C₆-cycloalkyloxy: cyclopropyloxy, cyclobutyloxy, cyclopentyloxy or cyclohexyloxy;

C₃-C₆-cycloalkylthio: cyclopropylthio, cyclobutylthio, cyclopentylthio or cyclohexylthio;

C₃-C₆-cycloalkylcarbonyloxy: cyclopropylcarbonyloxy, cyclobutylcarbonyloxy, cyclopentylcarbonyloxy or cyclohexylcarbonyloxy;

C₃-C₆-cycloalkylsulfonyloxy: cyclopropylsulfonyloxy, cyclobutylsulfonyloxy, cyclopentylsulfonyloxy or cyclohexylsulfonyloxy;

C₅-C₇-cycloalkenyloxy: cyclopent-1-enyloxy, cyclopent-2-enyloxy, cyclopent-3-enyloxy, cyclohex-1-enyloxy, cyclohex-2-enyloxy, cyclohex-3-enyloxy, cyclohept-1-enyloxy, cyclohept-2-enyloxy, cyclohept-3-enyloxy or cyclohept-4-enyloxy.

3- to 7-membered azaheterocycles which, in addition to carbon ring members, may also contain an oxygen or sulfur atom as ring member, are, for example,

pyrrolidin-1-yl, isoxazolidin-2-yl, isothiazolidin-2-yl, oxazolidin-3-yl, thiazolidin-3-yl, piperidin-1-yl, morpholin-1-yl, thiomorpholin-1-yl and azepin-1-yl.

3- to 7-membered heterocyclyl—which may be attached directly or via an oxygen, alkoxy, alkenyloxy or alkynyloxy bridge—are to be understood as including both saturated, partially or fully unsaturated and aromatic heterocycles having one to three heteroatoms, selected from the group consisting of

one to three nitrogen atoms,

one or two oxygen atoms and

one or two sulfur atoms.

Examples of saturated heterocycles which may contain a carbonyl or thiocarbonyl ring member are:

oxiranyl, thiiranyl, aziridin-1-yl, aziridin-2-yl, diaziridin-1-yl, diaziridin-3-yl, oxetan-2-yl, oxetan-3-yl, thietan-2-yl, thietan-3-yl, azetidin-1-yl, azetidin-2-yl, azetidin-3-yl, tetrahydrofuran-2-yl, tetrahydrofuran-3-yl, tetrahydrothiophen-2-yl, tetrahydrothiophen-3-yl, pyrrolidin-1-yl, pyrrolidin-2-yl, pyrrolidin-3-yl, 1,3-dioxolan-2-yl, 1,3-dioxolan-4-yl, 1,3-oxathiolan-2-yl, 1,3-oxathiolan-4-yl, 1,3-oxathiolan-5-yl, 1,3-oxazolidin-2-yl, 1,3-oxazolidin-3-yl, 1,3-oxazolidin-4-yl, 1,3-oxazolidin-5-yl, 1,2-oxazolidin-2-yl, 1,2-oxazolidin-3-yl, 1,2-oxazolidin-4-yl, 1,2-oxazolidin-5-yl, 1,3-dithiolan-2-yl, 1,3-dithiolan-4-yl, pyrrolidin-1-yl, pyrrolidin-2-yl, pyrrolidin-5-yl, tetrahydropyrazol-1-yl, tetrahydropyrazol-3-yl, tetrahydropyrazol-4-yl, tetrahydropyran-2-yl, tetrahydropyran-3-yl, tetrahydropyran-4-yl, tetrahydrothiopyran-2-yl, tetrahydrothiopyran-3-yl, tetrahydrothiopyran-4-yl, piperidin-1-yl, piperidin-2-yl, piperidin-3-yl, piperidin-4-yl, 1,3-dioxan-2-yl, 1,3-dioxan-4-yl, 1,3-dioxan-5-yl, 1,4-dioxan-2-yl, 1,3-oxathian-2-yl, 1,3-oxathian-4-yl, 1,3-oxathian-5-yl, 1,3-oxathian-6-yl, 1,4-oxathian-2-yl, 1,4-oxathian-3-yl, morpholin-2-yl, morpholin-3-yl, morpholin-4-yl, hexahydropyridazin-1-yl, hexahydropyridazin-3-yl, hexahydropyridazin-4-yl, hexahydropyrimidin-1-yl, hexahydropyrimidin-2-yl, hexahydropyrimidin-4-yl, hexahydropyrimidin-5-yl, piperazin-1-yl, piperazin-2-yl, piperazin-3-yl, hexahydro-1,3,5-triazin-1-yl, hexahydro-1,3,5-triazin-2-yl, oxepan-2-yl, oxepan-3-yl, oxepan-4-yl, thiepan-2-yl, thiepan-3-yl, thiepan-4-yl, 1,3-dioxepan-2-yl, 1,3-dioxepan-4-yl, 1,3-dioxepan-5-yl, 1,3-dioxepan-6-yl, 1,3-dithiepan-2-yl, 1,3-dithiepan-2-yl, 1,3-dithiepan-2-yl, 1,3-dithiepan-2-yl, 1,4-dioxepan-2-yl, 1,4-dioxepan-7-yl, hexahydroazepin-1-yl, hexahydroazepin-2-yl, hexahydroazepin-3-yl, hexahydroazepin-4-yl, hexahydro-1,3-diazepin-1-yl, hexahydro-1,3-diazepin-2-yl, hexahydro-1,3-diazepin-4-yl, hexahydro-1,4-diazepin-1-yl and hexahydro-1,4-diazepin-2-yl.

Examples of unsaturated heterocycles which may contain a carbonyl or thiocarbonyl ring member are:

dihydrofuran-2-yl, 1,2-oxazolin-3-yl, 1,2-oxazolin-5-yl, 1,3-oxazolin-2-yl.

Among the heteroaromatic radicals, preference is given to 5- and 6-membered radicals, i.e., for example, furyl, such as 2-furyl and 3-furyl, thienyl, such as 2-thienyl and 3-thienyl, pyrrolyl, such as 2-pyrrolyl and 3-pyrrolyl, isoxazolyl, such as 3-isoxazolyl, 4-isoxazolyl and 5-isoxazolyl, isothiazolyl, such as 3-isothiazolyl, 4-isothiazolyl and 5-isothiazolyl, pyrazolyl, such as 3-pyrazolyl, 4-pyrazolyl and 5-pyrazolyl, oxazolyl, such as 2-oxazolyl, 4-oxazolyl and 5-oxazolyl, thiazolyl, such as 2-thiazolyl, 4-thiazolyl and 5-thiazolyl, imidazolyl, such as 2-imidazolyl and 4-imidazolyl, oxadiazolyl, such as 1,2,4-oxadiazol-3-yl, 1,2,4-oxadiazol-5-yl and 1,3,4-oxadiazol-2-yl, thiadiazolyl, such as 1,2,4-thiadiazol-3-yl, 1,2,4-thiadiazol-5-yl and 1,3,4-thiadiazol-2-yl, triazolyl, such as 1,2,4-triazol-1-yl, 1,2,4-triazol-3-yl and 1,2,4-triazol-4-yl, pyridinyl, such as 2-pyridinyl, 3-pyridinyl and 4-pyridinyl, pyridazinyl, such as 3-pyridazinyl and 4-pyridazinyl, pyrimidinyl, such as 2-pyrimidinyl, 4-pyrimidinyl and 5-pyrimidinyl, furthermore 2-pyrazinyl, 1,3,5-triazin-2-yl and 1,2,4-triazin-3-yl, in particular pyridyl, pyrimidyl, furanyl and thienyl.

If Q is phenyl which has a fused heterocycle, the radical Q is, for example, a radical which is derived from indole, benzimidazole, benzopyrazole, benzoxazole, benzisoxazole, benzothiophene, benzothiazole, benzoisothiazole, benzothiadiazole, benzoisothiadiazole, benzoxazolidinone, benzoxazolidinthione, benzothiazolidinone, benzothiadiazolidinethione, benzoquinoline, 1,2,3,4-tetrahydrobenzo-1,4-oxazin-3-one, 1,2,3,4-tetrahydrobenzo-1,4-thiazin-3-one, 1,2,3,4-tetrahydrobenzoquinoline, 1,2,3,4-tetrahydrobenzoquinolin-2-one, benzopyridazine, 1,2,3,4-tetrahydrobenzopyridazine or 1,2,3,4-tetrahydrobenzopyridazin-2-one, in particular from benzoxazole, benzothiazole, benzoisothiazole, benzoxazolidinone, benzoxazolidinethione, benzothiazolidinone, 1,2,3,4-tetrahydrobenzo-1,4-oxazin-3-one, 1,2,3,4-tetrahydrobenzo-1,4-thiazin-3-one or 1,2,3,4-tetrahydrobenzoquinoline, which may be unsubstituted or substituted. Suitable substituents are the radicals mentioned under R³, R⁴, UR⁶, TR⁷ and R³⁰.

Suitable substituents for C₁-C₆-alkyl in R^{1a} are, for example:

COOH, CN, C₁-C₆-alkoxy, C₁-C₆-alkoxycarbonyl, C₁-C₆-alkylthio, C₁-C₆-alkylsulfinyl, C₁-C₆-alkylsulfonyl, C₃-C₆-cycloalkyl, C₃-C₆-alkenyloxy, C₃-C₆-alkenyloxycarbonyl, C₃-C₆-alkynyloxy, C₃-C₆-alkynyloxycarbonyl, C₃-C₆-cycloalkoxy, C₃-C₆-cycloalkylthio, C₁-C₆-haloalkoxy, C₃-C₆-haloalkenyloxy, C₃-C₆-haloalkynyloxy, C₃-C₆-cycloalkylthio, C₃-C₆-alkenylthio, C₃-C₆-alkynylthio and C₃-C₆-halocycloalkyl, COR¹, P(O)(OR¹)₂, P(S)(OR¹)₂, C(O)N(R¹)₂, C(O)NH₂ and also phenyl, phenoxy and benzyloxy, where the benzene rings of the three last-mentioned groups for their part may be substituted by halogen, C₁-C₄-alkyl or C₁-C₄-haloalkyl.

Suitable substituents for C₃-C₆-alkenyl and C₃-C₆-alkynyl in R^{1a} are, for example: COOH, C₁-C₆-alkoxy, C₁-C₆-alkoxycarbonyl, C₁-C₆-alkylthio, C₁-C₆-alkylsulfinyl, C₁-C₆-alkylsulfonyl, C₃-C₆-cycloalkyl, C₃-C₆-cycloalkoxy, C₁-C₆-haloalkoxy and C₃-C₆-halocycloalkyl, and also phenyl, benzyl, phenoxy and benzyloxy, where the benzene rings of the 4 last-mentioned groups for their part may be substituted by halogen, C₁-C₄-alkyl or C₁-C₄-haloalkyl.

The meaning of the substituent R^{a} is of minor importance for the process according to the invention. Preferably, R^{a} is CO₂R¹, halogen, cyano, OR^{1a} and in particular halogen or C₁-C₃-alkyl. Here, R¹ and R^{1a} have the meanings given above. R¹ is in particular hydrogen or C₁-C₃-alkyl.

R^{1a} is in particular C₁-C₃-alkyl, C₃-C₆-cycloalkyl, C₃-C₆-alkenyl, C₃-C₆-alkynyl, C₁-C₃-haloalkyl, C₁-C₃-alkoxycarbonyl-C₁-C₃-alkyl, cyano-C₁-C₃-alkyl, benzyl which may be substituted by halogen, C₁-C₄-alkyl or trifluoromethyl, or phenyl which may be substituted by halogen, C₁-C₄-alkyl, trifluoromethyl or C₁-C₄-alkoxy.

Preferred radicals R are C(O)OR² and C(S)OR². Here, R² is as defined above and is preferably C₁-C₆-alkyl, C₃-C₆-alkenyl or C₃-C₆-alkynyl, which radicals may be unsubstituted or substituted. With a view to the substituents on C₁-C₆-alkyl, C₃-C₆-alkenyl and C₃-C₆-alkynyl in R², there are no limitations in principle. Substituents which are suitable in principle are all those substituents mentioned as substituents for C₁-C₆-alkyl, C₃-C₆-alkenyl or C₃-C₆-alkynyl in R^{1a}.

R² is in particular C₁-C₆-alkyl, C₃-C₆-cycloalkyl, C₂-C₆-alkenyl, C₃-C₆-alkynyl, C₁-C₆-haloalkyl, C₁-C₆-alkoxy-C₁-C₆-alkyl, C₁-C₆-alkoxycarbonyl-C₁-C₆-alkyl, C₃-C₆-alkenyloxy-C₁-C₆-alkyl, C₃-C₆-alkynyloxy-C₁-C₆-alkyl, cyano-C₁-C₆-alkyl, phenyl or benzyl, where phenyl and benzyl may each be mono- to pentasubstituted by halogen, C₁-C₄-alkyl, C₁-C₄-haloalkyl, C₁-C₄-alkoxy, C₁-C₄-haloalkoxy, amino, C₂-C₄-monoalkylamino, C₁-C₄-dialkylamino, C₁-C₄-alkoxycarbonyl, nitro or cyano. R² is in particular C₁-C₆-alkyl and particularly preferably C₁-C₄-alkyl which is preferably linear and in particular unsubstituted.

R is in particular C₁-C₄-alkyloxycarbonyl or C₁-C₄-alkyloxythiocarbonyl.

Z or Z¹ is preferably oxygen or sulfur.

The variable n is preferably 0 or 1. In a particularly preferred embodiment of the invention, n has the value 0.

Q is, for example,

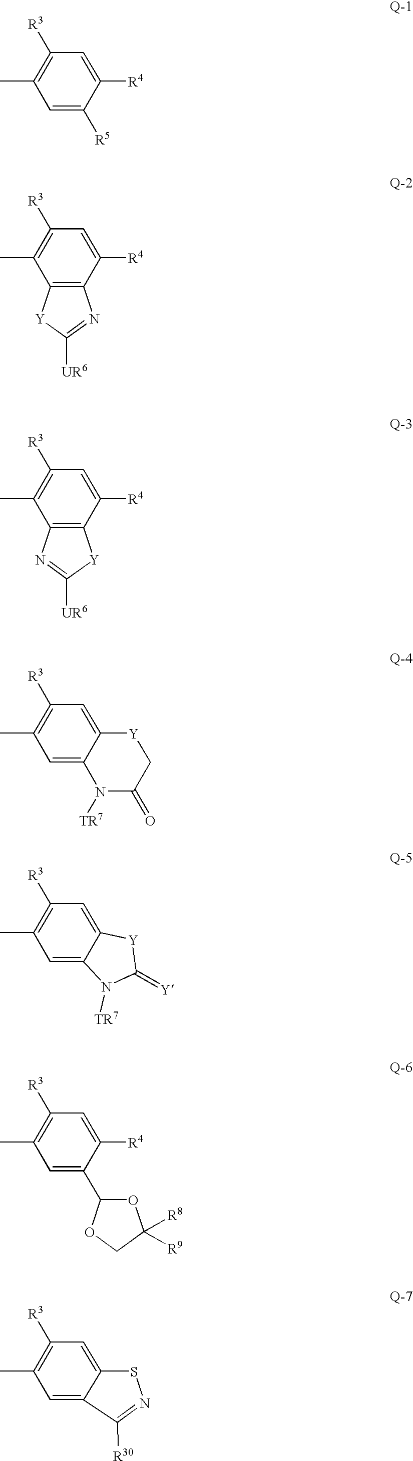

In the formulae Q-1-Q-7, variables Y and Y′, T, U and the radicals R³, R⁴, R⁵, R⁶, R⁷, R⁸, R⁹ and R³⁰ are as defined below:

Y and Y′ independently of one another are oxygen or sulfur;

T is a chemical bond or oxygen;

U is a chemical bond, C₁-C₄-alkylene, O, S, SO or SO₂;

R³ is hydrogen or halogen;

R⁴ is C₁-C₄-alkyl, C₁-C₄-haloalkyl, C₁-C₄-alkoxy, C₁-C₄-alkylthio, C₁-C₄-haloalkoxy, halogen, cyano or NO₂;

R⁵ is hydroxyl, mercapto, cyano, nitro, halogen, C₁-C₆-alkyl, C₂-C₆-alkenyl, C₂-C₆-alkynyl, C₁-C₆-haloalkyl, C₁-C₆-alkoxy-(C₁-C₆-alkyl)carbonyl, C₁-C₆-alkylthio-(C₁-C₆-alkyl)carbonyl, (C₁-C₆-alkyl)iminooxycarbonyl, C₁-C₆-alkoxy-C₁-C₆-alkyl, C₁-C₆-alkoxyamino-C₁-C₆-alkyl, C₁-C₆-alkoxy-C₁-C₆-alkylamino-C₁-C₆-alkyl, C₁-C₃-alkoxy-C₃-C₆-alkenyl, C₃-C₆-haloalkenyl, cyano-C₃-C₆-alkenyl, C₃-C₆-alkynyl, C₁-C₃-alkoxy-C₃-C₆-alkynyl, C₃-C₆-haloalkynyl, cyano-C₃-C₆-alkynyl,

C₁-C₆-alkoxy, C₁-C₆-alkylthio, C₃-C₆-cycloalkoxy, C₃-C₆-cycloalkylthio, C₂-C₆-alkenyloxy, C₂-C₆-alkenylthio, C₂-C₆-alkynyloxy, C₂-C₆-alkynylthio, (C₁-C₆-alkyl)carbonyloxy, (C₁-C₆-alkyl)carbonylthio, (C₁-C₆-alkoxy)carbonyloxy, (C₂-C₆-alkenyl)carbonyloxy, (C₂-C₆-alkenyl)carbonylthio, (C₂-C₆-alkynyl)carbonyloxy, (C₂—C₆-alkynyl)carbonylthio, C₁-C₆-alkylsulfonyloxy or C₁-C₆-alkylsulfonyl, where each of the 17 last-mentioned radicals may, if desired, carry one, two or three substituents selected from the group consisting of:

halogen, nitro, cyano, hydroxyl, C₃-C₆-cycloalkyl, C₁-C₆-alkoxy, C₃-C₆-cycloalkoxy, C₃-C₆-alkenyloxy, C₃-C₆-alkynyloxy, C₁-C₆-alkoxy-C₁-C₆-alkoxy, C₁-C₆-alkylthio, C₁-C₆-alkylsulfinyl, C₁-C₆-alkylsulfonyl, C₁-C₆-alkylideneaminooxy, oxo, ═N—OR¹⁰

phenyl, phenoxy or phenylsulfonyl, where the three last-mentioned groups may optionally carry one, two or three substituents selected from the group consisting of halogen, nitro, cyano, C₁-C₆-alkyl, C₁-C₆-haloalkyl, C₁-C₆-alkoxy and (C₁-C₆-alkoxy)carbonyl;

—CO—R¹¹, —CO—OR¹¹, —CO—SR¹¹, —CO—N(R¹¹)—R¹², —OCO—R¹¹, —OCO—OR^{11′}, —OCO—SR^{11′}, —OCO—N(R¹¹)—R¹², —N(R¹¹)—R¹², and —C(R¹³)═N—OR¹⁰;

C(Z²)—R¹⁴, —C(═NR¹⁵)R¹⁴, C(R¹⁴)(Z³R¹⁶)(Z⁴R¹⁷), C(R¹⁴)═C(R¹⁸)—CN, C(R¹⁴)═C(R¹⁸)—CO—R¹⁹, —CH(R¹⁴)—CH(R¹⁸)—COR¹⁹, —C(R¹⁴)═C(R¹⁸)—CH₂—CO—R¹⁹, —C(R¹⁴)═C(R¹⁸)—C(R²⁰)═C(R²¹)—CO—R¹⁹, —C(R¹⁴)═C(R^{18)—CH}₂—CH(R²²)—CO—R¹⁹, —CO—OR²³, —CO—SR²³, —CON(R²³)—OR¹⁰, —C≡C—CO—NHOR¹⁰, —C≡C—CO—N(R²³)—OR¹⁰, —C≡C—CS—NH—OR¹⁰, —C≡C—CS—N(R²³)—OR¹⁰, —C(R¹⁴)═C(R¹⁸)—CO—NHOR¹⁰, —C(R¹⁴)═C(R¹⁸)—CO—N(R²³)—OR¹⁰, —C(R¹⁴)═C(R¹⁸)—CS—NHOR¹⁰, —C(R¹⁴)═C(R¹⁸)—CS—N(R²³)—OR¹⁰, —C(R¹⁴)═C(R¹⁸)—C(R¹³)═N—OR¹⁰, C(R¹³)═N—OR¹⁰, —C≡C—C(R¹³)═NOR¹⁰, C(Z³R¹⁶) (Z⁴R¹⁷)—OR²³, —C(Z³R¹⁶)(Z⁴R¹⁷)SR²³, C(Z³R¹⁶)(Z⁴R¹⁷)—N(R²⁴)R²⁵, —N(R²⁴)—R²⁵, —CO—N(R²⁴)—R²⁵ or —C(R¹⁴)═C(R¹⁸)CO—N(R²⁴)R²⁵ and Z², Z³, Z⁴ independently of one another are oxygen or sulfur;

R⁶ is CO₂H, C₁-C₆-alkyl, C₁-C₆-haloalkyl, hydroxy-C₁-C₄-alkyl, cyano-C₁-C₄-alkyl, C₁-C₄-alkoxy-C₁-C₄-alkyl, amino-C₁-C₄-alkyl, C₁-C₄-alkylamino-C₁-C₄-alkyl, di(C₁-C₄-alkyl)amino-C₁-C₄-alkyl, C₁-C₄-alkylthio-C₁-C₄-alkyl, hydroxycarbonyl-C₁-C₄-alkyl, (C₁-C₄-alkoxy)carbonyl-C₁-C₄-alkyl, (C₁-C₄-alkylthio)carbonyl-C₁-C₄-alkyl, aminocarbonyl-C₁-C₄-alkyl, (C₁-C₄-alkylamino)carbonyl-C₁-C₄-alkyl, di(C₁-C₄-alkyl)aminocarbonyl-C₁-C₄-alkyl, C₃-C₆-alkenyl, C₁-C₃-alkoxy-C₃-C₆-alkenyl, C₃-C₆-haloalkenyl, cyano-C₃-C₆-alkenyl, C₃-C₆-alkynyl, C₁-C₃-alkoxy-C₃-C₆-alkynyl, C₃-C₆-haloalkynyl, cyano-C₃-C₆-alkynyl,

phenyl, phenyl-C₁-C₄-alkyl, where the phenyl rings optionally carry one, two or three substituents selected from the group consisting of halogen, cyano, C₁-C₆-alkyl, C₁-C₆-haloalkyl, C₁-C₆-alkoxy or C₁-C₆-haloalkoxy;

C₃-C₇-cycloalkyl, 3- to 7-membered saturated heterocyclyl, where each cycloalkyl and each heterocyclyl ring may contain a carbonyl or thiocarbonyl ring member and where each cycloalkyl and heterocyclyl ring may be unsubstituted or may carry one, two, three or four substituents selected from the group consisting of cyano, nitro, amino, hydroxyl, halogen, C₁-C₄-alkyl, C₁-C₄-haloalkyl, C₁-C₄-cyanoalkyl, C₁-C₄-hydroxyalkyl, C₁-C₄-aminoalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-alkylsulfonyl, C₁-C₄-haloalkylsulfonyl, (C₁-C₄-alkoxy)carbonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, di(C₁-C₄-alkyl)amino, C₃-C₆-alkenyl, C₃-C₆-alkynyl, C₃-C₄-alkenyloxy, C₃-C₄-alkenylthio, C₃-C₄-alkynyloxy and C₃-C₄-alkynylthio;

or, if U (or T) is a chemical bond, R⁶ is also hydrogen, hydroxyl, cyano, mercapto, amino, C₁-C₄-alkylamino, di-C₁-C₄-alkylamino, saturated 5- or 6-membered nitrogen heterocyclyl which is attached via nitrogen, C₃-C₆-cycloalkylamino, halogen, —(CH₂)ₙ—CH(OH)—CH₂-R²⁸ —(CH₂)ₙ—CH(halogen)—CH₂-R²⁸, —(CH₂)ₙ—CH₂—CH(halogen)—R²⁸, —(CH₂)ₙ—CH═CH—R²⁸ or —(CH₂)ₙ—CH═C(halogen)—R²⁸, where R²⁸ is hydroxycarbonyl, (C₁-C₄-alkoxy)carbonyl, (C₁-C₄-alkylthio)carbonyl, aminocarbonyl, (C₁-C₄-alkylamino)carbonyl or di(C₁-C₄-alkyl)aminocarbonyl and n is 0 or 1;

R⁷ has the meanings given for R⁶;

R⁸ is hydrogen, C₁-C₃-alkyl, C₁-C₃-haloalkyl or halogen;;

R⁹ is hydrogen, C₁-C₃-alkyl, C₁-C₃-haloalkyl; or

R⁸ and R⁹ together are C═O;

R¹⁰ is hydrogen, C₁-C₆-alkyl, C₁-C₆-haloalkyl, C₃-C₆-cycloalkyl, C₃-C₆-alkenyl, C₃-C₆-alkynyl, hydroxy-C₁-C₆-alkyl, C₁-C₆-alkoxy-C₁-C₆-alkyl, C₁-C₆-alkylthio-C₁-C₆-alkyl, cyano-C₁-C₆-alkyl, (C₁-C₆-alkyl)carbonyl-C₁-C₆-alkyl, (C₁-C₆-alkoxy)carbonyl-C₁-C₆-alkyl, (C₁-C₆-alkoxy)carbonyl-C₂-C₆-alkenyl, (C₁-C₆-alkyl)carbonyloxy-C₁-C₆-alkyl or phenyl-C₁-C₆-alkyl, where the phenyl ring may, if desired, carry one, two or three substituents selected from the group consisting of cyano, nitro, halogen, C₁-C₆-alkyl, C₁-C₆-haloalkyl, C₁-C₆-alkoxy and (C₁-C₆-alkoxy)carbonyl;

R¹¹ is hydrogen, C₁-C₆-alkyl, C₃-C₆-cycloalkyl, C₃-C₆-alkenyl, C₃-C₆-alkynyl, C₁-C₆-alkoxy-C₁-C₆-alkyl, (C₁-C₆-alkoxy) carbonyl-C₁-C₆-alkyl, (C₃-C₆-alkenyloxy)carbonyl-C₁-C₆-alkyl, phenyl or phenyl-C₁-C₆-alkyl, where the phenyl ring of the two last-mentioned groups may be unsubstituted or may carry one, two or three radicals selected from the group consisting of halogen, nitro, cyano, C₁-C₆-alkyl, C₁-C₆-haloalkyl, C₁-C₆-alkoxy and (C₁-C₆-alkyl)carbonyl;

R^{11′} has the meanings given for R¹¹ except for hydrogen;

R¹² is hydrogen, hydroxyl, C₁-C₆-alkyl, C₃-C₆-cycloalkyl, C₃-C₆-cycloalkylaminocarbonyl, C₁-C₆-alkylaminocarbonyl, C₁-C₆-alkoxy, (C₁-C₆-alkoxy)carbonyl-C₁-C₆-alkoxy, C₃-C₆-alkenyl or C₃-C₆-alkenyloxy;

R¹³ is hydrogen, halogen, C₁-C₆-alkyl, C₁-C₆-haloalkyl, C₁-C₆-alkoxy, C₁-C₆-haloalkoxy, C₃-C₆-alkenyloxy, C₃-C₆-alkynyloxy, C₁-C₆-alkylthio, C₁-C₆-haloalkylthio, (C₁-C₆-alkyl)carbonyloxy, (C₁-C₆-haloalkyl)carbonyloxy, C₁-C₆-alkylsulfonyloxy or C₁-C₆-haloalkylsulfonyloxy, where the 12 last-mentioned radicals may carry one of the following substituents: hydroxyl, cyano, hydroxycarbonyl, C₁-C₆-alkoxy, C₁-C₆-alkylthio, (C₁-C₆-alkyl)carbonyl, (C₁-C₆-alkoxy)carbonyl, (C₁-C₆-alkyl)aminocarbonyl, di(C₁-C₆-alkyl)aminocarbonyl, (C₁-C₆-alkyl)carbonyloxy, C₁-C₆-alkoxy- (C₁-C₆-alkyl) aminocarbonyl;

(C₁-C₆-alkyl)carbonyl, (C₁-C₆-haloalkyl)carbonyl, (C₁-C₆-alkoxy)carbonyl, (C₁-C₆-alkoxy)carbonyloxy, (C₁-C₆-alkyl)carbonylthio, (C₁-C₆-haloalkyl)carbonylthio, (C₁-C₆-alkoxy)carbonylthio, C₂-C₆-alkenyl, (C₂-C₆-alkenyl)carbonyloxy, C₂-C₆-alkenylthio, C₃-C₆-alkynyl, C₃-C₆-alkynyloxy, C₃-C₆-alkynylthio, (C₂-C₆-alkynyl)carbonyloxy, C₃-C₆-alkynylsulfonyloxy, C₃-C₆-cycloalkyl, C₃-C₆-cycloalkyloxy, C₃-C₆-cycloalkylthio, (C₃-C₆-cycloalkyl)carbonyloxy, C₃-C₆-cycloalkylsulfonyloxy;

phenyl, phenoxy, phenylthio, benzoyloxy, phenylsulfonyloxy, phenyl-C₁-C₆-alkyl, phenyl-C₁-C₆-alkoxy, phenyl-C₁-C₆-alkylthio, phenyl-(C₁-C₆-alkyl)carbonyloxy or phenyl-(C₁-C₆-alkyl)sulfonyloxy, where the phenyl rings of the 10 last-mentioned radicals may be unsubstituted or may for their part carry one to three substituents, in each case selected from the group consisting of cyano, nitro, halogen, C₁-C₆-alkyl, C₁-C₆-haloalkyl, C₁-C₆-alkoxy and (C₁-C₆-alkoxy)carbonyl;

R¹⁴ is hydrogen, cyano, C₁-C₆-alkyl, C₁-C₆-haloalkyl, C₂-C₆-alkenyl, C₂-C₆-alkynyl, C₃-C₆-cycloalkyl, C₁-C₆-alkoxy-C₁-C₆-alkyl or (C₁-C₆-alkoxy)carbonyl;

R¹⁵ is hydrogen, hydroxyl, C₁-C₆-alkyl, C₃-C₆-alkenyl, C₃-C₆-alkynyl, C₃-C₆-cycloalkyl, C₁-C₆-haloalkyl, C₁-C₆-alkoxy-C₁-C₆-alkyl, C₁-C₆-alkoxy, C₃-C₆-alkenyloxy, C₃-C₆-alkynyloxy, C₃-C₆-cycloalkoxy, C₅-C₇-cycloalkenyloxy, C₁-C₆-haloalkoxy, C₃-C₆-haloalkenyloxy, hydroxy-C₁-C₆-alkoxy, cyano-C₁-C₆-alkoxy, C₃-C₆-cycloalkyl-C₁-C₆-alkoxy, C₁-C₆-alkoxy-C₁-C₆-alkoxy, C₁-C₆-alkoxy-C₃-C₆-alkenyloxy, (C₁-C₆-alkyl)carbonyloxy, (C₁-C₆-haloalkyl)carbonyloxy, (C₁-C₆-alkyl)carbamoyloxy, (C₁-C₆-haloalkyl)carbamoyloxy, (C₁-C₆-alkyl)carbonyl-C₁-C₆-alkyl, (C₁-C₆-alkyl)carbonyl-C₁-C₆-alkoxy, (C₁-C₆-alkoxy)carbonyl-C₁-C₆-alkyl, (C₁-C₆-alkoxy)carbonyl-C₁-C₆-alkoxy, C₁-C₆-alkylthio-C₁-C₆-alkoxy, di(C₁-C₆-alkyl)amino-C₁-C₆-alkoxy, —N(R²⁶)R²⁷, phenyl, which for its part may carry one, two or three substituents, in each case selected from the group consisting of cyano, nitro, halogen, C₁-C₆-alkyl, C₁-C₆-haloalkyl, C₂-C₆-alkenyl, C₁-C₆-alkoxy and (C₁-C₆-alkoxy)carbonyl; phenyl-C₁-C₆-alkoxy, phenyl-(C₁-C₆-alkyl), phenyl-C₃-C₆-alkenyloxy or phenyl-C₃-C₆-alkynyloxy, where in each case one or two methylene groups of the carbon chains in the four last-mentioned groups may be replaced by —O—, —S—, or —N(C₁-C₆-alkyl)- and where the phenyl rings in the four last-mentioned groups may be unsubstituted or may for their part carry one to three substituents selected from the group consisting of cyano, nitro, halogen, C₁-C₆-alkyl, C₁-C₆-haloalkyl, C₂-C₆-alkenyl, C₁-C₆-alkoxy and (C₁-C₆-alkoxy)carbonyl;

C₃-C₇-heterocyclyl, C₃-C₇-heterocyclyl-C₁-C₆-alkyl, C₃-C₇-heterocyclyl-C₁-C₆-alkoxy, C₃-C₇-heterocyclyl-C₃-C₆-alkenyloxy or C₃-C₇-heterocyclyl-C₃-C₆-alkynyloxy, where in each case one or two methylene groups of the carbon chains in the four last-mentioned groups may be replaced by —O—, —S— or —N(C₁-C₆-alkyl)- and where each heterocycle may be saturated, unsaturated or aromatic and is either unsubstituted or for its part carries one to three substituents selected from the group consisting of cyano, nitro, halogen, C₁-C₆-alkyl, C₁-C₆-haloalkyl, C₂-C₆-alkenyl, C₁-C₆-alkoxy and (C₁-C₆-alkoxy)carbonyl;

R¹⁶, R¹⁷ independently of one another are C₁-C₆-alkyl, C₁-C₆-haloalkyl, C₃-C₆-alkenyl, C₃-C₆-alkynyl, C₁-C₆-alkoxy-C₁-C₆-alkyl, or together are a saturated or unsaturated 2- to 4-membered carbon chain which may carry an oxo substituent, where a member of this chain which is not adjacent to the variables Z³ and Z⁴ may be replaced by —O—, —S—, —N═, —NH— or —N(C₁-C₆-alkyl)- and where the carbon chain may carry one to three radicals selected from the group consisting of cyano, nitro, amino, halogen, C₁-C₆-alkyl, C₂-C₆-alkenyl, C₁-C₆-alkoxy, C₂-C₆-alkenyloxy, C₂-C₆-alkynyloxy, C₁-C₆-haloalkyl, cyano-C₁-C₆-alkyl, hydroxy-C₁-C₆-alkyl, C₁-C₆-alkoxy-C₁-C₆-alkyl, C₃-C₆-alkenyloxy-C₁-C₆-alkyl, C₃-C₆-alkynyloxy-C₁-C₆-alkyl, C₃-C₆-cycloalkyl, C₃-C₆-cycloalkoxy, carboxyl, (C₁-C₆-alkoxy)carbonyl, (C₁-C₆-alkyl)carbonyloxy-C₁-C₆-alkyl and phenyl; optionally substituted phenyl, where the carbon chain may also be substituted by a fused-on or spiro-linked 3- to 7-membered ring which may contain one or two heteroatoms selected from the group consisting of oxygen, sulfur, nitrogen and C₁-C₆-alkyl-substituted nitrogen as ring members and which may, if desired, carry one or two of the following substituents: cyano, C₁-C₆-alkyl, C₂-C₆-alkenyl, C₁-C₆-alkoxy, cyano-C₁-C₆-alkyl, C₁-C₆-haloalkyl and (C₁-C₆-alkoxy)carbonyl;

R¹⁸ is hydrogen, cyano, halogen, C₁-C₆-alkyl, C₁-C₆-haloalkyl, C₁-C₆-alkoxy, (C₁-C₆-alkyl)carbonyl or (C₁-C₆-alkoxy)carbonyl;

R¹⁹ is hydrogen, O—R²⁸, S—R²⁸, C₁-C₆-alkyl which may carry one or two C₁-C₆-alkoxy substituents, C₂-C₆-alkenyl, C₂-C₆-alkynyl, C₁-C₆-haloalkyl, C₃-C₆-cycloalkyl, C₁-C₆-alkylthio-C₁-C₆-alkyl, C₁-C₆-alkyliminooxy, —N(R²⁴)R²⁵ or phenyl which may be unsubstituted or may carry one to three substituents, in each case selected from the group consisting of cyano, nitro, halogen, C₁-C₆-alkyl, C₂-C₆-alkenyl, C₁-C₆-haloalkyl, C₁-C₆-alkoxy and (C₁-C₆-alkoxy)carbonyl;

R²⁰ is hydrogen, cyano, halogen, C₁-C₆-alkyl, C₃-C₆-alkenyl, C₃-C₆-alkynyl, C₁-C₆-alkoxy-C₁-C₆-alkyl, (C₁-C₆-alkyl)carbonyl, (C₁-C₆-alkoxy)carbonyl, —N(R²⁴)R²⁵ or phenyl which for its part may carry one to three substituents selected from the group consisting of cyano, nitro, halogen, C₁-C₆-alkyl, C₁-C₆-haloalkyl, C₃-C₆-alkenyl, C₁-C₆-alkoxy and (C₁-C₆-alkoxy)carbonyl;

R²¹ is hydrogen, cyano, halogen, C₁-C₆-alkyl, C₁-C₆-alkoxy, C₁-C₆-haloalkyl, (C₁-C₆-alkyl)carbonyl or (C₁-C₆-alkoxy)carbonyl;

R²² is hydrogen, cyano, C₁-C₆-alkyl or (C₁-C₆-alkoxy)carbonyl; R²³, R²⁸ independently of one another are hydrogen, C₁-C₆-alkyl, C₁-C₆-haloalkyl, C₂-C₆-alkenyl or C₂-C₆-alkynyl, where the 4 last-mentioned groups may each carry one or two of the following radicals: cyano, halogen, hydroxyl, hydroxycarbonyl, C₁-C₆-alkoxy, C₁-C₆-alkylthio, (C₁-C₆-alkyl)carbonyl, (C₁-C₆-alkoxy)carbonyl, (C₁-C₆-alkyl)carbonyloxy, (C₃-C₆-alkenyloxy)carbonyl;

(C₁-C₆-haloalkyl)carbonyl, (C₁-C₆-alkoxy)carbonyl, C₁-C₆-alkylaminocarbonyl, di(C₁-C₆-alkyl)aminocarbonyl, C₁-C₆-alkyloximino-C₁-C₆-alkyl, C₃-C₆-cycloalkyl;

phenyl or phenyl-C₁-C₆-alkyl, where the phenyl rings may be unsubstituted or may for their part carry one to three substituents, in each case selected from the group consisting of cyano, nitro, halogen, C₁-C₆-alkyl, C₁-C₆-haloalkyl, C₁-C₆-alkoxy and (C₁-C₆-alkoxy)carbonyl;

R²⁴, R²⁵, R²⁶, R²⁷ independently of one another are hydrogen, C₁-C₆-alkyl, C₃-C₆-alkenyl, C₂-C₆-alkynyl, C₃-C₆-cycloalkyl, C₁-C₆-haloalkyl, C₁-C₆-alkoxy-C₁-C₆-alkyl, (C₁-C₆-alkyl)carbonyl, (C₁-C₆-alkoxy)carbonyl, (C₁-C₆-alkoxy)carbonyl-C₁-C₆-alkyl,

(C₁-C₆-alkoxy)carbonyl-C₂-C₆-alkenyl, where the alkenyl chain may additionally carry one to three halogen and/or cyano radicals, C₁-C₆-alkylsulfonyl, (C₁-C₆-alkoxy)carbonyl-C₁-C₆-alkylsulfonyl, phenyl or phenylsulfonyl, where the phenyl rings of the two last-mentioned radicals may be unsubstituted or may for their part carry one to three substituents, in each case selected from the group consisting of cyano, nitro, halogen, C₁-C₆-alkyl, C₁-C₆-haloalkyl, C₃-C₆-alkenyl, C₁-C₆-alkoxy and (C₁-C₆-alkoxy)carbonyl; or

R²⁴ and R²⁵ and/or

R²⁶ and R²⁷ together with the respective common nitrogen atom are a saturated or unsaturated 4- to 7-membered azaheterocycle which, in addition to carbon ring members, may, if desired, contain one of the following members: —O—, —S—, —N═, —NH— or —N(C₁-C₆-alkyl)-;

R³⁰ is hydrogen, C₁-C₆-alkyl, C₃-C₈-cycloalkyl, CH₂O—C₁-C₆-alkyl, CH₂O—C₂-C₄-alkenyl, CH₂O—C₂-C₄-alkynyl, CH₂CH₂O—C₁-C₄-alkyl, CH₂CH₂O—C₂-C₄-alkenyl, CH₂CH₂O—C₂-C₄-alkynyl, (C₁-C₆-alkoxy)carbonyl, (C₃-C₄-alkenyloxy)carbonyl, (C₃-C₄-alkynyloxy)carbonyl, (C₃-C₆-cycloalkyloxy)carbonyl, (C₁-C₆-alkylthio)carbonyl, (C₁-C₄-alkoxy)carbonyl-C₁-C₄-alkyl, (C₃-C₄-alkenyloxy)carbonyl-C₁-C₄-alkyl, (C₃-C₄-alkynyloxy)carbonyl-C₁-C₄-alkyl, (C₁-C₄-alkylamino)carbonyl, (C₁-C₄-dialkylamino)carbonyl, (C₃-C₄-alkenylamino)carbonyl, (C₃-C₄-alkynylamino)carbonyl, (C₃-C₄-dialkenylamino)carbonyl, (C₃-C₄-dialkynylamino)carbonyl, (C₃-C₄-alkenyloxy) carbonyl-C₁-C₄-alkyl, (C₃-C₄-alkynyloxy) carbonyl-C₁-C₄-alkyl, C₁-C₄-alkylsulfonylamidocarbonyl, CH(O—C₁-C₄-alkyl)₂, CH[O(CH₂)₃O, CH[O(CH₂)₄O] or phenyl, which may be unsubstituted or for its part may carry one to three substituents in each case selected from the group consisting of cyano, nitro, halogen, C₁-C₆-alkyl, C₁-C₆-haloalkyl, C₁-C₆-alkoxy, (C₁-C₆-alkoxy)carbonyl and C₁-C₄-alkoxycarbonyl-C₁-C₄-alkyl, where each alkyl radical of the abovementioned radicals may be unsubstituted or carry one, two or three substituents selected from the group consisting of halogen, cyano, nitro, C₁-C₄-alkoxy and C₁-C₄-alkylthio and each cycloalkyl radical of the abovementioned radicals may be unsubstituted or may carry one, two or three substituents independently of one another selected from the group consisting of halogen, cyano, nitro, C₁-C₄-alkyl, C₁-C₄-alkoxy and C₁-C₄-alkylthio.

Hereinbelow, compounds in which Z is an optionally R^{a}-substituted methylene group and the variables R^{a}, W, X, Q and n are as defined above are also referred to as compounds Ia.

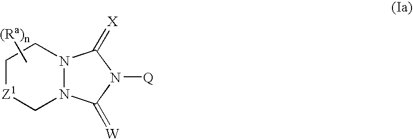

Hereinbelow, compounds in which Z¹ is an optionally R^{a}-substituted methylene group and the variables R^{a}, W, X, Q and n are as defined above are also referred to as compounds Ib.

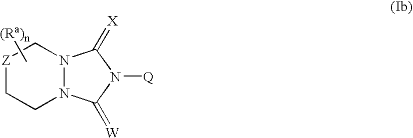

Accordingly, in the compounds IIa, Z is an optionally R^{a}-substituted methylene group and the variables R^{a}, R, W, Q and n are as defined above.

In the compounds IIb, Z¹ is an optionally R^{a}-substituted methylene group and the variables R^{a}, R, W, Q and n are as defined above.

The reaction of the compounds II with a base according to Scheme 3, where the variables R^{a}, Z, Z¹, W, X, R², n and Q are as defined above, is generally carried out at temperatures in the range from 0-150° C., preferably 10-100° C., particularly preferably 20-60° C. The reaction can be carried out at atmospheric pressure or superatmospheric pressure, continuously or batchwise.

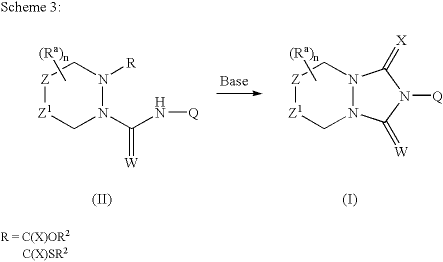

The reaction of II with a base is preferably carried out in a solvent. Suitable solvents are, depending on the temperature range: for example hydrocarbons, such as pentane, hexane, heptane, cyclohexane, aromatic compounds, for example benzene, toluene, xylene, heteroaromatic compounds, such as pyridine, α-, β- or γ-picoline and quinoline, chlorinated hydrocarbons, such as dichloromethane, 1,1-dichloroethane, 1,2-dichloroethane, 1,1,2,2-tetrachloroethane, 1,1-dichloroethylene, chlorobenzene, 1,2-, 1,3-, 1,4-dichlorobenzene, 1-chloronaphthalene and 1,2,4-trichlorobenzene, ethers, such as diethyl ether, tert-butyl methyl ether, tetrahydrofuran, 1,4-dioxane, anisole, glycol ethers, such as dimethyl glycol ether, esters, such as ethyl acetate, propyl acetate, methyl isobutyrate, isobutyl acetate, carboxamides, such as dimethylformamide (DMF), N-methylpyrrolidone (NMP), nitrated hydrocarbons, such as nitromethane, nitroethane, nitropropane and nitrobenzene, ureas, such as tetraethylurea, tetrabutylurea, dimethylethyleneurea, dimethylpropyleneurea, sulfoxides, such as dimethyl sulfoxide, sulfones, such as dimethyl sulfone, diethyl sulfone, tetramethylene sulfone, nitriles, such as acetonitrile, propionitrile, butyronitrile or isobutyronitrile; water or else mixtures of individual solvents.

Suitable bases are, in principle, all compounds capable of abstracting the acidic proton of the NH group of the urea function in the compounds of the formula II. These include oxo bases, nitrogen bases and hydride bases.

Oxo bases include, for example, inorganic bases, such as alkali metal or alkaline earth metal hydroxides, alkali metal and alkaline earth metal bicarbonates, and also alkali metal and alkaline earth metal carbonates, for example lithium hydroxide, sodium hydroxide, potassium hydroxide, calcium hydroxide or magnesium hydroxide, lithium bicarbonate, sodium bicarbonate, potassium bicarbonate, calcium bicarbonate or magnesium bicarbonate, or lithium carbonate, sodium carbonate, potassium carbonate, calcium carbonate or magnesium carbonate. Other suitable oxo bases are alkali metal alkoxides, in particular of lithium, sodium or potassium, the alkoxides which are used generally being alkoxides of C₁-C₆-alkanols, preferably C₁-C₄-alkanols, such as sodium methoxide, ethoxide, n-butoxide or tert-butoxide or potassium methoxide, ethoxide, n-butoxide or tert-butoxide.

The nitrogen bases include primary, secondary or, preferably, tertiary amines, for example trialkylamines, such as triethylamine, tri-n-propylamine, N-ethyldiisopropylamine, cycloaliphatic amines, such as N,N-dimethylcyclohexylamine, cyclic amines, such as azabicyclo[2.2.2]octane (=triethylenediamine), N-methylpyrrolidine, N-ethylpiperidine, dialkylanilines, such as dimethylaminoaniline, p-dimethylaminopyridine, furthermore aromatic nitrogen heterocycles, such as pyridine, α-, β- or γ-picoline, 2,4- and 2,6-lutidine, quinoline, quinazoline, quinoxaline, p-dimethylaminopyridine, pyrimidine, and also tertiary amides, for example dimethylformamide, N-methylformamide, N-methylpyrolidone or tetramethylurea.

Hydride bases are, for example, alkali metal hydrides, such as sodium hydride or potassium hydride.

Preferred bases are tertiary amines, in particular trialkylamines.

The molar ratio of compound II to base is preferably from 0.9 to 1.4, in particular from 0.95 to 1.2 and particularly preferably from 0.98 to 1.15.

For the reaction of compound II with the base according to Scheme 3, the compound II is preferably initially charged in one of the abovementioned solvents or a solvent mixture, and the base is added to the reaction mixture with mixing, for example with stirring. The base is preferably added at a temperature in the range from 0 to 50° C. and in particular from 10 to 30° C.

In general, to bring the reaction to completion, the components are allowed to react at 20-150° C., preferably 20-100° C. and in particular 20-60° C. for another 10 min to 48 h. In the case of thioureas of the formula II (X═S), the reaction is generally substantially complete (conversion >90%) after 0.5-10 h, in the case of ureas of the formula II (X═O) after 4-48 h and in particular after 8-24 h. However, it is also possible to initially charge the base, preferably in one of the solvents mentioned above, or, if the base is a liquid, neat, followed by addition of the compound II and completion of the reaction as above.

The concentration of the starting materials in the solvent is generally in the range from 0.5 to 5 mol/l, preferably in the range fro 0.2 to 2 mol/l.

Work-up of the reaction is carried out in a customary manner, for example by aqueous extraction, by dialysis and/or chromatographically. For the preferred extractive work-up, the reaction mixture containing the fused tetrahydro-[1H]-triazole compound I is—if appropriate after removal of the solvent—taken up in a water-immiscible solvent, basic or acidic compounds are extracted with dilute acid and dilute alkali, respectively, or with water, the organic phase is, if appropriate, dried and the solvent is then removed, preferably under reduced pressure. Here, the product can be obtained by methods known per se using filtration, crystallization or solvent extraction.

The fused triasoles of the formula I may contain one or more centers of chirality, in which case they are usually obtained as mixtures of enantiomers or diastereomers. If desired, the mixtures can be separated into substantially pure isomers using methods customary for this purpose, such as crystallization or chromatography, including chromatography on an optically active adsorbate. It is also possible, for example, to prepare pure optically active isomers from the corresponding optically active starting materials.

The substituted ureas of the formula II required as starting materials for the process according to the invention and a process for their preparation are described in PCT/EP00/05794 which is expressly included herein in its entirety by reference.

Compounds of the formula II can be prepared, for example, according to Scheme 4 by reacting 1H,2H-perhydrodiazines of the formula III with an isocyanate (W═O) or an isothiocyanate (W═S) of the formula IV. In Scheme 4, n, R, R⁴, Z, Z¹, W and Q are as defined above. The procedure shown in Scheme 4 has been found to be advantageous in particular for preparing compounds II in which Z is a methylene group, optionally substituted by R^{a} (compounds IIa). In Scheme 4, Z¹ is preferably oxygen or sulfur.

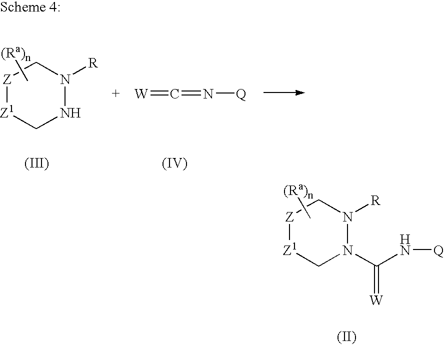

The molar ratios in which the starting materials of the formulae III and IV are reacted with one another according to Scheme 4 are generally from 0.9 to 1.4, preferably from 0.95 to 1.2, particularly preferably from 0.98 to 1.15, for the ratio of III to iso(thio)cyanate IV.

The iso(thio)cyanate IV is preferably added over 5-30 min to a mixture of compound III in one of the abovementioned solvents at 10-25° C., and the mixture is then stirred at 20-80° C. for another 0.5 to 24 hours, preferably 1 to 10 hours, to bring the reaction to completion. It is, of course, also possible to initially charge the iso(thio)cyanate IV in one of the abovementioned solvents, to add the N-substituted perhydrodiazine of the formula III and then to complete the reaction as described above.

The iso(thio)cyanates IV used in Scheme 4 are known or can be prepared analogously to known processes; see, for example, Houben-Weyl, “Methoden der Organischen Chemie” [Methods of Organic Chemistry], Vol. VIII, p. 120 (1952), Vol. IX, pp. 875, 869 (1955), EP 304920, EP 238711 and the literature references given in WO 94/10173.

It is possible, for example, to prepare isothiocyanates IV by reacting an aromatic amine Q—NH₂, hereinbelow also referred to as aniline compound IX, with phosgene or thiophosgene X, according to Scheme 5. In Scheme 5, Q and W are as defined above.

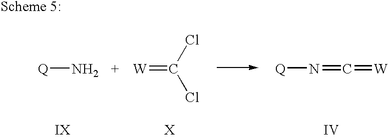

The reaction according to Scheme 5 is usually carried out in an inert organic solvent. The reaction temperature is generally in the range from 10 to 200° C.

The reaction time is generally 1-20 hours, preferably 2-15 hours, particularly preferably 3-10 hours. Solvents used for these reactions are—depending on the temperature range—hydrocarbons, such as pentane, hexane, cyclopentane, cyclohexane, toluene, xylene, chlorinated hydrocarbons, such as methylene chloride, chloroform, 1,2-dichloroethane, 1,1,2,2-tetrachloroethane, chlorobenzene, 1,2-, 1,3- or 1,4-dichlorobenzene, ethers, such as 1,4-dioxane, anisole, glycol ethers, such as dimethyl glycol ether, diethyl glycol ether, diethylene glycol dimethyl ether, esters, such as ethyl acetate, propyl acetate, methyl isobutyrate, isobutyl acetate, carboxamides, such as DMF, N-methylpyrrolidone, nitrated hydrocarbons, such as nitrobenzene, ureas, such as tetraethylurea, tetrabutylurea, dimethylethyleneurea, dimethylpropyleneurea, nitriles, such as acetonitrile, propionitrile, butyronitrile or isobutyronitrile, or else mixtures of individual solvents.

Frequently, a basic reaction auxiliary is employed. Suitable for this purpose are, for example, basic inorganic compounds, for example alkali metal or alkaline earth metal hydroxides or basic alkali metal or alkaline earth metal bicarbonates or carbonates. However, it is also possible to carry out the reaction in the presence of an organic base, for example triethylamine, tri-n-propylamine, N-ethyldiisopropylamine, pyridine, α-, β-, γ-picoline, 2,4-, 2,6-lutidine, N-methylpyrrolidine, dimethylaniline, N,N-dimethylcyclohexylamine, quinoline or acridine.

For the reaction of the amine IX with thiophosgene X (W═S), the amine is usually initially charged in an inert solvent, and the thiophosgene is then added. The addition is usually carried out over a period of 10-60 min at a temperature in the range from 10 to 40° C., preferably from 20 to 30° C. In general, to bring the reaction to completion, the components are allowed to react further at 50-180° C., preferably 60-120° C., particularly preferably 70-100° C. The reaction time is generally in the range from 10 min to 15 hours. The molar ratio of aniline IX to thiophosgene X (W═S) is preferably from 0.9 to 5, with preference from 0.95 to 3, particularly preferably from 0.98 to 1.3. If appropriate, the reaction can be carried out in the presence of an auxiliary base, for example calcium-carbonate.

If phosgene X (W═O) is used, it is expedient to treat the amine IX first with hydrogen chloride at 10-40° C., preferably 20-30° C. This is followed by the introduction of phosgene at 60-150° C., preferably 70-120° C., if appropriate in the presence of the catalyst activated carbon. Instead of phosgene, it is also possible to use diphosgene. The diphosgene is advantageously added over 2-20 min with stirring at from 0 to −5° C. to a mixture of the starting material and one of the solvents mentioned above, if appropriate with addition of activated carbon, DMF or the organic base, the mixture is allowed to warm to 10° C. over a period of one hour and stirring is then continued at 10-60° C. for another 1 to 12 hours. The molar amount of phosgene or diphosgene is from 0.98 to 5, preferably from 1 to 3, particularly preferably from 1 to 1.3, per mole of starting material.

The concentration of the starting materials in the solvent is generally from 0.1 to 5 mol/l, preferably from 0.2 to 2 mol/l.

The reaction can be carried out under atmospheric pressure or superatmospheric pressure, continuously or batchwise.

For work-up, excess phosgene or thiophosgene and the solvent are removed under reduced pressure, and the residue is then employed for the next reaction, Scheme 4.

Suitable aniline compounds IX are described, for example, in WO 01/05775.

In the case of anilines IX having a free phenol or thiophenol function, the process according to Scheme 5 is surprising, since what would have been expected was the formation of the corresponding O-aryl or S-aryl chlorothionoformates. Both free phenols and thiophenols react with thiophosgene at their phenol function, as described, for example, in JP 60 67 467, Collect. Czech. Chem. Commun., 1979, 44, 918 (Phenols) and J. Chem. Soc. Perkin Trans. 1981 Part 1, 413, J. Chem. Commun. 1975, 926 (thiophenols). Furthermore, in the case of simultaneous amino and thiophenol substitution, the formation of benzothiazole derivatives is known, see Heterocycl. Chem. 1991, 28, 359.

EP 648 772 describes, in a general manner, the formation of phenyl isothiocyanates simultaneously substituted by a free hydroxyl or amino group. Since thiophosgene does generally not differentiate between amino groups and the hydroxyl function, the examples of EP 648 772 only describe the reaction of a protected aniline.

A particularly interesting variant of the conversion shown in Scheme 5 accordingly relates to the preparation of thioisocyanates of the formula IVb

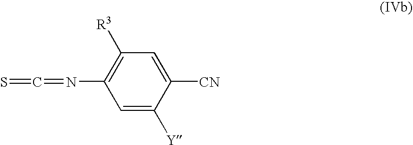

in which R³ is halogen and Y″ is hydroxyl or mercapto. These compounds are novel, and they are also important as interesting precursors for the process according to the invention.

Another particularly interesting variant of the conversion shown in Scheme 5 furthermore relates to the preparation of isocyanates of the formula IVc

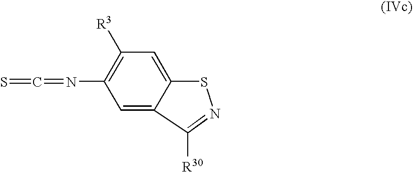

where

R³ is halogen and

R³⁰ has the meanings mentioned above under Q-7 by reacting anilines IXb

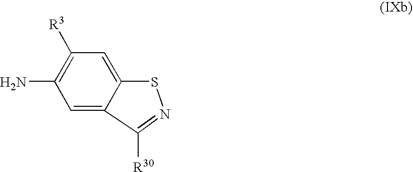

in which R³ is halogen and R³⁰ is as defined above with thiophosgene. The reaction is carried out in the manner described above. The compounds IVc are novel and, as interesting precursors for the process according to the invention, also form part of the subject matter of the invention.

Preference is given to isothiocyanates IVc in which

R³ is halogen, in particular chlorine or fluorine,

R³⁰ is hydrogen, C₁-C₆-alkyl, C₃-C₈-cycloalkyl, CH₂O—C₁-C₄-alkyl, CH₂O—C₃-C₄-alkenyl, CH₂O—C₃-C₄-alkynyl, CH₂CH₂O—C₁-C₄-alkyl, CH₂CH₂O—C₃-C₄-alkenyl, CH₂CH₂O—C₃-C₄-alkynyl, (C₁-C₄-alkoxy)carbonyl, (C₃-C₄-alkenyloxy)carbonyl, (C₃-C₄-alkynyloxy)carbonyl, (C₁-C₄-alkoxy)carbonyl-C₁-C₂-alkyl, (C₃-C₄-alkenyloxy)carbonyl-C₁-C₂-alkyl, (C₃₋C₄-alkynyloxy)carbonyl-C₁-C₂-alkyl, C₁-C₄-alkylsulfonylamidocarbonyl, CH(O—C₁-C₄-alkyl)₂, CH[O(CH₂)₃O], CH[O(CH₂)₄O] or phenyl which may be unsubstituted or may for its part carry one to three substituents, in each case selected from the group consisting of cyano, nitro, halogen, C₁-C₂-alkyl, CF₃, C₁-C₂-alkoxy, (C₁-C₂-alkoxy)carbonyl and C₁-C₂-alkoxycarbonyl-C₁-C₂-alkyl.

Each of the alkyl radicals in the radicals mentioned above may be unsubstituted or may carry one, two or three, preferably only one, substituents independently of one another selected from the group consisting of halogen, cyano and methoxy. Each cycloalkyl radical may be unsubstituted or may carry one, two or three substituents independently of one another selected from the group consisting of halogen, cyano, methoxy and methyl.

Particularly preferably, R³⁰ is one of the radicals below:

R³⁰ is C₁-C₆-alkyl, C₃-C₈-cycloalkyl, CH₂O—C₁-C₄-alkyl, CH₂O—C₃-C₄-alkenyl, CH₂O—C₃-C₄-alkynyl, (C₁-C₄-alkoxy)carbonyl, (C₃-C₄-alkenyloxy)carbonyl, (C₃-C₄-alkynyloxy)carbonyl, (C₁-C₄-alkoxy)carbonyl-C₁-C₂-alkyl, (C₃-C₄-alkenyloxy)carbonyl-C₁-C₂-alkyl, (C₃-C₄-alkynyloxy)carbonyl-C₁-C₂-alkyl, C₁-C₄-alkylsulfonylamidocarbonyl,

CH(O—C₁-C₄-alkyl)₂, CH[O(CH₂)₃O], CH[O(CH₂)₄O], phenyl, 2-, 3-, 4-chlorophenyl, 2,4-dichlorophenyl, 2-, 3-, 4-CF₃-phenyl, 2-, 3-, 4-methoxycarbonylphenyl, 2-, 3-, 4-tolyl, 2-, 3-, 4-anisyl, 2-, 3-, 4-methoxycarbonylphenyl.

During the preparation of the ureas II according to Scheme 4, it was surprisingly found that the reaction of the perhydrodiazines of the formula III in which R is a group C(S)OR² (perhydrodiazine III′) with an isothiocyanate S═C═N—Q IVa leads directly to the compounds of the formula I′ in which X and W are both sulfur, without the addition of a base being required (see Scheme 6), if the reaction is carried out in an aprotic polar solvent, for example a cyclic ether, such as tetrahydrofuran or dioxane. Analogously, the compound Ia′ is obtained from IIIa′.

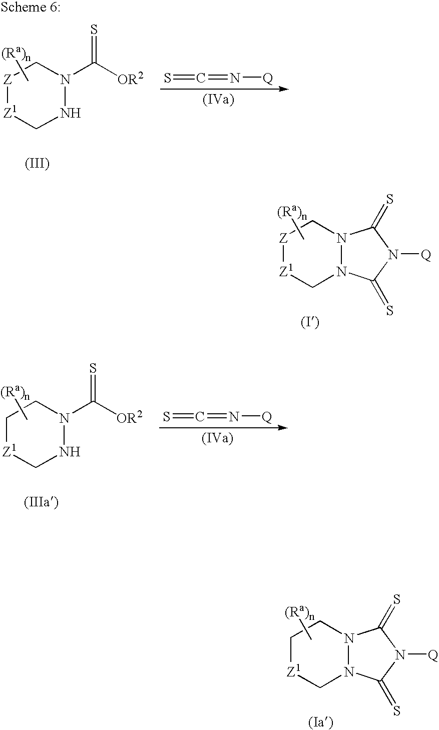

Compounds II can also be prepared by the process shown in Scheme 7a or 7b by reacting a urea derivative VIIa or VIIb with a compound of the formula R²—O—C(X)—A or of the formula R²—S—C(X)—A where A is a leaving group, for example halogen. The reaction is preferably carried out in the presence of a base. In Schemes 7a and 7b, n, R², X, R^{a}, Z, Z¹, W and Q are as defined above. Hereinbelow, the compound of the formula R²—O—C(X)—A is referred to as compound VIIIa, and the compound of the formula R²—S—C(X)—A is referred to as compound VIIIb.

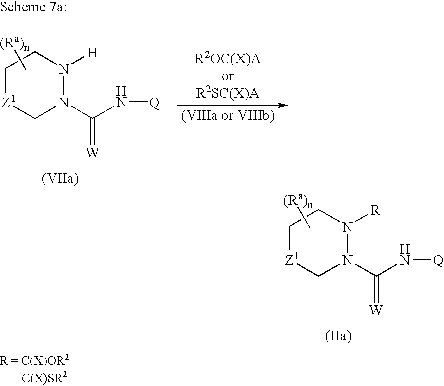

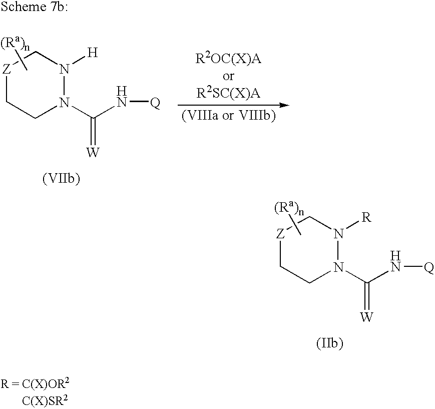

Some of the urea compounds VIIb used in Scheme 7b are known from WO 94/10173 and WO 00/01700. Moreover, the urea compounds of the formulae VIIa and VIIb used in Scheme 7a and Scheme 7b, respectively, are known from the earlier application PCT/EP00/05794.

The oxazine derivatives, used as starting materials according to Scheme 4, of the formula IIIa (compounds III, in which Z is a methylene group which is optionally substituted by R^{a}) are, according to a preferred embodiment, prepared by reacting, in a first reaction step, a substituted hydrazine of the formula V

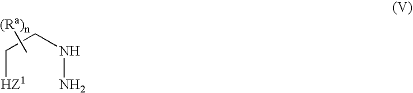

in which R^{a} and n are as defined above and Z¹ is oxygen or sulfur with a compound of the formula R²—O—C(X)—A or of the formula R²—S—C(X)—A (VIIIa and VIIIb, respectively) in which R² and X are as defined above and A is a nucleophilically displaceable leaving group, in particular a halogen atom and specifically chlorine. This gives a hydrazine derivative of the formula VI

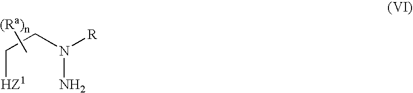

in which Z¹, R, R^{a} and n are as defined above.

In a second step, the compound VI is cyclized with formaldehyde in the presence of an acid to the substituted perhydrodiazines of the formula IIIa where Z¹=O or S, which are, if appropriate, in the case that Z¹=S, oxidized in a further reaction step to give the sulfoxides where Z¹=SO or sulfones where Z¹=SO₂.

Examples of suitable nucleophilically displaceable leaving groups A are halogen, preferably chlorine or bromine, furthermore C₁-C₆-alkoxy, such as methoxy, ethoxy, n-propoxy, n-butoxy, C₁-C₄-haloalkoxy, such as trichloromethoxy, trifluoromethoxy, pentafluoroethoxy, N-bonded heterocyclyl, such as imidazolyl, C₁-C₆-alkylcarbonyloxy (or C₁-C₆-alkanoate), such as acetate, propionate, n-butyrate, isobutyrate, pivalate and caproate, C₁-C₆-haloalkylcarbonyloxy, such as mono—, di- and trichloroacetate, C₁-C₆-alkylsulfonyloxy, such as methylsulfonyloxy, C₁-C₆-haloalkylsulfonyloxy, such as trifluoromethylsulfonyloxy, phenylsulfonyloxy, where the phenyl radical may, if appropriate, be mono- or disubstituted by halogen or C₁-C₆-alkyl, such as phenylsulfonyloxy, p-toluenesulfonyloxy and p-Cl-phenylsulfonyloxy, N-bonded nitrogen-C₅-C₆-heterocyclyl, such as N-imidazolyl.

Preferred leaving groups A are halogen, in particular chlorine or bromine, and furthermore acetate or trifluoroacetate.

The cyclization of the 2nd preparation step can be carried out using both formaldehyde or a compound which releases formaldehyde under acidic conditions, such as paraformaldehyde or 1,3,5-trioxane, in the presence of an acid.

However, it is also possible to react the hydrazides obtained in the 1st reaction step with formaldehyde to give the Schiff base which is then cyclized by addition of an acid.

The reaction described in Scheme 8 below is an example of the preparation of the compounds IIIa where, starting from 2-hydrazinoethanol and methyl chloroformate as acid derivative, firstly the N-amino-N-methoxycarbonyl-2-hydrazinoethanol is prepared, which is cyclized in a subsequent reaction with formaldehyde to give tetrahydro-4-methoxycarbonyl-4H-1-oxa-3,4-diazine.

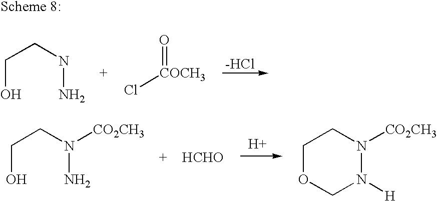

Preferred embodiments of the process are mentioned below:

The first reaction step is explained in more detail below: the reaction of the hydrazinoethanols/-thiols V with the compounds VIIIa or VIIIb is advantageously carried out in the presence of a solvent at from −30 to 100° C., preferably from −10 to 80° C., particularly preferably from 0 to 60° C.

The solvents used for these reactions are—depending on the temperature range—hydrocarbons, such as pentane, hexane, cyclopentane, cyclohexane, toluene, xylene, chlorinated hydrocarbons, such as methylene chloride, chloroform, 1,2-dichloroethane, 1,1,2,2-tetrachloroethane, chlorobenzene, 1,2-, 1,3- or 1,4-dichlorobenzene, ethers, such as 1,4-dioxane, anisole, glycol ethers, such as dimethyl glycol ether, diethyl glycol ether, diethylene glycol dimethyl ether, esters, such as ethyl acetate, propyl acetate, methyl isobutyrate, isobutyl acetate, carboxamides, such as DMF, N-methylpyrrolidone, nitrated hydrocarbons, such as nitrobenzene, ureas, such as tetraethylurea, tetrabutylurea, dimethylethyleneurea, dimethylpropyleneurea, sulfoxides, such as dimethyl sulfoxide, sulfones, such as dimethyl sulfone, diethyl sulfone, tetramethylene sulfone, nitriles, such as acetonitrile, propionitrile, butyronitrile or isobutyronitrile; water or else mixtures of individual solvents.

The molar ratios in which the starting materials V and VIIIa or VIIIb are reacted with one another are generally from 0.9 to 1.2, preferably 0.95 to 1.1, particularly preferably 0.98 to 1.04, for the ratio of VIIIa or VIIIb to hydrazinoethanol/-thiol V.

The first reaction step is advantageously carried out under neutral conditions. If an acidic reaction product is formed in the reaction, for example hydrogen halide if A in the formula VIIIa or VIIIb is halogen, this is removed by addition of basic compounds, for example alkali metal or alkaline earth metal hydroxides or bicarbonates or carbonates. However, the reaction can also be carried out in the presence of an organic base, for example triethylamine, tri-n-propylamine, N-ethyldiisopropylamine, pyridine, α-, β-, γ-picoline, 2,4-, 2,6-lutidine, N-methylpyrrolidine, dimethylaniline, N,N-dimethylcyclohexylamine, quinoline or acridine.

Finally, the reaction can also be carried out in an aqueous two-phase system, preferably in the presence of phase-transfer catalysts, such as quaternary ammonium or phosphonium salts. The reaction conditions mentioned above and in EP-A 556737, as well as the abovementioned phase-transfer catalysts, are suitable for the two-phase reaction.

Advantageously, the compound VIIIa or VIIIb is added, at 0 to 60° C. and over a period of 0.25 to 2 hours, to a mixture of the hydrazinoethanol/thiol V and the base in one of the abovementioned solvents, and stirring at 0 to 60° C. is continued for 0.5 to 16 hours, preferably 2 to 8 hours, for the reaction to go to completion.

If an aqueous two-phase system is used, the starting materials V and VIIIa or VIIIb can be added with stirring, in any order, to a mixture of the phase-transfer catalyst in the two phases, and the reaction can then be completed in the temperature range mentioned by adding base.

The reaction can be carried out under atmospheric pressure or under superatmospheric pressure, continuously or batchwise.

For work-up, any precipitated salts are separated off, or their removal is completed by addition of nonpolar solvents, and the hydrazides are thus accumulated in the filtrate.

The second reaction step is explained below: the hydrazides are subsequently reacted, advantageously under acidic conditions, with a formaldehyde solution or paraformaldehyde in one of the abovementioned solvents.

For the subsequent step, advantageously 0.9 to 1.2, preferably 0.95 to 1.1, particularly preferably 0.98 to 1.04, molar equivalents of formaldehyde or paraformaldehyde are employed per mole of hydrazide derivative VI. The concentration of the starting materials in the solvent is 0.1 to 5 mol/l, preferably 0.2 to 2 mol/l.

The acid used can be an aromatic sulfonic acid, for example benzenesulfonic acid, p-chloro- or p-toluenesulfonic acid, an aliphatic sulfonic acid, such as methanesulfonic acid, trifluoromethanesulfonic acid, ethanesulfonic acid and n-propylsulfonic acid, a sulfaminic acid, such as methylsulfaminic acid, ethylsulfaminic acid or isopropylsulfaminic acid, an aliphatic carboxylic acid, such as acetic acid, trifluoroacetic acid, propionic acid, butyric acid or isobutyric acid, or an inorganic acid, such as hydrochloric acid, sulfuric acid, nitric acid or boric acid. Advantageously, it is also possible to use an acid such as acetic acid or propionic acid directly as reaction medium. The acidic catalyst is advantageously employed in an amount of from 1 to 20 mol %, preferably 3 to 15 mol %, particularly preferably 5 to 10 mol %, of acid per mole of hydrazide.

Preferably, a formaldehyde solution or paraformaldehyde is added over a period of 2 to 60 min to a mixture of hydrazide and the acidic catalyst in one of the abovementioned solvents at 0 to 100° C., advantageously 10 to 80° C., particularly preferably 20 to 50° C., and stirring is continued at 40 to 50° C. for 10 to 50 hours, preferably 15 to 30 hours, to bring the reaction to completion.

If an aqueous formaldehyde solution is used, the water is advantageously removed, for example using a water separator.

However, it is also possible to add the acidic catalyst to a mixture of hydrazide and paraformaldehyde in one of the abovementioned solvents and then to complete the reaction as described.

The reaction can be carried out under atmospheric pressure or under superatmospheric pressure, continuously or batchwise.

The oxidation of the compounds III where Z or Z¹=S to the sulfoxides (Z or Z¹=SO₂), which follows, if appropriate, is preferably carried out using hydrogen peroxide, the sulfoxides being obtained with approximately equivalent amounts of oxidizing agent, and the sulfones being obtained with about double the molar quantities.

The oxidation with hydrogen peroxide can be catalyzed by suitable metal compounds, for example transition metal oxides, such as vanadium pentoxide, sodium tungstate, potassium dichromate, iron oxide tungstate, sodium tungstate/molybdic acid, osmic acid, titanium trichloride, selenium dioxide, phenyleneselenic acid, oxovanadinyl-2,4-pentanedionate. The catalysts are generally employed in an amount of from 0.5 to 10% by weight, based on the substrate used, but it is also possible to employ stoichiometric amounts because the inorganic catalysts can easily be filtered off and recovered.

Solvents which are suitable for the oxidation with hydrogen peroxide are, for example water, acetonitrile, alcohols, such as methanol, ethanol, isopropanol, tert-butanol, chlorinated hydrocarbons, such as methylene chloride, 1,1,2,2-tetrachloroethane, or ketones such as acetone or methyl ethyl ketone.

In addition to hydrogen peroxide, it is also possible to use, as oxidizing agents, peracids, such as perbenzoic acid, monoperphthalic acid or 3-chloroperbenzoic acid. The reaction with peracids is expediently carried out in chlorinated hydrocarbons, such as methylene chloride or 1,2-dichloroethane.

Also very suitable for oxidizing the thiols to sulfoxides or sulfones are chlorine and bromine. This oxidation is expediently carried out in polar solvents, such as water, acetonitrile, dioxane, or in two-phase systems, such as aqueous potassium bicarbonate solution/dichloromethane, and also acetic acid. It is furthermore possible to employ as sources of active halogen tert-butyl hypochlorite, hypochlorous and hypobromous acid, their salts, and also N-halo compounds, such as N-bromo- and N-chlorosuccinimide, or else sulfuryl chloride.

Also suitable for the oxidation is photosensitized oxygen transfer, in which case the photosensitizers used are usually organic dyes, for example porphyrines, such as tetraphenylporphyrine, chlorophyll, protoporphyrine, xanthene dyes, such as Bengal Rose or phenothiazine dyes, such as Methylene Blue.

Suitable inert solvents are hydrocarbons, such as pentane, hexane, heptane, cyclohexane, chlorinated hydrocarbons, such as methylene chloride, 1,2-dichloroethane, 1,1,2,2-tetrachloroethane, alcohols, such as methanol, ethanol, n-propanol or isopropanol, ketones, such as acetone, methyl ethyl ketone, polar aprotic solvents, such as acetonitrile, propionitrile or aromatic hydrocarbons, such as benzene, toluene, chlorobenzene or xylene. In place of oxygen, it is also possible to use ozone in the abovementioned solvents, plus ether, 1,4-dioxane or tetrahydrofuran (THF).

Besides photosensitization, catalysts are also suitable for the oxidation with oxygen, for example oxides and sulfides of nickel, copper, aluminum, tungsten, chromium, vanadium, ruthenium, titanium, manganese, molybdenum, magnesium and iron.

Either the sulfoxides (IIIa where Z¹=SO) or their sulfones (IIIa where Z¹=SO₂) are obtained, depending on the stoichiometry of the oxidizing agents used. The molar ratios in which the starting materials are reacted with one another are generally from 0.9 to 1.8, preferably 1.05 to 1.3, for the ratio of tetrahydrothiadiazine to oxidizing agent in the case of the oxidation to the sulfoxide and generally 1.9 to 3.5, preferably 2.05 to 2.9, in the case of oxidation to the sulfone.

The concentration of the starting materials in the solvent is generally 0.1 to 5 mol/l, preferably 0.2 to 2 mol/l. It is advantageous to initially charge the 1-thiadiazine of the formula IIIa where Z¹=S or the sulfoxide, if appropriate together with one of the abovementioned catalysts, in one of the abovementioned solvents, and then to add the oxidizing agent over a period of 0.25 to 20 hours with stirring. The addition and the reaction temperature depends on the optimum efficiency of the oxidizing agent in question and on avoiding side reactions. If photosensitized oxygen is used, the reaction is generally carried out at from −20 to 80° C.; however, if metal catalysis is employed, the reaction is generally carried out at from 50 to 140° C., and if ozone is used, the reaction is generally carried out at from −78 to 60° C. Owing to the limited solubility of the oxygen derivatives, they are preferably introduced continuously into the reaction mixture over a relatively long period of time (up to 20 h) until the oxidation has been completed at the sulfoxide or sulfone stage. Liquid or easily soluble oxidizing agents, such as hydrogen peroxide, hypochlorous or hypobromous acid, tert-butyl hypochlorite, chlorine or bromine, furthermore N-chloro- or N-bromosuccinimide, can be added to the reaction mixture of the thiadiazine or thiadiazine sulfoxide over shorter periods of time, such as 0.25 to 6 h, depending on the exothermic character of the reaction, and the reaction is ended after a further 1 to 60 h. Preference is furthermore given to adding the liquid or dissolved oxidizing agent gradually. In the case of hydrogen peroxide, the reaction is generally carried out at from 0 to 90° C., with tert-butyl hypochlorite generally at from −78 to 30° C., and with N-halo compounds generally at from 0 to 30° C. In the case of chlorine or bromine, a reaction temperature of from 0 to 40° C. is recommended.

The oxidations can be carried out under atmospheric pressure or under superatmospheric pressure, continuously or batchwise. The multistep reaction can advantageously also be carried out as a one-pot process, where the thiadiazines IIIa (Z¹=S) are converted directly, without isolation and purification, into the sulfoxides IIIa (Z¹=SO) or the sulfones IIIa (Z¹=SO₂). Accordingly, the reaction product Ia is, if appropriate, allowed to cool to from 90 to 20° C., a solvent, for example methylene chloride and/or water, is added, if appropriate, and the oxidizing agent is then added at the rate of its consumption. Particularly preferred oxidizing agents are hydrogen peroxide and sodium hypochlorite.

For work-up of the oxidation mixture, the end products IIIa are generally taken up in a water-immiscible solvent, acidic impurities and/or oxidizing agents are extracted using dilute alkali or water, the mixture is dried and the solvent is removed under reduced pressure.

It is, of course, also possible to prepare compounds of the formula I in which X is oxygen and Q is Q-2 or Q-3 by the processes for acidic cyclization known from the prior art.

The compounds of the formulae VIIa and VIIb defined in Scheme 7a and 7b, respectively,

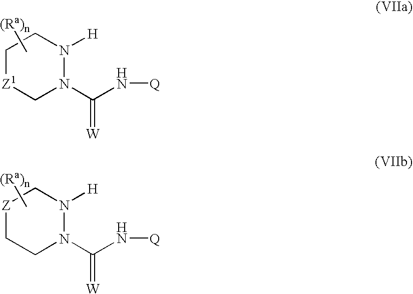

can, for example, be cyclized with phosgene or a phosgene equivalent, such as diphosgene, to give the compounds I according to the invention. The reaction of compound VIIa with phosgene or a phosgene equivalent is novel and also forms part of the subject matter of the present invention.

The cyclization of VIIa or VIIb with phosgene or a phosgene derivative is advantageously carried out in the presence of one of the anhydrous solvents mentioned above, at temperatures in the range from −10 to 120° C., preferably from 0 to 80° C., particularly preferably from 10 to 60° C.

Advantageously, the phosgene is, at 10-60° C., introduced with stirring into a mixture of a 4-(phenylcarbamoyl)tetrahydro-4H-1,3,4-oxadiazine (or thiadiazine) and an amount of from 0.5 to 5% by weight, based on the starting material, of activated carbon as catalyst in one of the abovementioned anhydrous solvents over a period of from 0.5 to 20 hours, preferably from 1 to 12 hours.

The reaction may additionally be accelerated by a basic amide catalyst, for example DMF, which can usually be employed in an amount of from 0.3 to 10% by weight, based on the starting material. It is also possible to use organic bases, such as triethylamine, tri-n-propylamine, N,N-dimethylaniline or N,N-dimethylcyclohexylamine as basic catalyst. Pyridine may also be used advantageously, if appropriate directly as solvent.

Instead of phosgene, it is also possible to diphosgene. Advantageously, the diphosgene is, over 2-20 min, added with stirring at from 0 to −5° C. to the mixture of the starting material and one of the solvents mentioned above, if appropriate with addition of activated carbon, DMF or the organic base, the mixture is allowed to warm to 10° C. over a period of 1 hour and is then stirred for another 1 to 12 hours at 10-60° C. The molar amount of phosgene or diphosgene is from 0.98 to 5, preferably from 1 to 3, particularly preferably from 1 to 1.3, per mole of starting material.

The concentration of the starting materials in the solvent is generally from 0.1 to 5 mol/l, preferably from 0.2 to 2 mol/l.

The reaction can be carried out under atmospheric pressure or superatmospheric pressure, continuously or batchwise.

Compared to the acidic cyclization processes known from the prior art for preparing fused tetrahydrotriazoles, the basic cyclization process of Scheme 3 according to the invention has the advantage that it is not necessary to use phosgene. A further important advantage of the process according to the invention is the fact that by this route it is possible to prepare compounds of the formula I in which Z is an optionally R^{a}-substituted methylene group and W is sulfur, which in principle cannot be prepared by the processes of the prior art as described in WO 94/10173 and WO 00/01700 and which hitherto could also not be prepared by other routes, as mentioned at the outset.

Moreover, it is possible to prepare compounds I {R⁵=C₁-C₆-alkoxy, C₁-C₆-alkylthio, C₃-C₆-cycloalkoxy, C₃-C₆-cycloalkylthio, C₂-C₆-alkenyloxy, C₂-C₆-alkenylthio, C₂-C₆-alkynyloxy, C₂-C₆-alkynylthio, (C₁-C₆-alkyl)carbonyloxy, (C₁-C₆-alkyl)carbonylthio, (C₁-C₆-alkoxy)carbonyloxy, (C₂-C₆-alkenyl)carbonyloxy, (C₂-C₆-alkenyl)carbonylthio, (C₂-C₆-alkynyl)carbonyloxy, (C₂-C₆-alkynyl)carbonylthio or C₁-C₆-alkylsulfonyloxy, where each radical may, if desired, carry one of the radicals mentioned under R⁵} by reacting the corresponding hydroxy or mercapto compound {R⁵=OH, SH} or an alkali metal or alkaline earth metal salt thereof with a reactive alkylating agent G-R^{5′} of the formula XI, if appropriate in the presence of an acid acceptor and if appropriate in the presence of a diluent. In the formula XI, G is a nucleophilically displaceable leaving group and R^{5′} is a C₁-C₆-alkyl, C₃-C₆-cycloalkyl, C₂-C₆-alkenyl, C₂-C₆-alkynyl, (C₁-C₆-alkyl)carbonyl, (C₁-C₆-alkoxy)carbonyl, (C₂-C₆-alkenyl)carbonyl, (C₂-C₆-alkynyl)carbonyl or C₁-C₆-alkylsulfonyl radical which may carry the substituents mentioned under R⁵.

Examples of nucleophilically displaceable leaving groups are halogen, preferably chlorine or bromine, C₁-C₆-alkylcarbonyloxy (or C₁-C₆-alkanoate) such as acetate, propionate, n-butyrate, isobutyrate, pivalate, C₁-C₆-haloalkylcarbonyloxy, such as mono-, di- and trichloroacetate, C₁-C₆-alkylsulfonyloxy, such as methylsulfonyloxy, C₁-C₆-haloalkylsulfonyloxy, such as trifluoromethylsulfonyloxy, phenylsulfonyloxy, where the phenyl radical may, if appropriate, be mono- or polysubstituted by halogen or C₁-C₆-alkyl, such as phenylsulfonyloxy, p-tolylsulfonyloxy and p-chlorophenylsulfonyloxy.

Preferred leaving groups are halogen, in particular chlorine or bromine, and furthermore acetate or trifluoroacetate and methylsulfonate or trifluoromethylsulfonate.

The reaction of the triazoles I {R⁵=OH, SH or an alkali metal or alkaline earth metal salt thereof} with the compounds of the formula XI is advantageously carried out in the presence of a solvent at temperatures in the range from −20 to 120° C., preferably from −10 to 100° C., particularly preferably from 10 to 90° C.

The solvents used for these reactions are—depending on the temperature range—hydrocarbons, such as pentane, hexane, cyclopentane, cyclohexane, toluene, xylene, chlorinated hydrocarbons, such as methylene chloride, chloroform, 1,2-dichloroethane, 1,1,2,2-tetrachloroethane, chlorobenzene, 1,2-, 1,3- or 1,4-dichlorobenzene, ethers, such as 1,4-dioxane, anisole, glycol ethers, such as dimethyl glycol ether, diethyl glycol ether, diethylene glycol dimethyl ether, esters, such as ethyl acetate, propyl acetate, methyl isobutyrate, isobutyl acetate, carboxamides, such as DMF, N-methylpyrrolidone, nitrated hydrocarbons, such as nitrobenzene, ureas, such as tetraethylurea, tetrabutylurea, dimethylethyleneurea, dimethylpropyleneurea, nitriles, such as acetonitrile, propionitrile, butyronitrile or isobutyronitrile, or else mixtures of individual solvents.

The molar ratios in which the starting materials I {R⁵=OH, SH or an alkali metal or alkaline earth metal salt thereof} and XI are reacted with one another are generally from 0.9 to 1.2, preferably from 0.95 to 1.1, particularly preferably from 0.98 to 1.04.

The alkylation is advantageously carried out under neutral conditions. If an acidic reaction product is formed in the reaction, for example hydrogen halide if G in formula XI is halogen, this is removed by addition of basic compounds, for example alkali metal or alkaline earth metal hydroxides or bicarbonates or carbonates. However, the reaction can also be carried out in the presence of an organic base, for example triethylamine, tri-n-propylamine, N-ethyldiisopropylamine, pyridine, α-, β-, γ-picoline, 2,4-, 2,6-lutidine, N-methylpyrrolidine, dimethylaniline, N,N-dimethylcyclohexylamine, quinoline or acridine.

Finally, the reaction can also be carried out in an aqueous two-phase system, preferably in the presence of phase-transfer catalysts, such as quaternary ammonium or phosphonium salts. The reaction conditions mentioned in EP-A 556737 are suitable for the two-phase reaction.

Suitable phase-transfer catalysts are quaternary ammonium or phosphonium salts. Suitable compounds which may be mentioned are: tetraalkyl-(C₁-C₁₈)ammonium chlorides, bromides or fluorides, N-benzyltrialkyl-(C₁-C₁₈)ammonium chlorides, bromides or fluorides, tetraalkyl-(C₁-C₁₈)phosphonium chlorides or bromides, tetraphenylphosphonium chloride or bromide, (phenyl)ₒ(alkyl-(C₁-C₁₈)ₚ-phosphonium chlorides or bromides, where o=1 to 3, p=3 to 1 and o+p=4. Particular preference is given to tetraethylammonium chloride and N-benzyltriethylammonium chloride. The amount of phase-transfer catalyst is generally up to 20% by weight, preferably between 1 and 15% by weight aid particularly preferably between 2 and 8% by weight, based on the triazole I {R⁵=OH, SH or an alkali metal or alkaline earth metal salt thereof}.

Advantageously, the alkylating agent XI is added over a period of from 0.15 to 2 hours to a mixture of the triazole I {R⁵=OH, SH or an alkali metal or alkaline earth metal salt thereof} and the base in one of the abovementioned solvents at 10-60° C., and the mixture is stirred for another 0.5 to 16 hours, preferably 2 to 8 hours, at 10-90° C. to bring the reaction to completion.

If an aqueous two-phase system is used, the starting materials I {R⁵=OH, SH or an alkali metal or alkaline earth metal salt thereof} and XI can be added in any order, with stirring, to a mixture of the phase-transfer catalyst in the two phases, and the reaction can then be brought to completion in the temperature range mentioned, with addition of base.

The reaction can be carried out under atmospheric pressure or superatmospheric pressure, continuously or batchwise.

For work-up, any salts which may have precipitated are separated off, or their separation is brought to completion by addition of nonpolar solvents, and in this manner the triazoles I are enriched in the filtrate.

Compounds of the formula Ia in which Z is optionally R^{a}-substituted methylene, W is sulfur and Q is one of the radicals Q-1, Q-4, Q-5 or Q-6 defined above, and the agriculturally compatible salts of these compounds are, surprisingly, effective herbicides and accordingly also form part of the subject matter of the present invention. With respect to their herbicidal activity, they are superior to the compounds of the formula I in which W is an oxygen atom.

Moreover, compounds of the formula Ia in which Q is one of the radicals Q-2, Q-3 or Q-7 defined above and the agriculturally compatible salts of these compounds are likewise herbicidally active and accordingly also form part of the subject matter of the present invention. With respect to their herbicidal activity, in these compounds W is likewise preferably sulfur.

Depending on the substitution pattern, the novel compounds of the formula Ia may contain one or more centers of chirality, in which case they are present as mixtures of enantiomers or diastereomers. In the case of compounds Ia having at least one olefinic radical, E/Z isomers may also be possible, if appropriate. The invention provides both the pure enantiomers or diastereomers and mixtures thereof.

Suitable agriculturally useful salts are especially the salts of those cations or the acid addition salts of those acids whose cations and anions, respectively, do not adversely affect the herbicidal action of the compounds Ia. Thus, suitable cations are in particular the ions of the alkali metals, preferably lithium, sodium and potassium, of the alkaline earth metals, preferably calcium, magnesium and barium, and of the transition metals, preferably manganese, copper, zinc and iron, and also the ammonium ion which, if desired, may carry one to four C₁-C₄-alkyl substituents and/or one phenyl or benzyl substituent, preferably diisopropylammonium, tetramethylammonium, tetrabutylammonium, trimethylbenzylammonium, furthermore phosphonium ions, sulfonium ions, preferably tri(C₁-C₄-alkyl)sulfonium, and sulfoxonium ions, preferably tri(C₁-C₄-alkyl)sulfoxonium.

Anions of useful acid addition salts are primarily chloride, bromide, fluoride, hydrogensulfate, sulfate, dihydrogenphosphate, hydrogenphosphate, phosphate, nitrate, hydrogencarbonate, carbonate, hexafluorosilicate, hexafluorophosphate, benzoate, and the anions of C₁-C₄-alkanoic acids, preferably formate, acetate, propionate and butyrate. They can be formed by reacting the compounds of the formula Ia with an acid of the corresponding anion, preferably hydrochloric acid, hydrobromic acid, sulfuric acid, phosphoric acid or nitric acid.

With a view to the use of the compounds Ia according to the invention as herbicides, the variables are preferably as defined below, in each case on their own or in combination:

Q is Q-1, Q-2, Q-3, Q-4 or Q-7;

X, Y and Y′ independently of one another are O or S;

T is a chemical bond or O;

U is a chemical bond, C₁-C₄-alkylene, O or S;

R³ is hydrogen, fluorine or chlorine;

R⁴ is chlorine, trifluoromethyl or cyano;

R⁵ is hydroxyl, mercapto, cyano, nitro, halogen, C₁-C₆-alkyl, C₁-C₆-haloalkyl, C₁-C₆-alkoxy-(C₁-C₆-alkyl)carbonyl, C₁-C₆-alkylthio-(C₁-C₆-alkyl)carbonyl, (C₁-C₆-alkyl)iminooxycarbonyl, C₁-C₆-alkoxy-C₁-C₆-alkyl, C₁-C₆-alkoxyamino-C₁-C₆-alkyl, C₁-C₆-alkoxy-C₁-C₆-alkylamino-C₁-C₆-alkyl,

C₁-C₆-alkoxy, C₁-C₆-alkylthio, C₃-C₆-cycloalkoxy, C₃-C₆-cycloalkylthio, C₂-C₆-alkenyloxy, C₂-C₆-alkenylthio, C₂-C₆-alkynyloxy, C₂-C₆-alkynylthio, (C₁-C₆-alkyl)carbonyloxy, (C₁-C₆-alkyl)carbonylthio, (C₁-C₆-alkoxy)carbonyloxy, (C₂-C₆-alkenyl)carbonyloxy, (C₂-C₆-alkenyl)carbonylthio, (C₂-C₆-alkynyl)carbonyloxy, (C₂-C₆-alkynyl)carbonylthio, C₁-C₆-alkylsulfonyloxy or C₁-C₆-alkylsulfonyl, where each of these 17 radicals may, if desired, carry one, two or three substituents selected from the group consisting of:

halogen, nitro, cyano, hydroxyl, C₃-C₆-cycloalkyl, C₁-C₆-alkoxy, C₃-C₆-cycloalkoxy, C₃-C₆-alkenyloxy, C₃-C₆-alkynyloxy, C₁-C₆-alkoxy-C₁-C₆-alkoxy, C₁-C₆-alkylthio, C₁-C₆-alkylsulfinyl, C₁-C₆-alkylsulfonyl, C₁-C₆-alkylideneaminooxy, oxo, ═N—OR¹⁰

phenyl, phenoxy or phenylsulfonyl, where the three last-mentioned substituents for their part may carry one, two or three substituents, in each case selected from the group consisting of halogen, nitro, cyano, C₁-C₆-alkyl, C₁-C₆-haloalkyl, C₁-C₆-alkoxy and (C₁-C₆-alkoxy)carbonyl;

—CO—R¹¹, —CO—OR¹¹, —CO—SR¹¹, —CO—N(R¹¹)—R¹², —OCO—R¹¹, —OCO—OR^{11′}, —OCO—SR^{11′}, —OCO—N(R¹¹)—R¹², —N(R¹¹)—R¹², and —C(R¹³)═N—OR¹⁰;

C(Z²)—R¹⁴, —C(═NR¹⁵)R¹⁴, C(R¹⁴)(Z²R¹⁶)(Z⁴R¹⁷), C(R¹⁴)═C(R¹⁸)—CN, C(R¹⁴)═C(R¹⁸)—CO—R¹⁹, —CH(R¹⁴)—CH(R¹⁸)—COR¹⁹, —C(R¹⁴)═C(R¹⁸)—CH₂-CO—R¹⁹, —C(R¹⁴)═C(R¹⁸)—C(R²⁰)═C(R²¹)—CO—R¹⁹, —C(R¹⁴)═C(R¹⁸)—CH₂-CH(R²¹)—CO—R²¹, —CO—R²³, —CO—SR²³, —CON(R²³)—OR¹⁰, —C≡C—CO—NHOR¹⁰, —C≡C—CO—N(R²³)—OR¹⁰, —C≡C—CS—NH—OR¹⁰, —C≡C—CS—N(R²³)—OR¹⁰, —C(R¹⁴)═C(R¹⁸)—CO—NHOR¹⁰, —C(R¹⁴)═C(R¹⁸)—CO—N(R²³)—OR¹⁰, —C(R¹⁴)═C(R¹⁸)—CS—NHOR¹⁰, —C(R¹⁴)═C(R¹⁸)—CS—N(R²³)—OR¹⁰, —C(R¹⁴)═C(R¹⁸)—C(R¹³)═N—OR¹⁰, C(R¹³)═N—OR¹⁰, —C≡C—C(R¹³)═NOR¹⁰, C(Z³R¹⁶)(Z⁴R¹⁷)—OR²³, —C(Z³R¹⁶)(Z⁴R¹⁷)SR²³, C(Z³R¹⁶)(Z⁴R¹⁷)—N(R²⁴)R²⁵, —N(R²⁴)—R²⁵, —CO—N(R²⁴)—R²⁵ or —C(R¹⁴)═C(R¹⁸)CO—N(R²⁴)R²⁵; where Z², Z³, Z⁴ independently of one another are oxygen or sulfur;

R⁶ is hydrogen, halogen, cyano, C₁-C₆-alkyl, C₁-C₆-haloalkyl, C₃-C₇-cycloalkyl, saturated C₃-C₇-heterocyclyl which has one or two heteroatoms selected from the group consisting of oxygen and sulfur in the ring, C₁-C₆-alkoxyalkyl, cyano-C₁-C₆-alkyl, CO₂H, C₁-C₆-alkoxycarbonyl and C₁-C₆-alkoxycarbonyl-C₁-C₆-alkyl, C₃-C₆-alkenyl or C₃-C₆-alkynyl;

R⁷ is hydrogen, halogen, cyano, C₁-C₆-alkyl, C₁-C₆-haloalkyl, C₃-C₇-cycloalkyl, saturated C₃-C₇-heterocyclyl which has one or two heteroatoms selected from the group consisting of oxygen and sulfur in the ring, C₁-C₆-alkoxyalkyl, cyano-C₁-C₆-alkyl, CO₂H, C₁-C₆-alkoxycarbonyl and C₁-C₆-alkoxycarbonyl-C₁-C₆-alkyl, C₃-C₆-alkenyl or C₃-C₆-alkynyl;

R⁸ is hydrogen or C₁-C₃-alkyl;

R⁹ is hydrogen, C₁-C₃-alkyl;

R⁸ and R⁹ together are C═O;

R¹⁰ is hydrogen, C₁-C₆-alkyl, C₁-C₆-haloalkyl, C₃-C₆-cycloalkyl, C₃-C₆-alkenyl, C₃-C₆-alkynyl, C₁-C₆-alkoxy-C₁-C₆-alkyl, cyano-C₁-C₆-alkyl, (C₁-C₆-alkoxy)carbonyl-C₁-C₆-alkyl or phenylalkyl, where the phenyl ring may be mono- to trisubstituted by halogen, cyano, nitro, C₁-C₃-alkyl, C₁-C₃-haloalkyl or C₁-C₃-alkoxy;

R¹¹ is hydrogen, C₁-C₆-alkyl, C₃-C₆-cycloalkyl, C₃-C₆-alkenyl, C₃-C₆-alkynyl, C₁-C₆-alkoxy-C₁-C₆-alkyl, (C₁-C₆-alkoxy)carbonyl-C₁-C₆-alkyl, C₃-C₆-alkenyloxycarbonyl-C₁-C₆-alkyl, phenyl or benzyl which may be unsubstituted or mono- to trisubstituted on the phenyl ring by halogen, cyano, nitro, C₁-C₃-alkyl, C₁-C₃-haloalkyl or C₁-C₃-alkoxy;

R^{11′} has the meanings mentioned for R¹¹, except for hydrogen;

R¹² is hydrogen, hydroxyl, C₁-C₆-alkyl, C₃-C₇-cycloalkyl, C₃-C₆-cycloalkylaminocarbonyl, C₁-C₆-alkylaminocarbonyl, C₁-C₆-alkoxy, (C₁-C₃-alkoxy)carbonyl-C₁-C₃-alkoxy, C₃-C₆-alkenyl, C₃-C₆-alkenyloxy, C₃-C₆-alkynyl or C₃-C₆-alkynyloxy;

R¹³ is hydrogen, halogen, C₁-C₆-alkyl, C₁-C₆-haloalkyl, C₁-C₆-alkoxy, C₃-C₆-alkenyloxy, (C₁-C₆-alkoxy)carbonylalkoxy, C₂-C₆-alkenyl, (C₂-C₆-alkenyl)carbonyloxy, C₃-C₆-alkynyl, (C₂-C₆-alkynyl)carbonyloxy, phenyl, phenoxy or benzyl, where the phenyl rings of the 3 last-mentioned radicals may be unsubstituted or mono- to trisubstituted by halogen, cyano, nitro, C₁-C₃-alkyl, C₁-C₃-haloalkyl, C₁-C₃-alkoxy or (C₁-C₃-alkoxy)carbonyl;

R¹⁴ is hydrogen, cyano, C₁-C₆-alkyl, C₁-C₆-haloalkyl, C₂-C₆-alkenyl, C₂-C₆-alkynyl, C₁-C₆-alkoxy-C₁-C₆-alkyl or (C₁-C₆-alkoxy)carbonyl;

R¹⁵ is hydrogen, C₁-C₆-alkyl, C₃-C₆-alkenyl, C₃-C₆-alkynyl, C₃-C₆-cycloalkyl, C₁-C₆-haloalkyl, C₁-C₆-alkoxy-C₁-C₆-alkyl, C₁-C₆-alkoxy, (C₁-C₆-alkoxy)carbonyl-C₁-C₆-alkyl, phenyl or phenyl-(C₁-C₆-alkyl), where the two last-mentioned phenyl radicals may be substituted by halogen, cyano, nitro, C₁-C₃-alkyl, C₁-C₃-haloalkyl, C₁-C₃-alkoxy or (C₁-C₃-alkoxy)carbonyl;

R¹⁶, R¹⁷ independently of one another are C₁-C₆-alkyl, C₁-C₆-haloalkyl, C₃-C₆-alkenyl, C₃-C₆-alkynyl, C₁-C₆-alkoxy-C₁-C₆-alkyl, or

R¹⁶ and R¹⁷ together are a saturated 2- to 4-membered carbon chain which may carry an oxo substituent, where a carbon atom of this chain which is not adjacent to the variables Z³ and Z⁴ may be replaced by —O—, —S—, —N═, —NH— or —N(C₁-C₆-alkyl)- and where the carbon chain may additionally be mono- to trisubstituted by halogen or C₁-C₆-alkyl;

R¹⁸ is hydrogen, cyano, halogen, C₁-C₆-alkyl, C₁-C₆-haloalkyl or C₁-C₆-alkoxy;

R¹⁹ is hydrogen, OR²⁸, S—R²⁸, C₁-C₆-alkyl which may carry one or two C₁-C₆-alkoxy substituents, C₂-C₆-alkenyl, C₂-C₆-alkynyl, C₁-C₆-haloalkyl or C₃-C₆-cycloalkyl;

R²⁰ is hydrogen, cyano, halogen, C₁-C₆-alkyl, C₃-C₆-alkenyl or C₃-C₆-alkynyl;

R²¹ is hydrogen, cyano, halogen, C₁-C₆-alkyl, C₁-C₆-alkoxy or C₁-C₆-haloalkyl;

R²² is hydrogen, cyano or C₁-C₆-alkyl;

R²³, R²⁸ independently of one another are hydrogen, C₁-C₆-alkyl, C₁-C₆-haloalkyl, C₂-C₆-alkenyl or C₂-C₆-alkynyl, where the 4 last-mentioned groups may in each case carry one or two of the following radicals: cyano, halogen, C₁-C₆-alkoxy, (C₁-C₆-alkyl)carbonyl, (C₁-C₆-alkoxy)carbonyl, phenyl or phenyl-C₁-C₆-alkyl;

R²⁴, R²⁵, R²⁶, R²⁷ independently of one another are hydrogen, C₁-C₆-alkyl, C₃-C₆-alkenyl, C₂-C₆-alkynyl, C₃-C₆-cycloalkyl, C₁-C₆-haloalkyl, C₁-C₆-alkoxy-C₁-C₆-alkyl, C₁-C₆-alkylcarbonyl, (C₁-C₆-alkoxy)carbonyl, or

R²⁴ and R²⁵ and/or R²⁶ and R²⁹ together with the respective common nitrogen atom are a saturated or unsaturated 4- to 7-membered azaheterocycle which, in addition to carbon ring members, may, if desired, contain an oxygen atom or an —NH— group.

In particular, R⁵ in Q-1 is:

C₁-C₆-alkoxy, C₂-C₆-alkenyloxy or C₂-C₆-alkynyloxy, where each of the 3 last-mentioned radicals may, if desired, carry one to three substituents, in each case selected from the group consisting of halogen, C₁-C₆-alkoxy, C₃-C₆-alkenyloxy, C₃-C₆-alkynyloxy, C₁-C₆-alkylsulfonyl, —CO—R¹¹, —CO—OR¹¹, —CO—N(R¹¹)—R¹², —N(R¹¹)—R¹², and —C(R¹³)═N—OR¹⁰;

—CO—R¹⁴, —C(═NR¹⁵)—R¹⁴, —C(R¹⁴)(OR¹⁶)(OR¹⁷) —C(R¹⁴)═C(R¹⁸)—CO—R¹⁹, —CH(R¹⁴)—CH(R¹⁸)—CO—R¹⁹, —CO—OR²³, —CO—N(R²³)—OR¹⁰, —C(R¹⁴)═C(R¹⁸)—CO—N(R²³)—OR¹⁰, —C(R¹³)═N—OR¹⁰, —C(OR¹⁶)(OR¹⁷)—OR²³, —N(R²⁴)R²⁵, —CON(R²⁴)R²⁵ or —C(R¹⁴)═C(R¹⁸)CO—N(R²⁴)R²⁵;

and specifically C₂-C₆-alkenyloxy, C₂-C₆-alkynyloxy, —C(R¹⁴)(OR¹⁶)(OR¹⁷), —C(R¹⁴)═C(R¹⁸)—C(O)R¹⁹, —CH(R¹⁴)—CH(R¹⁸)—C(O)R¹⁹, C(O)OR²³, —C(O)—N(R²³)—OR¹⁰, —C(R¹³)═N—OR¹⁰ and C(O)N(R²⁴)R²⁵, where R¹⁰ to R¹⁹ and R²³ to R²⁵ are as defined above and have, in particular, the meanings mentioned below:

R¹⁰ is C₁-C₆-alkyl, C₁-C₆-haloalkyl, C₃-C₆-alkenyl, C₃-C₆-haloalkenyl, C₃-C₆-alkynyl, C₁-C₆-cyanoalkyl and C₁-C₆-alkoxycarbonyl-C₁-C₆-alkyl;

R¹³ is hydrogen, C₁-C₆-alkyl, C₁-C₆-alkoxy, C₃-C₆-alkenyloxy, C₃-C₆-alkynyloxy, C₁-C₆-alkoxycarbonyl-C₁-C₆-alkyl and C₁-C₆-alkoxycarbonyl-C₁-C₆-alkoxy;

R¹⁴ is hydrogen, C₁-C₆-alkyl;

R¹⁵ is C₁-C₆-alkoxy;

R¹⁶ and R¹⁷ independently of one another are C₁-C₆-alkyl;

R¹⁸ is hydrogen, halogen, C₁-C₆-alkyl;

R¹⁹ is hydroxyl, C₁-C₆-alkoxy, C₁-C₆-alkylthio, C₁-C₆-alkoxycarbonyl-C₁-C₆-alkyl;

R²³ is C₁-C₆-alkyl, C₃-C₆-haloalkyl, C₃-C₆-alkenyl, C₃-C₆-alkynyl, C₃-C₆-alkenyloxy, C₃-C₆-alkynyloxy, C₁-C₆-alkoxycarbonyl-C₁-C₆-alkyl, C₃-C₆-alkenyloxycarbonyl-C₁-C₆-alkyl, C₃-C₆-alkynyloxycarbonyl-C₁-C₆-alkyl, C₁-C₆-alkoxyalkyl;

R²⁴ is hydrogen, C₁-C₆-alkyl;

R²⁵ is hydrogen, C₁-C₆-alkyl, C₁-C₆-alkoxy, or

R²⁴ and R²⁵ together are a 6-membered saturated azaheterocycle which has optionally one or two non-adjacent oxygen atoms in the ring.

With a view to the herbicidal activity of compounds Ia in which Q is Q-7, R³⁰ preferably has the meanings given for the isothiocyanates IVc as being preferred. In particular, R³⁰ in Q-7 is:

C₁-C₆-alkyl, C₃-C₈-cycloalkyl, CH₂O—C₁-C₄-alkyl, CH₂O—C₃-C₄-alkenyl, CH₂O—C₃-C₄-alkynyl, (C₁-C₄-alkoxy)carbonyl, (C₃-C₄-alkenyloxy)carbonyl, (C₃-C₄-alkynyloxy)carbonyl, (C₁-C₄-alkoxy)carbonyl-C₁-C₂-alkyl, (C₃-C₄-alkenyloxy)carbonyl-C₁-C₂-alkyl, (C₃-C₄-alkynyloxy)carbonyl-C₁-C₂-alkyl, C₁-C₄-alkylsulfonylamidocarbonyl, where each alkyl radical may be unsubstituted or may carry one, two or three substituents selected from the group consisting of halogen, cyano and methoxy and each cycloalkyl radical may be unsubstituted or may carry one, two or three substituents selected from the group consisting of halogen, cyano, methoxy and methyl,

CH(O—C₁-C₄-alkyl)₂, CH(O(CH₂)₃O], CH[O(CH₂)₄O] or phenyl which may be unsubstituted or may for its part carry one, two or three substituents, in each case selected from the group consisting of cyano, nitro, halogen, C₁-C₂-alkyl, CF₃, C₁-C₂-alkoxy, (C₁-C₂-alkoxy)carbonyl and C₁-C₂-alkoxycarbonyl-C₁-C₂-alkyl.

A special class relates to compounds Ia in which Q is Q-1, W is sulfur and X is oxygen or sulfur. Here, the variables have the meanings given above and particularly preferably the following meanings:

Z is O or S, in particular O,

n has the value 0,

R³ is hydrogen or halogen, in particular fluorine or chlorine,

R⁴ is hydrogen, halogen, in particular fluorine or chlorine, or cyano, and

R⁵ is C₁-C₆-alkyl, C₃-C₆-alkenyl, C₁-C₆-haloalkyl, C₃-C₆-haloalkenyl, C₁-C₆-alkoxy, C₁-C₆-haloalkoxy, C₁-C₆-alkylthio, C₁-C₆-haloalkylthio, C₃-C₆-alkenyloxy, C₃-C₆-alkynyloxy, C₁-C₆-haloalkenyloxy, C₃-C₆-alkenylthio, C₃-C₆-haloalkenylthio, C₁-C₆-alkoxycarbonyl-C₁-C₆-alkoxy, C₃-C₆-alkenyloxycarbonyl-C₁-C₆-alkoxy, C₃-C₆-alkynyloxycarbonyl-C₁-C₆-alkoxy, [C₁-C₆-alkoxy]-C₁-C₆-alkoxycarbonyl-C₁-C₆-alkoxy, C₃-C₆-alkenyloxycarbonyl-C₁-C₆-alkoxycarbonyl-C₁-C₄-alkoxy, C₁-C₆-alkoxycarbonyl-C₁-C₆-alkylthio, C₁-C₆-alkenyloxycarbonyl-C₁-C₆-alkylthio, C₁-C₆-alkynyloxycarbonyl-C₁-C₆-alkylthio, [C₁-C₆-alkoxy]-C₁-C₆-alkoxycarbonyl-C₁-C₆-alkylthio, C₁-C₆-alkoxyimino-C₁-C₆-alkyl, N—C₁-C₆-alkoxy-N—(C₁-C₆-alkyl)amino-C₁-C₆-alkyl, C₁-C₆-alkylsulfonylamino, —COOR²³, —CONR²⁴R²⁵, —C(═NR¹⁵)R¹⁴, —C(R¹³)═NOR¹⁰, C(R¹⁴)═C(R¹⁸)—CO—R¹⁹, where the variables R¹⁰, R¹³ to R¹⁵, R¹⁸, R¹⁹, R²³ to R²⁵ are as defined below:

R¹⁰ is C₁-C₆-alkyl, C₁-C₆-haloalkyl, C₃-C₆-alkenyl, C₃-C₆-haloalkenyl, C₃-C₆-alkynyl, C₁-C₆-cyanoalkyl and C₁-C₆-alkoxycarbonyl-C₁-C₆-alkyl;

R¹³ is hydrogen, C₁-C₆-alkyl, C₁-C₆-alkoxy, C₁-C₆-alkoxycarbonyl-C₁-C₆-alkyl, C₁-C₆-alkoxycarbonyl-C₁-C₆-alkoxy and phenoxycarbonyl-C₁-C₆-alkoxy;

R¹⁴ is hydrogen, C₁-C₆-alkyl;

R¹⁵ is C₁-C₆-alkoxy;

R¹⁸ is hydrogen, halogen, C₁-C₆-alkyl;

R¹⁹ is hydroxyl, C₁-C₆-alkoxy, C₁-C₆-alkylthio, C₁-C₆-alkoxycarbonyl-C₁-C₆-alkyl;

R²³ is C₁-C₆-alkyl, C₃-C₆-haloalkyl, C₃-C₆-alkenyl, C₃-C₆-alkynyl, C₁-C₆-alkoxycarbonyl-C₁-C₆-alkyl, C₃-C₆-alkenyloxycarbonyl-C₁-C₆-alkyl, C₃-C₆-alkynyloxycarbonyl-C₁-C₆-alkyl, C₁-C₆-alkoxyalkyl;

R²⁴ is hydrogen, C₁-C₆-alkyl;

R²⁵ is hydrogen, C₁-C₆-alkyl, C₁-C₆-alkoxy,

or R²⁴ and R²⁵ together are a 6-membered saturated azaheterocycle which optionally has one or two non-adjacent oxygen atoms in the ring.

R⁵ is in particular as defined below:

R⁵ is CN, COOH, C₁-C₄-alkoxyiminomethyl, C₁-C₄-alkoxy, C₃-C₆-cycloalkyloxy, C₃-C₆-alkenyloxy, C₃-C₆-alkynyloxy, C₃-C₆-alkenyloxyiminomethyl, (C₁-C₄-alkoxycarbonyl)-C₂-C₆-alkenyloxy, C₃-C₆-alkynyloxyiminomethyl, 2-[C₁-C₄-alkoxycarbonyl]-2-chloroethyl, 2-[C₁-C₄-alkoxycarbonyl]-2-chloroethenyl, C₁-C₄-alkoxycarbonyl, (C₁-C₆-alkoxycarbonyl)-C₁-C₄-alkoxy, (C₁-C₆-alkoxycarbonyl)-C₁-C₄-thioalkyl, COOR²³ where R²³=C₁-C₄-alkoxy-C₁-C₄-alkyl or C₃-C₆-alkenyloxycarbonyl-C₁-C₄-alkyl, CONR²⁴R²⁵ where R²⁴=hydrogen or C₁-C₄-alkyl and R²⁵=hydrogen, C₁-C₄-alkyl or C₁-C₄-alkoxy.

Two further classes relate to compounds of the formula Ia where Q is Q-2 or Q-3. Here, the variables independently of one another particularly preferably have the following meanings:

W is oxygen or, preferably, sulfur,

X is oxygen or sulfur,

Z is O or S, in particular O,

n has the value 0,

R³ is hydrogen or halogen,

R⁴ is hydrogen or halogen,

Y is O or S,

U is a single bond, oxygen or C₁-C₄-alkylene and

R⁶ is hydrogen, halogen, cyano, C₁-C₆-alkyl, C₁-C₆-haloalkyl, C₃-C₇-cycloalkyl, saturated C₃-C₇-heterocyclyl which has one or two heteroatoms selected from the group consisting of oxygen and sulfur in the ring, C₁-C₆-alkoxyalkyl, cyano-C₁-C₆-alkyl, CO₂H, C₁-C₆-alkoxycarbonyl and C₁-C₆-alkoxycarbonyl-C₁-C₆-alkyl, C₃-C₆-alkenyl or C₃-C₆-alkynyl.

Two further classes relate to compounds of the formula Ia where Q is Q-4 or Q-5. Here, the variables independently of one another particularly preferably have the following meanings:

W is sulfur,

X is oxygen or sulfur,

Z is O or S, in particular O,

X is O or S,

n has the value 0,

R³ is hydrogen or halogen,

Y is O or S,

Y′ in formula Q-5 is oxygen or sulfur,

T is a single bond, oxygen or C₁-C₄-alkylene and

R⁷ is hydrogen, halogen, cyano, C₁-C₆-alkyl, C₁-C₆-haloalkyl, C₃-C₇-cycloalkyl, saturated C₃-C₇-heterocyclyl, which has one or two heteroatoms selected from the group of oxygen and sulfur in the ring, C₁-C₆-alkoxyalkyl, cyano-C₁-C₆-alkyl, CO₂H, C₁-C₆-alkoxycarbonyl and C₁-C₆-alkoxycarbonyl-C₁-C₆-alkyl, C₃-C₆-alkenyl or C₃-C₆-alkynyl.

A further class relates compounds of the formula Ia where Q is Q-6. Here, the variables have the meanings mentioned above and, independently of one another, the following meanings:

W is sulfur,

X is oxygen or sulfur,

Z is O or S, in particular O,

n has the value 0,

R³ is hydrogen or halogen,

R⁴ is hydrogen or halogen, where

R⁸ and R⁹ independently of one another are hydrogen, C₁-C₆-alkyl, halogen, cycloalkyl or C₁-C₆-haloalkyl, or

R⁸ and R⁹ together with the carbon atom to which they are attached are a carbonyl group.

A special class relates to compounds of the formula Ia where n, R^{a}, Z¹, X and W are as defined above and Q is the radical Q-7 defined above. Among these compounds, preference is given to those in which the variables n, R^{a}, Z¹, X and W independently of one another, preferably in combination, are as defined below:

W is oxygen or, in particular, sulfur,

X is oxygen or sulfur,

Z is O or S, in particular O,

n has the value 0,

X is oxygen or sulfur,

W is sulfur.

In the radical Q-7, R³ is preferably halogen, in particular fluorine or chlorine. R³⁰ has the meanings given above, in particular the meanings given as being preferred.

R³⁰ in Q-7 is in particular:

hydrogen, C₁-C₆-alkyl, C₃-C₈-cycloalkyl, CH₂O—C₁-C₄-alkyl, CH₂O—C₃-C₄-alkenyl, CH₂O—C₃-C₄-alkynyl, CH₂CH₂O—C₁-C₄-alkyl, CH₂CH₂O—C₃-C₄-alkenyl, CH₂CH₂O—C₃-C₄-alkynyl, (C₁-C₄-alkoxy)carbonyl, (C₃-C₄-alkenyloxy)carbonyl, (C₃-C₄-alkynyloxy)carbonyl, (C₁-C₄-alkoxy)carbonyl-C₁-C₂-alkyl, (C₃-C₄-alkenyloxy)carbonyl-C₁-C₂-alkyl, (C₃-C₄-alkynyloxy)carbonyl-C₁-C₂-alkyl, C₁-C₄-alkylsulfonylamidocarbonyl, CH(O—C₁-C₄-alkyl)₂, CH[O(CH₂)₃O], CH[O(CH₂)₄O] or phenyl which may be unsubstituted or may for its part carry one, two or three substituents, in each case selected from the group consisting of cyano, nitro, halogen, C₁-C₂-alkyl, CF₃, C₁-C₂-alkoxy, (C₁-C₂-alkoxy)carbonyl and C₁-C₂-alkoxycarbonyl-C₁-C₂-alkyl, where each alkyl radical of the radicals mentioned above may be unsubstituted or may carry one, two or three, preferably only one, substituents selected from the group consisting of halogen, cyano and methoxy, and where each cycloalkyl radical may be unsubstituted or may carry one, two or three substituents selected from the group consisting of halogen, cyano, methoxy and methyl.

Particularly preferably, R³⁰ is one of the following radicals:

C₁-C₆-alkyl, C₃-C₈-cycloalkyl, CH₂O—C₁-C₄-alkyl, CH₂O—C₃-C₄-alkenyl, CH₂O—C₃-C₄-alkynyl, (C₁-C₄-alkoxy)carbonyl, (C₃-C₄-alkenyloxy)carbonyl, (C₃-C₄-alkynyloxy)carbonyl, (C₁-C₄-alkoxy)carbonyl-C₁-C₂-alkyl, (C₃-C₄-alkenyloxy)carbonyl-C₁-C₂-alkyl, (C₃-C₄-alkynyloxy)carbonyl-C₁-C₂-alkyl, C₁-C₄-alkylsulfonylamidocarbonyl,

CH(O—C₁-C₄-alkyl)₂, CH[O(CH₂)₃O], CH[O(CH₂)₄O], phenyl, 2-, 3-, 4-chlorophenyl, 2,4-dichlorophenyl, 2-, 3-, 4-CF₃-phenyl, 2-, 3-, 4-methoxycarbonylphenyl, 2-, 3-, 4-tolyl, 2-, 3-, 4-anisyl, 2-, 3-, 4-methoxycarbonylphenyl.

Particularly preferred compounds of the formula Ia are compounds of the formula Ia-1 where R³, R⁴ and R⁵ have the meanings given in each case in one row of Table 1 (compounds Ia-1.1 to Ia-1.206).

TABLE 1

| | |
|---|---|
| | (Ia-1) |
| 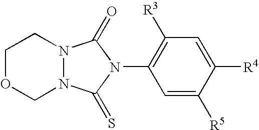 | |

| | | | |
|---|---|---|---|
| No. | R³ | R⁴ | R⁵ |

| | | | |
|---|---|---|---|
| Ia-1.1 | H | Cl | OCH₂C≡CH |
| Ia-1.2 | H | Cl | OCH₂CH═CH₂ |
| Ia-1.3 | H | Cl | OCH(CH₃)C≡CH |
| Ia-1.4 | F | Cl | OCH₂C≡CH |
| Ia-1.5 | F | Cl | OCH₂CH═CH₂ |
| Ia-1.6 | F | Cl | OCH(CH₃)C≡CH |
| Ia-1.7 | H | Cl | CO₂CH₃ |
| Ia-1.8 | H | Cl | CO₂CH₂CH═CH₂ |
| Ia-1.9 | H | Cl | CO₂CH₂C≡CH |
| Ia-1.10 | H | Cl | CO₂CH₂CH₂OCH₃ |
| Ia-1.11 | F | Cl | CO₂CH₃ |
| Ia-1.12 | F | Cl | CO₂CH₂CH═CH₂ |
| Ia-1.13 | F | Cl | CO₂CH₂C≡CH |
| Ia-1.14 | F | Cl | CO₂CH₂CH₂OCH₃ |
| Ia-1.15 | H | CN | OCH₂C≡CH |
| Ia-1.16 | F | CN | OCH₂C≡CH |
| Ia-1.17 | H | Cl | OCH₂CO₂CH₃ |
| Ia-1.18 | H | Cl | OCH₂CO₂CH₂C≡CH |
| Ia-1.19 | H | Cl | OCH₂CO₂CH₂CH₂OCH₃ |
| Ia-1.20 | H | Cl | OCH₂CO₂C(CH₃)₂CO₂CH₂CH═CH₂ |
| Ia-1.21 | F | Cl | OCH₂CO₂CH₃ |
| Ia-1.22 | F | Cl | OCH₂CO₂CH₂C≡CH |
| Ia-1.23 | F | Cl | OCH₂CO₂CH₂CH₂OCH₃ |
| Ia-1.24 | F | Cl | OCH(CH₃)CO₂CH₃ |
| Ia-1.25 | F | Cl | OCH(CH₃)CO₂C₂H₅ |
| Ia-1.26 | F | Cl | OCH(CH₃)CO₂CH₂CH═CH₂ |
| Ia-1.27 | F | Cl | OCH(CH₃)CO₂CH₂C≡CH |
| Ia-1.28 | F | Cl | OCH(CH₃)CO₂CH₂CH₂OCH₃ |
| Ia-1.29 | F | Cl | OCH₂CO₂-nC₅H₁₁ |
| Ia-1.30 | F | Cl | OCH(CH₃)CO₂-nC₅H₁₁ |
| Ia-1.31 | H | Cl | CO₂CH₂CO₂CH₃ |
| Ia-1.32 | H | Cl | CO₂CH₂CO₂C₂H₅ |
| Ia-1.33 | H | Cl | CO₂C(CH₃)₂CO₂CH₂CH═CH₂ |
| Ia-1.34 | F | Cl | CO₂CH₂CO₂CH₃ |
| Ia-1.35 | F | Cl | CO₂CH₂CO₂C₂H₅ |
| Ia-1.36 | F | Cl | CO₂C(CH₃)₂CO₂CH₂CH═CH₂ |
| Ia-1.37 | F | CN | CO₂CH₃ |
| Ia-1.38 | F | CN | CO₂C₂H₅ |
| Ia-1.39 | F | CN | CO₂CH₂CH₂OCH₃ |
| Ia-1.40 | F | CN | CO₂CH₂CO₂CH₃ |
| Ia-1.41 | F | CN | CO₂CH₂CO₂C₂H₅ |
| Ia-1.42 | F | CN | CO₂C(CH₃)₂CO₂CH₂CH═CH₂ |
| Ia-1.43 | F | CN | OCH₂CO₂CH₃ |
| Ia-1.44 | F | CN | OCH₂CO₂C₂H₅ |
| Ia-1.45 | F | CN | OCH₂CO₂CH₂C≡CH |
| Ia-1.46 | F | CN | OCH₂CO₂CH₂CH₂OCH₃ |
| Ia-1.47 | F | CN | OCH(CH₃)CO₂CH₃ |
| Ia-1.48 | F | CN | OCH(CH₃)CO₂C₂H₅ |
| Ia-1.49 | F | CN | OCH(CH₃)CO₂CH₂CH₂OCH₃ |
| Ia-1.50 | H | Cl | NHSO₂CH₃ |
| Ia-1.51 | H | Cl | NHSO₂C₂H₅ |
| Ia-1.52 | H | Cl | NHSO₂-nC₃H₇ |
| Ia-1.53 | H | Cl | NHSO₂-iC₃H₇ |
| Ia-1.54 | F | Cl | NHSO₂CH₃ |
| Ia-1.55 | F | Cl | NHSO₂C₂H₅ |
| Ia-1.56 | F | Cl | NHSO₂-nC₃H₇ |
| Ia-1.57 | F | Cl | NHSO₂-iC₃H₇ |
| Ia-1.58 | F | CN | NHSO₂CH₃ |
| Ia-1.59 | F | CN | NHSO₂C₂H₅ |
| Ia-1.60 | F | CN | NHSO₂-nC₃H₇ |
| Ia-1.61 | F | CN | NHSO₂-iC₃H₇ |
| Ia-1.62 | H | Cl | OCH₂C(Cl)═CH₂ |
| Ia-1.63 | Cl | Cl | OCH₂C(Cl)═CH₂ |
| Ia-1.64 | F | Cl | OCH₂C(Cl)═CH₂ |
| Ia-1.65 | F | CN | OCH₂C(Cl)═CH₂ |
| Ia-1.66 | Cl | Cl | OCH₂C≡CH |
| Ia-1.67 | Cl | Cl | OCH₂CH═CH₂ |
| Ia-1.68 | Cl | Cl | OCH₂CO₂CH₃ |
| Ia-1.69 | Cl | Cl | OCH₂CO₂nC₅H₁₁ |
| Ia-1.70 | Cl | Cl | OCH(CH₃)CO₂CH₃ |
| Ia-1.71 | Cl | Cl | OCH(CH₃)CO₂CH₂CH═CH₂ |
| Ia-1.72 | Cl | Cl | OCH(CH₃)CO₂CH₂CH₂OCH₃ |
| Ia-1.73 | Cl | Cl | CO₂CH₃ |
| Ia-1.74 | Cl | Cl | CO₂C₂H₅ |
| Ia-1.75 | Cl | Cl | CO₂CH₂CO₂CH₃ |
| Ia-1.76 | Cl | Cl | CO₂C(CH₃)₂CO₂CH₃ |
| Ia-1.77 | H | Cl | SCH₂C≡CH |
| Ia-1.78 | H | Cl | SCH₂CH═CH₂ |
| Ia-1.79 | H | Cl | SCH₂CO₂CH₃ |
| Ia-1.80 | H | Cl | SCH₂CO₂-nC₅H₁₁ |
| Ia-1.81 | H | Cl | SCH(CH₃)CO₂CH₃ |
| Ia-1.82 | H | Cl | SCH(CH₃)CO₂CH₂CH₂OCH₃ |
| Ia-1.83 | H | Cl | SCH₂CO₂CH₂CH₂OCH₃ |
| Ia-1.84 | H | Cl | OCF₂CHFCl |
| Ia-1.85 | Cl | Cl | SCH₂C≡CH |
| Ia-1.86 | Cl | Cl | SCH₂CH═CH₂ |
| Ia-1.87 | Cl | Cl | SCH₂CO₂CH₃ |
| Ia-1.88 | Cl | Cl | SCH₂CO₂-nC₅H₁₁ |
| Ia-1.89 | Cl | Cl | SCH(CH₃)CO₂CH₃ |
| Ia-1.90 | Cl | Cl | SCH(CH₃)CO₂CH₂CH₂OCH₃ |
| Ia-1.91 | Cl | Cl | SCH₂CO₂CH₂CH₂OCH₃ |
| Ia-1.92 | Cl | Cl | OCF₂CHFCl |
| Ia-1.93 | F | Cl | SCH₂C≡CH |
| Ia-1.94 | F | Cl | SCH₂CH═CH₂ |
| Ia-1.95 | F | Cl | SCH₂CO₂CH₃ |
| Ia-1.96 | F | Cl | SCH₂CO₂-nC₅H₁₁ |
| Ia-1.97 | F | Cl | SCH(CH₃)CO₂CH₃ |
| Ia-1.98 | F | Cl | SCH(CH₃)CO₂CH₂CH₂OCH₃ |
| Ia-1.99 | F | Cl | SCH₂CO₂CH₂CH₂OCH₃ |
| Ia-1.100 | F | Cl | OCF₂CHFCl |
| Ia-1.101 | F | CN | SCH₂C≡CH |
| Ia-1.102 | F | CN | SCH₂CH═CH₂ |
| Ia-1.103 | F | CN | SCH₂CO₂CH₃ |
| Ia-1.104 | F | CN | SCH₂CO₂-nC₅H₁₁ |
| Ia-1.105 | F | CN | SCH(CH₃)CO₂CH₃ |
| Ia-1.106 | F | CN | SCH(CH₃)CO₂CH₂CH₂OCH₃ |
| Ia-1.107 | F | CN | SCH₂CO₂CH₂CH₂OCH₃ |
| Ia-1.108 | H | Cl | C(O)N(CH₃)₂ |
| Ia-1.109 | F | Cl | C(O)N(CH₃)₂ |
| Ia-1.110 | F | CN | C(O)N(CH₃)₂ |
| Ia-1.11l | H | Cl | C(O)—N(C₂H₄)₂O |
| Ia-1.112 | H | F | C(O)—N(C₂H₄)₂O |
| Ia-1.113 | H | Cl | CH═N—OCH₃ |
| Ia-1.114 | H | Cl | CH═N—OC₂H₅ |
| Ia-1.115 | H | Cl | C(O)NHOCH₃ |
| Ia-1.116 | H | Cl | C(O)NHOC₂H₅ |
| Ia-1.117 | H | Cl | C(═N—OCH₃)OCH₃ |
| Ia-1.118 | H | Cl | C(═N—OCH₃)OC₂H₅ |
| Ia-1.119 | H | Cl | C(═N—OCH₃)OCH₂CO₂CH₃ |
| Ia-1.120 | H | Cl | C(═N—OCH₃)OCH(CH₃)CO₂CH₃ |
| Ia-1.12l | H | Cl | CH═CH—CO₂CH₃ |
| Ia-1.122 | H | Cl | CH═CH—CO₂C₂H₅ |
| Ia-1.123 | H | Cl | CH═C(CH₃)CO₂CH₃ |
| Ia-1.124 | H | Cl | CH═C(CH₃)CO₂C₂H₅ |
| Ia-1.125 | H | Cl | CH═C(Cl)CO₂CH₃ |
| Ia-1.126 | H | Cl | CH═C(Cl)CO₂C₂H₅ |
| Ia-1.127 | H | Cl | CH═C(Br)CH₂C₂H₅ |
| Ia-1.128 | H | Cl | CH₂N(CH₃)OCH₃ |
| Ia-1.129 | H | Cl | C(═N—OCH₃)OCH₂CO₂phenyl |
| Ia-1.130 | H | F | CH═N—OC₂H₅ |
| Ia-1.13l | H | F | C(O)NHOCH₃ |
| Ia-1.132 | H | F | C(O)NHOC₂H₅ |
| Ia-1.133 | H | F | C(═N—OCH₃)OCH₃ |
| Ia-1.134 | H | F | C(═N—OCH₃)OC₂H₅ |
| Ia-1.135 | H | F | C(═N—OCH₃)OCH₂CO₂CH₃ |
| Ia-1.136 | H | F | C(═N—OCH₃)OCH(CH₃)CO₂CH₃ |
| Ia-1.137 | H | F | CH═CH—CO₂CH₃ |
| Ia-1.138 | H | F | CH═CH—CO₂C₂H₅ |
| Ia-1.139 | H | F | CH═C(CH₃)CO₂CH₃ |
| Ia-1.140 | H | F | CH═C(CH₃)CO₂C₂H₅ |
| Ia-1.14l | H | F | CH═C(Cl)CO₂CH₃ |
| Ia-1.142 | H | F | CH═C(Cl)CO₂C₂H₅ |
| Ia-1.143 | H | F | CH═C(Br)CO₂C₂H₅ |
| Ia-1.144 | H | F | CH₂N(CH₃)OCH₃ |
| Ia-1.145 | H | F | C(═N—OCH₃)OCH₂CO₂phenyl |
| Ia-1.146 | H | F | CH═N—OCH₃ |
| Ia-1.147 | F | CN | CH═N—OCH₃ |
| Ia-1.148 | F | CN | CH═N—OC₂H₅ |
| Ia-1.149 | F | CN | C(O)NHOCH₃ |
| Ia-1.150 | F | CN | C(O)NHOC₂H₅ |
| Ia-1.151 | F | CN | C(═N—OCH₃)OCH₃ |
| Ia-1.152 | F | CN | C(═N—OCH₃)OC₂H₅ |
| Ia-1.153 | F | CN | C(═N—OCH₃)OCH₂CO₂CH₃ |
| Ia-1.154 | F | CN | C(═N—OCH₃)OCH(CH₃)CO₂CH₃ |
| Ia-1.155 | F | CN | CH═CH—CO₂CH₃ |
| Ia-1.156 | F | CN | CH═CH—CO₂C₂H₅ |
| Ia-1.157 | F | CN | CH═C(CH₃)CO₂CH₃ |
| Ia-1.158 | F | CN | CH═C(CH₃)CO₂C₂H₅ |
| Ia-1.159 | F | CN | CH═C(Cl)CO₂CH₃ |
| Ia-1.160 | F | CN | CH═C(Cl)CO₂C₂H₅ |
| Ia-1.161 | F | CN | CH═C(Br)CO₂C₂H₅ |
| Ia-1.162 | F | CN | CH₂—N(CH₃)OCH₃ |
| Ia-1.163 | F | CN | C(N—OCH₃)OCH₂CO₂C₆H₅ |
| Ia-1.164 | H | Cl | CH═N—OCH₂—C≡CH |
| Ia-1.165 | H | Cl | CH═N—OCH₂—C(Cl)═CH₂ |
| Ia-1.166 | F | Cl | CH₂—CH(Cl)CO₂C₂H₅ |
| Ia-1.167 | H | Cl | CH═N—OCH₂CO₂CH₃ |
| Ia-1.168 | H | Cl | CH═N—OCH₂CO₂C₂H₅ |
| Ia-1.169 | H | Cl | CH═N—OCH₂CH₂Cl |
| Ia-1.170 | H | Cl | CH═N—OCH₂CN |
| Ia-1.171 | H | Cl | CH═N—OCH(CH₃)CO₂CH₃ |
| Ia-1.172 | H | Cl | CH═C(Cl)COSCH₃ |
| Ia-1.173 | H | Cl | CH═C(Br)COSCH₃ |
| Ia-1.174 | H | Cl | CH═C(Cl)CO₂CH₂CO₂CH₃ |
| Ia-1.175 | H | Cl | CH═C(Cl)CO₂CH(CH₃)CO₂CH₃ |
| Ia-1.176 | H | Cl | C(CH₃)═NOCH₃ |
| Ia-1.177 | H | Cl | C(CH₃)═NOC₂H₅ |
| Ia-1.178 | H | Cl | C(CH₃)═NOCH₂CO₂CH₃ |
| Ia-1.179 | F | Cl | CH═N—OCH₂C≡CH |
| Ia-1.180 | F | Cl | CH═N—OCH₂—C(Cl)═CH₂ |
| Ia-1.181 | F | Cl | CH═N—OCH₂CO₂CH₃ |
| Ia-1.182 | F | Cl | CH═N—OCH₂CO₂C₂H₅ |
| Ia-1.183 | F | Cl | CH═N—OCH₂CH₂Cl |
| Ia-1.184 | F | Cl | CH═N—OCH₂CN |
| Ia-1.185 | F | Cl | CH═N—OCH(CH₃)CO₂CH₃ |
| Ia-1.186 | F | Cl | CH═C(Cl)COSCH₃ |
| Ia-1.187 | F | Cl | CH═C(Br)COSCH₃ |
| Ia-1.188 | F | Cl | CH═C(Cl)CO₂CH₂CO₂CH₃ |
| Ia-1.189 | F | Cl | CH═C(Cl)CO₂CH(CH₃)CO₂CH₃ |
| Ia-1.190 | F | Cl | C(CH₃)═N—OCH₃ |
| Ia-1.191 | F | Cl | C(CH₃)═N—OC₂H₅ |
| Ia-1.192 | F | Cl | C(CH₃)═N—OCH₂CO₂CH₃ |
| Ia-1.193 | Cl | Cl | CH═N—OCH₂C≡CH |
| Ia-1.194 | Cl | Cl | CH═N—OCH₂—C(Cl)═CH₂ |
| Ia-1.195 | Cl | Cl | CH═N—OCH₂CO₂CH₃ |
| Ia-1.196 | Cl | Cl | CH═N—OCH₂CO₂C₂H₅ |
| Ia-1.197 | Cl | Cl | CH═N—OCH₂CH₂Cl |
| Ia-1.198 | Cl | Cl | CH═N—OCH₂CN |
| Ia-1.199 | Cl | Cl | CH═N—OCH(CH₃)CO₂CH₃ |
| Ia-1.200 | Cl | Cl | CH═C(Cl)COSCH₃ |
| Ia-1.201 | Cl | Cl | CH═C(Br)COSCH₃ |
| Ia-1.202 | Cl | Cl | CH═C(Cl)CO₂CH₂CO₂CH₃ |
| Ia-1.203 | Cl | Cl | CH═C(Cl)CO₂CH(CH₃)CO₂CH₃ |
| Ia-1.204 | Cl | Cl | C(CH₃)═NOCH₃ |
| Ia-1.205 | Cl | Cl | C(CH₃)═NOC₂H₅ |
| Ia-1.206 | Cl | Cl | C(CH₃)═NOCH₂CO₂CH₃ |

Particularly preferred compounds of the formula Ia are furthermore compounds of the formula Ia-2 where R³, R⁴ and R⁵ have the meanings given in each case in one row of Table 1 (compounds Ia-2.1 to Ia-2.206).

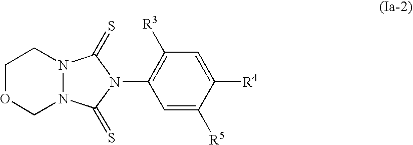

Particularly preferred compounds of the formula Ia are furthermore compounds of the formula Ia-3 where R³, R⁴ and R⁵ have the meanings given in each case in one row of Table 1 (compounds Ia-3.1 to Ia-3.206).

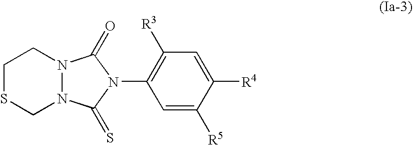

Particularly preferred compounds of the formula Ia are furthermore compounds of the formula Ia-4 where R³, R⁴ and R⁵ have the meanings given in each case in one row of Table 1 (compounds Ia-4.1 to Ia-4.206).

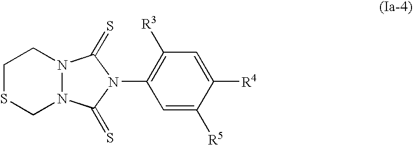

Particularly preferred compounds of the general formula Ia are furthermore compounds of the formula Ia-5 where Q is Q-2 where Y=oxygen and Z¹, X, U and R⁶ have in each case the meanings given in one row of Table 2 (compounds Ia-5.1 to Ia-5.224)

TABLE 2

| | |
|---|---|
| | (Ia-5) |
| 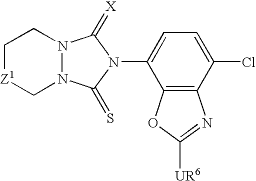 | |

| No. | X | Z¹ | U | R⁶ |
|---|---|---|---|---|
| Ia-5.1 | S | S | — | H |
| Ia-5.2 | S | S | — | CH₃ |
| Ia-5.3 | S | S | — | C₂H₅ |
| Ia-5.4 | S | S | — | n-C₃H₇ |
| Ia-5.5 | S | S | — | i-C₃H₇ |
| Ia-5.6 | S | S | — | cyclopropyl |
| Ia-5.7 | S | S | — | n-C₄H₉ |
| Ia-5.8 | S | S | — | sec-C₄H₉ |
| Ia-5 9 | S | S | — | i-C₄H₉ |
| Ia-5.10 | S | S | — | t-C₄H₉ |
| Ia-5.11 | S | S | CH₂ | cyclopropyl |
| Ia-5.12 | S | S | — | cyclopentyl |
| Ia-5.13 | S | S | — | cyclohexyl |
| Ia-5.14 | S | S | — | oxiran-2-yl |
| Ia-5.15 | S | S | — | oxetan-2-yl |
| Ia-5.16 | S | S | — | tetrahydrofuran-2-yl |
| Ia-5.17 | S | S | — | tetrahydropyran-2-yl |
| Ia-5.18 | S | S | — | oxepan-2-yl |
| Ia-5.19 | S | S | — | thiiran-2-yl |
| Ia-5.20 | S | S | — | thietan-2-yl |
| Ia-5.21 | S | S | — | tetrahydrothiofuran-2-yl |
| Ia-5.22 | S | S | — | tetrahydrothiopyran-2-yl |
| Ia-5.23 | S | S | — | thiepan-2-yl |
| Ia-5.24 | S | S | — | oxetan-3-yl |
| Ia-5.25 | S | S | — | tetrahydrofuran-3-yl |
| Ia-5.26 | S | S | — | tetrahydropyran-3-yl |
| Ia-5.27 | S | S | — | oxepan-3-yl |
| Ia-5.28 | S | S | — | thiethan-3-yl |
| Ia-5.29 | S | S | — | tetrahydrothiofuran-3-yl |
| Ia-5.30 | S | S | — | tetrahydrothiopyran-3-yl |
| Ia-5.31 | S | S | — | thiepan-3-yl |
| Ia-5.32 | S | S | — | tetrahydropyran-4-yl |
| Ia-5.33 | S | S | — | oxepan-4-yl |
| Ia-5.34 | S | S | — | tetrahydrothiopyran-4-yl |
| Ia-5.35 | S | S | — | oxepan-4-yl |
| Ia-5.36 | S | S | — | tetrahydrothiopyran-4-yl |
| Ia-5.37 | S | S | O | CH₃ |
| Ia-5.38 | S | S | O | C₂H₅ |
| Ia-5.39 | S | S | O | n-C₃H₇ |
| Ia-5.40 | S | S | O | i-C₃H₇ |
| Ia-5.41 | S | S | O | cyclopropyl |
| Ia-5.42 | S | S | — | CO₂H |
| Ia-5.43 | S | S | — | CO₂CH₃ |
| Ia-5.44 | S | S | — | CO₂C₂H₅ |
| Ia-5.45 | S | S | — | CH₂CO₂CH₃ |
| Ia-5.46 | S | S | — | CH(CH₃)CO₂CH₃ |
| Ia-5.47 | S | S | O | CH₂CO₂CH₃ |
| Ia-5.48 | S | S | O | CH(CH₃)CO₂CH₃ |
| Ia-5.49 | S | S | — | Cl |
| Ia-5.50 | S | S | — | CN |
| Ia-5.51 | S | S | — | CH₂C≡CH |
| Ia-5.52 | S | S | O | CH₂C≡CH |
| Ia-5.53 | S | S | — | CH₂OCH₃ |
| Ia-5.54 | S | S | — | CH₂CH₂OCH₃ |
| Ia-5.55 | S | S | O | CH₂CH₂OCH₃ |
| Ia-5.56 | S | S | CH₂ | cyclopentyl |
| Ia-5.57 | S | O | — | H |
| Ia-5.58 | S | O | — | CH₃ |
| Ia-5.59 | S | O | — | C₂H₅ |
| Ia-5.60 | S | O | — | n-C₃H₇ |
| Ia-5.61 | S | O | — | i-C₃H₇ |
| Ia-5.62 | S | O | — | cyclopropyl |
| Ia-5.63 | S | O | — | n-C₄H₉ |
| Ia-5.64 | S | O | — | sec-C₄H₉ |
| Ia-5.65 | S | O | — | i-C₄H₉ |
| Ia-5.66 | S | O | — | t-C₄H₉ |
| Ia-5.67 | S | O | CH₂ | cyclopropyl |
| Ia-5.68 | S | O | — | cyclopentyl |
| Ia-5.69 | 5 | O | — | cyclohexyl |
| Ia-5.70 | S | O | — | oxiran-2-yl |
| Ia-5.71 | S | O | — | oxetan-2-yl |
| Ia-5.72 | S | O | — | tetrahydrofuran-2-yl |
| Ia-5.73 | S | O | — | tetrahydropyran-2-yl |
| Ia-5.74 | S | O | — | oxepan-2-yl |
| Ia-5.75 | S | O | — | thiiran-2-yl |
| Ia-5.76 | S | O | — | thietan-2-yl |
| Ia-5.77 | S | O | — | tetrahydrothiofuran-2-yl |
| Ia-5.78 | S | O | — | tetrahydrothiopyran-2-yl |
| Ia-5.79 | S | O | — | thiepan-2-yl |
| Ia-5.80 | 5 | O | — | oxetan-3-yl |
| Ia-5.81 | S | O | — | tetrahydrofuran-3-yl |
| Ia-5.82 | S | O | — | tetrahydropyran-3-yl |
| Ia-5.83 | S | O | — | oxepan-3-yl |
| Ia-5.84 | S | O | — | thiethan-3-yl |
| Ia-5.85 | S | O | — | tetrahydrothiofuran-3-yl |
| Ia-5.86 | S | O | — | tetrahydrothiopyran-3-yl |
| Ia-5.87 | S | O | — | thiepan-3-yl |
| Ia-5.88 | S | O | — | tetrahydropyran-4-yl |
| Ia-5.89 | S | O | — | oxepan-4-yl |
| Ia-5.90 | S | O | — | tetrahydrothiopyran-4-yl |
| Ia-5.91 | S | O | — | oxepan-4-yl |
| Ia-5.92 | S | O | — | tetrahydrothiopyran-4-yl |
| Ia-5.93 | S | O | O | CH₃ |
| Ia-5.94 | S | O | O | C₂H₅ |
| Ia-5.95 | S | O | O | n-C₃H₇ |
| Ia-5.96 | S | O | O | i-C₃H₇ |
| Ia-5.97 | S | O | O | cyclopropyl |
| Ia-5.98 | S | O | — | CO₂H |
| Ia-5.99 | S | O | — | CO₂CH₃ |
| Ia-5.100 | S | O | — | CO₂C₂H₅ |
| Ia-5.101 | S | O | — | CH₂CO₂CH₃ |
| Ia-5.102 | S | O | — | CH(CH₃)CO₂CH₃ |
| Ia-5.103 | S | O | O | CH₂CO₂CH |
| Ia-5.104 | S | O | O | CH(CH₃)CO₂CH₃ |
| Ia-5.105 | S | O | — | Cl |
| Ia-5.106 | S | O | — | CN |
| Ia-5.107 | S | O | — | CH₂C≡CH |
| Ia-5.108 | S | O | O | CH₂C≡CH |
| Ia-5.109 | S | O | — | CH₂OCH₃ |
| Ia-5.110 | S | O | — | CH₂CH₂OCH₃ |
| Ia-5.111 | S | O | O | CH₂CH₂OCH₃ |
| Ia-5.112 | S | O | CH₂ | cyclopentyl |
| Ia-5.113 | O | S | — | H |
| Ia-5.114 | O | S | — | CH₃ |
| Ia-5.115 | O | S | — | C₂H₅ |
| Ia-5.116 | O | S | — | n-C₃H₇ |
| Ia-5.117 | O | S | — | i-C₃H₇ |
| Ia-5.118 | O | S | — | cyclopropyl |
| Ia-5.119 | O | S | — | n-C₄H₉ |
| Ia-5.120 | O | 5 | — | sec-C₄H₉ |
| Ia-5.121 | O | 5 | — | i-C₄H₉ |
| Ia-5.122 | O | 5 | — | t-C₄H₉ |
| Ia-5.123 | O | S | CH₂ | cyclopropyl |
| Ia-5.124 | O | 5 | — | cyclopentyl |
| Ia-5.125 | O | S | — | cyclohexyl |
| Ia-5.126 | O | S | — | oxiran-2-yl |
| Ia-5.127 | O | S | — | oxetan-2-yl |
| Ia-5.128 | O | S | — | tetrahydrofuran-2-yl |
| Ia-5.129 | O | S | — | tetrahydropyran-2-yl |
| Ia-5.130 | O | S | — | oxepan-2-yl |
| Ia-5.131 | O | S | — | thiiran-2-yl |
| Ia-5.132 | O | 5 | — | thietan-2-yl |
| Ia-5.133 | O | S | — | tetrahydrothiofuran-2-yl |
| Ia-5.134 | O | S | — | tetrahydrothiopyran-2-yl |
| Ia-5.135 | O | S | — | thiepan-2-yl |
| Ia-5.136 | O | S | — | oxetan-3-yl |
| Ia-5.137 | O | S | — | tetrahydrofuran-3-yl |
| Ia-5.138 | O | S | — | tetrahydropyran-3-yl |
| Ia-5.139 | O | S | — | oxepan-3-yl |
| Ia-5.140 | O | S | — | thiethan-3-yl |
| Ia-5.141 | O | S | — | tetrahydrothiofuran-3-yl |
| Ia-5.142 | O | S | — | tetrahydrothiopyran-3-yl |
| Ia-5.143 | O | S | — | thiepan-3-yl |
| Ia-5.144 | O | S | — | tetrahydropyran-4-yl |
| Ia-5.145 | O | S | — | oxepan-4-yl |
| Ia-5.146 | O | S | — | tetrahydrothiopyran-4-yl |
| Ia-5.147 | O | S | — | oxepan-4-yl |
| Ia-5.148 | O | S | — | tetrahydrothiopyran-4-yl |
| Ia-5.149 | O | S | O | CH₃ |
| Ia-5.150 | O | S | O | C₂H₅ |
| Ia-5.151 | O | S | O | n-C₃H₇ |
| Ia-5.152 | O | S | O | i-C₃H₇ |
| Ia-5.153 | O | S | O | cyclopropyl |
| Ia-5.154 | O | S | — | CO₂H |
| Ia-5.155 | O | S | — | CO₂CH₃ |
| Ia-5.156 | O | S | — | CO₂C₂H₅ |
| Ia-5.157 | O | S | — | CH₂CO₂CH₃ |
| Ia-5.158 | O | S | — | CH(CH₃)CO₂CH₃ |
| Ia-5.159 | O | S | O | CH₂CO₂CH₃ |
| Ia-5.160 | O | S | O | CH(CH₃)CO₂CH₃ |
| Ia-5.161 | O | S | — | Cl |
| Ia-5.162 | O | S | — | CN |
| Ia-5.163 | O | S | — | CH₂C≡CH |
| Ia-5.164 | O | S | O | CH₂C≡CH |
| Ia-5.165 | O | S | — | CH₂OCH₃ |
| Ia-5.166 | O | S | — | CH₂CH₂OCH₃ |
| Ia-5.167 | O | S | O | CH₂CH₂OCH₃ |
| Ia-5.168 | O | S | CH₂ | cyclopentyl |
| Ia-5.169 | O | O | — | H |
| Ia-5.170 | O | O | — | CH₃ |
| Ia-5.17l | O | O | — | n-C₃H₇ |
| Ia-5.172 | O | O | — | i-C₃H₇ |
| Ia-5.173 | O | O | — | cyclopropyl |
| Ia-5.174 | O | O | — | n-C₄H₉ |
| Ia-5.175 | O | O | — | sec-C₄H₉ |
| Ia-5.176 | O | O | — | i-C₄H₉ |
| Ia-5.177 | O | O | — | t-C₄H₉ |
| Ia-5.178 | O | O | CH₂ | cyclopropyl |
| Ia-5.179 | O | O | — | cyclopentyl |
| Ia-5.180 | O | O | — | cyclohexyl |
| Ia-5.181 | O | O | — | oxiran-2-yl |
| Ia-5.182 | O | O | — | oxetan-2-yl |
| Ia-5.183 | O | O | — | tetrahydrofuran-2-yl |
| Ia-5.184 | O | O | — | tetrahydropyran-2-yl |
| Ia-5.185 | O | O | — | oxepan-2-yl |
| Ia-5.186 | O | O | — | thiiran-2-yl |
| Ia-5.187 | O | O | — | thietan-2-yl |
| Ia-5.188 | O | O | — | tetrahydrothiofuran-2-yl |
| Ia-5.189 | O | O | — | tetrahydrothiopyran-2-yl |
| Ia-5.190 | O | O | — | thiepan-2-yl |
| Ia-5.191 | O | O | — | oxetan-3-yl |
| Ia-5.192 | O | O | — | tetrahydrofuran-3-yl |
| Ia-5.193 | O | O | — | tetrahydropyran-3-yl |
| Ia-5.194 | O | O | — | oxepan-3-yl |
| Ia-5.195 | O | O | — | thiethan-3-yl |
| Ia-5.196 | O | O | — | tetrahydrothiofuran-3-yl |
| Ia-5.197 | O | O | — | tetrahydrothiopyran-3-yl |
| Ia-5.198 | O | O | — | thiepan-3-yl |
| Ia-5.199 | O | O | — | tetrahydropyran-4-yl |
| Ia-5.200 | O | O | — | oxepan-4-yl |
| Ia-5.201 | O | O | — | tetrahydrothiopyran-4-yl |
| Ia-5.202 | O | O | — | oxepan-4-yl |
| Ia-5.203 | O | O | — | tetrahydrothiopyran-4-yl |
| Ia-5.204 | O | O | O | CH₃ |
| Ia-5.205 | O | O | O | C₂H₅ |
| Ia-5.206 | O | O | O | n-C₃H₇ |
| Ia-5.207 | O | O | O | i-C₃H₇ |
| Ia-5.208 | O | O | O | cyclopropyl |
| Ia-5.209 | O | O | — | CO₂H |
| Ia-5.210 | O | O | — | CO₂CH₃ |
| Ia-5.211 | O | O | — | CO₂C₂H₅ |
| Ia-5.212 | O | O | — | CH₂CO₂CH₃ |
| Ia-5.213 | O | O | — | CH(CH₃)CO₂CH₃ |
| Ia-5.214 | O | O | O | CH₂CO₂CH₃ |
| Ia-5.215 | O | O | O | CH(CH₃)CO₂CH₃ |
| Ia-5.216 | O | O | — | Cl |
| Ia-5.217 | O | O | — | CN |
| Ia-5.218 | O | O | — | CH₂C≡CH |
| Ia-5.219 | O | O | O | CH₂C≡CH |
| Ia-5.220 | O | O | — | CH₂OCH₃ |
| Ia-5.221 | O | O | — | C₂H₅ |
| Ia-5.222 | O | O | — | CH₂CH₂OCH₃ |
| Ia-5.223 | O | O | O | CH₂CH₂OCH₃ |
| Ia-5.224 | O | O | CH₂ | cyclopentyl |

Particularly preferred compounds of the formula Ia are furthermore compounds of the formula Ia-6 where Q is Q-2 where Y=oxygen and Z¹, X, U and R⁶ have in each case the meanings given in one row of Table 2 (compounds Ia-6.1 to Ia-6.224)

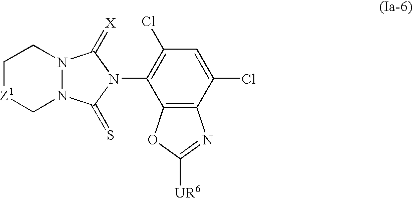

Particularly preferred compounds of the formula Ia are furthermore compounds of the formula Ia-7 where Q is Q-2 where Y=oxygen and Z¹, X, U and R⁶ have in each case the meanings given in one row of Table 2 (compounds Ia-7.1 to Ia-7.224)

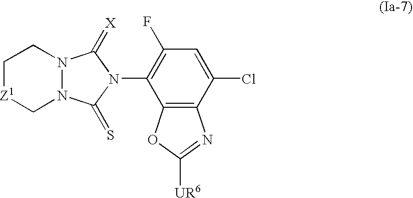

Particularly preferred compounds of the formula Ia are furthermore compounds of the formula Ia-8 where Q is Q-2 where Y=sulfur and Z¹, X, U and R⁶ have in each case the meanings given in one row of Table 2 (compounds Ia-8.1 to Ia-8.224)

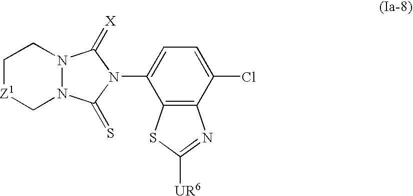

Particularly preferred compounds of the formula Ia are furthermore compounds of the formula Ia-9 where Q is Q-2 where Y=sulfur and Z¹, X, U and R⁶ have in each case the meanings given in one row of Table 2 (compounds Ia-9.1 to Ia-9.224)

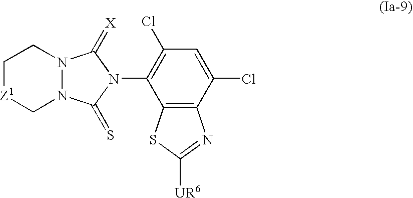

Particularly preferred compounds of the formula Ia are furthermore compounds of the formula Ia-10 where Q is Q-2 where Y=sulfur and Z¹, X, U and R⁶ have in each case the meanings given in one row of Table 2 (compounds Ia-10.1 to Ia-10.224)

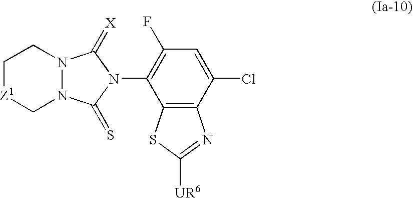

Particularly preferred compounds of the formula Ia are furthermore compounds of the formula Ia-l1 where Q is Q-2 where Y=oxygen and Z¹, X, U and R⁶ have in each case the meanings given in one row of Table 2 (compounds Ia-11.1 to Ia-11.224)

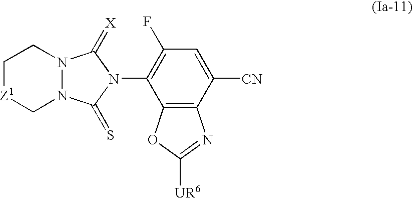

Particularly preferred compounds of the formula Ia are furthermore compounds of the formula Ia-12 where Q is Q-2 where Y=sulfur and Z¹, X, U and R⁶ have in each case the meanings given in one row of Table 2 (compounds Ia-12.1 to Ia-12.224)

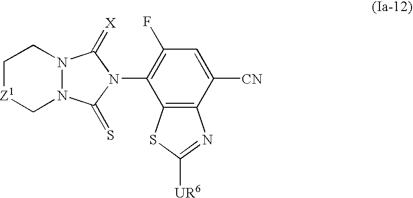

Particularly preferred compounds of the formula Ia are furthermore compounds of the formulae Ia-13 to Ia-20 below where Q is Q-2 where Y=oxygen or sulfur and Z¹, X, U and R⁶ have in each case the meanings given in one row of Table 2 (compounds Ia-13.1 to Ia-20.224)

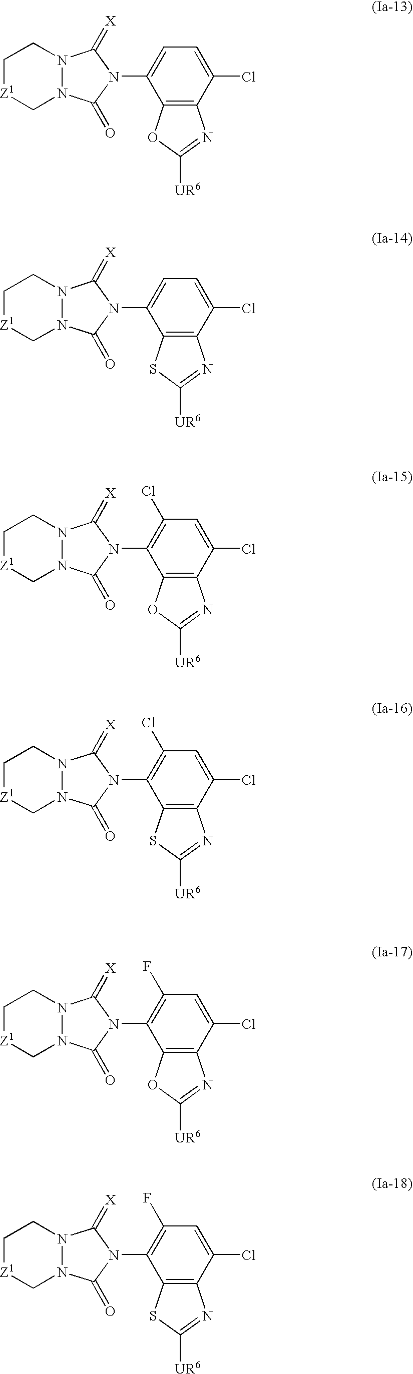

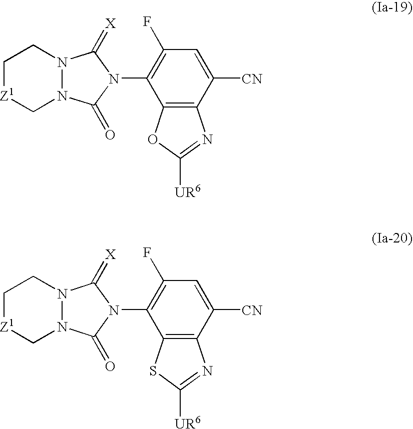

Particularly preferred compounds of the formula Ia are furthermore compounds of the formulae Ia-21 to Ia-44 below where Q is Q-3 where Y=oxygen or sulfur and Z¹, X, U and R⁶ have in each case the meanings given in one row of Table 2 (compounds Ia-21.1 to Ia-44.224)

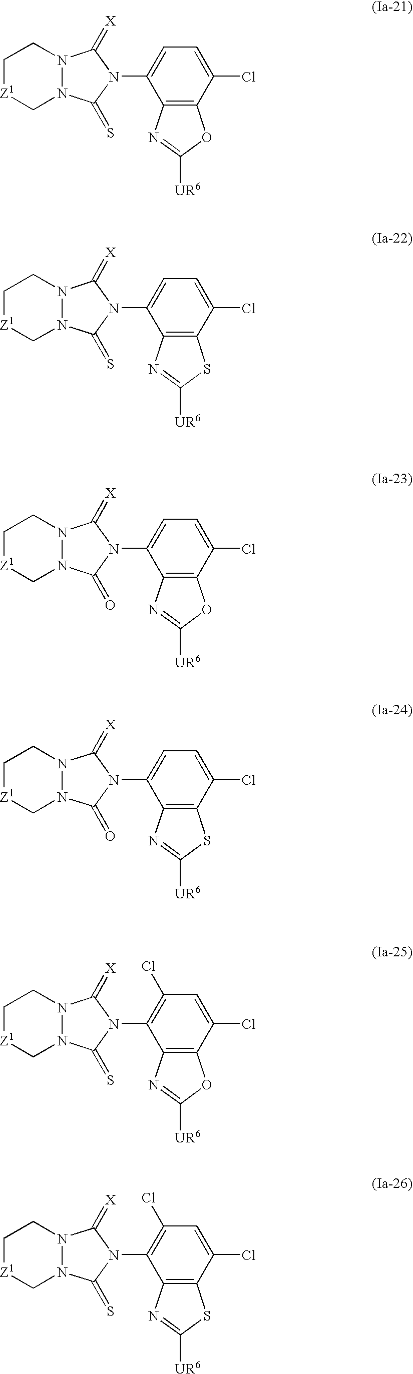

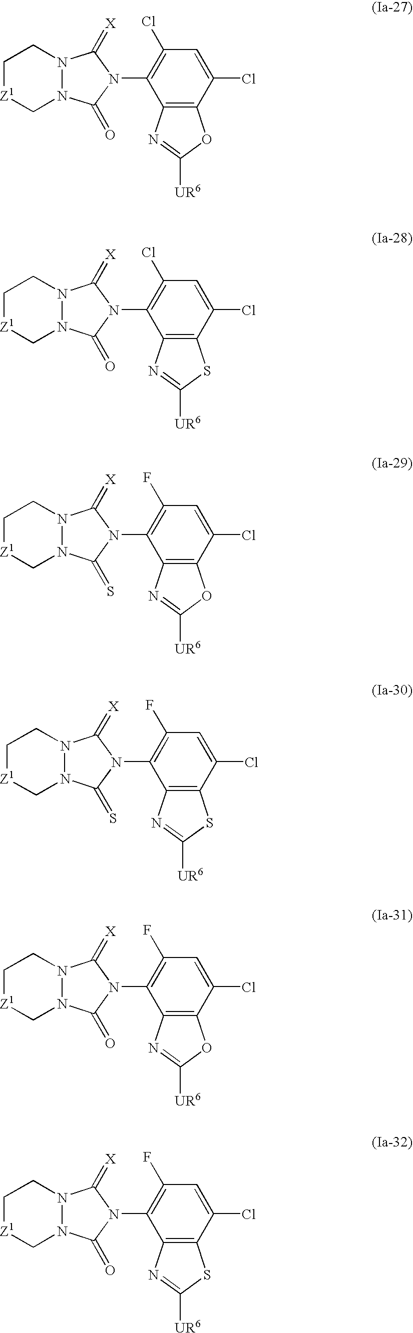

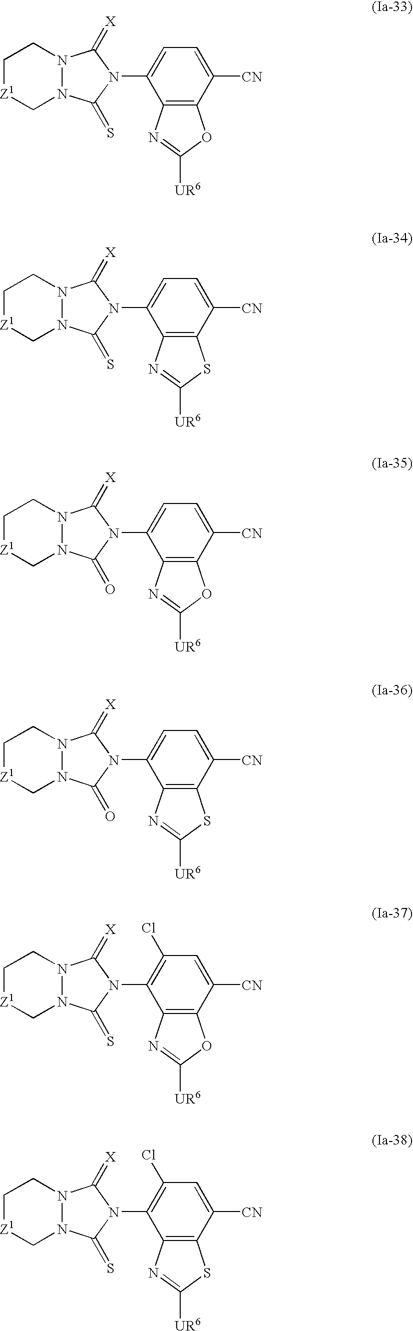

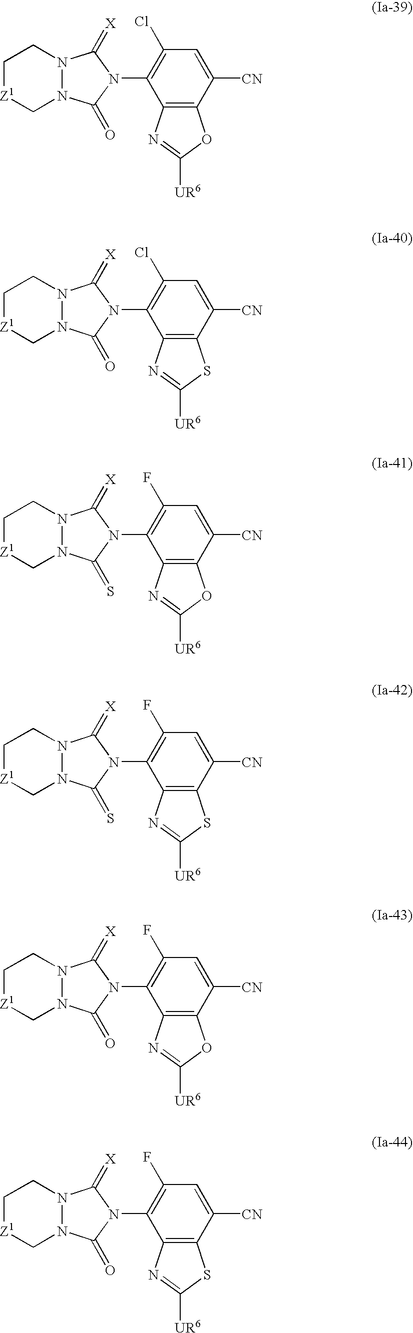

Particularly preferred compounds of the formula Ia are furthermore compounds of the formula Ia-45 below where Q is Q-4 where Y=oxygen and Z¹, X, T and R⁷ have in each case the meanings given in one row of Table 3 (compounds Ia-45.1 to Ia-45.140)

TABLE 3

| | |
|---|---|
| | (Ia-45) |
| 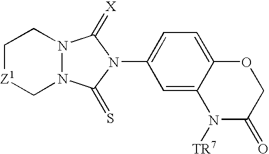 | |

| No. | Z¹ | X | T | R⁷ |
|---|---|---|---|---|
| Ia-45.1 | O | S | — | CH₃ |
| Ia-45.2 | O | S | — | C₂H₅ |
| Ia-45.3 | O | S | — | n-C₃H₇ |
| Ia-45.4 | O | S | — | cyclopropyl |
| Ia-45.5 | O | S | — | n-C₄H₉ |
| Ia-45.6 | O | S | — | sec-C₄H₉ |
| Ia-45.7 | O | S | — | t-C₄H₉ |
| Ia-45.8 | O | S | — | CH₂—CH═CH₂ |
| Ia-45.9 | O | S | — | CH₂—C≡CH |
| Ia-45.10 | O | S | — | CH₂CH₂Cl |
| Ia-45.11 | O | S | — | CH₂CH₂OCH₃ |
| Ia-45.12 | O | S | — | CH₂CH₂CN |
| Ia-45.13 | O | S | — | H |
| Ia-45.14 | O | S | O | H |
| Ia-45.15 | O | S | O | CH₃ |
| Ia-45.16 | O | S | O | C₂H₅ |
| Ia-45.17 | O | S | O | n-C₃H₇ |
| Ia-45.18 | O | S | O | cyclopropyl |
| Ia-45.19 | O | S | O | n-C₄H₉ |
| Ia-45.20 | O | S | O | sec-C₄H₉ |
| Ia-45.21 | O | S | O | t-C₄H₉ |
| Ia-45.22 | O | S | O | CH₂—CH═CH₂ |
| Ia-45.23 | O | S | O | CH₂—C≡CH |
| Ia-45.24 | O | S | O | CH₂CH₂Cl |
| Ia-45.25 | O | S | O | CH₂CH₂OCH₃ |
| Ia-45.26 | O | S | O | CH₂CH₂CN |
| Ia-45.27 | O | S | O | i-C₃H₇ |
| Ia-45.28 | O | S | O | i-C₄H₉ |
| Ia-45.29 | O | S | — | i-C₃H₇ |
| Ia-45.30 | O | S | — | i-C₄H₉ |
| Ia-45.31 | O | S | O | CH₂CO₂CH₃ |
| Ia-45.32 | O | S | O | CH(CH₃)CO₂CH₃ |
| Ia-45.33 | O | S | O | CH(CH₃)CO₂C₂H₅ |
| Ia-45.34 | O | S | — | CH₂CO₂CH₃ |
| Ia-45.35 | O | S | — | CH₂CO₂-n-C₃H₇ |
| Ia-45.36 | S | O | — | CH₃ |
| Ia-45.37 | S | O | — | C₂H₅ |
| Ia-45.38 | S | O | — | n-C₃H₇ |
| Ia-45.39 | S | O | — | cyclopropyl |
| Ia-45.40 | S | O | — | n-C₄H₉ |
| Ia-45.41 | S | O | — | sec-C₄H₉ |
| Ia-45.42 | S | O | — | t-C₄H₉ |
| Ia-45.43 | S | O | — | CH₂—CH═CH₂ |
| Ia-45.44 | S | O | — | CH₂—C≡CH |
| Ia-45.45 | S | O | — | CH₂CH₂Cl |
| Ia-45.46 | S | O | — | CH₂CH₂OCH₃ |
| Ia-45.47 | S | O | — | CH₂CH₂CN |
| Ia-45.48 | S | O | — | H |
| Ia-45.49 | S | O | O | H |
| Ia-45.50 | S | O | O | CH₃ |
| Ia-45.51 | S | O | O | C₂H₅ |
| Ia-45.52 | S | O | O | n-C₃H₇ |
| Ia-45.53 | S | O | O | cyclopropyl |
| Ia-45.54 | S | O | O | n-C₄H₉ |
| Ia-45.55 | S | O | O | sec-C₄H₉ |
| Ia-45.56 | S | O | O | t-C₄H₉ |
| Ia-45.57 | S | O | O | CH₂—CH═CH₂ |
| Ia-45.58 | S | O | O | CH₂—C≡CH |
| Ia-45.59 | S | O | O | CH₂CH₂Cl |
| Ia-45.60 | S | O | O | CH₂CH₂OCH₃ |
| Ia-45.61 | S | O | O | CH₂CH₂CN |
| Ia-45.62 | S | O | O | i-C₃H₇ |
| Ia-45.63 | S | O | O | i-C₄H₉ |
| Ia-45.64 | S | O | — | i-C₃H₇ |
| Ia-45.65 | S | O | — | i-C₄H₉ |
| Ia-45.66 | S | O | O | CH₂CO₂CH₃ |
| Ia-45.67 | S | O | O | CH(CH₃)CO₂CH₃ |
| Ia-45.68 | S | O | O | CH(CH₃)CO₂C₂H₅ |
| Ia-45.69 | S | O | — | CH₂CO₂CH₃ |
| Ia-45.70 | S | O | — | CH₂CO₂-n-C₃H₇ |
| Ia-45.71 | S | S | — | CH₃ |
| Ia-45.72 | S | S | — | C₂H₅ |
| Ia-45.73 | S | S | — | n-C₃H₇ |
| Ia-45.74 | S | S | — | cyclopropyl |
| Ia-45.75 | S | S | — | n-C₄H₉ |
| Ia-45.76 | S | S | — | sec-C₄H₉ |
| Ia-45.77 | S | S | — | t-C₄H₉ |
| Ia-45.78 | S | S | — | CH₂—CH═CH₂ |
| Ia-45.79 | S | S | — | CH₂—C≡CH |
| Ia-45.80 | S | S | — | CH₂CH₂Cl |
| Ia-45.81 | S | S | — | CH₂CH₂OCH₃ |
| Ia-45.82 | S | S | — | CH₂CH₂CN |
| Ia-45.83 | S | S | — | H |
| Ia-45.84 | S | S | O | H |
| Ia-45.85 | S | S | O | CH₃ |
| Ia-45.86 | S | S | O | C₂H₅ |
| Ia-45.87 | S | S | O | n-C₃H₇ |
| Ia-45.88 | S | S | O | cyclopropyl |
| Ia-45.89 | S | S | O | n-C₄H₉ |
| Ia-45.90 | S | S | O | sec-C₄H₉ |
| Ia-45.91 | S | S | O | t-C₄H₉ |
| Ia-45.92 | S | S | O | CH₂—CH═CH₂ |
| Ia-45.93 | S | S | O | CH₂—C≡CH |
| Ia-45.94 | S | S | O | CH₂CH₂Cl |
| Ia-45.95 | S | S | O | CH₂CH₂OCH₃ |
| Ia-45.96 | S | S | O | CH₂CH₂CN |
| Ia-45.97 | S | S | O | i-C₃H₇ |
| Ia-45.98 | S | S | O | i-C₄H₉ |
| Ia-45.99 | S | S | — | i-C₃H₇ |
| Ia-45.100 | S | S | — | i-C₄H₉ |
| Ia-45.101 | S | S | O | CH₂CO₂CH₃ |
| Ia-45.102 | S | S | O | CH(CH₃)CO₂CH₃ |
| Ia-45.103 | S | S | O | CH(CH₃)CO₂C₂H₅ |
| Ia-45.104 | S | S | — | CH₂CO₂CH₃ |
| Ia-45.105 | S | S | — | CH₂CO₂-n-C₃H₇ |
| Ia-45.106 | O | O | — | CH₃ |
| Ia-45.107 | O | O | — | C₂H₅ |
| Ia-45.108 | O | O | — | n-C₃H₇ |
| Ia-45.109 | O | O | — | cyclopropyl |
| Ia-45.110 | O | O | — | n-C₄H₉ |
| Ia-45.111 | O | O | — | sec-C₄H₉ |
| Ia-45.112 | O | O | — | t-C₄H₉ |
| Ia-45.113 | O | O | — | CH₂—CH═CH₂ |
| Ia-45.114 | O | O | — | CH₂—C≡CH |
| Ia-45.115 | O | O | — | CH₂CH₂Cl |
| Ia-45.116 | O | O | — | CH₂CH₂OCH₃ |
| Ia-45.117 | O | O | — | CH₂CH₂CN |
| Ia-45.118 | O | O | — | H |
| Ia-45.119 | O | O | O | H |
| Ia-45.120 | O | O | O | CH₃ |
| Ia-45.121 | O | O | O | C₂H₅ |
| Ia-45.122 | O | O | O | n-C₃H₇ |
| Ia-45.123 | O | O | O | cyclopropyl |
| Ia-45.124 | O | O | O | n-C₄H₉ |
| Ia-45.125 | O | O | O | sec-C₄H₉ |
| Ia-45.126 | O | O | O | t-C₄H₉ |
| Ia-45.127 | O | O | O | CH₂—CH═CH₂ |
| Ia-45.128 | O | O | O | CH₂—C≡CH |
| Ia-45.129 | O | O | O | CH₂CH₂Cl |
| Ia-45.130 | O | O | O | CH₂CH₂OCH₃ |
| Ia-45.131 | O | O | O | CH₂CH₂CN |
| Ia-45.132 | O | O | O | i-C₃H₇ |
| Ia-45.133 | O | O | O | i-C₄H₉ |
| Ia-45.134 | O | O | — | i-C₃H₇ |
| Ia-45.135 | O | O | — | i-C₄H₉ |
| Ia-45.136 | O | O | O | CH₂CO₂CH₃ |
| Ia-45.137 | O | O | O | CH(CH₃)CO₂CH₃ |
| Ia-45.138 | O | O | O | CH(CH₃)CO₂C₂H₅ |
| Ia-45.139 | O | O | — | CH₂CO₂CH₃ |
| Ia-45.140 | O | O | — | CH₂CO₂-n-C₃H₇ |

Particularly preferred compounds of the formula Ia are furthermore compounds of the formulae Ia-46 and Ia-47 below where Q is Q-4 where Y=oxygen and Z¹, X, T and R⁷ have in each case the meanings given in one row of Table 3 (compounds Ia-46.1 to Ia-46.140 and Ia-47.1 to Ia-47.140)

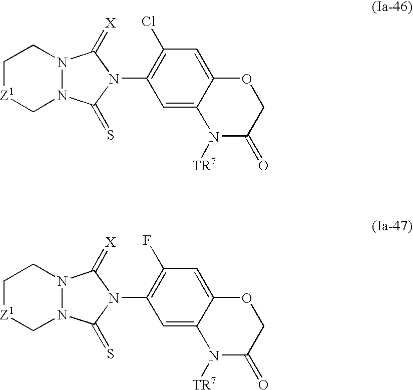

Particularly preferred compounds of the formula Ia are furthermore compounds of the formula Ia-48 below where Q is Q-5 where Y=Y′=oxygen and Z¹, X, T and R⁷ have in each case the meanings given in one row of Table 3 (compounds Ia-48.1 to Ia-48.140)

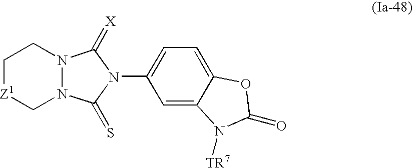

Particularly preferred compounds of the formula Ia are furthermore compounds of the formulae Ia-49 and Ia-50 below where Q is Q-5 where Y=Y′=oxygen and Z¹, X, T and R⁷ have in each case the meanings given in one row of Table 3 (compounds Ia-49.1 to Ia-49.140 and Ia-50.1 to Ia-50.140)

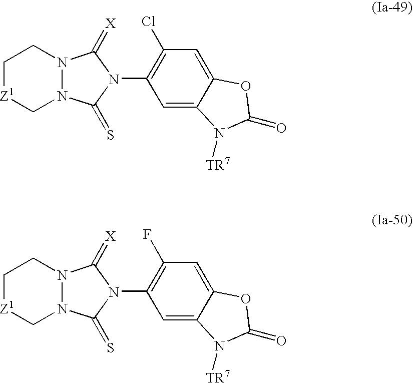

Particularly preferred compounds of the formula Ia are furthermore compounds of the formula Ia-51 below where Q is Q-6 and Z¹, X, R⁴, R⁸ and R⁹ have in each case the meanings given in one row of Table 4 (compounds Ia-51.1 to Ia-51.168)

TABLE 4

| | |
|---|---|
| | (Ia-51) |
| 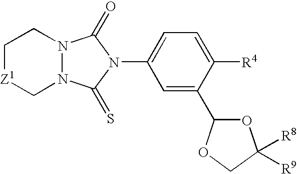 | |

| No. | Z¹ | X | R⁴ | R⁸ | R⁹ |
|---|---|---|---|---|---|
| Ia-51.1 | O | S | Cl | H | H |
| Ia-51.2 | O | S | Cl | H | CH₃ |
| Ia-51.3 | O | S | Cl | H | C₂H₅ |
| Ia-51.4 | O | S | Cl | H | n-C₃H₇ |
| Ia-51.5 | O | S | Cl | H | i-C₃H₇ |
| Ia-51.6 | O | S | Cl | H | n-C₄H₉ |
| Ia-51.7 | O | S | Cl | cyclopropyl | H |
| Ia-51.8 | O | S | Cl | CH₃ | H |
| Ia-51.9 | O | S | Cl | CH₃ | CH₃ |
| Ia-51.10 | O | S | Cl | C₂H₅ | H |
| Ia-51.11 | O | S | Cl | C₂H₅ | CH₃ |
| Ia-51.12 | O | S | Cl | n-C₃H₇ | C₂H₅ |
| Ia-51.13 | O | S | Cl | n-C₃H₇ | n-C₃H₇ |
| Ia-51.14 | O | S | Cl | Cl | H |
| Ia-51.15 | O | S | Cl | H | Cl |
| Ia-51.16 | O | S | Cl | Cl | CH₃ |
| Ia-51.17 | O | S | Cl | C(O) | |
| Ia-51.18 | O | S | Cl | CH₃ | CH₂CH₂Cl |
| Ia-51.19 | O | S | Cl | C₂H₅ | C₂H₅ |
| Ia-51.20 | O | S | Cl | i-C₃H₇ | i-C₃H₇ |
| Ia-51.21 | O | S | Cl | i-C₃H₇ | H |
| Ia-51.22 | O | O | Cl | H | H |
| Ia-51.23 | O | O | Cl | H | CH₃ |
| Ia-51.24 | O | O | Cl | H | C₂H₅ |
| Ia-51.25 | O | O | Cl | H | n-C₃H₇ |
| Ia-51.26 | O | O | Cl | H | i-C₃H₇ |
| Ia-51.27 | O | O | Cl | H | n-C₄H₉ |
| Ia-51.28 | O | O | Cl | cyclopropyl | H |
| Ia-51.29 | O | O | Cl | CH₃ | H |
| Ia-51.30 | O | O | Cl | CH₃ | CH₃ |
| Ia-51.31 | O | O | Cl | C₂H₅ | H |
| Ia-51.32 | O | O | Cl | C₂H₅ | CH₃ |
| Ia-51.33 | O | O | Cl | n-C₃H₇ | C₂H₅ |
| Ia-51.34 | O | O | Cl | n-C₃H₇ | n-C₃H₇ |
| Ia-51.35 | O | O | Cl | Cl | H |
| Ia-51.36 | O | O | Cl | H | Cl |
| Ia-51.37 | O | O | Cl | Cl | CH₃ |
| Ia-51.38 | O | O | Cl | C(O) | |
| Ia-51.39 | O | O | Cl | CH₃ | CH₂CH₂Cl |
| Ia-51.40 | O | O | Cl | C₂H₅ | C₂H₅ |
| Ia-51.41 | O | O | Cl | i-C₃H₇ | i-C₃H₇ |
| Ia-51.42 | O | O | Cl | i-C₃H₇ | H |
| Ia-51.43 | S | S | Cl | H | H |
| Ia-51.44 | S | S | Cl | H | CH₃ |
| Ia-51.45 | S | S | Cl | H | C₂H₅ |
| Ia-51.46 | S | S | Cl | H | n-C₃H₇ |
| Ia-51.47 | S | S | Cl | H | i-C₃H₇ |
| Ia-51.48 | S | S | Cl | H | n-C₄H₉ |
| Ia-51.49 | S | S | Cl | cyclopropyl | H |
| Ia-51.50 | S | S | Cl | CH₃ | H |
| Ia-51.51 | S | S | Cl | CH₃ | CH₃ |
| Ia-51.52 | S | S | Cl | C₂H₅ | H |
| Ia-51.53 | S | S | Cl | C₂H₅ | CH₃ |
| Ia-51.54 | S | S | Cl | n-C₃H₇ | C₂H₅ |
| Ia-51.55 | S | S | Cl | n-C₃H₇ | n-C₃H₇ |
| Ia-51.56 | S | S | Cl | Cl | H |
| Ia-51.57 | S | S | Cl | H | Cl |
| Ia-51.58 | S | S | Cl | Cl | CH₃ |
| Ia-51.59 | S | S | Cl | C(O) | |
| Ia-51.60 | S | S | Cl | CH₃ | CH₂CH₂Cl |
| Ia-5l.61 | S | S | Cl | C₂H₅ | C₂H₅ |
| Ia-51.62 | S | S | Cl | i-C₃H₇ | i-C₃H₇ |
| Ia-51.63 | S | S | Cl | i-C₃H₇ | H |
| Ia-51.64 | S | O | Cl | H | H |
| Ia-51.65 | S | O | Cl | H | CH₃ |
| Ia-5l.66 | S | O | Cl | H | C₂H₅ |
| Ia-51.67 | S | O | Cl | H | n-C₃H₇ |
| Ia-51.68 | S | O | Cl | H | i-C₃H₇ |
| Ia-51.69 | S | O | Cl | H | n-C₄H₉ |
| Ia-51.70 | S | O | Cl | cyclopropyl | H |
| Ia-51.7l | S | O | Cl | CH₃ | H |
| Ia-5l.72 | S | O | Cl | CH₃ | CH₃ |
| Ia-51.73 | S | O | Cl | C₂H₅ | H |
| Ia-51.74 | S | O | Cl | C₂H₅ | CH₃ |
| Ia-51.75 | S | O | Cl | n-C₃H₇ | C₂H₅ |
| Ia-51.76 | S | O | Cl | n-C₃H₇ | n-C₃H₇ |
| Ia-5l.77 | S | O | Cl | Cl | H |
| Ia-51.78 | S | O | Cl | H | Cl |
| Ia-51.79 | S | O | Cl | Cl | CH₃ |
| Ia-51.80 | S | O | Cl | C(O) | |
| Ia-51.81 | S | O | Cl | CH₃ | CH₂CH₂Cl |
| Ia-51.82 | S | O | Cl | C₂H₅ | C₂H₅ |
| Ia-51.83 | S | O | Cl | i-C₃H₇ | i-C₃H₇ |
| Ia-51.84 | S | O | Cl | i-C₃H₇ | H |
| Ia-51.85 | O | S | CN | H | H |
| Ia-51.86 | O | S | CN | H | CH₃ |
| Ia-51.87 | O | S | CN | H | C₂H₅ |
| Ia-51.88 | O | S | CN | H | n-C₃H₇ |
| Ia-51.89 | O | S | CN | H | i-C₃H₇ |
| Ia-51.90 | O | S | CN | H | n-C₄H₉ |
| Ia-51.91 | O | S | CN | cyclopropyl | H |
| Ia-51.92 | O | S | CN | CH₃ | H |
| Ia-51.93 | O | S | CN | CH₃ | CH₃ |
| Ia-51.94 | O | S | CN | C₂H₅ | H |
| Ia-51.95 | O | S | CN | C₂H₅ | CH₃ |
| Ia-51.96 | O | S | CN | n-C₃H₇ | C₂H₅ |
| Ia-51.97 | O | S | CN | n-C₃H₇ | n-C₃H₇ |
| Ia-51.98 | O | S | CN | Cl | H |
| Ia-51.99 | O | S | CN | H | Cl |
| Ia-51.100 | O | S | CN | Cl | CH3 |
| Ia-51.101 | O | S | CN | C(O) | |
| Ia-51.102 | O | S | CN | CH₃ | CH₂CH₂Cl |
| Ia-51.103 | O | S | CN | C₂H₅ | C₂H₅ |
| Ia-51.104 | O | S | CN | i-C₃H₇ | i-C₃H₇ |
| Ia-51.105 | O | S | CN | i-C₃H₇ | H |
| Ia-51.106 | O | O | CN | H | H |
| Ia-51.107 | O | O | CN | H | CH₃ |
| Ia-51.108 | O | O | CN | H | C₂H₅ |
| Ia-51.109 | O | O | CN | H | n-C₃H₇ |
| Ia-51.110 | O | O | CN | H | i-C₃H₇ |
| Ia-51.111 | O | O | CN | H | n-C₄H₉ |
| Ia-51.112 | O | O | CN | cyclopropyl | H |
| Ia-51.113 | O | O | CN | CH₃ | H |
| Ia-51.114 | O | O | CN | CH₃ | CH₃ |
| Ia-51.115 | O | O | CN | C₂H₅ | H |
| Ia-51.116 | O | O | CN | C₂H₅ | CH₃ |
| Ia-51.117 | O | O | CN | n-C₃H₇ | C₂H₅ |
| Ia-51.118 | O | O | CN | n-C₃H₇ | n-C₃H₇ |
| Ia-51.119 | O | O | CN | Cl | H |
| Ia-51.120 | O | O | CN | H | Cl |
| Ia-51.121 | O | O | CN | Cl | CH₃ |
| Ia-51.122 | O | O | CN | C(O) | |
| Ia-51.123 | O | O | CN | CH₃ | CH₂CH₂Cl |
| Ia-51.124 | O | O | CN | C₂H₅ | C₂H₅ |
| Ia-51.125 | O | O | CN | i-C₃H₇ | i-C₃H₇ |
| Ia-51.126 | O | O | CN | i-C₃H₇ | H |
| Ia-51.127 | S | S | CN | H | H |
| Ia-51.128 | S | S | CN | H | CH₃ |
| Ia-51.129 | S | S | CN | H | C₂H₅ |
| Ia-51.130 | S | S | CN | H | n-C₃H₇ |
| Ia-51.131 | S | S | CN | H | i-C₃H₇ |
| Ia-51.132 | S | S | CN | H | n-C₄H₉ |
| Ia-51.133 | S | S | CN | cyclopropyl | H |
| Ia-51.134 | S | S | CN | CH₃ | H |
| Ia-51.135 | S | S | CN | CH₃ | CH₃ |
| Ia-51.136 | S | S | CN | C₂H₅ | H |
| Ia-51.137 | S | S | CN | C₂H₅ | CH₃ |
| Ia-51.138 | S | S | CN | n-C₃H₇ | C₂H₅ |
| Ia-51.139 | S | S | CN | n-C₃H₇ | n-C₃H₇ |
| Ia-51.140 | S | S | CN | Cl | H |
| Ia-51.141 | S | S | CN | H | Cl |
| Ia-51.142 | S | S | CN | Cl | CH₃ |
| Ia-51.143 | S | S | CN | C(O) | |
| Ia-51.144 | S | S | CN | CH₃ | CH₂CH₂Cl |
| Ia-51.145 | S | S | CN | C₂H₅ | C₂H₅ |
| Ia-51.146 | S | S | CN | i-C₃H₇ | i-C₃H₇ |
| Ia-51.147 | S | S | CN | i-C₃H₇ | H |
| Ia-51.148 | S | O | CN | H | H |
| Ia-51.149 | S | O | CN | H | CH₃ |
| Ia-51.150 | S | O | CN | H | C₂H₅ |
| Ia-51.151 | S | O | CN | H | n-C₃H₇ |
| Ia-51.152 | S | O | CN | H | i-C₃H₇ |
| Ia-51.153 | S | O | CN | H | n-C₄H₉ |
| Ia-51.154 | S | O | CN | cyclopropyl | H |
| Ia-51.155 | S | O | CN | CH₃ | H |
| Ia-51.156 | S | O | CN | CH₃ | CH₃ |
| Ia-51.157 | S | O | CN | C₂H₅ | H |
| Ia-51.158 | S | O | CN | C₂H₅ | CH₃ |
| Ia-51.159 | S | O | CN | n-C₃H₇ | C₂H₅ |
| Ia-51.160 | S | O | CN | n-C₃H₇ | n-C₃H₇ |
| Ia-51.161 | S | O | CN | Cl | H |
| Ia-51.162 | S | O | CN | H | Cl |
| Ia-51.163 | S | O | CN | Cl | CH₃ |
| Ia-51.164 | S | O | CN | C(O) | |
| Ia-51.165 | S | O | CN | CH₃ | CH₂CH₂Cl |
| Ia-51.166 | S | O | CN | C₂H₅ | C₂H₅ |
| Ia-51.167 | S | O | CN | i-C₃H₇ | i-C₃H₇ |
| Ia-51.168 | S | O | CN | i-C₃H₇ | H |

Particularly preferred compounds of the formula Ia are compounds of the formulae Ia-52 and Ia-53 below where Q is Q-6 and Z¹, X, R⁴, R⁸ and R⁹ have in each case the meanings given in one row of Table 4 (compounds Ia-52.1 to Ia-52.168 and Ia-53.1 to Ia-53.168)

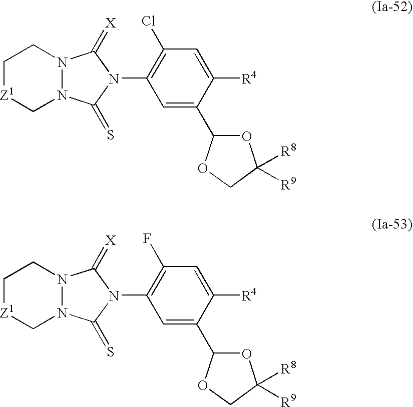

Particularly preferred compounds of the formula Ia are furthermore compounds of the formulae Ia-54 to Ia-57 below where Q is Q-7 and Z¹, X and R³⁰ have in each case the meanings given in one row of Table 5 (compounds Ia-54.1 to Ia-57.56)

TABLE 5

| | |
|---|---|
| | (Ia-54) |
| 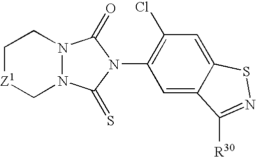 | |
| | (Ia-55) |
| 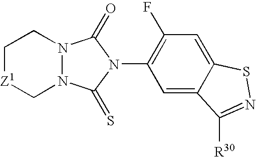 | |
| | (Ia-56) |
| 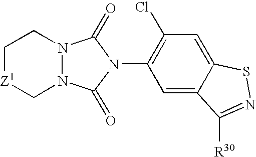 | |
| | (Ia-57) |
| 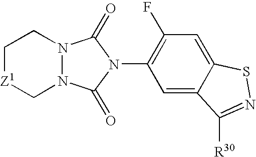 | |

| No. | Z¹ | X | R³⁰ |
|---|---|---|---|
| Ia-54.1 | O | O | CH₃ |
| Ia-54.2 | O | O | C₂H₅ |
| Ia-54.3 | O | O | n-C₃H₇ |
| Ia-54.4 | O | O | i-C₃H₇ |
| Ia-54.5 | O | O | c-C₃H₅ |
| Ia-54.6 | O | O | CH₂OCH₃ |
| Ia-54.7 | O | O | CH₂OC₂H₅ |
| Ia-54.8 | O | O | CH₂O-(n-C₃H₇) |
| Ia-54.9 | O | O | CH₂O-(i-C₃H₇) |
| Ia-54.10 | O | O | CH₂OCH₂CH═CH₂ |
| Ia-54.11 | O | O | CH₂OCH₂C≡CH |
| Ia-54.12 | O | O | CH₂CH₂OCH₃ |
| Ia-54.13 | O | O | CH₂CH₂OC₂H₅ |
| Ia-54.14 | O | O | CH₂CH₂O-(n-C₃H₇) |
| Ia-54.15 | O | O | CH₂CH₂OCH₂CH═CH₂ |
| Ia-54.16 | O | O | CH₂CH₂OCH₂C≡CH |
| Ia-54.17 | O | O | CO₂CH₃ |
| Ia-54.18 | O | O | CO₂C₂H₅ |
| Ia-54.19 | O | O | CO₂-(n-C₃H₇) |
| Ia-54.20 | O | O | CO₂-(i-C₃H₇) |
| Ia-54.21 | O | O | CO₂CH₂CH═CH₂ |
| Ia-54.22 | O | O | CO₂CH₂C≡CH |
| Ia-54.23 | O | O | CH₂CO₂CH₃ |
| Ia-54.24 | O | O | CH₂CO₂C₂H₅ |
| Ia-54.25 | O | O | CH(CH₃)CO₂CH₃ |
| Ia-54.26 | O | O | CH₂CO₂CH₂CH═CH₂ |
| Ia-54.27 | O | O | CH(CH₃)CO₂CH₂CH═CH₂ |
| Ia-54.28 | O | O | CH(OCH₃)₂ |
| Ia-54.29 | O | O | CH(O(C₂H₅)₂ |
| Ia-54.30 | O | O | CH[O(CH₂)₃O] |
| Ia-54.31 | O | O | CH[(O(CH₂)₄O] |
| Ia-54.32 | O | O | C(O)NHSO₂CH₃ |
| Ia-54.33 | O | O | C(O)NHSO₂C₂H₅ |
| Ia-54.34 | O | O | C(O)NHSO₂C₆H₅ |
| Ia-54.35 | O | O | C₆H₅ |
| Ia-54.36 | O | O | (2-CH₃OC(O)CH₂)C₆H₄ |
| Ia-54.37 | O | O | 2-CH₃OC(O)CH(CH₃)C₆H₄ |
| Ia-54.38 | O | O | 2-chlorophenyl |
| Ia-54.39 | O | O | 3-chlorophenyl |
| Ia-54.40 | S | O | CH₃ |
| Ia-54.41 | S | O | C₂H₅ |
| Ia-54.42 | S | O | i-C₃H₇ |
| Ia-54.43 | S | O | c-C₃H₅ |
| Ia-54.44 | S | O | CH₂OCH₃ |
| Ia-54.45 | S | O | CH₂OC₂H₅ |
| Ia-54.46 | O | O | CH₂O-cyclopropyl |
| Ia-54.47 | S | O | CH₂O-cyclopropyl |
| Ia-54.48 | S | O | CH₂O-(n-C₃H₇) |
| Ia-54.49 | S | O | CH₂O-(i-C₃H₇) |
| Ia-54.50 | S | O | CO₂CH₃ |
| Ia-54.51 | 5 | O | CO₂C₂H₅ |
| Ia-54.52 | S | O | CH₂CO₂CH₃ |
| Ia-54.53 | S | O | CH(OCH₃)₂ |
| Ia-54.54 | S | O | CH(OC₂H₅)₂ |
| Ia-54.55 | S | O | C(O)NHSO₂CH₃ |
| Ia-54.56 | S | O | C(O)NHSO₂C₂H₅ |

The novel compounds Ia and their agriculturally useful salts are suitable, both in the form of isomer mixtures and in the form of the pure isomers, as herbicides. Herbicidal compositions comprising the compounds Ia control vegetation on non-crop areas very efficiently, especially at high rates of application. They act against broad-leaved weeds and harmful grasses in crops such as wheat, rice, maize, soybean and cotton without causing any significant damage to the crop plants. This effect is mainly observed at low rates of application.

Depending on the application method used, the compounds Ia, or the compositions comprising them, can additionally be employed in a further number of crop plants for eliminating undesirable plants. Examples of suitable crops are the following:

Allium cepa, Ananas comosus, Arachis hypogaea, Asparagus officinalis, Beta vulgaris spec. altissima, Beta vulgaris spec. rapa, Brassica napus var. napus, Brassica napus var. napobrassica, Brassica rapa var. silvestris, Camellia sinensis, Carthamus tinctorius, Carya illinoinensis, Citrus limon, Citrus sinensis, Coffea arabica (Coffea canephora, Coffea liberica), Cucumis sativus, Cynodon dactylon, Daucus carota, Elaeis guineensis, Fragaria vesca, Glycine max, Gossypium hirsutum, (Gossypium arboreum, Gossypium herbaceum, Gossypium vitifolium), Helianthus annuus, Hevea brasiliensis, Hordeum vulgare, Humulus lupulus, Ipomoea batatas, Juglans regia, Lens culinaris, Linum usitatissimum, Lycopersicon lycopersicum, Malus spec., Manihot esculenta, Medicago sativa, Musa spec., Nicotiana tabacum (N. rustica), Olea europaea, Oryza sativa, Phaseolus lunatus, Phaseolus vulgaris, Picea abies, Pinus spec., Pisum sativum, Prunus avium, Prunus persica, Pyrus communis, Ribes sylvestre, Ricinus communis, Saccharum officinarum, Secale cereale, Solanum tuberosum, Sorghum bicolor (s. vulgare), Theobroma cacao, Trifolium pratense, Triticum aestivum, Triticum durum, Vicia faba, Vitis vinifera and Zea mays.

In addition, the compounds Ia may also be used in crops which tolerate the action of herbicides owing to breeding, including genetic engineering methods.

Furthermore, the fused triazoles Ia are also suitable for the desiccation and/or defoliation of plants.

As desiccants, they are particularly suitable for desiccating the aerial parts of crop plants such as potatoes, oilseed rape, sunflowers and soybeans. This allows completely mechanical harvesting of these important crop plants.

Also of economical interest is facilitating harvesting, which is made possible by concentrating, in the course of time, fruit drop or reducing the adhesion to the tree in the case of citrus fruit, olives or other species and varieties of pomaceous fruit, stone fruit and hard-shelled fruit. The same mechanism, i.e. promotion of the formation of abscission tissue between fruits or leaves and the shoot of the plants is also essential for the targeted defoliation of useful plants, in particular cotton.

Moreover, the reduced period of time within which the individual cotton plants mature results in better fiber quality post-harvest.

The compounds Ia, or the compositions comprising them, can be applied, for example, in the form of directly sprayable aqueous solutions, powders, suspensions, also highly concentrated aqueous, oily or other suspensions or dispersions, emulsions, oil dispersions, pastes, dusts, spreading materials or granules, by means of spraying, atomizing, dusting, scattering, pouring, seed dressing or mixing with seeds. The use forms depend on the intended purposes; in any case, they should ensure the finest possible distribution of the active ingredients according to the invention. The herbicidal compositions comprise a herbicidally effective amount of at least one compound of the formula Ia or an agriculturally useful salt of Ia and auxiliaries customary for formulating crop protection agents.

Essentially, suitable inert auxiliaries include:

mineral oil fractions of medium to high boiling point, such as kerosene and diesel oil, furthermore coal tar oils and oils of vegetable or animal origin, aliphatic, cyclic and aromatic hydrocarbons, e.g. paraffin, tetrahydronaphthalene, alkylated naphthalenes and their derivatives, alkylated benzenes and their derivatives, alcohols such as methanol, ethanol, propanol, butanol and cyclohexanol, ketones such as cyclohexanone, or strongly polar solvents, e.g. amines such as N-methylpyrrolidone, and water.

Aqueous use forms can be prepared from emulsion concentrates, suspensions, pastes, wettable powders or water-dispersible granules by adding water. To prepare emulsions, pastes or oil dispersions, the fused triazoles Ia, either as such or dissolved in an oil or solvent, can be homogenized in water by means of a wetting agent, tackifier, dispersant or emulsifier. Alternatively, it is possible to prepare concentrates comprising active substance, wetting agent, tackifier, dispersant or emulsifier and, if desired, solvent or oil, which are suitable for dilution with water.

Suitable surfactants are the alkali metal salts, alkaline earth metal salts and ammonium salts of aromatic sulfonic acids, e.g. ligno-, phenol-, naphthalene- and dibutylnaphthalenesulfonic acid, and of fatty acids, alkyl- and alkylarylsulfonates, alkyl sulfates, lauryl ether sulfates and fatty alcohol sulfates, and salts of sulfated hexa-, hepta- and octadecanols, and also of fatty alcohol glycol ethers, condensates of sulfonated naphthalene and its derivatives with formaldehyde, condensates of naphthalene, or of the naphthalenesulfonic acids with phenol and formaldehyde, polyoxyethylene octylphenol ether, ethoxylated isooctyl-, octyl- or nonylphenol, alkylphenyl or tributylphenyl polyglycol ether, alkylaryl polyether alcohols, isotridecyl alcohol, fatty alcohol/ethylene oxide condensates, ethoxylated castor oil, polyoxyethylene alkyl ethers or polyoxypropylene alkyl ethers, lauryl alcohol polyglycol ether acetate, sorbitol esters, lignosulfite waste liquors or methylcellulose.

Powders, materials for broadcasting and dusts can be prepared by mixing or grinding the active substances together with a solid carrier.

Granules, e.g. coated granules, impregnated granules and homogeneous granules, can be prepared by binding the active compounds to solid carriers. Solid carriers are mineral earths, such as silicas, silica gels, silicates, talc, kaolin, limestone, lime, chalk, bole, loess, clay, dolomite, diatomaceous earth, calcium sulfate, magnesium sulfate, magnesium oxide, ground synthetic materials, fertilizers such as ammonium sulfate, ammonium phosphate and ammonium nitrate, ureas, and products of vegetable origin, such as cereal meal, tree bark meal, wood meal and nutshell meal, cellulose powders, or other solid carriers.

The concentrations of the active compounds Ia in the ready-to-use preparations can be varied within wide ranges. In general, the formulations comprise from 0.001 to 98% by weight, preferably 0.01 to 95% by weight, of at least one active compound. The active compounds are employed in a purity of from 90% to 100%, preferably 95% to 100% (according to the NMR spectrum).

The compounds according to the invention can be formulated, for example, as follows:

I 20 parts by weight of the compound from Example 8 (see Table 10) are dissolved in a mixture composed of 80 parts by weight of alkylated benzene, 10 parts by weight of the adduct of 8 to 10 mol of ethylene oxide to 1 mol of oleic acid N-monoethanolamide, 5 parts by weight of the calcium salt of dodecylbenzenesulfonic acid and 5 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound.

II 20 parts by weight of the compound from Example 5 (see Table 10) are dissolved in a mixture composed of 40 parts by weight of cyclohexanone, 30 parts by weight of isobutanol, 20 parts by weight of the adduct of 7 mol of ethylene oxide to 1 mol of isooctylphenol and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound.

III 20 parts by weight of the active compound from Example 30 (see Table 10) are dissolved in a mixture composed of 25 parts by weight of cyclohexanone, 65 parts by weight of a mineral oil fraction of boiling point 210 to 280° C. and 10 parts by weight of the adduct of 40 mol of ethylene oxide to 1 mol of castor oil. Pouring the solution into 100 000 parts by weight of water and finely distributing it therein gives an aqueous dispersion which comprises 0.02% by weight of the active compound.

IV 20 parts by weight of the active compound from Example 123 (see Table 11) are mixed thoroughly with 3 parts by weight of the sodium salt of diisobutylnaphthalenesulfonic acid, 17 parts by weight of the sodium salt of a lignosulfonic acid from a sulfite waste liquor and 60 parts by weight of pulverulent silica gel, and the mixture is ground in a hammer mill. Finely distributing the mixture in 20 000 parts by weight of water gives a spray mixture which comprises 0.1% by weight of the active compound.

V 3 parts by weight of the active compound from Example 3 (see Table 10) are mixed with 97 parts by weight of finely divided kaolin. This gives a dust which comprises 3% by weight of the active compound.

VI 20 parts by weight of the active compound from Example 26 (see Table 10) are mixed intimately with 2 parts by weight of the calcium salt of dodecylbenzenesulfonic acid, 8 parts by weight of fatty alcohol polyglycol ether, 2 parts by weight of the sodium salt of a phenol/urea/formaldehyde condensate and 68 parts by weight of a paraffinic mineral oil. This gives a stable oily dispersion.

VII 1 part by weight of the compound from Example 57 (see Table 10) is dissolved in a mixture composed of 70 parts by weight of cyclohexanone, 20 parts by weight of ethoxylated isooctylphenol and 10 parts by weight of ethoxylated castor oil. This gives a stable emulsion concentrate.

VIII 1 part by weight of the compound from Example 134 (see Table 12) is dissolved in a mixture composed of 80 parts by weight of cyclohexanone and 20 parts by weight of Wettol® EM 31 (nonionic emulsifier based on ethoxylated castor oil). This gives a stable emulsion concentrate.

The herbicidal compositions or the active compounds can be applied pre- or post-emergence or together with the seeds of a crop plant. It is also possible to apply the herbicidal compositions or active compounds by sowing crop plant seed pre-treated with the herbicidal compositions or active compounds. If the active ingredients are less well tolerated by certain crop plants, application techniques may be used where the herbicidal compositions are sprayed, with the aid of the spraying apparatus, in such a manner that the active ingredients come into as little contact as possible with the leaves of the sensitive crop plants while reaching the leaves of undesirable plants which grow thereunder, or the naked soil surface (post-directed, lay-by).

Depending on the intended control target, the season, the target plants and the growth stage, the application rates of active ingredient are from 0.001 to 3.0, preferably 0.01 to 1.0, kg of active substance (a.s.) per ha.

To widen the spectrum of action and to achieve synergistic effects, the fused triazoles of the formula Ia may be mixed with a large number of representatives of other groups of herbicidal or growth-regulating active compounds and then applied concomitantly. Suitable components for mixtures are for example 1,2,4-thiadiazoles, 1,3,4-thiadiazoles, amides, aminophosphoric acid and its derivatives, aminotriazoles, anilides, (het)aryloxyalkanoic acids and their derivatives, benzoic acid and its derivatives, benzothiadiazinones, 2-aroyl-1,3-cyclohexanediones, 2-hetaroyl-1,3-cyclohexanediones, hetaryl aryl ketones, benzylisoxazolidinones, meta-CF₃-phenyl derivatives, carbamates, quinolinecarboxylic acid and its derivatives, chloroacetanilides, cyclohexenone oxime ether derivatives, diazines, dichloropropionic acid and its derivatives, dihydrobenzofurans, dihydrofuran-3-ones, dinitroanilines, dinitrophenols, diphenyl ethers, dipyridyls, halocarboxylic acids and their derivatives, ureas, 3-phenyluracils, imidazoles, imidazolinones, N-phenyl-3,4,5,6-tetrahydrophthalimides, oxadiazoles, oxiranes, phenols, aryloxy- and hetaryloxyphenoxypropionic esters, phenylacetic acid and its derivatives, phenylpropionic acid and its derivatives, pyrazoles, phenylpyrazoles, pyridazines, pyridinecarboxylic acid and its derivatives, pyrimidyl ethers, sulfonamides, sulfonylureas, triazines, triazinones, triazolinones, triazolecarboxamides and uracils.

Furthermore, it may be advantageous to apply the compounds of the formula Ia, alone or in combination with other herbicides, together with other crop protection agents, for example with pesticides or agents for controlling phytopathogenic fungi or bacteria. Also of interest is the miscibility with mineral salt solutions, which are employed for treating nutrient and trace element deficiencies. Non-phytotoxic oils and oil concentrates may also be added.

The examples and comparative examples below serve to illustrate the invention.

I Experiments for preparing compounds of the formula I where X and/or W=sulfur by cyclization with thiophosgene

### COMPARATIVE EXAMPLE 1

### Reaction of 4-[(4-chloro-2-fluoro-5-methoxyanilino)carbonyl]-1,3,4-oxadiazinane with Thiophosgene in Pyridine Under Atmospheric Pressure

3.0 g (10.4 mmol) of the title compound of m.p. 140-148° C. were dissolved in 100 ml of pyridine. 0.05 g of activated carbon and then, over a period of 30 min and with stirring, 2.4 g (20.7 mmol) of thiophosgene in 8 ml of toluene were added at 22° C. The mixture was stirred at 22° C. for 36 h. The resulting suspension was poured into 300 ml of 1N hydrochloric acid. The mixture was extracted 4 times with methylene chloride, and the organic extract was washed with 1N hydrochloric acid and saturated sodium chloride solution, stirred with activated carbon and dried over magnesium sulfate. Filtration with suction and concentration under reduced pressure gave 1.8 g of a tacky residue. This was dissolved in methylene chloride and chromatographed on a commercial silica gel column using dichloromethane. The resulting fractions were examined by ¹H-NMR, IR and mass spectrometry.

The mass spectra of all the fractions showed inter alia molecular peaks at 287 and 289 for the starting material, but in no case peaks for the desired thioxaimide. The intensive C═O/C═S band at 1758 cm⁻¹ measured in the authentic end product was present in none of the IR spectra.

In addition to isothiocyanates and decomposition products which were not characterized in any more detail, 1.7 g (56.6%) of starting material were recovered.

### COMPARATIVE EXAMPLE 2

### Reaction of 4[(2,4-dichloro-5-methoxyanilino)carbonyl]-1,3,4-oxadiazinane with Thiophosgene Under Pressure

At 22° C., 2.00 g (6.533 mmol) of the title compound of m.p. 128-130° C., 1.1 ml (7.601 mmol) of triethylamine and a spatula tip of activated carbon were initially charged with stirring in 30 ml of toluene. Over a period of 1 h, 0.83 g (7.186 mmol) of thiophosgene in 30 ml of toluene were added at 0-5° C., and the mixture was stirred at 22° C. for another 2 h. According to HPLC analysis, at this point no reaction had occurred. The reaction mixture was transferred into an autoclave and stirred at 110° C. under intrinsic pressure for 12 h. After cooling, the reaction mixture was concentrated under reduced pressure. According to HPLC, the residue consisted of 13 components.

For work-up, the residue was dissolved in methylene chloride:diethyl ether 4:1 and chromatographed on a flash silica gel column using methylene chloride:ether 2:1 from fraction 30 onward. The resulting fractions were concentrated and examined by IR spectrometry.

In addition to isothiocyanates and decomposition products which were not characterized in any more detail, the starting material was recovered in a yield of 30%. In none of the fractions was the intensive C═O/C═S band at 1761 cm⁻¹ characteristic for the 3-thioxotriazole target product observed.

According to HPLC analysis [25 cm RP 18 column (Merck), 254 nm; acetonitrile/H₂O 60:40, 1 ml/min], too, none of the fractions contained a substance having the retention time measured for the target product (5.14 min).

II Experiments for preparing compounds of formula I where X and/or W=sulfur by treating triazolinediones with sulfurizing agents

### COMPARATIVE EXAMPLE 3

### Reaction of 2-[4-chloro-2-fluoro-5-(2-propynyloxy)phenyl]dihydro-1H-[1,2,4]-triazolo[1,2-c][1,3,4]oxadiazine-1,3(2H)-dione with Phosphorus Pentasulfide/Sodium Carbonate (Analogously to the Procedure in Synth. Comm. 1990, 20, 3085)

With stirring 0.28 g (2.679 mmol) of sodium carbonate and 1.19 g (2.679 mmol) of phosphorus pentasulfide were initially charged in 40 ml of tetrahydrofuran at 22° C., and the mixture was stirred for 30 min. 0.7 g (2.061 mmol) of the title compound, dissolved in 40 ml of THF, was added to the clear solution, followed by 20 ml of THF. The mixture was stirred at 22° C. for 30 minutes and then heated at 50° C. for 5 h and then for another 8½ h at 65° C. The reaction mixture was cooled and the precipitate was filtered off with suction and washed with methylene chloride, giving 1.5 g of an inorganic residue which was soluble neither in dimethyl sulfoxide nor in a 1:1 mixture of acetonitrile/water.

The filtrate was concentrated, giving 1.1 g of a residue having the following HPLC signals: 1.16, 3.24, 3.74 (starting material), 4.16 and 4.44. According to HPLC, the solution contained neither the 1-thioxo-3-oxo-tetrahydrotriazole derivative (HPLC 4.6) nor the isomeric 1-oxo-3-thiotetrahydrotriazole derivative (HPLC 4.7). [HPLC conditions (25 cm RP-18 column (Merck), 254 nm; acetonitrile/water 60:40, 1 ml/min].

The organic residue was triturated with 30 ml of ethyl acetate and insoluble material was filtered off with suction and dried, giving 0.45 g of starting material of melting point 196-199° C.

The filtrate was re-concentrated and chromatographed on a flash silica gel column using cyclohexane:ethyl acetate 1:1.

In addition to aliphatic and aromatic impurities, 0.089 g of the following compounds a and b

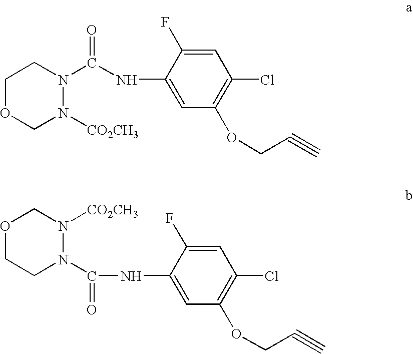

were isolated which were identified by their characteristic ¹H-NMR signals (CDCl₃) at δ 5.8 (doublet) for compound a and at δ 5.5 (doublet) for compound b. Moreover, 0.2 g of starting material was recovered. Thus, in total, 0.65 g (93%) of the starting material was recovered. It was not possible to detect the desired target compounds.

III Preparation of the substituted ureas of the formula II:

a) Methyl N-amino-N-2-hydroxyethylcarbamate

At 0-5° C., 248.4 g (2.628 mol) of methyl chloroformate were, over a period of 30 min, added with stirring to a mixture of 200 g (2.628 mol) of 2-hydrazinoethanol and 266 g (2.628 mol) of triethylamine in 1600 ml of methylene chloride. After 3 h of stirring at 3-22° C., the precipitated hydrochloride was filtered off with suction and washed with THF, and the filtrate was concentrated under reduced pressure. The residue was triturated with 800 ml of THF, filtered off with suction and washed with 1 l of THF, and the filtrate was concentrated under reduced pressure. This gave 366 g of the title compound as a colorless oil having a purity according to HPLC of 95.3%, which corresponds to a yield of 98.9% of theory. According to GC, the purity was 85.2%.

¹H-NMR (400 MHz, d₆-DMSO) δ(ppm): 4.4-4.8 (broad/3H) NH₂/OH; 3.6 (s/3H) CH₃O; 3.52 (t/2H) and 3.35 (t/2H) CH₂—CH₂

b) Methyl N-amino-N-2-hydroxyethylthiocarbamate

The reaction of 23.3 g (0.306 mol) of 2-hydrazinoethanol with 33.8 g (0.306 mol) of methyl thioformate in the presence of 31 g of triethylamine according to the procedure given under IIIa gave 40.7 g (88.5% of theory) of the title compound as a colorless oil having a refractive index n_{D}(²³)=1.5625.

c) Methyl tetrahydro-4H-1,3,4-oxdiazine-4-carboxylate

Over a period of 2 min, 22.4 g (0.746 mol) of paraformaldehyde were added with stirring to a mixture of 100 g (0.746 mol) of methyl N-amino-N-2-hydroxyethylcarbamate in 1500 ml of methylene chloride. 8.5 g (0.045 mol) of p-toluenesulfonic acid were added, and the mixture was then stirred at 42° C. for 21 h until the precipitate had dissolved. The mixture was cooled to 20° C., magnesium sulfate was added the mixture was filtered and the filtrate was concentrated under reduced pressure. This gave 111.8 g of the title compound as a colorless resin having a purity according to GC of 85%, which corresponds to a yield of 85.8% of theory.

¹H-NMR (500 MHz, CF₃CO₂D) δ(ppm): 5.09 (s/2H) CH₂; 4.02 (s/3H) CH₃O; 3.8-4.25 (m/4H) CH₂CH₂ IR v (cm⁻¹): C═O 1703

d) Methyl tetrahydro-4H-1,3,4-oxadiazine-4-thiocarboxylate

The reaction of 7.82 g (0.26 mol) of paraformaldehyde with 39.8 g (0.26 mol) of methyl N-amino-N-2-hydroxyethylthiocarbamate in the presence of 2.97 g (0.015 mol) of p-toluenesulfonic acid monohydrate gave 42.3 g of the title compound of boiling point 110-125° C./1 mbar, which corresponds to a yield of 99.8% of theory (GC: 7.14 min on a 30 m CP-Sil-5 column from Chrommpack).

e) Methyl tetrahydro-N-(2,4-dichloro-5-methoxyimino-methylphenyl)-4H-1,3,4-oxdiazine-3-thiocarboxamide-4-carboxylate (intermediate 96 from Table 6)

Over a period of 5 min, 9.11 g (0.035 mol) of 2,4-dichloro-5-methoxyiminomethylphenyl isothiocyanate were added with stirring to 10.22 g (0.07 mol) of methyl tetrahydro-4H-1,3,4-oxadiazine-4-carboxylate in 150 ml of tetrahydrofuran, and the mixture was stirred at 22° C. for 5 h and at 40-50° C. for 2 h. The reaction mixture was concentrated under reduced pressure. The residue was taken up in methylene chloride and fractionated on silica gel. This gave 11.9 g (78% of theory) of the title compound of m.p. 80-83° C.

f) 6-Fluoro-3-methyl-1,2-benzisothiazole 5-isothiocyanate

Over a period of 25 min, 22.7 g (0.198 mol) of thiophosgene in 50 ml of methylene chloride were added with stirring to a mixture of 300 ml of methylene chloride and 33.2 g (0.395 mol) of sodium bicarbonate in 200 ml of water. The mixture was stirred at 22° C. for 30 min, and at 22-26° C., 18 g (98.8 mmol) of 5-amino-6-fluoro-3-methyl-1,2-benzisothiazole in 150 ml of methylene chloride were then added with stirring, over a period of 5 min. The mixture was then stirred at 22° C. for 12 h. The precipitate insoluble in the two phases was filtered off with suction. The organic phase was washed with water and dried over magnesium sulfate. Concentration under reduced pressure gave 20.0 g (90.3% of theory) of the title compound of m.p. 84-88° C.

g) 6-Fluoro-3-methoxymethyl-1,2-benzisothiazole 5-isothiocyanate

Reaction of 2.8 g (13.19 mmol) of 5-amino-6-fluoro-3-methoxymethyl-1,2-benzisothiazole, 4.4 g (52.77 mmol) of sodium bicarbonate and 3.0 g (26.38 mmol) of thiophosgene by the procedure described under f gave 3.3 g (98.4% of theory) of the title compound of m.p. 115-117° C.

h) 6-Fluoro-3-ethoxycarbonyl-1,2-benzisothiazole 5-isothiocyanate

Reaction of 3.0 g (12.49 mmol) of 5-amino-6-fluoro-3-ethoxycarbonyl-1,2-benzisothiazole, 4.2 g (49.95 mmol) of sodium bicarbonate and 2.9 g (24.97 mmol) of thiophosgene by the procedure described under f gave 3.6 g (97% of theory) of the title compound of m.p. 122-123° C.

The intermediates 1 to 153 listed in Tables 6, 7, 8 and 9 were prepared analogously.

TABLE 6
(Intermediates 1 to 103)

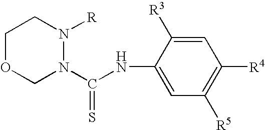

| Inter- | | | | | m.p.: [° C.] or |
|---|---|---|---|---|---|
| mediate | R | R³ | R⁴ | R⁵ | IR: ν [cm⁻¹] |
| 1 | CO₂CH₃ | Cl | Cl | OCH₂C≡CH | 141-143° C. |
| 2 | CO₂CH₃ | F | Cl | OC(═CH₂)CO₂CH₃ | 68-71° C. |
| 3 | CO₂CH₃ | F | Cl | CO₂CH(CH₃)₂ | 149-151° C. |
| 4 | CO₂CH₃ | F | Cl | SCH(CH₃)CO₂CH₃ | 1735 cm⁻¹ |
| 5 | CO₂CH₃ | F | Cl | OCH₂C≡CH | 156-160° C. |
| 6 | C(S)OCH₃ | F | Cl | OCH₂C≡CH | |
| 7 | CO₂CH₃ | F | Cl | O—C(═CH₂)CO₂CH₂CH₃ | |
| 8 | CO₂CH₃ | F | Cl | CH═NOCH₃ | 150-153° C. |
| 9 | C(S)OCH₃ | F | Cl | CH═NOCH₃ | |
| 10 | CO₂CH₃ | F | Cl | CO—O—C(CH₃)₃ | |
| 11 | CO₂CH₃ | H | Cl | CH═NOCH₃ | |
| 12 | CO₂CH₃ | H | Cl | OCH₂C≡CH | |
| 13 | C(S)OCH₃ | H | Cl | OCH₂C≡CH | |
| 14 | C(S)OCH₃ | Cl | Cl | OCH₂C≡CH | |
| 15 | C(S)OCH₃ | H | Cl | OCH₂CO₂CH₃ | |
| 16 | CO₂CH₃ | H | Cl | OCH₂CO₂CH₃ | 142° C. |
| 17 | C(S)OCH₃ | Cl | Cl | OCH₂CO₂CH₃ | |
| 18 | CO₂CH₃ | Cl | Cl | OCH₂CO₂CH₃ | |
| 19 | C(S)OCH₃ | F | Cl | OCH₂CO₂CH₃ | |
| 20 | CO₂CH₃ | F | Cl | OCH₂CO₂CH₃ | 143° C. |
| 21 | C(S)OCH₃ | F | Cl | OCH₂CO₂-nC₅H₁₁ | |
| 22 | CO₂CH₃ | F | Cl | OCH₂CO₂-nC₅H₁₁ | |
| 23 | C(S)OCH₃ | Cl | Cl | SCH₂CO₂CH₃ | |
| 24 | CO₂CH₃ | Cl | Cl | SCH₂CO₂CH₃ | |
| 25 | C(S)OCH₃ | F | Cl | SCH₂CO₂CH₃ | |
| 26 | CO₂CH₃ | F | Cl | SCH₂CO₂CH₃ | 129-132° C. |
| 27 | C(S)OCH₃ | H | Cl | OCH(CH₃)CO₂CH₃ | |
| 28 | CO₂CH₃ | H | Cl | OCH(CH₃)CO₂CH₃ | |
| 29 | C(S)OCH₃ | F | Cl | OCH(CH₃)CO₂CH₃ | |
| 30 | CO₂CH₃ | F | Cl | OCH(CH₃)CO₂CH₃ | 72-76° C. |
| 31 | C(S)OCH₃ | F | Cl | SCH(CH₃)CO₂CH₃ | |
| 32 | C(S)OCH₃ | H | Cl | CO₂CH₃ | |
| 33 | CO₂CH₃ | H | Cl | CO₂CH₃ | |
| 34 | C(S)OCH₃ | F | Cl | CO₂CH₃ | |
| 35 | CO₂CH₃ | F | Cl | CO₂CH₃ | 185-187° C. |
| 36 | C(S)OCH₃ | H | Cl | CO₂C(CH₃)₂CO₂CH₂CH═CH₂ | |
| 37 | CO₂CH₃ | H | Cl | CO₂C(CH₃)₂CO₂CH₂CH═CH₂ | |
| 38 | C(S)OCH₃ | Cl | Cl | CO₂C(CH₃)₂CO₂CH₂CH═CH₂ | |
| 39 | CO₂CH₃ | Cl | Cl | CO₂C(CH₃)₂CO₂CH₂CH═CH₂ | |
| 40 | C(S)OCH₃ | F | Cl | CO₂C(CH₃)₂CO₂CH₂CH═CH₂ | |
| 41 | CO₂CH₃ | F | Cl | CO₂C(CH₃)₂CO₂CH₂CH═CH₂ | 69-71° C. |
| 42 | C(S)OCH₃ | F | Cl | CO₂CH₂CH₂OCH₃ | |
| 43 | CO₂CH₃ | F | Cl | CO₂CH₂CH₂OCH₃ | |
| 44 | C(S)OCH₃ | F | Cl | C(═NOCH₃)OCH₃ | |
| 45 | CO₂CH₃ | F | Cl | C(═NOCH₃)OCH₃ | |
| 46 | C(S)OCH₃ | F | Cl | C(═NOCH₃)OCH₂CO₂CH₃ | |
| 47 | CO₂CH₃ | F | Cl | C(═NOCH₃)OCH₂CO₂CH₃ | |
| 48 | C(S)OCH₃ | F | Cl | C(O)N(CH₃)OCH₃ | |
| 49 | CO₂CH₃ | F | Cl | C(O)N(CH₃)OCH₃ | |
| 50 | C(S)OCH₃ | Cl | Cl | CH═NOCH₃ | |
| 51 | CO₂CH₃ | F | Cl | CH═NOC₂H₅ | |
| 52 | C(S)OCH₃ | F | Cl | CH═NOC₂H₅ | |
| 53 | CO₂CH₃ | F | Cl | CH═NOCH₂CO₂CH₃ | |
| 54 | C(S)OCH₃ | F | Cl | CH═NOCH₂CO₂CH₃ | |
| 55 | CO₂CH₃ | F | Cl | CH═NOCH(CH₃)CO₂CH₃ | |
| 56 | C(S)OCH₃ | F | Cl | CH═NOCH(CH₃)CO₂CH₃ | |
| 57 | CO₂CH₃ | F | Cl | CH═C(Cl)CO₂C₂H₅ | 78-80° C. |
| 58 | C(S)OCH₃ | F | Cl | CH═C(Cl)CO₂C₂H₅ | |
| 59 | CO₂CH₃ | Cl | Cl | CH═C(Cl)CO₂C₂H₅ | |
| 60 | C(S)OCH₃ | Cl | Cl | CH═C(Cl)CO₂C₂H₅ | |
| 61 | CO₂CH₃ | H | Cl | CH═C(Cl)CO₂C₂H₅ | |
| 62 | C(S)OCH₃ | H | Cl | CH═C(Cl)CO₂C₂H₅ | |
| 63 | CO₂CH₃ | F | Cl | CH₂—CH(Cl)CO₂C₂H₅ | |
| 64 | C(S)OCH₃ | F | Cl | CH₂—CH(Cl)CO₂C₂H₅ | |
| 65 | CO₂CH₃ | F | Cl | CH═NOCH₂C≡CH | |
| 66 | C(S)OCH₃ | F | Cl | CH═NOCH₂C≡CH | |
| 67 | CO₂CH₃ | F | Cl | O-cyclopentyl | 177-179° C. |
| 68 | C(S)OCH₃ | F | Cl | O-cyclopentyl | |
| 69 | CO₂CH₃ | F | Cl | OCH₂CH═CH₂ | 158-159° C. |
| 70 | C(S)OCH₃ | F | Cl | OCH₂CH═CH₂ | |
| 71 | CO₂CH₃ | F | Cl | OCH₂CH═CHCl | |
| 72 | C(S)OCH₃ | F | Cl | OCH₂CH═CHCl | |
| 73 | CO₂CH₃ | Cl | Cl | COOH | |
| 74 | CO₂i-C₃H₇ | Cl | Cl | CN | 140-143° C. |
| 75 | CO₂CH₃ | F | CN | OCH₂C≡CH | |
| 76 | CO₂CH₃ | F | CN | OCH₂C≡CH | |
| 77 | CO₂CH₃ | F | CN | OCH₂CO₂H | |
| 78 | CO₂CH₃ | F | CN | OCH(CH₃)CO₂CH₃ | |
| 79 | C(S)OCH₃ | F | CN | OCH(CH₃)CO₂CH₃ | |
| 80 | CO₂CH₃ | F | CN | SCH₂CO₂H | |
| 81 | C(S)OCH₃ | F | CN | SCH₂CO₂H | |
| 82 | CO₂CH₃ | F | CN | SCH(CH₃)CO₂CH₃ | |
| 83 | C(S)OCH₃ | F | CN | SCH(CH₃)CO₂CH₃ | |
| 84 | CO₂CH₃ | F | Cl | OCH(CH₃)CO₂H | |
| 85 | CO₂CH₃ | F | Cl | OCH₂CO₂H | |
| 86 | CO₂CH₃ | F | Cl | CO₂H | |
| 87 | CO₂CH₃ | F | Cl | CHO | |
| 88 | CO₂CH₃ | F | Cl | CO₂CH₂CH₃ | |
| 89 | C(S)OCH₃ | F | Cl | CO₂CH₂CH₃ | |
| 90 | CO₂CH₃ | F | Cl | OCH(CH₃)CO₂CH₂C≡CH | |
| 91 | CO₂CH₃ | F | Cl | OCH₂CO₂CH₂CO₂CH₃ | |
| 92 | CO₂CH₃ | F | Cl | O-cyclopentyl | |
| 93 | CO₂CH₃ | F | CN | OH | 204° C. |
| 94 | CO₂CH₃ | F | Cl | CO₂CH₂C≡CH | |
| 95 | CO₂CH₃ | F | Cl | SCH₂CO₂H | |
| 96 | CO₂CH₃ | Cl | Cl | CH═NOCH₃ | 80-83° C. |
| 97 | CO₂CH₃ | H | Cl | CH═NOCH₃ | |
| 98 | CO₂CH₃ | Cl | Cl | OCH₃ | |
| 99 | CO₂CH₃ | F | Cl | SCH(CH₃)CO₂CH₃ | CO/CS = 1735 |
| 100 | CO₂CH₃ | F | F | NO₂ | 127° C. |
| 101 | CO₂CH₃ | F | Cl | CH═C(Cl)CO₂CH₃ | 169-170° C. |
| 102 | CO₂CH₃ | F | CN | F | 175-176° C. |
| 103 | CO₂CH₃ | Cl | Cl | OCH₃ | CO/CS = 1730, 1704 |

TABLE 7
(Intermediates 104 to 111)

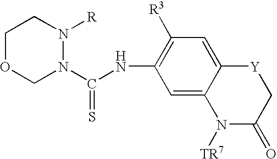

| Inter- | | | | | | m.p.: [° C.] or |
|---|---|---|---|---|---|---|
| mediate | R | R³ | Y | T | R³ | IR: ν [cm⁻¹] |
| 104 | CO₂CH₃ | Cl | O | — | CH₂C≡CH | |
| 105 | C(S)OCH₃ | Cl | O | — | CH₂C≡CH | |
| 106 | CO₂CH₃ | F | O | — | CH₂C≡CH | |
| 107 | C(S)OCH₃ | F | O | — | CH₂C≡CH | |
| 108 | CO₂CH₃ | F | O | O | CH₂C≡CH | |
| 109 | C(S)OCH₃ | F | O | O | CH₂C≡CH | |
| 110 | CO₂CH₃ | F | O | O | CH(CH₃)₂ | |
| 111 | C(S)OCH₃ | F | O | O | CH(CH₃)₂ | |

TABLE 8
(Intermediates 112 to 119)

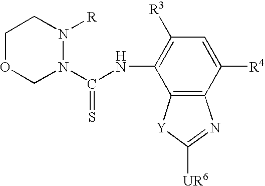

| Inter- | | | | | | | m.p.: [° C.] or |
|---|---|---|---|---|---|---|---|
| mediate | R | R³ | R⁴ | Y | U | R⁶ | IR: ν [cm⁻¹] |
| 112 | CO₂CH₃ | F | Cl | S | — | CH₂CH₃ | |
| 113 | CO₂CH₃ | F | Cl | S | — | cyclo-C₃H₅ | |
| 114 | CO₂CH₃ | F | Cl | S | O | CH₂CH₃ | |
| 115 | CO₂CH₃ | Cl | Cl | S | — | CH₂CH₃ | |
| 116 | CO₂CH₃ | F | Cl | O | — | cyclo-C₃H₅ | 187-190° C. |
| 117 | C(S)OCH₃ | F | Cl | O | — | cyclo-C₃H₅ | |
| 118 | CO₂CH₃ | F | Cl | O | — | CH₂CH₃ | |
| 119 | CO₂CH₃ | F | Cl | O | O | CH₂CH₃ | |

TABLE 9
(Intermediates 120-153)

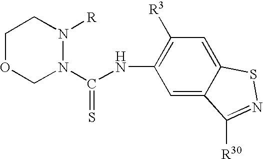

| Inter- | | | | m.p.: [° C.] or |
|---|---|---|---|---|
| mediate | R | R³ | R³⁰ | IR: ν [cm⁻¹] |
| 120 | CO₂CH₃ | F | H | |
| 121 | CO₂CH₃ | F | CH₃ | 148-151° C. |
| 122 | CO₂CH₃ | F | C₂H₅ | |
| 123 | CO₂CH₃ | F | n-C₃H₇ | |
| 124 | CO₂CH₃ | F | CH₂OCH₃ | 189-190° C. |
| 125 | CO₂CH₃ | F | CH₂OC₂H₅ | |
| 126 | CO₂CH₃ | F | CH₂OCH₂CH═CH₂ | |
| 127 | CO₂CH₃ | F | CH₂OCH₂C≡CH | |
| 128 | CO₂CH₃ | F | CH₂CH₂OCH₃ | |
| 129 | CO₂CH₃ | F | CO₂CH₃ | |
| 130 | CO₂CH₃ | F | CO₂C₂H₅ | 86-89° C. |
| 131 | CO₂CH₃ | F | CO₂-(n-C₃H₇) | |
| 132 | CO₂CH₃ | F | CO₂-(i-C₃H₇) | |
| 133 | CO₂CH₃ | F | CO₂CH₂CH═CH₂ | |
| 134 | CO₂CH₃ | F | CO₂CH₂C≡CH | |
| 135 | CO₂CH₃ | F | CO₂-cyclopropyl | |
| 136 | CO₂CH₃ | F | CH₂CO₂CH₃ | |
| 137 | CO₂CH₃ | F | CH₂CO₂C₂H₅ | |
| 138 | CO₂CH₃ | F | CH(CH₃)CO₂CH₃ | |
| 139 | CO₂CH₃ | F | CH₂CO₂CH₂CH═CH₂ | |
| 140 | CO₂CH₃ | F | CH(CH₃)CO₂CH₂CH═CH₂ | |
| 141 | CO₂CH₃ | F | CH(OCH₃)₂ | |
| 142 | CO₂CH₃ | F | CH(OC₂H₅₎₂ | |
| 143 | CO₂CH₃ | F | CH[O(CH₂)₃O] | |
| 144 | CO₂CH₃ | F | CH[(O(CH₂)₄O] | |
| 145 | CO₂CH₃ | F | C(O)NHSO₂CH₃ | |
| 146 | CO₂CH₃ | F | C(O)NHSO₂C₂H₅ | |
| 147 | CO₂CH₃ | F | C(O)NHSO₂-i-C₃H₇ | |
| 148 | CO₂CH₃ | F | C₆H₅ | |
| 149 | CO₂CH₃ | F | (2-CH₃OC(O)CH₂)C₆H₄ | |
| 150 | CO₂CH₃ | F | (2-CH₃OCOCH(CH₃))C₆H₄ | |
| 151 | CO₂CH₃ | F | 2-chlorophenyl | |
| 152 | CO₂CH₃ | F | 3-chlorophenyl | |
| 153 | CO₂CH₃ | F | 4-CF₃—C₆H₄ | |

IV Preparation of the fused tetrahydro-[1H]-triazoles I

### EXAMPLE 1

### 2-[2,4-Dichloro-5-propynyloxyphenyl]-3-thioxotetrahydro-1H-[1,2,4]triazolo[1,2-c][1,3,4]oxadiazin-1-one

3.5 g (8.657 mmol) of methyl 3-[(2,4-dichloro-5-propynyloxyanilino)carbothioyl]-1,3,4-oxadiazinane-4-carboxylate were initially charged in a mixture of 200 ml of methanol and 70 ml of water. At 22° C., 1.00 g (9.523 mmol) of triethylamine was added with stirring. After 3 h, the reaction mixture was concentrated under reduced pressure, the residue was taken up in methylene chloride and the organic phase was washed with saturated sodium chloride solution. Drying, filtration with suction through silica gel and concentration under reduced pressure gave 2.8 g (84.3% of theory) of the title compound of m.p. 188-190° C.

### EXAMPLE 2

### 2-[2-Chloro-4-fluoro-5-(1-oxo-3-thioxodihydro-1H-[1,2,4]-triazolo-[1,2-c][1,3,4]-oxadiazin-2(3H)-yl)phenoxy]acrylic acid

2.2 g [5.071 mmol] of methyl 3-[(4-chloro-2-fluoro-5{[1-(methoxycarbonyl)vinyl]oxy}anilino)carbothioyl]-1,3,4-oxadiazinone-4-carboxylate were dissolved in 40 ml of methanol, and at 5-15° C., a solution of 0.2 g (5.1 mmol) of sodium hydroxide in 15 ml of water was added with stirring, over a period of 25 min. The reaction mixture was stirred at 10-15° C. for 2 h and then left at 22° C. overnight. With stirring, the mixture was acidified with 1N hydrochloric acid and extracted with methylene chloride, and the organic phase was dried and concentrated under reduced pressure. This gave 2.0 g (96.6% of theory, calc. 95% pure) of the title compound of melting point 130° C. (decomposition).

### EXAMPLE 3

### Isopropyl 2-chloro-4-fluoro-5-(1-oxo-3-thioxodihydro-1H-[1,2,4]triazolo[1,2-c][1,3,4]oxadiazin-2(3H)-yl)benzoate

0.24 g (2.4 mmol) of triethylamine was added to a mixture of 1.0 g (2.4 mmol) of methyl 3-{[4-chloro-2-fluoro-5-(isopropoxycarbonyl)anilino]carbothioyl}-1,3,4-oxadiazinane-4-carboxylate in a mixture of 40 ml of isopropanol and 10 ml of water, and the mixture was stirred at 22° C. for 12 h. The clear reaction solution was concentrated under reduced pressure and the residue was taken up in methylene chloride and extracted with dilute hydrochloric acid. The organic phase was washed with saturated sodium chloride solution and dried. Concentration under reduced pressure gave 0.9 g (95.5% of theory, calc. 98% pure) of the title compound of melting point 67-69° C.

### EXAMPLE 4

### 2-[4-Chloro-2-fluoro-5-(1-methoxycarbonylethyl-1-thio)phenyl]-3-thioxotetrahydro-1H-[1,2,4]triazolo-[1,2-c][1,3,4]oxadiazin-1-one

Analogously to Example 1, 3.3 g (96.4% of theory) of the title compound of melting point 129-134° C. were obtained from 3.5 g (7.745 mmol) of methyl 3-{[4-chloro-2-fluoro-5-(1-methoxycarbonylethyl-1-thio)anilino]carbothioyl}-1,3,4-oxadiazinane-4-carboxylate and 0.78 g (7.745 mmol) of triethylamine in 240 ml of methanol and 40 ml of water.

### EXAMPLE 5

### 2-[4-Chloro-2-fluoro-5-(2-propynyloxy)phenyl]-3-thiotetrahydro-1H-(1,2,4)triazolo[1,2-c][1,3,4]oxadiazin-1-one

Analogously to Example 1, the title compound of melting point 165-167° C. was obtained from methyl 3-{[4-chloro-2-fluoro-5-(2-propynyloxy)anilino]carbothioyl}-1,3,4-oxadiazinane-4-carboxylate.

### EXAMPLE 6

### 2-[4-Chloro-2-fluoro-5-(2-propynyloxy)phenyl]tetrahydro-1H-[1,2,4]triazolo[1,2-c][1,3,4]oxadiazin-1,3-dithione

At room temperature, 2.0 g (8.28 mmol) of 4-chloro-2-fluoro-5-propynyloxyphenyl isothiocycanate were added with stirring, over a period of 2 min, to a mixture of 4.03 g (12.4 mmol) of methyl tetrahydro-4H-1,3,4-oxdiazine-4-thiocarboxylate (50% by weight) in 150 ml of tetrahydrofuran. After 12 h at room temperature, the reaction mixture was concentrated under reduced pressure and the residue was taken up in methylene chloride and chromatographed on silica gel using the same solvent. The residue obtained after concentration of the eluate was crystallized from ethyl acetate/cyclohexane (1:4 v/v). This gave 0.35 g (11% of theory) of the title compound of melting point 167-169° C.

The compounds of Examples 7 to 160 listed in Tables 10, 11, 12 and 13 can be prepared analogously to the methods described in Examples 1 to 6:

TABLE 10
(Examples 1 to 121):

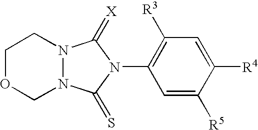

| Ex- | | | | | m.p.: [° C.] or |
|---|---|---|---|---|---|
| ample | X | R³ | R⁴ | R⁵ | IR: ν [cm⁻¹] |
| 1 | O | Cl | Cl | OCH₂C═CH | 188-190° C. |
| 2 | O | F | Cl | OC(CH₂)CO₂H | 130° C., |
| | | | | | decomposition |
| 3 | O | F | Cl | CO₂CH(CH₃)₂ | 67-69° C. |
| 4 | O | F | Cl | SCH(CH₃)CO₂CH₃ | 129-134° C. |
| 5 | O | F | Cl | OCH₂C═CH | 165-167° C. |
| 6 | S | F | Cl | OCH₂C═CH | 167-169° C. |
| 7 | O | F | Cl | O—C(═CH₂)CO₂CH₃ | 69-71° C. |
| 8 | O | F | Cl | CH═NOCH₃ | 79-80° C. |
| 9 | S | F | Cl | CH═NOCH₃ | |
| 10 | O | F | Cl | CO—O—C(CH₃)₃ | |
| 11 | O | H | Cl | CH═NOCH₃ | |
| 12 | O | H | Cl | OCH₂C═CH | |
| 13 | S | H | Cl | OCH₂C═CH | |
| 14 | S | Cl | Cl | OCH₂C═CH | |
| 15 | S | H | Cl | OCH₂CO₂CH₃ | |
| 16 | O | H | Cl | OCH₂CO₂CH₃ | |
| 17 | S | Cl | Cl | OCH₂CO₂CH₃ | |
| 18 | O | Cl | Cl | OCH₂CO₂CH₃ | |
| 19 | S | F | Cl | OCH₂CO₂CH₃ | |
| 20 | O | F | Cl | OCH₂CO₂CH₃ | 65-66° C. |
| 21 | S | F | Cl | OCH₂CO₂-nC₅H₁₁ | |
| 22 | O | F | Cl | OCH₂CO₂-nC₅H₁₁ | |
| 23 | S | Cl | Cl | SCH₂CO₂CH₃ | |
| 24 | O | Cl | Cl | SCH₂CO₂CH₃ | |
| 25 | S | F | Cl | SCH₂CO₂CH₃ | |
| 26 | O | F | Cl | SCH₂CO₂CH₃ | 1756 cm⁻¹ |
| 27 | S | H | Cl | OCH(CH₃)CO₂CH₃ | |
| 28 | O | H | Cl | OCH(CH₃)CO₂CH₃ | |
| 29 | S | F | Cl | OCH(CH₃)CO₂CH₃ | |
| 30 | O | F | Cl | OCH(CH₃)CO₂CH₃ | 68-71° C. |
| 31 | S | F | Cl | SCH(CH₃)CO₂CH₃ | |
| 32 | S | H | Cl | CO₂CH₃ | |
| 33 | O | H | Cl | CO₂CH₃ | |
| 34 | S | F | Cl | CO₂CH₃ | 170-172° C. |
| 35 | O | F | Cl | CO₂CH₃ | 146-147° C. |
| 36 | S | H | Cl | CO₂C(CH₃)₂CO₂CH₂CH═CH₂ | |
| 37 | O | H | Cl | CO₂C(CH₃)₂CO₂CH₂CH═CH₂ | |
| 38 | S | Cl | Cl | CO₂C(CH₃)₂CO₂CH₂CHCH₂ | |
| 39 | O | Cl | Cl | CO₂C(CH₃)₂CO₂CH₂CHCH₂ | |
| 40 | S | F | Cl | CO₂C(CH₃)₂CO₂CH₂CHCH₂ | |
| 41 | O | F | Cl | CO₂C(CH₃)₂CO₂CH₂CHCH₂ | C═O/ |
| | | | | | C═S 1756 |
| 42 | S | F | Cl | CO₂CH₂CH₂OCH₃ | |
| 43 | O | F | Cl | CO₂CH₂CH₂OCH₃ | resin, |
| | | | | | HPLC: 4.74 |
| | | | | | min.²) |
| 44 | 5 | F | Cl | C(═NOCH₃)OCH₃ | |
| 45 | O | F | Cl | C(═NOCH₃)OCH₃ | |
| 46 | S | F | Cl | C(═NOCH₃)OCH₂CO₂CH₃ | |
| 47 | O | F | Cl | C(═NOCH₃)OCH₂CO₂CH₃ | |
| 48 | S | F | Cl | C(O)N(CH₃)OCH₃ | |
| 49 | O | F | Cl | C(O)N(CH₃)OCH₃ | |
| 50 | S | Cl | Cl | CH═NOCH₃ | |
| 51 | O | F | Cl | CH═NOC₂H₅ | |
| 52 | S | F | Cl | CH═NOC₂H₅ | |
| 53 | O | F | Cl | CH═NOCH₂CO₂CH₃ | 139-140° C. |
| 54 | S | F | Cl | CH═NOCH₂CO₂CH₃ | |
| 55 | O | F | Cl | CH═NOCH(CH₃)CO₂CH₃ | |
| 56 | S | F | Cl | CH═NOCH(CH₃)CO₂CH₃ | |
| 57 | O | F | Cl | CH═C(Cl)CO₂C₂H₅ | 118-120° C. |
| 58 | S | F | Cl | CH═C(Cl)CO₂C₂H₅ | |
| 59 | O | Cl | Cl | CH═C(Cl)CO₂C₂H₅ | |
| 60 | S | Cl | Cl | CH═C(Cl)CO₂C₂H₅ | |
| 61 | O | H | Cl | CH═C(Cl)CO₂C₂H₅ | |
| 62 | S | H | Cl | CH═C(Cl)CO₂C₂H₅ | |
| 63 | O | F | Cl | CH₂—CH(Cl)CO₂C₂H₅ | |
| 64 | S | F | Cl | CH₂—CH(Cl)CO₂C₂H₅ | |
| 65 | O | F | Cl | CH═NOCH₂C═CH | |
| 66 | S | F | Cl | CH═NOCH₂C═CH | |
| 67 | O | F | Cl | O-cyclopentyl | 74-77° C. |
| 68 | S | F | Cl | O-cyclopentyl | |
| 69 | O | F | Cl | OCH₂CH═CH₂ | 108-110° C. |
| 70 | S | F | Cl | OCH₂CH═CH₂ | |
| 71 | O | F | Cl | OCH₂CH═CHCl | |
| 72 | S | F | Cl | OCH₂CH═CHCl | |
| 73 | O | Cl | Cl | COOH | |
| 74 | O | Cl | Cl | CN | |
| 75 | O | F | CN | OCH₂C═CH | 226-227° C. |
| 76 | O | F | CN | OCH₂CH═CH₂ | 163-164° C. |
| 77 | O | F | CN | OCH₂CO₂H | |
| 78 | O | F | CN | OCH(CH₃)CO₂CH₃ | 187-188° C. |
| 79 | S | F | CN | OCH(CH₃)CO₂CH₃ | |
| 80 | O | F | CN | SCH₂CO₂H | |
| 81 | S | F | CN | SCH₂CO₂H | |
| 82 | O | F | CN | SCH(CH₃)CO₂CH₃ | |
| 83 | S | F | CN | SCH(CH₃)CO₂CH₃ | |
| 84 | O | F | Cl | OCH(CH₃)CO₂H | 81° C., |
| | | | | | decomposition |
| 85 | O | F | Cl | OCH₂CO₂H | 195° C., |
| | | | | | decomposition |
| 86 | O | F | Cl | CO₂H | 90-95° C. |
| 87 | O | F | Cl | CHO | 185-188° C. |
| 88 | O | F | Cl | CO₂CH₂CH₃ | 70-72° C. |
| 89 | S | F | Cl | CO₂CH₂CH₃ | |
| 90 | O | F | Cl | OCH(CH₃)CO₂CH₂C═CH | 60-62° C. |
| 91 | O | F | Cl | OCH₂CO₂CH₂CO₂CH₃ | 48-51° C. |
| 92 | O | F | Cl | CONH-cyclopentyl | 108-110° C. |
| 93 | O | F | Cl | OH | 188-189° C. |
| 94 | O | F | Cl | CO₂CH₂C═CH | 71-74° C. |
| 95 | O | F | Cl | SCH₂CO₂H | 182-185° C. |
| 96 | O | Cl | Cl | CH═NOCH₃ | 88-90° C. |
| 97 | O | H | Cl | CH═NOCH₃ | 232-233° C. |
| 98 | S | Cl | Cl | OCH₃ | |
| 99 | O | F | Cl | OCH₂CO₂CH₂C═CH | 162-163° C. |
| 100 | O | F | Cl | OCH(CH₃)CO₂CH₂CO₂CH₃ | 65-67° C. |
| 101 | O | F | Cl | OCH(CH₃)CO₂CH₂CH₂OCH₃ | 61-63° C. |
| 102 | O | F | CN | F | 187-189° C. |
| 103 | O | F | Cl | CH═C(Cl)CO₂CH₃ | 109-112° C. |
| 104 | O | F | Cl | OCH₃ | 128-135° C. |
| 105 | O | Cl | Cl | OCH₃ | 96° C. |
| 106 | O | F | Cl | SCH₂CO₂CH₂CO₂CH₃ | 58-62° C. |
| 107 | S | F | Cl | SCH₂CO₂CH₂CO₂CH₃ | |
| 108 | O | F | Cl | CH═N—OH | 180-185° C. |
| 109 | O | F | Cl | OCH₂—C₆H₅ | 118-120° C. |
| 110 | O | F | CN | OCH₂CO₂CH₃ | 162-165° C. |
| 111 | O | F | CN | OH | 224-227° C. |
| 112 | O | F | Cl | OCH₂C₂O)N(C₆H₁₁)C(O)— | 160-165° C. |
| | | | | NHC₆H₁₁ | |
| 113 | O | F | Cl | SN | |
| 114 | O | F | CN | SN | |
| 115 | O | Cl | CN | OH | |
| 116 | O | Cl | CN | OCH₂C═CH | |
| 117 | O | F | CN | SCH₂CO₂CH₃ | |
| 118 | O | F | CN | OCH₂CO₂CH₂C═CH | |
| 119 | O | F | CN | OCH₂CO₂CH₂CH₂OCH₃ | |
| 120 | O | F | CN | OCH₂CO₂CH₂CO₂CH₃ | |
| 121 | O | F | CN | CH═NOCH₃ | |

2) Experimental conditions as in Comparative Example 2

TABLE 11
(Examples 122 to 129)

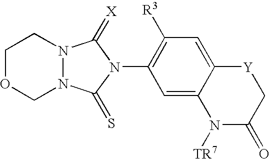

| | | | | | | m.p.: [° C.] or |
|---|---|---|---|---|---|---|
| Example | X | R³ | Y | T | R⁷ | IR: ν [cm⁻¹] |
| 122 | O | Cl | O | — | CH₂C═CH | |
| 123 | S | Cl | O | — | CH₂C═CH | 230° C. |
| 124 | O | F | O | — | CH₂C═CH | |
| 125 | S | F | O | — | CH₂C═CH | |
| 126 | O | F | O | O | CH₂C═CH | |
| 127 | S | F | O | O | CH₂C═CH | |
| 128 | O | F | O | O | CH(CH₃)₂ | |
| 129 | S | F | O | O | CH(CH₃)₂ | |

TABLE 12
(Examples 130 to 137)

| | | | | | | | m.p.: [° C.] or |
|---|---|---|---|---|---|---|---|
| Example | X | R³ | R⁴ | Y | U | R⁶ | IR: ν [cm⁻¹] |
| 130 | O | F | Cl | S | — | CH₂CH₃ | 182-185° C. |
| 131 | O | F | Cl | S | — | cyclo-C₃H₅ | |
| 132 | O | F | Cl | S | O | CH₂CH₃ | |
| 133 | O | Cl | Cl | S | — | CH₂CH₃ | |
| 134 | O | F | Cl | O | — | cyclo-C₃H₅ | 176-179° C. |
| 135 | S | F | Cl | O | — | cyclo-C₃H₅ | |
| 136 | O | F | Cl | O | — | CH₂CH₃ | |
| 137 | S | F | Cl | O | — | CH₂CH₃ | |

TABLE 13
(Examples 138 to 160)

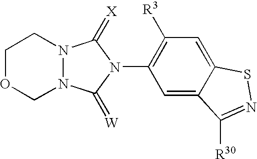

| Example | X | W | R³ | R³⁰ | m.p.: [° C.] |
|---|---|---|---|---|---|
| 138 | O | S | Cl | H | |
| 139 | O | S | F | H | |
| 140 | O | S | Cl | CH₃ | |
| 141 | O | S | F | CH₃ | 283-284° C. |
| 142 | O | S | Cl | C₂H₅ | |
| 143 | O | S | F | C₂H₅ | |
| 144 | O | O | F | CH₃ | 249-251° C. |
| 145 | O | S | F | n-C₃H₇ | |
| 146 | O | S | Cl | i-C₃H₇ | |
| 147 | O | S | F | CH₂OCH₃ | 220-222° C. |
| 148 | O | S | F | CH₂OC₂H₅ | |
| 149 | O | S | F | CH₂O-(n-C₃H₇) | |
| 150 | O | S | Cl | CH₂OCH₃ | |
| 151 | O | O | F | CH₂OCH₃ | |
| 152 | O | S | Cl | CH₂OC₂H₅ | |
| 153 | O | S | F | CH₂OCH₂CH═CH₂ | |
| 154 | O | S | F | CH₂OCH₂C═CH | |
| 155 | O | S | Cl | CO₂CH₃ | |
| 156 | O | S | F | CO₂CH₃ | |
| 157 | O | S | F | CO₂C₂H₅ | 220-221° C. |
| 158 | O | S | F | CO₂(n-C₃H₇) | |
| 159 | O | S | F | CO₂CH₂CH═CH₂ | |
| 160 | O | S | F | CO₂CH₂C≡CH | |

### Process Example 1

2-[4-Chloro-2-fluoro-5-(2-propynyloxy)phenyl]tetrahydro-1H-(1,2,4)triazolo[1,2-c][1,3,4]oxadiazine-1,3-dione by Base-Catalyzed Cyclization

At 22° C., 1.0 g (9.87 mmol) of triethylamine was added with stirring to a mixture of methyl 3-{[4-chloro-2-fluoro-5-(2-propynyloxy)anilino]carbonyl}-1,3,4-oxadiazinone-4-carboxylate in 100 ml of methanol and 25 ml of water, and the mixture was stirred at 22° C. for 12 h. The solvent was removed under reduced pressure and the residue was then partitioned between methylene chloride and water and the organic phase was dried and concentrated. The residue was chromatographed on silica gel using cyclohexane/ethyl acetate 9:1, giving 2.94 g (87.6% of theory) of the title compound of m.p. 197-199° C.

### Process Example 2

2-[4-Chloro-2-fluoro-5-(2-propynyloxy)phenyl]tetrahydro-1H-[1,2,4]triazolo[1,2-c][1,3,4]oxadiazine-1,3-dione by Phosgene Cyclization.

7.5 g (23.907 mmol) of methyl tetrahydro-N-(4′-chloro-2′-fluoro-5′-propargyloxyphenyl)-4H-1,3,4-oxadiazine-3-carboxamide-4-carboxylate were initially charged as a suspension in 250 ml of ethanol, and a solution of 1.55 g (38.735 mmol) of sodium hydroxide in 80 ml of water was then added with stirring, at 60-70° C., over a period of 20 min. After 30 min of stirring at 60° C., hydrolysis was complete. The reaction mixture was concentrated, water and methylene chloride were added to the residue and the mixture was adjusted to pH 1-4 using 1N hydrochloric acid. Following phase separation, the organic phase was once more washed with water, dried and concentrated. This gave 8.0 g (98.7%) of tetrahydro-N-(4′-chloro-2′-fluoro-5′-propargyloxyphenyl)-4H-1,3,4-oxadiazine-3- and -4-carboxamide as an approximately 1:1 isomer mixture of m.p. 139-142° C.

7.5 g (23.907 mmol) of this mixture were initially charged as a solution in 80 ml of pyridine, a spatula tip of activated carbon was added and 4.7 g (23.907 mmol) of diphosgene were then added with stirring at 0-5° C. The mixture was stirred at 0-5° C. for 30 min and then at 22° C. for 1 h.

The reaction mixture was concentrated, water and methylene chloride were added to the residue and the pH was adjusted to 3 using 1N hydrochloric acid. Following phase separation and re-extraction with methylene chloride, the organic extract was washed with saturated sodium chloride solution, dried and concentrated. This gave 7.5 g (92.3% of theory) of the title compound of m.p. 198-200° C.

### Process Example 3

2-[4-Cyano-2-fluoro-5-(propargyloxy)phenyl]-3-thioxotetrahydro-1H-[1,2,4]-triazolo-[1,2-c][1,3,4]-oxadiazin-1-one (Compound 75 from Table 10) by alkylation of the corresponding phenol (Compound 111 from Table 10).

Over a period of 2 min, 0.45 g (3.244 mmol) of potassium carbonate and 0.39 g (3.244 mmol) of propargyl bromide were added with stirring to a mixture of 1.0 g (3.244 mmol) of the Compound 111 from Table 10 in 70 ml of acetonitrile. The reaction mixture was heated at 82° C. for 1.5 h, cooled to 22° C. and dried over magnesium sulfate, and, after removal of the drying agent, the solution was concentrated under reduced pressure. The residue was stirred with diethyl ether, filtered off with suction, washed and dried, giving 1.1 g (98% of theory) of the title compound of melting point 226-227° C.

### Intermediate Example 1

4-Cyano-2-fluoro-5-hydroxyphenyl isothiocyanate

Over a period of 30 min, 19.8 g (0.173 mol) of thiophosgene in 50 ml of ethyl acetate were added with stirring, at 20-23° C., to a solution of 25 g (0.164 mol) of 3-amino-6-cyano-4-fluorophenol in 450 ml of ethyl acetate, and the mixture was stirred at 22° C. for 1 h and at 77° C. for 3 h. After cooling, the reaction mixture was concentrated under reduced pressure, giving 32 g (98.5% of theory) of the title compound of m.p. 178-180° C.

Use examples

The herbicidal action of the fused triazoles of the formula Ia was demonstrated by greenhouse experiments:

The culture containers used were plastic pots containing loamy sand with approximately 3.0% of humus as the substrate. The seeds of the test plants were sown separately for each species.

For the pre-emergence treatment, the active compounds, which had been suspended or emulsified in water, were applied directly after sowing by means of finely distributing nozzles. The containers were irrigated gently to promote germination and growth and subsequently covered with transparent plastic hoods until the plants had rooted. This cover caused uniform germination of the test plants, unless this was adversely affected by the active compounds.

For the post-emergence treatment, the test plants were first grown to a height of 3 to 15 cm, depending on the plant habit, and then treated with the active compounds which had been suspened or emulsified in water. To this end, the test plants were either sown directly and grown in the same containers, or they were first grown separately as seedlings and transplanted into the test containers a few days prior to the treatment. The application rates for the post-emergence treatments were 62.5, 31.2, 15.6, 7.8 and 3.9 g of a.s./ha.

Depending on the species, the plants were kept at 10-25° C. or 20-35° C. The test period extended over 2 to 4 weeks. During this time, the plants were tended, and their response to the individual treatments was evaluated.

Evaluation was carried out using a scale from 0 to 100. 100 means no emergence of the plants, or complete destruction of at least the above-ground parts, and O means no damage, or normal course of growth.

The plants used in the greenhouse experiments were of the following species:

| Bayer code | Common Name |
|---|---|
| ABUTH | velvet leaf |
| AMARE | common amaranth |
| BIDPI | common blackjack |
| CHEAL | lambsquarters |
| | (goosefoot) |
| COMBE | commelinal bengal |
| GALAP | harrit cleavers |
| POLPE | redshank |
| PHBPU | common morning glory |
| SETFA | giant foxtail |

a) Herbicidal activity:

TABLE 14
Investigated compounds

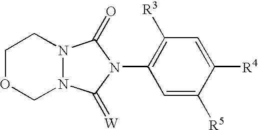

| Compound | W | R³ | R⁴ | R⁵ |
|---|---|---|---|---|
| Ex. 5 | S | F | Cl | OCH₂C═CH |
| Ex. 104 | S | F | Cl | OCH₃ |
| Comparison A | O | F | Cl | OCH₃ |
| Ex. 26 | S | F | Cl | SCH₂CO₂CH₃ |
| Comparison B | O | F | Cl | SCH₂CO₂CH₃ |
| Ex. 35 | S | F | Cl | CO₂CH₃ |
| Comparison C | O | F | Cl | CO₂CH₃ |
| Ex. 96 | S | Cl | Cl | CH═NOCH₃ |
| Comparison D | O | Cl | Cl | CH═NOCH₃ |

Applied by the post-emergence method, the compound from Example 5 showed very good herbicidal activity against ABUTH, AMARE, CHEAL and PHPBU at application rates of 7.8 and 3.9 g of a.s./ha.

Applied by the post-emergence method, the compound from Example 104 showed very good herbicidal activity against BIDPI, COMBE and POLPE at application rates of 15.6 and 7.8 g of a.s./ha.

Applied by the post-emergence method, the compound from Example 26 showed very good herbicidal activity against BIDPI, COMBE, GALAP and POLPE at application rates of 15.6 and 7.8 g of a.s./ha.

Applied by the post-emergence method, the compound from Example 35 showed very good herbicidal activity against BIDPI, COMBE, GALAP and POLPE at application rates of 7.8 and 3.9 g of a.s./ha.

Applied by the post-emergence method, the compound from Example 96 showed good to very good herbicidal activity against SETFA, COMBE and GALAP and POLPE at application rates of 31.2 and 62.5 g of a.s./ha.

Applied by the post-emergence method, the comparative compound A showed average to good herbicidal activity against BIDPI, COMBE and POLPE at application rates of 15.6 and 7.8 g of a.s./ha.

Applied by the post-emergence method, the comparative compound B showed average to poor herbicidal activity against BIDPI, COMBE, GALAP and POLPE at application rates of 15.6 and 7.8 g of a.s./ha.

Applied by the post-emergence method, the comparative compound C showed average to good herbicidal activity against BIDPI, GALAP and POLPE at application rates of 7.8 and 3.9 g of a.s./ha.

Applied by the post-emergence method, the comparative compound D showed average to moderate herbicidal activity against SETFA, COMBE and GALAP and POLPE at application rates of 31.2 and 62.5 g of a.s./ha.

b) Desiccant/Defoliant Action

The test plants used were young 4-leaf cotton plants (without cotyledons) which were grown under greenhouse conditions (rel. atmospheric humidity 50-70%; day/night temperature 27/20° C.).

The leaves of the young cotton plants were sprayed to run off point with aqueous preparations of the active compounds (with addition of 0.15% by weight of the fatty alcohol alkoxylate Plurafac ® LF 700, based on the spray liquor). The amount of water applied was 1000 l/ha (converted). After 13 days, the number of leaves that had been shed and the degree of defoliation in % were determined.

The untreated control plants did not lose any leaves.

We claim:

1. A process for preparing fused tetrahydro-[1H]-triazoles of the formula I 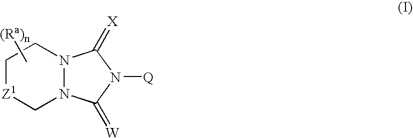
where the variables R^{a}, W, X, n and Q are as defined below:
R^{a} is hydroxyl, CO₂R¹, halogen, cyano, C(O)N(R¹)₂, where the radicals R¹ may be different fom one another, OR^{1a}, C₁-C₆-alkyl, C₁-C₆-haloalkyl, C₃-C₆-alkenyl, C₃-C₆-alkynyl, COR¹, S(O)ₙ^{R1} where n=0, 1 or 2 or C(O)SR¹; where
R¹ is hydrogen, C₁-C₆-alkyl, C₁-C₆-haloalkyl, C₁-C₃-alkoxy-C₁-C₃-alkyl, C₃-C₆-alkenyl or C₃-C₆-alkynyl; and
R^{1a} is C₁-C₆-alkyl, C₃-C₆-alkenyl, C₃-C₆-alkynyl which may be partially or fully halogenated or substituted, C₃-C₆-cycloalkyl, benzyl or phenethyl which may be substituted on the phenyl ring, and also optionally substituted phenyl or optionally substituted pyridyl;
n has the value 0, 1, 2 or 3;
X,W independently of one another are S or O;
Q is phenyl which has 1, 2, 3 or 4 substituents, where substituents attached to two adjacent carbon atoms may, together with these atoms, form a 5- or 6-membered saturated or unsaturated carbocycle or a 5- or 6-membered saturated or unsaturated heterocycle which has 1, 2 or 3 heteroatoms selected from the group consisting of O, N and S and which for its part may be substituted or unsubstituted;
and Z¹ is O, S, S═O or SO₂;
which comprises
i.) preparing a perhydrodiazine of the general formula IIIa 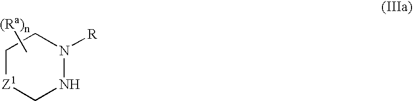
wherein the variables R^{a}, Z¹, and n are as defined above
R is C(X)OR² or C(X)SR², where
X is oxygen or sulfur and
R² is C₁-C₆-alkyl, C₃-C₈-cycloalkyl, C₂-C₆-alkenyl, C₃-C₆-alkynyl which may be partially or fully halogenated or substituted, P(O)(OR¹)₂, aryl or heteroaryl which may optionally be substituted, where R¹ is as defined above;
by reacting, in a first reaction step, a substituted hydrazine of the formula V 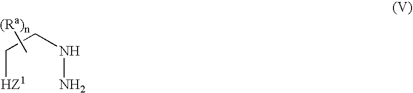
in which R^{a} and n are as defined above and Z¹ is oxygen or sulfur, with a compound of the formula R²—O—C(X)—A or of the formula R²—S—C(X)—A, in which R² and X are as defined above and A is a nucleophilically displaceable leaving group, whereby a hydrazine derivative of the formula VI is obtained 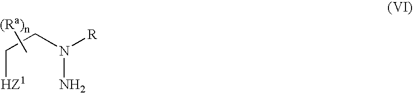
in which Z¹, R, R^{a} and n are as defined above, cyclizing, in a second step, the compound VI with formaldehyde in the presence of an acid to give the substituted perhydrodiazines of the formula IIIa where Z¹=O or S, and, for Z¹=S, if appropriate, oxidizing, in a further reaction step, to give sulfoxides where Z¹=SO or sulfones where Z¹=SO₂, whereby a perhydrodiazine of the general formula IIIa is obtained,
ii) reacting a perhydrodiazine of the formula IIIa with an isocyanate or an isothiocyanate of the formula IV
Q—N═C═W (IV)
in which Q and W are as defined above, whereby a compound of the general formula IIa is obtained, 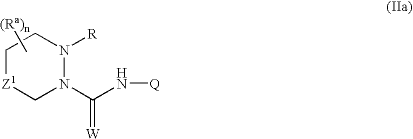
wherein R, R^{a}, W, Q, Z¹ and n are as defined above, and
iii) reacting the compound IIa with a base.

2. A process as claimed in claim 1 wherein the base is selected from tertiary amines.

3. A process as claimed in any of the preceding claims, wherein from 0.9 to 1.4 molar equivalent of base, based on the compound II, are used.

4. A process as claimed in any of the preceding claims, wherein the reaction with the base is carried out at a temperature in the range from O to 150° C.

5. A process as claimed in any of the preceding claims, wherein W in the formulae I and IIa is sulfur.

6. A process as claimed in any of the preceding claims, wherein R in the formula IIa is selected from the group consisting of C₁-C₄-alkyloxycarbonyl and C₁-C₄-alkyloxythiocarbonyl.

7. A process as claimed in any of the preceding claims, wherein Q in the formulae I and IIa is a radical of the formulae Q-1 to Q-7 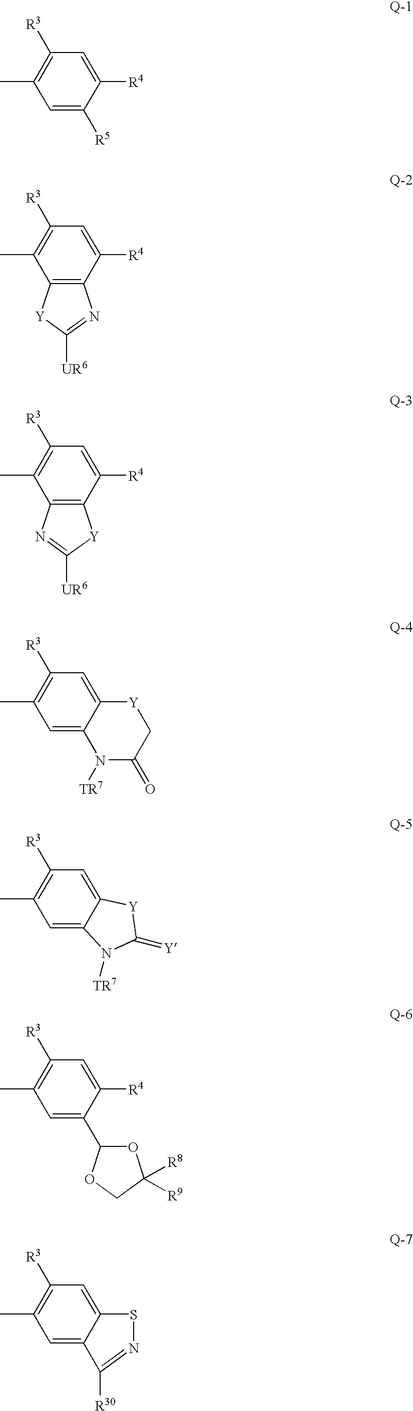
where the variables Y and Y′, T, U and the radicals R³, R⁴, R⁵, R⁶, R⁷, R⁸, R⁹ and R³⁰ are as defined below:
Y and Y′ independently of one another are oxygen or sulfur;
T is a chemical bond or oxygen;
U is a chemical bond, C₁-C₄-alkylene, O, S, SO or SO₂;
R³ is hydrogen or halogen;
R⁴ is C₁-C₄-alkyl, C₁-C₄-haloalkyl, C₁-C₄-alkoxy, C₁-C₄-alkylthio, C₁-C₄-haloalkoxy, halogen, cyano or NO₂;
R⁵ is hydroxyl, mercapto, cyano, nitro, halogen, C₁-C₆-alkyl, C₂-C₆-alkenyl, C₂-C₆-alkynyl, C₁-C₆-haloalkyl, C₂-C₆-haloalkenyl, C₂-C₆-haloalkynyl, C₁C₆-alkoxy-(C₁-C₆-alkyl) carbonyl, C₁-C₆-alkylthio-(C₁-C₆-alkyl)carbonyl, (C₁-C₆-alkyl)iminooxycarbonyl, C₁-C₆-alkoxy-C₁-C₆-alkyl, C₁-C₆-alkoxyamino-C₁-C₆-alkyl, C₁-C₆-alkoxy-C₁-C₆-alkylamino-C₁-C₆-alkyl, C₁-C₃-alkoxy-C₃-C₆-alkenyl, C₃-C₆-haloalkenyl, cyano-C₃-C₆-alkenyl, C₃-C₆-alkynyl, C₁-C₃-alkoxy-C₃-C₆-alkynyl, C₃-C₆-haloalkynyl, cyano-C₃-C₆-alkynyl,
C₁-C₆-alkoxy, C₁-C₆-alkylthio, C₃-C₆-cycloalkoxy, C₃-C₆-cycloalkylthio, C₂-C₆-alkenyloxy, C₂-C₆-alkenylthio, C₂-C₆-alkynyloxy, C₂-C₆-alkynylthio, (C₁-C₆-alkyl)carbonyloxy, (C₁-C₆-alkyl)carbonylthio, (C₁-C₆-alkoxy)carbonyloxy, (C₂-C₆-alkenyl)carbonyloxy, (C₂-C₆-alkenyl)carbonylthio, (C₂-C₆-alkynyl)carbonyloxy, (C₂-C₆-alkynyl)carbonylthio, C₁-C₆-alkylsulfonyloxy or C₁-C₆-alkylsulfonyl, where each of the 17 last-mentioned radicals may, if desired, carry one, two or three substituents selected from the group consisting of:
halogen, nitro, cyano, hydroxyl, C₃-C₆-cycloalkyl, C₁-C₆-alkoxy, C₃-C₆-cycloalkoxy, C₃-C₆-alkenyloxy, C₃-C₆-alkynyloxy, C₁-C₆-alkoxy-C₁-C₆-alkoxy, C₁-C₆-alkylthio, C₁-C₆-alkylsulfinyl, C₁-C₆-alkylsulfonyl, C₁-C₆-alkylideneaminooxy, oxo, ═N—OR¹⁰
phenyl, phenoxy or phenylsulfonyl, where the three last-mentioned groups may optionally carry one, two or three substituents selected from the group consisting of halogen, nitro, cyano, C₁-C₆-alkyl, C₁-C₆-haloalkyl, C₁-C₆-alkoxy and (C₁-C₆-alkoxy)carbonyl;
—CO—R¹¹, —CO—OR¹¹, —CO—SR¹¹, —CO—N(R¹¹)—R¹², —OCO—R¹¹, —OCO—OR^{11′}, —OCO—SR^{11′}, —OCO—N(R¹¹)—R¹², —N(R¹¹)—R¹², and —C(R¹³)═N—OR¹⁰;
C(Z²)—R¹⁴, —C(═NR¹⁵)R¹⁴, C(R¹⁴)(Z³R¹⁶)(Z⁴R¹⁷), C(R¹⁴)═C(R¹⁸)—CN, C(R¹⁴)═C(R¹⁸)—CO—R¹⁹, —CH(R¹⁴)—CH(R¹⁸)—COR¹⁹, —C(R¹⁴)═C(R¹⁸)—CH₂—CO—R¹⁹, —C(R¹⁴)═C(R¹⁸)—C(R²⁰)═C(R²¹)—CO—R¹⁹, —C(R¹⁴)═C(R¹⁸)—CH₂—CH(R²²)—CO—R¹⁹, —CO—OR²³, —CO—SR²³, —CON(R²³)—OR¹⁰, —C≡C—CO—NHOR¹⁰, —C≡C—CO—N(R²³)—OR¹⁰, —C—C—CS—NH—OR¹⁰, —C≡C—CS—N(R²³)—OR¹⁰, —C(R¹⁴)═C(R¹⁸)—CO—NHOR¹⁰, —C(R¹⁴)═C(R¹⁸)—CO—N(R²³)—OR¹⁰, —C(R¹⁴)═C(R¹⁸)—CS—NHOR¹⁰, —C(R¹⁴)═C(R¹⁸)—CS—N(R²³)—OR¹⁰, —C(R¹⁴)═C(R¹⁸)—C(R¹³)═N—OR¹⁰, C(R¹³)═N—OR¹⁰, —C≡C—C(R¹³)═NOR¹⁰, C(Z³R¹⁶)(Z⁴R¹⁷)—OR²³, —C(Z³R¹⁶)(Z⁴R¹⁷)SR²³, C(Z³R¹⁶)(Z⁴R¹⁷)—N(R²⁴)R²⁵, —N(R²⁴)—R²⁵, —CO—N(R²⁴)—R²⁵ or —C(R¹⁴)═C(R¹⁸)CO—N(R²⁴)R²⁵; where Z², Z³ and Z⁴ independently of one another are oxygen or sulfur;
R⁶ is CO₂H, C₁-C₆-alkyl, C₁-C₆-haloalkyl, hydroxy-C₁-C₄-alkyl, cyano-C₁-C₄-alkyl, C₁-C₄-alkoxy-C₁-C₄-alkyl, amino-C₁-C₄-alkyl, C₁-C₄-alkylamino-C₁-C₄-alkyl, di(C₁-C₄-alkyl)amino-C₁-C₄-alkyl, C₁-C₄-alkylthio-C₁-C₄-alkyl, hydroxycarbonyl-C₁-C₄-alkyl, (C₁-C₄-alkoxy)carbonyl-C₁-C₄-alkyl, (C₁-C₄-alkylthio)carbonyl-C₁-C₄-alkyl, aminocarbonyl-C₁-C₄-alkyl, (C₁-C₄-alkylamino)carbonyl-C₁-C₄-alkyl, di(C₁-C₄-alkyl)aminocarbonyl-C₁-C₄-alkyl, C₃-C₆-alkenyl, C₁-C₃-alkoxy-C₃-C₆-alkenyl, C₃-C₆-haloalkenyl, cyano-C₃-C₆-alkenyl, C₃-C₆-alkynyl, C₁-C₃-alkoxy-C₃-C₆-alkynyl, C₃-C₆-haloalkynyl, cyano-C₃-C₆-alkynyl, phenyl, phenyl-C₁-C₄-alkyl, where the phenyl rings optionally carry one, two or three substituents selected from the group consisting of halogen, cyano, C₁-C₆-alkyl, C₁-C₆-haloalkyl, C₁-C₆-alkoxy or C₁-C₆-haloalkoxy;
C₃-C₇-cycloalkyl, 3- to 7-membered saturated heterocyclyl, where each cycloalkyl and each heterocyclyl ring may contain a carbonyl or thiocarbonyl ring member and where each cycloalkyl and heterocyclyl ring may be unsubstituted or may carry two, three or four substituents selected from the group consisting of cyano, nitro, amino, hydroxyl, halogen, C₁-C₄-alkyl, C₁-C₄-haloalkyl, C₁-C₄-cyanoalkyl, C₁-C₄-hydroxyalkyl, C₁-C₄-aminoalkyl, C₁-C₄-alkoxy, C₁-C₄-haloalkoxy, C₁-C₄-alkylthio, C₁-C₄-haloalkylthio, C₁-C₄-alkylsulfinyl, C₁-C₄-alkylsulfonyl, C₁-C₄-haloalkylsulfonyl, (C₁-C₄-alkoxy)carbonyl, (C₁-C₄-alkyl)carbonyl, (C₁-C₄-haloalkyl)carbonyl, (C₁-C₄-alkyl)carbonyloxy, (C₁-C₄-haloalkyl)carbonyloxy, di(C₁-C₄-alkyl)amino, C₃-C₆-alkenyl, C₃-C₆-alkynyl, C₃-C₄-alkenyloxy, C₃-C₄-alkenylthio, C₃-C₄-alkynyloxy and C₃-C₄-alkynylthio;
or, if U or T is a chemical bond, R⁶ is also hydrogen, hydroxyl, cyano, mercapto, amino, C₁-C₄-alkylamino, di-C₁-C₄-alkylamino, saturated 5- or 6-membered nitrogen heterocyclyl which is attached via nitrogen, C₃-C₆-cycloalkylamino, halogen, —(CH₂)ₙ—CH(OH)—CH₂—R²⁸ —(CH₂)ₙ—CH(halogen)—CH₂—R²⁸, —(CH₂)ₙ—CH₂—CH(halogen)—R²⁸, —(CH₂)ₙ—CH═CH—R²⁸ or —(CH₂)ₙ—CH═C(halogen)—R²⁸, where R²⁸ is hydroxycarbonyl, (C₁-C₄-alkoxy)carbonyl, (C₁-C₄-alkylthio)carbonyl, aminocarbonyl, (C₁-C₄-alkylamino)carbonyl or di(C₁-C₄-alkyl)aminocarbonyl and n is 0 or 1;
R⁷ has the meanings given for R⁶;
R⁸ is hydrogen, C₁-C₃-alkyl, C₁-C₃-haloalkyl or halogen;
R⁹ is hydrogen, C₁-C₃-alkyl, C₁-C₃-haloalkyl; or
R⁸ and R⁹ together are C═O;
R¹⁰ is hydrogen, C₁-C₆-alkyl, C₁-C₆-haloalkyl, C₃-C₆-cycloalkyl, C₃-C₆-alkenyl, C₃-C₆-alkynyl, hydroxy-C₁-C₆-alkyl, C₁-C₆-alkoxy-C₁-C₆-alkyl, C₁-C₆-alkylthio-C₁-C₆-alkyl, cyano-C₁-C₆-alkyl, (C₁-C₆-alkyl)carbonyl-C₁-C₆-alkyl, (C₁-C₆-alkoxy)carbonyl-C₁-C₆-alkyl, (C₁-C₆-alkoxy)carbonyl-C₂-C₆-alkenyl, (C₁-C₆-alkyl)carbonyloxy-C₁-C₆-alkyl or phenyl-C₁-C₆-alkyl, where the phenyl ring may, if desired, carry one, two or three substituents selected from the group consisting of cyano, nitro, halogen, C₁-C₆-alkyl, C₁-C₆-halonalkyl, C₁-C₆-alkoxy and (C₁-C₆-alkoxy)carbonyl;
R¹¹ is hydrogen, C₁-C₆-alkyl, C₃-C₆-cycloalkyl, C₃-C₆-alkenyl, C₃-C₆-alkynyl, C₁-C₆-alkoxy-C₁-C₆-alkyl, (C₁-C₆-alkoxy)carbonyl-C₁-C₆-alkyl, (C₃-C₆-alkenyloxy)carbonyl-C₁-C₆-alkyl, phenyl or phenyl-C₁-C₆-alkyl, where the phenyl ring of the two last-mentioned groups may be unsubstituted or may carry one, two or three radicals selected from the group consisting of halogen, nitro, cyano, C₁-C₆-alkyl, C₁-C₆-haloalkyl, C₁-C₆-alkoxy and (C₁-C₆-alkyl)carbonyl;
R^{11′} has the meanings given for R¹¹, except for hydrogen;
R¹² is hydrogen, hydroxyl, C₁-C₆-alkyl, C₃-C₆-cycloalkyl, C₃-C₆-cycloalkylaminocarbonyl, C₁-C₆-alkylaminocarbonyl, C₁-C₆-alkoxy, (C₁-C₆-alkoxy)carbonyl-C₁-C₆-alkoxy, C₃-C₆-alkenyl or C₃-C₆-alkenyloxy;
R¹³ is hydrogen, halogen, C₁-C₆-alkyl, C₁-C₆-haloalkyl, C₁-C₆-alkoxy, C₁-C₆-haloalkoxy, C₃-C₆-alkenyloxy, C₃-C₆-alkynyloxy, C₁-C₆-alkylthio, C₁-C₆-haloalkylthio, (C₁-C₆-alkyl)carbonyloxy, (C₁-C₆-haloalkyl)carbonyloxy, C₁-C₆-alkylsulfonyloxy or C₁-C₆-haloalkylsulfonyloxy, where the 12 last-mentioned radicals may carry one of the following substituents: hydroxyl, cyano, hydroxycarbonyl, C₁-C₆-alkoxy, C₁-C₆-alkylthio, (C₁-C₆-alkyl)carbonyl, (C₁-C₆-alkoxy)carbonyl, (C₁-C₆-alkyl)aminocarbonyl, di(C₁-C₆-alkyl)aminocarbonyl, (C₁-C₆-alkyl)carbonyloxy, C₁-C₆-alkoxy- (C₁-C₆-alkyl) aminocarbonyl;
(C₁-C₆-alkyl)carbonyl, (C₁-C₆-haloalkyl)carbonyl, (C₁-C₆-alkoxy)carbonyl, (C₁-C₆-alkoxy)carbonyloxy, (C₁-C₆-alkyl)carbonylthio, (C₁-C₆-haloalkyl)carbonylthio, (C₁-C₆-alkoxy)carbonylthio, C₂-C₆-alkenyl, (C₂-C₆-alkenyl)carbonyloxy, C₂-C₆-alkenylthio, C₃-C₆-alkynyl, C₃-C₆-alkynyloxy, C₃-C₆-alkynylthio, (C₂-C₆-alkynyl)carbonyloxy, C₃-C₆-alkynylsulfonyloxy, C₃-C₆-cycloalkyl, C₃-C₆-cycloalkyloxy, C₃-C₆-cycloalkylthio, (C₃-C₆-cycloalkyl)carbonyloxy, C₃-C₆-cycloalkylsulfonyloxy;
phenyl, phenoxy, phenylthio, benzoyloxy, phenylsulfonyloxy, phenyl-C₁-C₆-alkyl, phenyl-C₁-C₆-alkoxy, phenyl-C₁-C₆-alkylthio, phenyl-(C₁-C₆-alkyl)carbonyloxy or phenyl-(C₁-C₆-alkyl)sulfonyloxy, where the phenyl rings of the 10 last-mentioned radicals may be unsubstituted or may for their part carry one to three substituents, in each case selected from the group consisting of cyano, nitro, halogen, C₁-C₆-alkyl, C₁-C₆-haloalkyl, C₁-C₆-alkoxy and (C₁-C₆-alkoxy)carbonyl;
R¹⁴ is hydrogen, cyano, C₁-C₆-alkyl, C₁-C₆-haloalkyl, C₂-C₆-alkenyl, C₂-C₆-alkynyl, C₃-C₆-cycloalkyl, C₁-C₆-alkoxy-C₁-C₆-alkyl or (C₁-C₆-alkoxy)carbonyl;
R¹⁵ is hydrogen, hydroxyl, C₁-C₆-alkyl, C₃-C₆-alkenyl, C₃-C₆-alkynyl, C₃-C₆-cycloalkyl, C₁-C₆-haloalkyl, C₁-C₆-alkoxy-C₁-C₆-alkyl, C₁-C₆-alkoxy, C₃-C₆-alkenyloxy, C₃-C₆-alkynyloxy, C₃-C₆-cycloalkoxy, C₅-C₇-cycloalkenyloxy, C₁-C₆-haloalkoxy, C₃-C₆-haloalkenyloxy, hydroxy-C₁-C₆-alkoxy, cyano-C₁-C₆-alkoxy, C₃-C₆-cycloalkyl-C₁-C₆-alkoxy, C₁-C₆-alkoxy-C₁-C₆-alkoxy, C₁-C₆-alkoxy-C₃-C₆-alkenyloxy, (C₁-C₆-alkyl)carbonyloxy, (C₁-C₆-haloalkyl)carbonyloxy, (C₁-C₆-alkyl)carbamoyloxy, (C₁-C₆-haloalkyl)carbamoyloxy, (C₁-C₆-alkyl)carbonyl-C₁-C₆-alkyl, (C₁-C₆-alkyl)carbonyl-C₁-C₆-alkoxy, (C₁-C₆-alkoxy) carbonyl-C₁-C₆-alkyl, (C₁-C₆-alkoxy)carbonyl-C₁-C₆-alkoxy, C₁-C₆-alkylthio-C₁-C₆-alkoxy, di(C₁-C₆-alkyl)amino-C₁-C₆-alkoxy, —N(R²⁶)R²⁷, phenyl, which for its part may carry one, two or three substituents, in each case selected from the group consisting of cyano, nitro, halogen, C₁-C₆-alkyl, C₁-C₆-haloalkyl, C₂-C₆-alkenyl, C₁-C₆-alkoxy and (C₁-C₆-alkoxy)carbonyl;
phenyl-C₁-C₆-alkoxy, phenyl-(C₁-C₆-alkyl), phenyl-C₃-C₆-alkenyloxy or phenyl-C₃-C₆-alkynyloxy, where in each case one or two methylene groups of the carbon chains in the four last-mentioned groups may be replaced by —O—, —S—, or —N(C₁-C₆-alkyl)- and where the phenyl rings in the four last-mentioned groups may be unsubstituted or may for their part carry one to three substituents selected from the group consisting of cyano, nitro, halogen, C₁-C₆-alkyl, C₁-C₆-haloalkyl, C₂-C₆-alkenyl, C₁-C₆-alkoxy and (C₁-C₆-alkoxy)carbonyl;
C₃-C₇-heterocyclyl, C₃-C₇-heterocyclyl-C₁-C₆-alkyl, C₃-C₇-heterocyclyl-C₁-C₆-alkoxy, C₃-C₇-heterocyclyl-C₃-C₆-alkenyloxy or C₃-C₇-heterocyclyl-C₃-C₆-alkynyloxy, where in each case one or two methylene groups of the carbon chains in the four last-mentioned groups may be replaced by —O—, —S— or —N(C₁-C₆-alkyl)- and where each heterocycle may be saturated, unsaturated or aromatic and is either unsubstituted or for its part carries one to three substituents selected from the group consisting of cyano, nitro, halogen, C₁-C₆-alkyl, C₁-C₆-haloalkyl, C₂-C₆-alkenyl, C₁-C₆-alkoxy and (C₁-C₆-alkoxy)carbonyl;
R¹⁶, R¹⁷ independently of one another are C₁-C₆-alkyl, C₁-C₆-haloalkyl, C₃-C₆-alkenyl, C₃-C₆-alkynyl, C₁-C₆-alkoxy-C₁-C₆-alkyl, or together are a saturated or unsaturated 2- to 4-membered carbon chain which may carry an oxo substituent, where a member of this chain which is not adjacent to the variables Z³ and Z⁴ may be replaced by —O—, —S—, —N═, —NH— or —N(C₁-C₆-alkyl)- and where the carbon chain may carry one to three radicals selected from the group consisting of cyano, nitro, amino, halogen, C₁-C₆-alkyl, C₂-C₆-alkenyl, C₁-C₆-alkoxy, C₂-C₆-alkenyloxy, C₂-C₆-alkynyloxy, C₁-C₆-haloalkyl, cyano-C₁-C₆-alkyl, hydroxy-C₁-C₆-alkyl, C₁-C₆-alkoxy-C₁-C₆-alkyl, C₃-C₆-alkenyloxy-C₁-C₆-alkyl, C₃-C₆-alkynyloxy-C₁-C₆-alkyl, C₃-C₆-cycloalkyl, C₃-C₆-cycloalkoxy, carboxyl, (C₁-C₆-alkoxy)carbonyl, (C₁-C₆-alkyl)carbonyloxy-C₁-C₆-alkyl and phenyl; optionally substituted phenyl, where the carbon chain may also be substituted by a fused-on or spiro-linked 3- to 7-membered ring which may contain one or two heteroatoms selected from the group consisting of oxygen, sulfur, nitrogen and C₁-C₆-alkyl-substituted nitrogen as ring members and which may, if desired, carry one or two of the following substituents: cyano, C₁-C₆-alkyl, C₂-C₆-alkenyl, C₁-C₆-alkoxy, cyano-C₁-C₆-alkyl, C₁-C₆-haloalkyl and (C₁-C₆-alkoxy)carbonyl;
R¹⁸ is hydrogen, cyano, halogen, C₁-C₆-alkyl, C₁-C₆-haloalkyl, C₁-C₆-alkoxy, (C₁-C₆-alkyl)carbonyl or (C₁-C₆-alkoxy)carbonyl;
R¹⁹ is hydrogen, O—R²⁸, S—R²⁸, C₁-C₆-alkyl which may carry one or two C₁-C₆-alkoxy substituents, C₂-C₆-alkenyl, C₂-C₆-alkynyl, C₁-C₆-haloalkyl, C₃-C₆-cycloalkyl, C₁-C₆-alkylthio-C₁-C₆-alkyl, C₁-C₆-alkyliminooxy, —N(R²⁴)R²⁵ or phenyl which may be unsubstituted or may carry one to three substituents, in each case selected from the group consisting of cyano, nitro, halogen, C₁-C₆-alkyl, C₂-C₆-alkenyl, C₁-C₆-haloalkyl, C₁-C₆-alkoxy and (C₁-C₆-alkoxy)carbonyl;
R²⁰ is hydrogen, cyano, halogen, C₁-C₆-alkyl, C₃-C₆-alkenyl, C₃-C₆-alkynyl, C₁-C₆-alkoxy-C₁-C₆-alkyl, (C₁-C₆-alkyl)carbonyl, (C₁-C₆-alkoxy)carbonyl, —N(R²⁴)R²⁵ or phenyl which for its part may carry one to three substituents selected from the group consisting of cyano, nitro, halogen, C₁-C₆-alkyl, C₁-C₆-haloalkyl, C₃-C₆-alkenyl, C₁-C₆-alkoxy and (C₁-C₆-alkoxy)carbonyl;
R²¹ is hydrogen, cyano, halogen, C₁-C₆-alkyl, C₁-C₆-alkoxy, C₁-C₆-haloalkyl, (C₁-C₆-alkyl)carbonyl or (C₁-C₆-alkoxy)carbonyl;
R²² is hydrogen, cyano, C₁-C₆-alkyl or (C₁-C₆-alkoxy)carbonyl;
R²³, R²⁸ independently of one another are hydrogen, C₁-C₆-alkyl, C₁-C₆-haloalkyl, C₂-C₆-alkenyl or C₂-C₆-alkynyl, where the 4 last-mentioned groups may each carry one or two of the following radicals: cyano, halogen, hydroxyl, hydroxycarbonyl, C₁-C₆-alkoxy, C₁-C₆-alkylthio, (C₁-C₆-alkyl)carbonyl, (C₁-C₆-alkoxy)carbonyl, (C₁-C₆-alkyl)carbonyloxy, (C₃-C₆-alkenyloxy)carbonyl, (C₃-C₆-alkynyloxy)carbonyl;
(C₁-C₆-haloalkyl)carbonyl, (C₁-C₆-alkoxy)carbonyl, C₁-C₆-alkylaminocarbonyl, di(C₁-C₆-alkyl)aminocarbonyl, C₁-C₆-alkyloximino-C₁-C₆-alkyl, C₃-C₆-cycloalkyl;
phenyl or phenyl-C₁-C₆-alkyl, where the phenyl rings may be unsubstituted or may for their part carry one to three substituents, in each case selected from the group consisting of cyano, nitro, halogen, C₁-C₆-alkyl, C₁-C₆-haloalkyl, C₁-C₆-alkoxy and (C₁-C₆-alkoxy)carbonyl;
R²⁴, R²⁵, R²⁶, R²⁷ independently of one another are hydrogen, C₁-C₆-alkyl, C₃-C₆-alkenyl, C₂-C₆-alkynyl, C₃-C₆-cycloalkyl, C₁-C₆-haloalkyl, C₁-C₆-alkoxy-C₁-C₆-alkyl, (C₁-C₆-alkyl)carbonyl, (C₁-C₆-alkoxy)carbonyl, (C₁-C₆-alkoxy)carbonyl-C₁-C₆-alkyl,
(C₁-C₆-alkoxy)carbonyl-C₂-C₆-alkenyl, where the alkenyl chain may additionally carry one to three halogen and/or cyano radicals, C₁-C₆-alkylsulfonyl, (C₁-C₆-alkoxy)carbonyl-C₁-C₆-alkylsulfonyl, phenyl or phenylsulfonyl, where the phenyl rings of the two last-mentioned radicals may be unsubstituted or may for their part carry one to three substituents, in each case selected from the group consisting of cyano, nitro, halogen, C₁-C₆-alkyl, C₁-C₆-haloalkyl, C₃-C₆-alkenyl, C₁-C₆-alkoxy and (C₁-C₆-alkoxy)carbonyl; or
R²⁴ and R²⁵ and/or
R²⁶ and R²⁷ together with the respective common nitrogen atom are a saturated or unsaturated 4- to 7-membered azaheterocycle which, in addition to carbon ring members, may, if desired, contain one of the following members:
—O—, —S—, —N═, —NH— or —N(C₁-C₆-alkyl)-;
R³⁰ is hydrogen, C₁-C₆-alkyl, C₃-C₈-cycloalkyl, CH₂O—C₁-C₆-alkyl, CH₂O—C₂-C₄-alkenyl, CH₂O—C₂-C₄-alkynyl, CH₂CH₂O—C₁-C₄-alkyl, CH₂CH₂O—C₂-C₄-alkenyl, CH₂CH₂O—C₂-C₄-alkynyl, (C₁-C₆-alkoxy)carbonyl, (C₃-C₄-alkenyloxy)carbonyl, (C₃-C₄-alkynyloxy)carbonyl, C₃-C₆-cycloalkyloxycarbonyl, (C₁-C₆-alkylthio)carbonyl, (C₁-C₄-alkoxy)carbonyl-C₁-C₄-alkyl, (C₃-C₄-alkenyloxy)carbonyl-C₁-C₄-alkyl, (C₃-C₄-alkynyloxy)carbonyl, (C₁-C₄-alkylamino)carbonyl, (C₁-C₄-dialkylamino)carbonyl, (C₃-C₄-alkenylamino)carbonyl, (C₃-C₄-alkynylamino)carbonyl, (C₃-C₄-dialkenylamino) carbonyl, (C₃-C₄-dialkynylamino)carbonyl, (C₃-C₄-alkenyloxy)carbonyl-C₁-C₄-alkyl, (C₃-C₄-alkynyloxy)carbonyl-C₁-C₄-alkyl, C₁-C₄-alkylsulfonylamidocarbonyl, CH(O—C₁-C₄-alkyl)₂, CH[O(CH₂)₃O], CH[O(CH₂)₄O], phenyl, which may be unsubstituted or for its part may carry one to three substituents in each case selected from the group consisting of cyano, nitro, halogen, C₁-C₆-alkyl, C₁-C₆-haloalkyl, C₁-C₆-alkoxy, (C₁-C₆-alkoxy)carbonyl and C₁-C₄-alkoxycarbonyl-C₁-C₄-alkyl, where each alkyl radical may be unsubstituted or may carry one, two or three substituents selected from the group consisting of halogen, cyano, nitro, C₁-C₄-alkoxy and C₁-C₄-alkylthio and each cycloalkyl radical may be unsubstituted or may carry one, two or three substituents selected from the group consisting of halogen, cyano, nitro, C₁-C₄-alkyl, C₁-C₄-alkoxy and C₁-C₄-alkylthio.

8. A process as claimed in claim 7, wherein Q in the formulae I and IIa is Q-1.

9. A fused tetrahydro-[1H]-triazole of the formula Ia 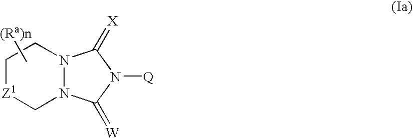
in which the variables R^{a}, Z¹, X and n are as defined in claim 1, w is sulfur and Q is one of the radicals Q-1 to Q-7 as defined in claim 7, and its agriculturally useful salts.

10. The fused tetrahydro-[1H]-triazole as claimed in claim 9, wherein n in formula Ia is 0, Z¹ and X independently of one another are selected from sulfur and oxygen, W is sulfur and Q is a radical of the formula Q1 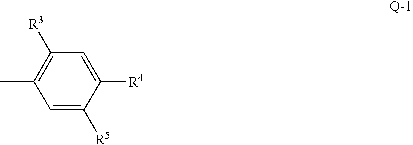
wherein
R³ is hydrogen or halogen,
R⁴ fluorine, chlorine or cyano, and
R⁵ is CN, COOH, C₁-C₄-alkoxyiminomethyl, C₁-C₄-alkoxy, C₃-C₆-cycloalkyloxy, C₃-C₆-alkenyloxy, C₃-C₆-alkynyloxy, C₃-C₆-alkenyloxyiminomethyl, (C₁-C₄-alkoxycarbonyl)-C₂-C₆-alkenyloxy, C₃-C₆-alkynyloxyiminomethyl, 2-[C₁-C₄-alkoxycarbonyl]-2-chloroethyl, 2-[C₁-C₄-alkoxycarbonyl]-2-chloroethenyl, C₁-C₄-alkoxycarbonyl, (C₁-C₆-alkoxycarbonyl)-C₁-C₄-alkoxy, (C₁-C₆-alkoxycarbonyl)-C₁-C₄-thioalkyl, COOR²³ where R²³=C₁-C₄-alkoxy-C₁-C₄-alkyl or C₃-C₆-alkenyloxycarbonyl-C₁-C₄-alkyl, CONR²⁴R²⁵ where R²⁴=hydrogen or C₁-C₄-alkyl and R²⁵=hydrogen, C₁-C₄-alkyl or C₁-C₄-alkoxy.

11. A composition, comprising at least one fused tetrahydro-[1H]-triazole of the formula Ia as claimed in any of claims 9 or 10 or an agriculturally useful salt of Ia and customary auxiliaries.

12. A method for controlling unwanted vegetation, which comprises allowing a herbicidally effective amount of at least one fused tetrahydro-[1H]-triazole of the formula Ia as claimed in any of claims 9 or 10 or an agriculturally useful salt of Ia to act on plants, their habitat and/or on seed.

13. The use of fused tetrahydro-[1H]-triazoles of the formula Ia as claimed in any of claims 9 or 10 or of agriculturally useful salts thereof as herbicides.

14. A process for preparing fused tetrahydro-[1H]-triazoles of the formula Ia′ 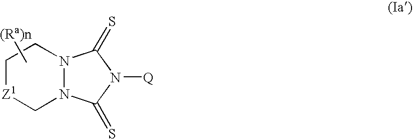
where the variables R^{a}, n and Q are as defined in claim 1 and Z¹ is O, S, S═O or SO₂, which comprises the preparation of a perhydrodiazine of the formula IIIa′ 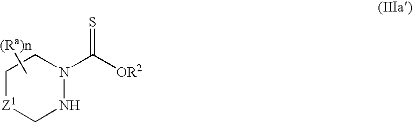
where R^{a}, n, Z¹ and R² are as defined in claim 1, according to the process as defined in claim 1 for the preparation of the compound IIIa and then reacting the perhydrodiazine IIIa′ with an isothiocyanate of the formula IVa
Q—N═C═S (IVa)
where Q is as defined above, in an aprotic polar solvent.
